(19) United States
(12) Patent Application Publication
(10) Pub. No.: US 2025/0296933 A1
(43) Pub. Date: Sep. 25, 2025

(54) METHODS AND INTERMEDIATES FOR PREPARING THERAPEUTIC COMPOUNDS

(71) Applicant: Gilead Sciences, Inc., Foster City, CA (US)

(72) Inventors: Sanberk S. Atalay, San Francisco, CA (US); Selcuk Calimsiz, Edmonton (CA); Andrei Chtchemelinine, Foster City, CA (US); Wing Chung, Edmonton (CA); Joshua Dahlke, Daly City, CA (US); Ian J. Doxsee, San Mateo, CA (US); Timothy G. Elford, Sherwood Park (CA); Sean Feng, San Mateo, CA (US); Kevin J. Garcia, San Mateo, CA (US); Olivia F. Goethe, New York, NY (US); Jordan B. Henrie, Sherwood Park (CA); Joshua R. LeDrew, Edmonton (CA); Jesse W. Li, Edmonton (CA); Lucas Man, Burlingame, CA (US); Jeffrey E. Merit, San Mateo, CA (US); Monalisa Munia, San Francisco, CA (US); Sean T. Neville, San Mateo, CA (US); Lyubov Roever, Leduc County (CA); Daniel C. Salgueiro, Burlingame, CA (US); Justin Y. Su, Foster City, CA (US); Yibai Sun, Edmonton (CA); Anna Michelle Wagner, Hayward, CA (US); Mohammad M. Zahedi, Dublin, CA (US)

(21) Appl. No.: 19/066,341

(22) Filed: Feb. 28, 2025

Related U.S. Application Data
(60) Provisional application No. 63/560,122, filed on Mar. 1, 2024.

(51) Int. Cl. C07D 471/18; A61K 31/551

(52) U.S. Cl. CPC C07D 471/18; A61K 31/551

(57) ABSTRACT

The present disclosure relates to methods and intermediates useful for preparing a compound of Formula I:

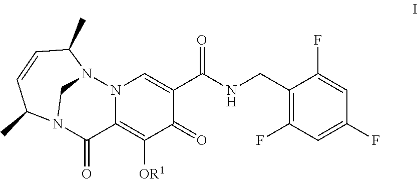

or a salt thereof.

### CROSS-REFERENCE TO RELATED APPLICATIONS

This application claims priority to U.S. Provisional Application No. 63/560,122, filed Mar. 1, 2024. The entire contents of this application are hereby incorporated by reference in their entirety.

### FIELD

The present disclosure relates to methods and intermediates for the synthesis of substituted pyridotriazine compounds for use in the treatment of human immunodeficiency virus (HIV) infection.

### BACKGROUND

The present disclosure relates generally to the field of organic synthetic methodology for the preparation of antiviral compounds and their synthetic intermediates.

Human immunodeficiency virus infection and related diseases are a major public health problem worldwide. Human immunodeficiency virus encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Engl. J Med. (1998) 338:853-860; Richman, D. D. Nature (2001) 410:995-1001). Accordingly, there is a need for new agents that inhibit the replication of HIV.

U.S. Pat. No. 11,613,546 discloses novel compounds useful for treating an infection caused by the HIV virus. One specific compound identified therein is a compound of Formula I-A:

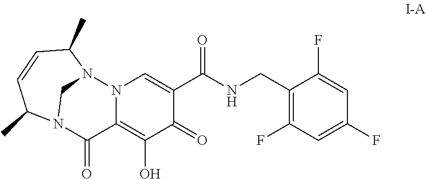

There is currently a need for synthetic methods and intermediates that can be used to prepare the compound of Formula I-A and salts thereof.

### SUMMARY

The present disclosure is directed to a novel synthetic process for preparing a compound of Formula I:

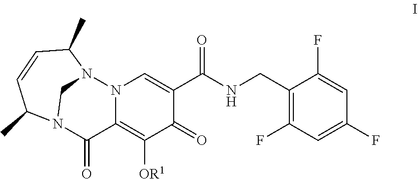

or a salt thereof, wherein R¹ is selected from hydrogen and a hydroxyl protecting group.

In some embodiments, the present disclosure provides a process for preparing a compound of Formula I-A:

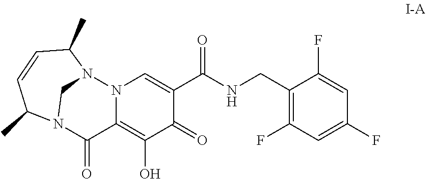

or a salt thereof. The compound of Formula I-A may also be named or identified as (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide.

The present disclosure is also directed to particular individual steps of this process and intermediates used in this process.

In some embodiments, disclosed herein is a process for preparing a compound of Formula I or a salt thereof, wherein R¹ is selected from hydrogen and a hydroxyl protecting group; comprising reacting a compound of Formula II:

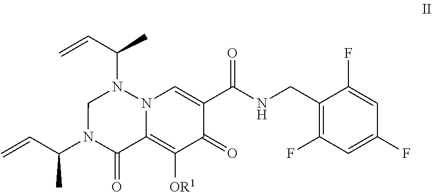

or a salt thereof, with a first catalyst to provide the compound of Formula I or a salt thereof.

In some embodiments, disclosed herein is a process for preparing a compound of Formula I-A:

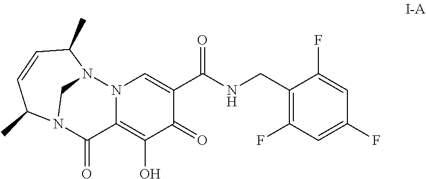

or a salt thereof, comprising deprotecting a compound of Formula I or a salt thereof, wherein R¹ is a hydroxyl protecting group, to provide the compound of Formula I-A or a salt thereof.

In some embodiments, disclosed herein is a process for preparing a compound of Formula I or a salt thereof, wherein R¹ is selected from hydrogen and a hydroxyl protecting group; comprising:
reacting a compound of Formula R-1:

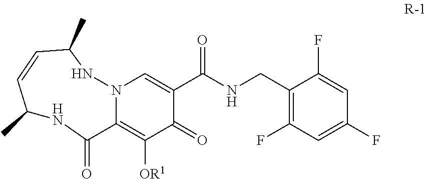

or a salt thereof, with:
   a methylene reagent, and
   an eighth acid or a fourteenth base,
to provide the compound of Formula I or a salt thereof.

Additional embodiments of the disclosure, including additional synthetic intermediates and methods for preparing such intermediates, are provided herein.

### DETAILED DESCRIPTION

The description below is made with the understanding that the present disclosure is to be considered as an exemplification of the claimed subject matter and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading. Unless defined otherwise, all technical and scientific terms used herein have the same meaning as commonly understood by one of ordinary skill in the art.

When trade names are used herein, it is intended to independently include the tradename product and the active pharmaceutical ingredient(s) of the tradename product.

### Definitions

The following description sets forth exemplary methods, parameters and the like. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The prefix “Cᵤ₋ᵥ” indicates that the following group has from u to v carbon atoms. For example, “C₁₋₈ alkyl” indicates that the alkyl group has from 1 to 8 carbon atoms.

“Isomers” are different compounds that have the same molecular formula. Isomers include stereoisomers, enantiomers and diastereomers.

“A “stereoisomer” refers to a compound made up of the same atoms bonded by the same bonds but having different three-dimensional structures, which are not interchangeable.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and/or hindered rotation about a bond axis and may thus give rise to enantiomers, diastereomers, and other stereoisomeric forms that may be defined, in terms of absolute stereochemistry, as (R)— or (S)—. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

Compounds described herein may be in any morphological form such as, for example, crystalline or amorphous, as well as disordered crystals, liquid crystals, plastic crystals, mesophases, and the like, or any combination thereof.

Also provided are pharmaceutically acceptable hydrates, solvates, co-crystals, tautomeric forms, polymorphs, and prodrugs of the compounds described herein.

Any formula or structure given herein, including Formula I, or any Formula disclosed herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C, and ¹⁴C are incorporated, may be useful to help determine or measure the effectiveness of the compounds by characterizing, for example, the site or mode of action, or binding affinity to pharmacologically important site of action. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds of Formula I, or any Formula disclosed herein, in which from 1 to “n” hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogen atoms have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life or reduced dosage requirements. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula I, or any Formula disclosed herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The modifier “about” used in connection with a quantity is inclusive of the stated value and has the meaning dictated by the context (e.g., includes the degree of error associated with measurement of the particular quantity).

The term “coupling agent” as used herein refers to a reagent used to couple an amine and a carboxylic acid to form an amide. Non-limiting examples of coupling agents include n-propyl phosphonic anhydride, oxalyl chloride, thionyl chloride, phosgene, triphosgene, propanephosphonic acid anhydride, acetic anhydride, pivalic anhydride, benzoic anhydride, 2,4,6-trichlorobenzoyl chloride, dicyclohexylcarbodiimide, diisopropylcarbodiimide, 1-ethyl-3-(3-dimethylaminopropyl)carbodiimide (EDC), 1,1′-carbonyldiimidazole, ethyl chloroformate, isobutyl chloroformate, 2-(7-aza-1H-benzotriazole-1-yl)-1,1,3,3-tetramethyluronium hexafluorophosphate (HATU), O-benzotriazole-N,N,N′,N′-tetramethyluronium-hexafluoro-phosphate (HBTU), O-(7-azabenzotriazole-1-yl)-N,N,N′,N′-tetramethyluronium tetrafluoroborate (TATU), O-(benzotriazol-1-yl)-N,N,N′,N′-tetramethyluronium tetrafluoroborate (TBTU), O-(6-chlorobenzotriazol-1-yl)-N,N,N′,N′-tetramethyluronium hexafluorophosphate (HCTU), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (1-cyano-2-ethoxy-2-oxoethylidenaminooxy)dimethylamino-morpholino-carbenium hexafluorophosphate (COMU), a peptide coupling agent, and the like.

A “peptide coupling agent” includes, but is not limited to, 2-(7-aza-1H-benzotriazole-1-yl)-1,1,3,3-tetramethyluronium hexafluorophosphate (HATU), O-benzotriazole-N,N,N′,N′-tetramethyluronium-hexafluoro-phosphate (HBTU), O-(7-azabenzotriazole-1-yl)-N,N,N′,N′-tetramethyluronium tetrafluoroborate (TATU), O-(benzotriazol-1-yl)-N,N,N′,N′-tetramethyluronium tetrafluoroborate (TBTU), O-(6-chlorobenzotriazol-1-yl)-N,N,N′,N′-tetramethyluronium hexafluorophosphate (HCTU), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (1-cyano-2-ethoxy-2-oxoethylidenaminooxy)dimethylamino-morpholino-carbenium hexafluorophosphate (COMU), and the like.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), 1 to 4 carbon atoms (i.e., C₁₋₄ alkyl), 1 to 3 carbon atoms (i.e., C₁₋₃ alkyl), or 1 to 2 carbon atoms (i.e., C₁₋₂ alkyl). Examples of alkyl groups include, but are not limited to, methyl, ethyl, propyl, isopropyl, n-butyl, sec-butyl, isobutyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Aryl” refers to an aromatic carbocyclic group having a single ring (e.g., monocyclic) or multiple rings (e.g., bicyclic or tricyclic), including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, but are not limited to, phenyl, naphthyl, fluorenyl, and anthryl.

The term “halo” or “halogen” includes fluoro, chloro, bromo and iodo.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Hydroxyl” and “hydroxy” are used interchangeably and refer to —OH. “Oxo” refers to the group (═O) or (O). Where tautomeric forms of the compound exist, hydroxyl and oxo groups are interchangeable.

The term “protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. The term “deprotecting” refers to removing the protecting group. The term “deprotecting agent” refers to a reagent useful for removing a protecting group from a compound.

The term “hydroxyl protecting group” refers to a moiety that masks or alters the properties of a hydroxyl group. Exemplary hydroxyl protecting groups include, but are not limited to, methyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p-nitrobenzyl, triphenylmethyl, trimethylsilyl, triethylsilyl, t-butyldimethylsilyl, t-butyl-diphenylsilyl (TBDPS), triphenylsilyl, benzoylformate, acetate, chloroacetate, trichloroacetate, trifluoroacetate, pivaloate, benzoate, p-phenylbenzoate, 9-fluorenylmethyl carbonate, mesylate, and tosylate.

The term “amine protecting group” refers to a moiety that masks or alters the properties of an amine group. Exemplary amine protecting groups include, but are not limited to, phthalimide, tert-butyloxycarbonyl (Boc), benzyloxycarbonyl (Cbz), fluorenylmethyloxycarbonyl (Fmoc), toluenesulfonyl (Ts), saccharin, methoxyphenyl, para-methoxyphenyl, ortho-methoxy phenyl, acetate, 2,5-dimethylpyrrole, 4-methoxybenzyl, 2,4-dimethoxybenzyl, 2,2-diphenyl, allyl, and triphenylmethyl.

### Abbreviations

Abbreviations as used herein have respective meanings as follows:
aq. aqueous
Bn benzyl
br broad
d doublet
DBU 1,8-diazabicyclo[5.4.0]undec-7-ene
DCM dichloromethane
dd doublet of doublets
ddd doublet of doublet of doublets
DMSO dimethylsulfoxide
dt doublet of triplets
Et ethyl
EtOH ethanol
h hour(s)
HATU 2-(7-aza-1H-benzotriazole-1-yl)-1,1,3,3-tetramethyluronium hexafluorophosphate
Hz hertz
J coupling constant
m multiplet
M Molar
MTBE methyl tert-butyl ether
Me methyl
MeOH methanol
MeTHF 2-methyltetrahydrofuran
MHz megahertz
min. minute(s)
MS mass spectroscopy
MTBE methyl-tert-butyl ether
m/z Mass to charge
N normal
NMR nuclear magnetic resonance
q quartet
s singlet
t triplet
tol toluene
TsOH or p-TsOH p-toluenesulfonic acid
wt % weight percent

### Methods and Intermediates

Except as otherwise noted, the methods and techniques of the present disclosure are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition, Wiley-Interscience, 2013.

In certain instances, the processes disclosed herein involve a step of forming a salt of a compound of the present disclosure.

Compounds as described herein can be purified by any of the means known in the art, including chromatographic means, such as high performance liquid chromatography (HPLC), preparative thin layer chromatography, flash column chromatography, supercritical fluid chromatography (SFC), and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2^{nd} ed., ed. L. R. Snyder and J. J. Kirkland, John Wiley and Sons, 1979; and Thin Layer Chromatography, E. Stahl (ed.), Springer-Verlag, New York, 1969.

Exemplary chemical entities useful in methods of the embodiments will now be described by reference to illustrative synthetic schemes for their general preparation herein and the specific examples that follow. One of skill in the art will recognize that the transformations shown in the schemes below may be performed in any order that is compatible with the functionality of the particular pendant groups. In some embodiments, each of the reactions depicted in the general schemes is run at a temperature from about −80° C. to the reflux temperature of the organic solvent used.

The compounds disclosed herein may display atropisomerism resulting from steric hindrance affecting the axial rotation rate around a single bond. The resultant conformational isomers may each be observed as distinct entities by characterization techniques such as NMR and HPLC. The compounds disclosed herein may exist as a mixture of atropisomers. However, the detection of atropisomers is dependent on factors such as temperature, solvent, conditions of purification, and timescale of spectroscopic technique. The interconversion rate at room temperature has a half-life of minutes to hours, hours to days, or days to years. The ratio of atropisomers at equilibrium may not be unity. Characterization data presented herein may not represent the equilibrium state depending on the conditions of isolation and characterization which may include but not limited to handling, solvents used, and temperature.

In some embodiments, the present disclosure provides processes and intermediates for preparing the compound of Formula I and salts thereof. In other embodiments, the disclosure provides processes for preparing intermediates that can be used to prepare the compound of Formula I and salts thereof.

Provided herein is a method of preparing a compound of Formula I:

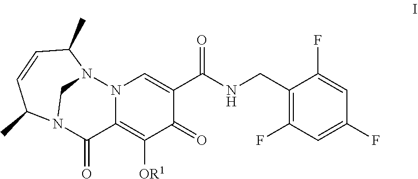

or a salt thereof, wherein R¹ is selected from hydrogen and a hydroxyl protecting group; comprising reacting a compound of Formula II:

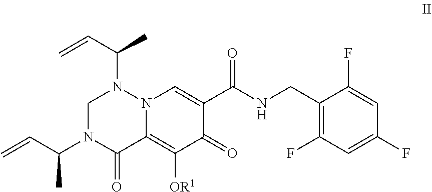

or a salt thereof,
with a first catalyst to provide the compound of Formula I or a salt thereof.

In some embodiments, R¹ is selected from hydrogen, C₁₋₄alkyl, and benzyl. In some embodiments, R¹ is hydrogen. In some embodiments, R¹ is C₁₋₄ alkyl. In some embodiments, R¹ is selected from hydrogen, methyl, ethyl, and benzyl. In some embodiments, R¹ is selected from methyl, ethyl, and benzyl. In some embodiments, R¹ is methyl. In some embodiments, R¹ is ethyl. In some embodiments, R¹ is benzyl.

In some embodiments, the compound of Formula I or a salt thereof is a compound of Formula I-A:

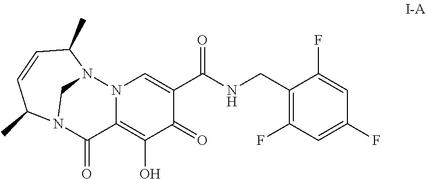

or a salt thereof.

In some embodiments, the compound of Formula I or a salt thereof is a compound of Formula I-B:

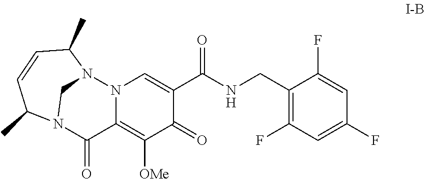

or a salt thereof.

In some embodiments, the compound of Formula II or a salt thereof is a compound of Formula II-A:

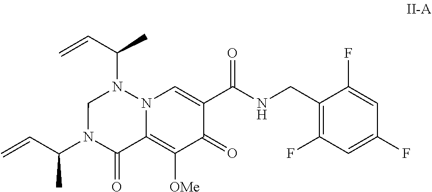 [SMILES: C=C[C@@H](C)N1CN([C@@H](C)C=C)C(=O)c2c(OC)c(=O)c(C(=O)NCc3c(F)cc(F)cc3F)cn21]

or a salt thereof.

In some embodiments, the first catalyst is a metathesis catalyst. In some embodiments, the first catalyst is selected from a ruthenium-based catalyst, a molybdenum-based catalyst, and a tungsten-based catalyst. In some embodiments, the first catalyst is a ruthenium-based catalyst. In some embodiments, the first catalyst is selected from Hoveyda-Grubbs second generation catalyst, Hoveyda-Grubbs first generation catalyst, Zhan 1B, nitro Grela, M721, M203, and M730. In some embodiments, the first catalyst is Hoveyda-Grubbs second generation catalyst.

In some embodiments, a molar ratio of the compound of Formula II or a salt thereof to the first catalyst is from about 1:0.001 to about 1:0.05. In some embodiments, a molar ratio of the compound of Formula II or a salt thereof to the first catalyst is from about 1:0.01 to about 1:0.03. In some embodiments, a molar ratio of the compound of Formula II or a salt thereof to the first catalyst is from about 1:0.01 to about 1:0.02. In some embodiments, a molar ratio of the compound of Formula II or a salt thereof to the first catalyst is about 1:0.015.

In some embodiments, the reacting of the compound of Formula II or a salt thereof is performed in the presence of a first additive. In some embodiments, the first additive can minimize the amount of impurity (i.e., reaction by-product) formed in the reaction. In some embodiments, the first additive comprises dimethyl maleate, 1,4-benzoquinone, 6-dichloro-1,4-benzoquinone, 2-chloro-1,4-benzoquinone, tetrafluoro-1,4-benzoquinone, 2-methylbenzoquinone, dimethyl fumarate, maleic acid, fumaric acid, maleic anhydride, trans-methyl cinnamate, acetic acid, trifluoroacetic acid, or a combination thereof. In some embodiments, the first additive comprises dimethyl maleate.

In some embodiments, the reacting of the compound of Formula II or a salt thereof is performed in a first solvent. In some embodiments, the first solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the first solvent comprises an ester. In some embodiments, the first solvent comprises ethyl acetate, methyl acetate, isopropyl acetate, dimethyl-2-methyl glutarate, propylene carbonate, or a combination thereof. In some embodiments, the reacting of the compound of Formula II or a salt thereof is performed in a first solvent comprising ethyl acetate.

In some embodiments, the reacting of the compound of Formula II or a salt thereof is carried out in a temperature range of from about 20° C. to about 120° C. In some embodiments, the reacting of the compound of Formula II is carried out in a temperature range of from about 50° C. to about 100° C. In some embodiments, the reacting of the compound of Formula II is carried out in a temperature range of from about 70° C. to about 90° C. In some embodiments, the reacting of the compound of Formula II is carried out at a temperature of about 80° C.

In some embodiments, R¹ is a hydroxyl protecting group; and the process further comprises deprotecting the compound of Formula I, or a salt thereof;

to provide a compound of Formula I-A:

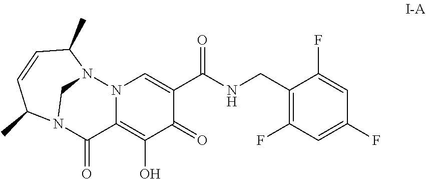

or a salt thereof.

In some embodiments, deprotecting the compound of Formula I or a salt thereof comprises treating the compound of Formula I or a salt thereof with a first deprotecting agent. Any suitable deprotecting agent can be used for the deprotection of the compound of Formula I. In some embodiments, suitable deprotecting agents include metal halide salts (e.g., magnesium bromide, lithium chloride, potassium iodide, sodium iodide, calcium chloride, copper (I) chloride, copper (II) chloride, zinc chloride), hydrogen halides (e.g., hydrogen chloride, hydrogen bromide, hydrogen iodide), boron reagents (e.g., boron trichloride, boron tribromide), aluminum reagents (e.g., aluminum trichloride, aluminum tribromide), titanium reagents (e.g., titanium tetrachloride, titanium tribromide, titanium tetrabromide), pyridinium halide salts (e.g., pyridinium chloride, pyridinium perbromide, pyridinium iodide), ammonium halides (e.g., ammonium chloride, tetrabutylammonium chloride, tetrabutylammonium iodide), and silyl halides (e.g., trimethylsilyl chloride, trimethylsilyl iodide).

In some embodiments, the first deprotecting agent is selected from a metal halide, a hydrogen halide, a boron reagent, an aluminum reagent, a titanium reagent, a pyridinium halide salt, an ammonium halide, and a silyl halide. In some embodiments, the first deprotecting agent is a metal halide. In some embodiments, the first deprotecting agent comprises magnesium bromide, lithium chloride, potassium iodide, sodium iodide, calcium chloride, copper (I) chloride, copper (II) chloride, zinc chloride, or a combination thereof. In some embodiments, the first deprotecting agent is lithium chloride.

In some embodiments, the deprotecting the compound of Formula I or a salt thereof is performed in a second solvent. In some embodiments, the second solvent comprises water, an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the second solvent comprises a polar aprotic solvent. In some embodiments, the second solvent comprises N,N-dimethylformamide, N,N-dimethylacetamide, N-methyl-2-pyrrolidone, N-butyl pyrrolidone (NBP), or a combination thereof. In some embodiments, the deprotecting the compound of Formula I or a salt thereof is performed in a second solvent comprising N-methyl-2-pyrrolidone.

In some embodiments, the deprotecting the compound of Formula I or a salt thereof is carried out in a temperature range of from about 0° C. to about 130° C. In some embodiments, the deprotecting the compound of Formula I or a salt thereof is carried out in a temperature range of from about 0° C. to about 100° C. In some embodiments, the deprotecting the compound of Formula I or a salt thereof is carried out in a temperature range of from about 70° C. to about 90° C. In some embodiments, the deprotecting the compound of Formula I or a salt thereof is carried out in a temperature of about 80° C.

In some embodiments, the process further comprising preparing the compound of Formula II or a salt thereof by a process comprising:
reacting a compound of Formula III:

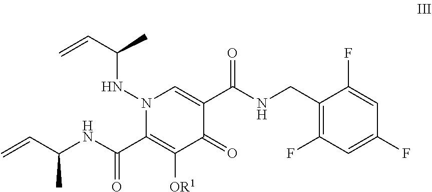

or a salt thereof, with
   a methylene reagent, and
   a first acid or a first base,
to provide the compound of Formula II or a salt thereof.

In some embodiments, the compound of Formula III is a compound of Formula III-A:

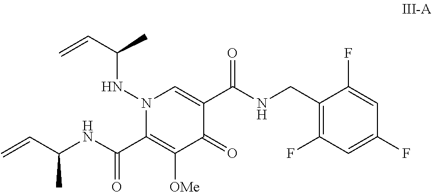 [SMILES: C=C[C@@H](C)Nn1cc(C(=O)NCc2c(F)cc(F)cc2F)c(=O)c(OC)c1C(=O)N[C@@H](C)C=C]

or a salt thereof.

In some embodiments, the methylene reagent is a formaldehyde reagent or a dihalomethane. In some embodiments, suitable formaldehyde reagents include formaldehyde acetals (e.g., dimethoxymethane, diethoxymethane), formaldehyde solution, paraformaldehyde, and 1,3,5-trioxane. In some embodiments, suitable methylene reagents include dihalomethanes (e.g., dibromomethane, diiodomethane).

In some embodiments, the methylene reagent is selected from 1,3,5-trioxane, a formaldehyde acetal, formaldehyde, paraformaldehyde, or a dihalomethane. In some embodiments, the methylene reagent is a formaldehyde reagent. In some embodiments, the methylene reagent is selected from 1,3,5-trioxane, a formaldehyde acetal, formaldehyde, and paraformaldehyde. In some embodiments, the methylene reagent is 1,3,5-trioxane.

In some embodiments, the compound of Formula III or a salt thereof is reacted with the first acid. In some embodiments, suitable first acids include carboxylic acids (e.g., oxalic acid, trichloroacetic acid, trifluoroacetic acid), sulfonic acids (e.g., methanesulfonic acid, benzenesulfonic acid, para-toluenesulfonic acid, camphorsulfonic acid), Lewis acids (e.g., boron trifluoride methyl etherate, boron trifluoride dibutyl etherate, boron trichloride, boron tribromide, aluminum chloride, aluminum bromide), inorganic acids (e.g., hydrochloric acid, sulfuric acid, phosphoric acid, hydrobromic acid), or a combination thereof.

In some embodiments, the first acid comprises a carboxylic acid, a sulfonic acid, a Lewis acid, an inorganic acid, or a combination thereof. In some embodiments, the first acid comprises a carboxylic acid. In some embodiments, the first acid is selected from oxalic acid, trichloroacetic acid, trifluoroacetic acid, or a combination thereof. In some embodiments, the first acid is trifluoroacetic acid.

In some embodiments, the compound of Formula III or a salt thereof is reacted with the first base. In some embodiments, suitable first bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN), etc.), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide, etc.), amides (e.g., lithium bis(trimethylsilyl)amide, potassium bis(trimethylsilyl)amide, lithium diisopropylamide), basic aromatic compounds (e.g., pyridine, 2,6-lutidine, imidazole), or a combination thereof.

In some embodiments, the first base comprises an amine, an amidine, a guanidine, a carboxylate, a carbonate, a metal hydride, a phosphate, a hydroxide, an alkoxide, an amide, a basic aromatic compound, or a combination thereof.

In some embodiments, the reacting of the compound of Formula III or a salt thereof is performed in a third solvent. In some embodiments, the third solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the third solvent comprises a hydrocarbon. In some embodiments, the third solvent comprises toluene, methylcyclohexane, n-heptane, hexanes, limonene, or a combination thereof. In some embodiments, the reacting of the compound of Formula III or a salt thereof is performed in a third solvent comprising toluene.

In some embodiments, the reacting of the compound of Formula III or a salt thereof is carried out in a temperature range of from about 60° C. to about 100° C. In some embodiments, the reacting of the compound of Formula III or a salt thereof is carried out in a temperature range of from about 75° C. to about 85° C. In some embodiments, the reacting of the compound of Formula III or a salt thereof is carried out at a temperature of about 80° C.

In some embodiments, the process further comprises preparing the compound of Formula III or a salt thereof by a process comprising:
deprotecting a compound of Formula IV:

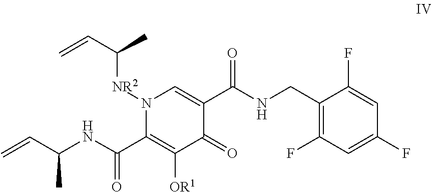

or a salt thereof, wherein R² is an amine protecting group;
to provide the compound of Formula III or a salt thereof.

In some embodiments, R² is tert-butyloxycarbonyl (Boc). In some embodiments, R¹ is selected from hydrogen, methyl, ethyl and benzyl and R² is tert-butyloxycarbonyl. In some embodiments, R¹ is C₁₋₄alkyl and R² is tert-butyloxycarbonyl. In some embodiments, R¹ is selected from methyl and ethyl and R² is tert-butyloxycarbonyl.

In some embodiments, the compound of Formula IV is a compound of Formula IV-A:

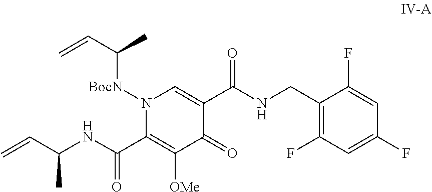 [SMILES: C=C[C@@H](C)N(C(=O)OC(C)(C)C)n1cc(C(=O)NCc2c(F)cc(F)cc2F)c(=O)c(OC)c1C(=O)N[C@@H](C)C=C]

or a salt thereof.

In some embodiments, deprotecting the compound of Formula IV or a salt thereof comprises treating the compound of Formula IV or a salt thereof with a second acid. Any suitable acid can be used for the deprotection of the compound of Formula IV or a salt thereof. In some embodiments, suitable second acids include sulfonic acids (e.g., p-toluenesulfonic acid, benzenesulfonic acid, camphorsulfonic acid), inorganic acids (e.g., hydrochloric acid, hydrobromic acid, sulfuric, phosphoric acid), organic acids (e.g., trifluoroacetic acid, formic acid, acetic acid, oxalic acid, citric acid), acid chlorides (e.g., acetyl chloride), Lewis acids (e.g., aluminum chloride, boron tribromide, lanthanum triflate)

In some embodiments, the second acid comprises a sulfonic acid, an inorganic acid, an organic acid, an acid chloride, a Lewis acid, or a combination thereof. In some embodiments, the second acid comprises a sulfonic acid. In some embodiments, the second acid is selected from p-toluenesulfonic acid, benzenesulfonic acid, camphorsulfonic acid, or a combination thereof. In some embodiments, the second acid comprises methanesulfonic acid.

In some embodiments, deprotecting the compound of Formula IV or a salt thereof is performed without an acid.

In some embodiments, deprotecting the compound of Formula IV or a salt thereof is performed in a fourth solvent. In some embodiments, the fourth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the fourth solvent comprises an ether. In some embodiments, the fourth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, deprotecting the compound of Formula IV or a salt thereof is performed in a fourth solvent comprising cyclopentyl methyl ether.

In some embodiments, deprotecting the compound of Formula IV or a salt thereof is carried out in a temperature range of from about 0° C. to about 120° C. In some embodiments, deprotecting the compound of Formula IV or a salt thereof is carried out in a temperature range of from about 10° C. to about 60° C. In some embodiments, deprotecting the compound of Formula IV or a salt thereof is carried out in a temperature range of from about 30° C. to about 40° C. In some embodiments, deprotecting the compound of Formula IV or a salt thereof is carried out at a temperature range of about 35° C.

In some embodiments, the process further comprises preparing the compound of Formula IV or a salt thereof by a process comprising:
reacting a compound of Formula V:

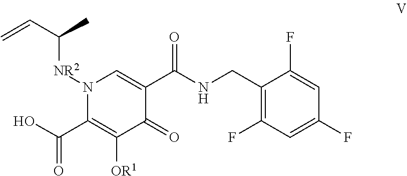

or a salt thereof, with:
   a compound of Formula VI:

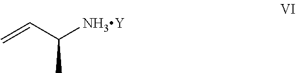

wherein Y is an anion or a salt,
a coupling agent, and
a second base,
to provide the compound of Formula V or a salt thereof.

In some embodiments, the compound of Formula V is a compound of Formula V-A:

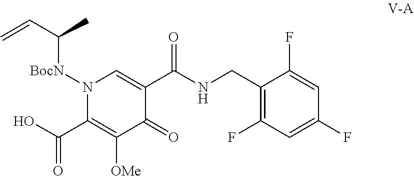 [SMILES: C=C[C@@H](C)N(C(=O)OC(C)(C)C)n1cc(C(=O)NCc2c(F)cc(F)cc2F)c(=O)c(OC)c1C(=O)O]

or a salt thereof.

In some embodiments, Y is selected from tosylate, mesylate, and HCl salt. In some embodiments, Y is tosylate. In some embodiments, Y is mesylate. In some embodiments, Y is an HCl salt.

In some embodiments, the compound of Formula VI or a salt thereof is a compound of Formula VI-A:

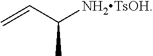

In some embodiments, suitable coupling agents include an acid chloride (e.g., thionyl chloride, oxalyl chloride, phosphorus(V) oxychloride), a mixed anhydride (e.g., with reagents such as acetic anhydride, pivaloyl chloride, diphenylphosphinic anhydride, propanephosphinic anhydride, boric acid, isobutyl chloroformate, methanesulfonyl chloride, p-toluenesulfonyl chloride), a carbonyl diimidazole, an activated ester, 4-(4,6-dimethoxy-1,3,5-triazin-2-yl)-4-methylmorpholinium chloride, 2-chloro-4,6-dimethoxy-1,3,5-triazine, cyanuric chloride, 1-ethyl-3-(3-dimethylaminopropyl)carbodiimide, benzotriazol-1-yloxytripyrrolidinophosphonium hexafluorophosphate, 1-[bis(dimethylamino)methylene]-1H-1,2,3-triazolo[4,5-b]pyridinium 3-oxide hexafluorophosphate, and other reagents known to promote the coupling of amines with carboxylic acids.

In some embodiments, the coupling agent comprises 1-[Bis(dimethylamino)methylene]-1H-1,2,3-triazolo[4,5-b]pyridinium 3-oxid hexafluorophosphate (HATU), an acid chloride, an acid anhydride, a carbonyl diimidazole, an activated ester, or a combination thereof. In some embodiments, the coupling agent comprises thionyl chloride, oxalyl chloride, phosphorus(V) oxychloride, acetic anhydride, pivaloyl chloride, diphenylphosphinic anhydride, propanephosphonic anhydride, boric acid, isobutyl chloroformate, methanesulfonyl chloride, p-toluenesulfonyl chloride, a carbonyl diimidazole, an activated ester, 4-(4,6-dimethoxy-1,3,5-triazin-2-yl)-4-methylmorpholinium chloride, 2-chloro-4,6-dimethoxy-1,3,5-triazine, cyanuric chloride, 1-ethyl-3-(3-dimethylaminopropyl)carbodiimide, benzotriazol-1-yloxytripyrrolidinophosphonium hexafluorophosphate, HATU, or a combination thereof. In some embodiments, the coupling reagent is a peptide coupling reagent. In some embodiments, the coupling agent is HATU.

In some embodiments, suitable second bases include amines (e.g., N-methyl morpholine, triethylamine, diisopropylethylamine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate, etc.), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), basic aromatic compounds (e.g., pyridine, 2,6-lutidine, imidazole, etc.), or a combination thereof.

In some embodiments, the second base comprises an amine, an amidine, a guanidine, a carboxylate, a carbonate, a metal hydride, a hydroxide, an alkoxide, a basic aromatic compound, or a combination thereof. In some embodiments, the second base comprises an amine. In some embodiments, the second base comprises N-methyl morpholine, triethylamine, diisopropylethylamine, or a combination thereof. In some embodiments, the second base is N-methyl morpholine.

In some embodiments, reacting of the compound of Formula V or a salt thereof is performed in a fifth solvent. In some embodiments, the fifth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the fifth solvent comprises an ether. In some embodiments, the fifth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the reacting of the compound of Formula V or a salt thereof is performed in a fifth solvent comprising N,N-dimethylacetamide and cyclopentyl methyl ether.

In some embodiments, the reacting of the compound of Formula V or a salt thereof is carried out in a temperature range of from about 0° C. to about 80° C. In some embodiments, the reacting of the compound of Formula V or a salt thereof is carried out in a temperature range of from about 10° C. to about 30° C. In some embodiments, the reacting of the compound of Formula V or a salt thereof is carried out in at a temperature of about 20° C.

In some embodiments, the process further comprises preparing the compound of Formula V or a salt thereof by a process comprising:
reacting a compound of Formula VII:

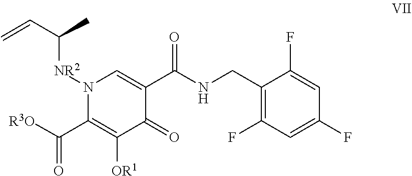

or a salt thereof, wherein R³ is a hydroxyl protecting group;
with a first reagent to provide the compound of Formula V or a salt thereof.

In some embodiments, R³ is selected from C₁₋₄alkyl and benzyl. In some embodiments, R³ is selected from methyl, ethyl, and benzyl. In some embodiments, R³ is C₁₋₄ alkyl. In some embodiments, R³ is selected from methyl and ethyl. In some embodiments, R³ is methyl. In some embodiments, R³ is ethyl. In some embodiments, R³ is benzyl.

In some embodiments, R¹ is selected from hydrogen, methyl, ethyl, and benzyl and R³ is selected from methyl, ethyl, and benzyl. In some embodiments, R¹ and R³ are each independently C₁₋₄alkyl. In some embodiments, R¹ and R³ are each independently selected from methyl and ethyl. In some embodiments, R¹ and R³ are each methyl.

In some embodiments, R² is tert-butyloxycarbonyl and R³ is selected from methyl, ethyl, and benzyl. In some embodiments, R² is tert-butyloxycarbonyl and R³ is C₁₋₄alkyl. In some embodiments, R² is tert-butyloxycarbonyl and R³ selected from methyl and ethyl. In some embodiments, R² is tert-butyloxycarbonyl and R³ selected from methyl.

In some embodiments, R² is tert-butyloxycarbonyl and R¹ and R³ are each independently selected from methyl, ethyl, and benzyl. In some embodiments, R² is tert-butyloxycarbonyl and R¹ and R³ are each independently C₁₋₄alkyl. In some embodiments, R² is tert-butyloxycarbonyl and R¹ and R³ are each independently selected from methyl and ethyl.

In some embodiments, the compound of Formula VII is a compound of Formula VII-B:

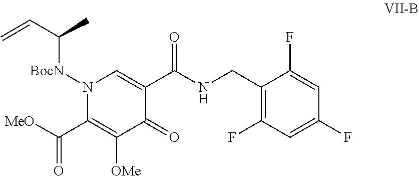 [SMILES: C=C[C@@H](C)N(C(=O)OC(C)(C)C)n1cc(C(=O)NCc2c(F)cc(F)cc2F)c(=O)c(OC)c1C(=O)OC]

In some embodiments, the first reagent is selected from a third base and a hydrolase enzyme.

In some embodiments, the first reagent is the third base. In some embodiments, suitable third bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN), etc.), guanidines (e.g., triazabicyclodecene (TBD), 1,1,3,3-tetramethylguanidine), carbonates (e.g., M(n)H(2-n)CO₃ such as sodium carbonate, potassium bicarbonate, cesium carbonate), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, lithium hydroxide potassium hydroxide, cesium hydroxide), alkoxides (e.g., sodium methoxide, sodium ethoxide, potassium tert-butoxide), amides (e.g., lithium bis(trimethylsilyl)amide, potassium bis(trimethylsilyl)amide, lithium diisopropylamide), or a combination thereof.

In some embodiments, the third base comprises an amine, an amidine, a guanidine, a carbonate, a phosphate, a hydroxide, an alkoxide, an amide, or a combination thereof. In some embodiments, the third base comprises a hydroxide. In some embodiments, the third base comprises an alkali metal hydroxide. In some embodiments, the third base is selected from sodium hydroxide, lithium hydroxide potassium hydroxide, cesium hydroxide, or a combination thereof. In some embodiments, the third base is lithium hydroxide.

In some embodiments, the reacting of the compound of Formula VII or a salt thereof is performed in a sixth solvent. In some embodiments, the sixth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the sixth solvent comprises an ether. In some embodiments, the sixth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the sixth solvent comprises cyclopentyl methyl ether. In some embodiments, the sixth solvent comprises an alcohol. In some embodiments, the sixth solvent comprises methanol, ethanol, 2-propanol, butanol, glycerol, iso-amyl alcohol, or a combination thereof. In some embodiments, the sixth solvent comprises methanol. In some embodiments, the sixth solvent comprises water. In some embodiments, the sixth solvent comprises an ether, an alcohol, and water.

In some embodiments, the reacting of the compound of Formula VII or a salt thereof is performed in a sixth solvent comprising cyclopentyl methyl ether, methanol, and water. In some embodiments, the reacting of the compound of Formula VII or a salt thereof is performed in a sixth solvent comprising cyclopentyl methyl ether, methanol, and water, wherein a volume ratio of the cyclopentyl methyl ether:methanol:water is about 1-3:3-5:1. In some embodiments, the reacting of the compound of Formula VII or a salt thereof is performed in a sixth solvent comprising cyclopentyl methyl ether, methanol, and water, wherein a volume ratio of the cyclopentyl methyl ether:methanol:water is about 2:4:1.

In some embodiments, the reacting of the compound of Formula VII or a salt thereof is carried out in a temperature range of from about 0° C. to about 80° C. In some embodiments, the reacting of the compound of Formula VII or a salt thereof is carried out in a temperature range of from about 10° C. to about 30° C. In some embodiments, the reacting of the compound of Formula VII or a salt thereof is carried out at a temperature of about 30° C.

In some embodiments, the process further comprises preparing the compound of Formula VII or a salt thereof by a process comprising:
reacting a compound of Formula VIII:

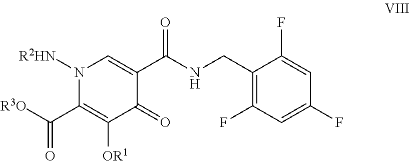

or a salt thereof, with:
   a compound of Formula IX:

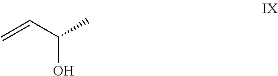

or a salt thereof,
a phosphorus-based reagent, and
an azodicarboxylate,
to provide the compound of Formula VII or a salt thereof.

In some embodiments, the compound of Formula VIII is a compound of Formula VIII-B:

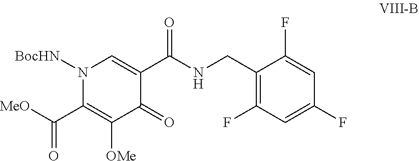 [SMILES: COC(=O)c1c(OC)c(=O)c(C(=O)NCc2c(F)cc(F)cc2F)cn1NC(=O)OC(C)(C)C]

or a salt thereof.

In some embodiments, suitable phosphorus-based reagents include tri-aryl phosphines (e.g., triphenyl phosphine, tris (2-pyridyl) phosphine, tris-(4-methoxyphenyl) phosphine, tris-(dimethylaminophenyl) phosphine, triphenylphosphoranylidene acetonitrile), tri-alkyl phosphines (e.g., tricyclohexyl phosphine, tributyl phosphine) and phosphoranes (e.g., (cyanomethylene) tributylphosphorane).

In some embodiments, the phosphorus-based reagent is selected from a tri-aryl phosphine, a tri-alkyl phosphine, and a phosphorane. In some embodiments, the phosphorus-based reagent is a phosphine. In some embodiments, the phosphorus-based reagent is a tri-aryl phosphine. In some embodiments, the phosphorus-based reagent is selected from triphenyl phosphine, tris (2-pyridyl) phosphine, tris-(4-methoxyphenyl) phosphine, tris-(dimethylaminophenyl) phosphine, and triphenylphosphoranylidene acetonitrile. In some embodiments, the phosphorus-based reagent is triphenylphosphine.

In some embodiments, the azodicarboxylate is selected from diisopropyl azodicarboxylate, di-tertbutyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the azodicarboxylate is diisopropyl azodicarboxylate.

In some embodiments, the reacting of the compound of Formula VIII or a salt thereof is performed in a seventh solvent. In some embodiments, the seventh solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the seventh solvent comprises an ether. In some embodiments, the seventh solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the reacting of the compound of Formula VIII or a salt thereof is performed in a seventh solvent comprising 2-methyltetrahydrofuran.

In some embodiments, the reacting of the compound of Formula VIII or a salt thereof is carried out in a temperature range of from about 0° C. to about 50° C. In some embodiments, the reacting of the compound of Formula VIII or a salt thereof is carried out in a temperature range of from about 10° C. to about 30° C. In some embodiments, the reacting of the compound of Formula VIII or a salt thereof is carried out in a temperature range of about 20° C.

### Alternative Process for Preparing the Compound of Formula VII

In some embodiments, the process comprises preparing the compound of Formula VII or a salt thereof by a process comprising:
reacting a compound of Formula VIII:

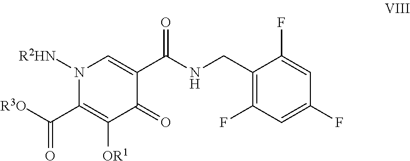

or a salt thereof, with:
   a second reagent comprising a crotyl group or an allyl group,
   a second catalyst, and
   a sixth base
to provide the compound of Formula VII or a salt thereof. In some embodiments, the compound of Formula VII is a compound of Formula VII-B.

In some embodiments, the second reagent comprises a crotyl group (i.e., but-2-en-1-yl). In some embodiments, the second reagent comprises an allyl group (i.e., but-3-en-2-yl).

In some embodiments, suitable second reagents include crotyl carbonates (e.g., crotyl methyl carbonate, crotyl ethyl carbonate), crotyl esters (e.g., crotyl acetate, crotyl trifluoromethylacetate), crotyl halides (e.g., crotyl chloride, crotyl bromide), crotyl alcohol, 1-methylallyl esters (e.g., 1-methylallyl acetate, 1-methylallyl trifluoroacetate), 1-methylallyl carbonates (e.g., 1-methylallyl methyl carbonate, 1-methylallyl ethyl carbonate), 1-methylallyl halides (e.g., 3-chloro-1-butene, 3-bromo-1-butene), 3-buten-2-ol, or a combination thereof.

In some embodiments, the second reagent is selected from a crotyl carbonate, a crotyl ester, a crotyl halide, crotyl alcohol, a 1-methylallyl ester, a 1-methyl allyl carbonate, a 1-methylallylhalide, and 3-buten-2-ol. In some embodiments, the second reagent is selected from crotyl methyl carbonate, crotyl ethyl carbonate, or a combination thereof. In some embodiments, the second reagent is a crotyl carbonate. In some embodiments, the second reagent is crotyl methyl carbonate.

In some embodiments, the second catalyst is a transition metal catalyst. In some embodiments, the transition metal catalyst is selected from an iridium compound, a palladium compound, a cobalt compound, and a rhodium compound. In some embodiments, the transition metal catalyst is an iridium compound.

In some embodiments, the second catalyst is formed in situ from a precatalyst and a ligand. In some embodiments, suitable precatalysts include iridium compounds (e.g., bis(1,5-cyclooctadiene)diiridium(I) dichloride, (1,5-cyclooctadiene)(methoxy)iridium(I) dimer), palladium compounds (e.g., tris(dibenzylideneacetone)dipalladium(0) [Pd₂(dba)₃], palladium(II) acetate, allylpalladium(II) chloride dimer), cobalt compounds (e.g., cobalt(II) tetrafluoroborate, cobalt(II) bromide), and rhodium compounds (e.g., chloro(1,5-cyclooctadiene)rhodium(I) dimer, hydroxy(cyclooctadiene)rhodium(I) dimer).

In some embodiments, the precatalyst is selected from an iridium compound, a palladium compound, a cobalt compound, and a rhodium compound. In some embodiments, the precatalyst is an iridium compound. In some embodiments, the precatalyst is bis(1,5-cyclooctadiene)diiridium(I) dichloride or (1,5-cyclooctadiene)(methoxy)iridium(I) dimer. In some embodiments, the precatalyst is bis(1,5-cyclooctadiene)diiridium(I) dichloride.

In some embodiments, a molar ratio of the compound of Formula VIII or a salt thereof to the precatalyst is from about 1:0.01 to about 1:0.10. In some embodiments, a molar ratio of the compound of Formula VIII or a salt thereof to the precatalyst is from about 1:0.01 to about 1:0.07. In some embodiments, a molar ratio of the compound of Formula VIII or a salt thereof to the precatalyst is about 1:0.04.

In some embodiments, suitable ligands include phosphoramidites (e.g., (S,S,S)-(+)-(3,5-dioxa-4-phosphacyclohepta[2,1-a:3,4-a′]dinaphthalene-4-yl)bis(1-phenylethyl)amine, (S)-(+)-N-(3,5-Dioxa-4-phosphacyclohepta[2,1-a;3,4-a′]dinaphthalen-4-yl)-dibenzo[b,f]azepine, (S)-(+)-(3,5-Dioxa-4-phosphacyclohepta[2,1-a;3,4-a′]dinaphthalen-4-yl)dimethylamine), and Trost ligands (e.g., (1S,2S)-(−)-1,2-diaminocyclohexane-N,N′-bis(2-diphenylphosphinobenzoyl) [(S,S)-DACH-phenyl Trost ligand], (1S,2S)-(−)-1,2-diaminocyclohexane-N,N′-bis(2-diphenylphosphino-1-naphthoyl) [(S,S)-DACH-naphthyl Trost ligand). In some embodiments, the ligand is selected from a phosphoramidite and a Trost ligand. In some embodiments, the ligand is a phosphoramidite. In some embodiments, the ligand is selected from (S,S,S)-(+)-(3,5-dioxa-4-phosphacyclohepta[2,1-a:3,4-a′]dinaphthalene-4-yl)bis(1-phenylethyl)amine, (S)-(+)-N-(3,5-Dioxa-4-phosphacyclohepta[2,1-a;3,4-a′]dinaphthalen-4-yl)-dibenzo[b,f]azepine, and (S)-(+)-(3,5-Dioxa-4-phosphacyclohepta[2,1-a;3,4-a′]dinaphthalen-4-yl)dimethylamine. In some embodiments, the ligand is (S,S,S)-(+)-(3,5-dioxa-4-phosphacyclohepta[2,1-a:3,4-a′]dinaphthalene-4-yl)bis(1-phenylethyl)amine.

In some embodiments, suitable sixth bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), amides (e.g., lithium bis(trimethylsilyl)amide, potassium bis(trimethylsilyl)amide, lithium diisopropylamide), and basic aromatic compounds (e.g., pyridine, 2,6-lutidine, imidazole). In some embodiments, the sixth base comprises an amine, an amidine, a guanidine, a carboxylate, a carbonate, a metal hydride, a phosphate, a hydroxide, an alkoxide, an amide, a basic aromatic compound. In some embodiments, the sixth base is an amine. In some embodiments, the sixth base comprises n-propylamine, triethylamine, N,N-diisopropylethylamine, 1,4-diazabicyclo[2.2.2]octane (DABCO), quinuclidine, or a combination thereof. In some embodiments, the sixth base is n-propylamine.

In some embodiments, the reacting of the compound of Formula VIII or a salt thereof is performed in a twelfth solvent. In some embodiments, the twelfth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the twelfth solvent comprises a polar aprotic solvent. In some embodiments, the twelfth solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula VIII or a salt thereof is performed in a twelfth solvent comprising N,N-dimethylacetamide.

In some embodiments, the reacting of the compound of Formula VIII or a salt thereof is carried out in a temperature range of from about 0° C. to about 80° C. In some embodiments, the reacting of the compound of Formula VIII or a salt thereof is carried out in a temperature range of from about 20° C. to about 30° C. In some embodiments, the reacting of the compound of Formula VIII or a salt thereof is carried out at a temperature of about 25° C.

### Process for Preparing the Compound of Formula VIII

In some embodiments, the process comprises preparing the compound of Formula VIII or a salt thereof by a process comprising:
reacting a compound of Formula X:

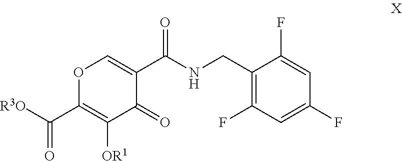

or a salt thereof, with a compound of Formula NH₂NHR₂ or a salt thereof,
to provide the compound of Formula VIII or a salt thereof.

In some embodiments, the compound of Formula X is a compound of Formula X-B:

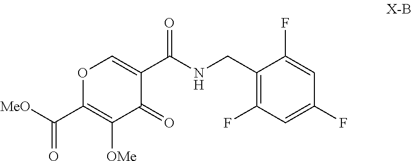 [SMILES: COC(=O)c1occ(C(=O)NCc2c(F)cc(F)cc2F)c(=O)c1OC]

or a salt thereof.

In some embodiments, the compound of Formula NH₂NHR₂ or a salt thereof is NH₂NHBoc.

In some embodiments, the reacting of the compound of Formula X or a salt thereof is carried out in the presence of a fourth base. In some embodiments, suitable fourth bases include amines (e.g., triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), amides (e.g., lithium bis(trimethylsilyl)amide, potassium bis(trimethylsilyl)amide, lithium diisopropylamide, etc.), basic aromatic compounds (e.g., pyridine, 2,6-lutidine, imidazole).

In some embodiments, the fourth base comprises an amine, an amidine, a guanidine, a carboxylate, a carbonate, a metal hydride, a phosphate, a hydroxide, an alkoxide, an amide, a basic aromatic compound, or a combination thereof. In some embodiments, the fourth base is a carbonate. In some embodiments, the fourth base is an alkali metal carbonate. In some embodiments, the fourth base comprises sodium carbonate, potassium bicarbonate, cesium carbonate, or a combination thereof. In some embodiments, the fourth base is sodium bicarbonate.

In some embodiments, the reacting of the compound of Formula X is performed in the absence of a base.

In some embodiments, the reacting of the compound of Formula X or a salt thereof is performed in an eighth solvent. In some embodiments, the eighth solvent comprises an alcohol (e.g., methanol, ethanol, 2-propanol, butanol, glycerol, iso-amyl alcohol), an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), an ester (e.g., ethyl acetate, methyl acetate, isopropyl acetate, dimethyl-2-methyl glutarate, propylene carbonate), a halogenated solvent (e.g., dichloromethane, 1,2-dichloroethane, chlorobenzene, α,α,α-trifluorotoluene), water, or a combination thereof. In some embodiments, the eight solvent comprises an alcohol. In some embodiments, the eighth solvent comprises methanol, ethanol, 2-propanol, butanol, glycerol, iso-amyl alcohol, or a combination thereof. In some embodiments, the eighth solvent comprises methanol. In some embodiments, the eighth solvent comprises water.

In some embodiments, the reacting of the compound of Formula X or a salt thereof is performed in an eighth solvent comprising methanol and water. In some embodiments, the reacting of the compound of Formula X or a salt thereof is performed in an eighth solvent comprising methanol and water, wherein a volume ratio of the methanol to the water is about 1-10:0.1-1.0. In some embodiments, the reacting of the compound of Formula X or a salt thereof is performed in an eighth solvent comprising methanol and water, wherein a volume ratio of the methanol to the water is about 6:0.4.

In some embodiments, the reacting of the compound of Formula X or a salt thereof is carried out in a temperature range of from about 0° C. to about 80° C. In some embodiments, the reacting of the compound of Formula X or a salt thereof is carried out in a temperature range of from about 35° C. to about 55° C. In some embodiments, the reacting of the compound of Formula X or a salt thereof is carried out at a temperature of about 45° C.

In some embodiments, the process further comprises preparing the compound of Formula X or a salt thereof by a process comprising:
reacting a compound of Formula XII:

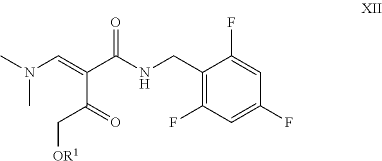

or a salt thereof, with:
   a promoter,
   a dialkyl oxalate, and
   a fifth base,
to provide the compound of Formula X or a salt thereof.

In some embodiments, the compound of Formula XII is a compound of Formula XII-A:

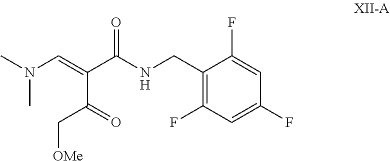 [SMILES: COCC(=O)/C(=C\N(C)C)C(=O)NCc1c(F)cc(F)cc1F]

In some embodiments, suitable promoters include carboxylic acids (e.g., formic acid, acetic acid, trifluoroacetic acid, trichloroacetic acid), sulfonic acids (e.g., methanesulfonic acid, p-toluenesulfonic acid), halogenated alcohols (e.g., 2,2,2-trichloroethanol, 1,1,1,3,3,3-hexafluoro-2-propanol), Lewis acids (e.g., boron trifluoride methyl etherate, boron trifluoride dibutyl etherate, boron trichloride, boron tribromide, aluminum chloride, aluminum bromide), and inorganic acids (e.g., hydrochloric acid, sulfuric acid).

In some embodiments, the promoter comprises a carboxylic acid, a sulfonic acid, a halogenated alcohol, a Lewis acid, an inorganic acid, or a combination thereof. In some embodiments, the promoter comprises a halogenated alcohol. In some embodiments, the promoter comprises 2,2,2-trichloroethanol, 1,1,1,3,3,3-hexafluoro-2-propanol, or a combination thereof. In some embodiments, the promoter is 2,2,2-trifluoroethanol.

In some embodiments, the reacting of the compound of Formula XII or a salt thereof is carried out in the presence of a third acid. In some embodiments, the third acid is an inorganic acid. In some embodiments, the third acid is hydrochloric acid.

In some embodiments, the dialkyl oxalate is a di-C₁₋₄alkyl oxalate. In some embodiments, the dialkyl oxalate is selected from dimethyl oxalate and diethyl oxalate. In some embodiments, the dialkyl oxalate is dimethyl oxalate.

In some embodiments, suitable fifth bases include amines (e.g., ethylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), alkyl and aryl lithium reagents (e.g., n-butyllithium, phenyllithium), amides (e.g., lithium bis(trimethylsilyl)amide, potassium bis(trimethylsilyl)amide, lithium diisopropylamide), and basic aromatic compounds (e.g., pyridine, 2,6-lutidine, imidazole).

In some embodiments, the fifth base comprises an amine, an amidine, a guanidine, a carboxylate, a carbonate, a metal hydride, a phosphate, a hydroxide, an alkoxide, an alkyl lithium reagent, an aryl lithium reagent, an amide, a basic aromatic compound, or a combination thereof. In some embodiments, the fifth base comprises a metal alkoxide. In some embodiments, the fifth base comprises sodium methoxide, sodium ethoxide, potassium tert-butoxide, or a combination thereof. In some embodiments, the fifth base is sodium alkoxide.

In some embodiments, the reacting of the compound of Formula XII or a salt thereof is performed in a tenth solvent. In some embodiments, the tenth solvent comprises an alcohol (e.g., methanol, ethanol, 2-propanol, butanol, glycerol, iso-amyl alcohol), an ether (e.g., tetrahydrofuran, 2-methyltetrahydrofuran, tert-butyl methyl ether, 1,3-dioxolane, cyclopentyl methyl ether, 1,4-dioxane), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), a halogenated solvent (e.g., dichloromethane, 1,2-dichloroethane, chlorobenzene, α,α,α-trifluorotoluene), water, or a combination thereof. In some embodiments, the tenth solvent comprises a polar aprotic solvent. In some embodiments, the tenth solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula XII or a salt thereof is performed in a tenth solvent comprising N,N-dimethylacetamide.

In some embodiments, the reacting of the compound of Formula XII or a salt thereof is carried out in a temperature range of from about −10° C. to about 50° C. In some embodiments, the reacting of the compound of Formula XII or a salt thereof is carried out in a temperature range of from about 0° C. to about 20° C. In some embodiments, the reacting of the compound of Formula XII or a salt thereof is carried out at a temperature of about 10° C.

In some embodiments, the process comprises preparing the compound of Formula X or a salt thereof by a process comprising:
reacting a compound of Formula XI:

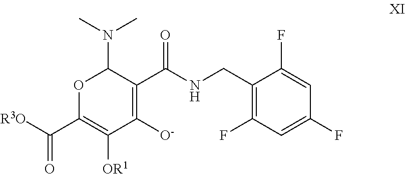

or a salt thereof, with a promoter to provide the compound of Formula X or a salt thereof.

In some embodiments, the promoter comprises a carboxylic acid, a sulfonic acid, a halogenated alcohol, a Lewis acid, an inorganic acid, or a combination thereof. In some embodiments, the promoter comprises a halogenated alcohol. In some embodiments, the promoter is 2,2,2-trifluoroethanol.

In some embodiments, the reacting of the compound of Formula XI or a salt thereof is carried out in the presence of a third acid. In some embodiments, the third acid is an inorganic acid. In some embodiments, the third acid is hydrochloric acid.

In some embodiments, the reacting of the compound of Formula XI or a salt thereof is performed in a ninth solvent comprising dichloromethane.

In some embodiments, the reacting of the compound of Formula XI or a salt thereof is carried out in a temperature range of from about −10° C. to about 50° C.

In some embodiments, the compound of Formula XI is a compound of Formula XI-A:

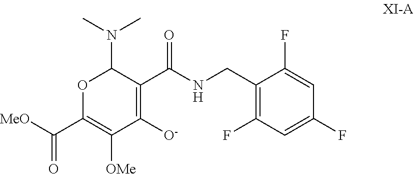 [SMILES: COC(=O)C1=C(OC)C([O-])=C(C(=O)NCc2c(F)cc(F)cc2F)C(N(C)C)O1]

or a salt thereof.

In some embodiments, the reacting of the compound of Formula XI or a salt thereof is carried out in the presence of a third acid. In some embodiments, the third acid is an inorganic acid. In some embodiments, the third acid is hydrochloric acid. In some embodiments, a weight ratio of the compound of Formula XI to the third acid is about 1:0.01-0.25. In some embodiments, a weight ratio of the compound of Formula XI to the third acid is about 1:0.1.

In some embodiments, the reacting of the compound of Formula XI or a salt thereof is performed in a ninth solvent comprising dichloromethane.

In some embodiments, the reacting of the compound of Formula XI or a salt thereof is carried out in a temperature range of from about −10° C. to about 50° C.

In some embodiments, the process further comprises preparing the compound of Formula XI or a salt thereof by a process comprising:
reacting a compound of Formula XII:

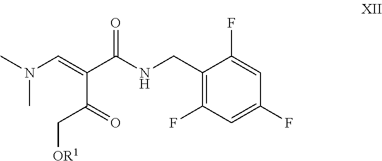

or a salt thereof, with:
   a dialkyl oxalate, and
   a fifth base,
to provide the compound of Formula XI or a salt thereof.

In some embodiments, the dialkyl oxalate is selected from dimethyl oxalate and diethyl oxalate. In some embodiments, the dialkyl oxalate is dimethyl oxalate. In some embodiments, the dialkyl oxalate is diethyl oxalate.

In some embodiments, the fifth base comprises an amine, an amidine, a guanidine, a carboxylate, a carbonate, a metal hydride, a phosphate, a hydroxide, an alkoxide, an alkyl lithium reagent, an aryl lithium reagent, an amide, a basic aromatic compound, or a combination thereof. In some embodiments, the fifth base comprises a metal alkoxide. In some embodiments, the fifth base is sodium alkoxide.

In some embodiments, the reacting of the compound of Formula XII or a salt thereof is performed in a tenth solvent comprising N,N-dimethylacetamide.

In some embodiments, the reacting of the compound of Formula XII or a salt thereof is carried out in a temperature range of from about −10° C. to about 50° C.

In some embodiments, the process further comprises preparing the compound of Formula XII or a salt thereof by a process comprising:
reacting a compound of Formula XIII:

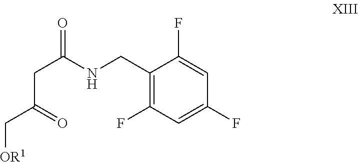

or a salt thereof, with N,N-dimethylformamide dimethyl acetal,
to provide the compound of Formula XII or a salt thereof. Methods of preparing the compound of Formula XII or a salt thereof are described in WO 2015195656, which is incorporated by reference herein.

In some embodiments, provided herein is a process for preparing a compound of Formula I:

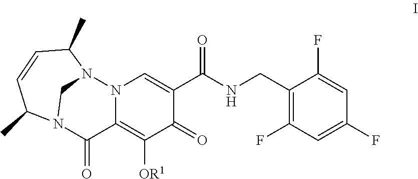

or a salt thereof, wherein R¹ is selected from hydrogen and a hydroxyl protecting group, comprising:
(a) reacting a compound of Formula VII:

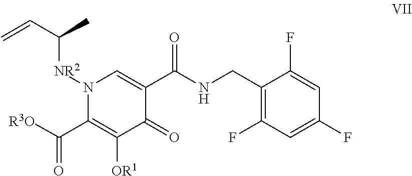

or a salt thereof, wherein R² is an amine protecting group and R³ is a hydroxyl protecting group, with a first reagent to provide a compound of Formula V:

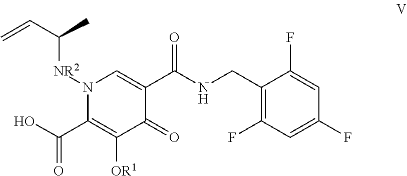

or a salt thereof;
(b) reacting the compound of Formula V or a salt thereof, with:
   a compound of Formula VI:

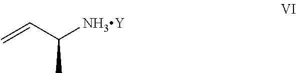

wherein Y is an anion or salt,
a coupling agent, and
a second base,
to provide a compound of Formula IV:

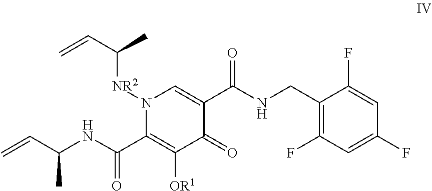

or a salt thereof;
(c) deprotecting the compound of Formula IV or a salt thereof to provide a compound of Formula III:

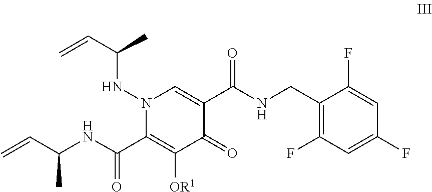

or a salt thereof;
(d) reacting the compound of Formula III or a salt thereof, with:
   a methylene reagent, and
   a first acid or a first base,
to provide a compound of Formula II:

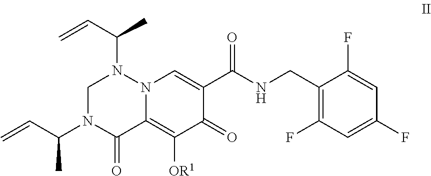

or a salt thereof; and
(e) reacting the compound of Formula II or a salt thereof with a first catalyst to provide the compound of Formula I or a salt thereof.

In some embodiments, provided herein is a process for preparing a compound of Formula I-A:

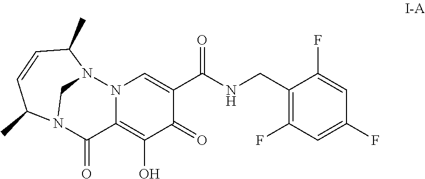

or a salt thereof, comprising:
(a) reacting a compound of Formula VII:

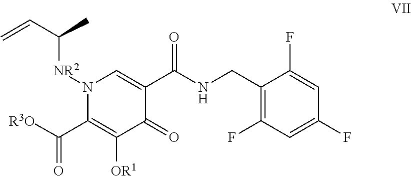

or a salt thereof, wherein R¹ and R³ are each independently hydroxyl protecting groups, and R² is an amine protecting group,
with a first reagent to provide a compound of Formula V:

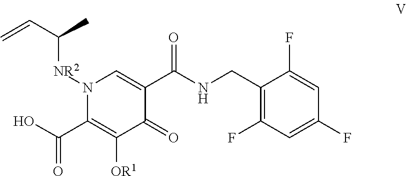

or a salt thereof;
(b) reacting the compound of Formula V or a salt thereof, with:
   a compound of Formula VI:

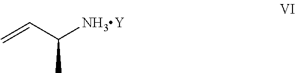

wherein Y is an anion or salt,
a coupling agent, and
a second base,
to provide a compound of Formula IV:

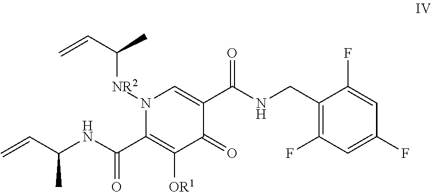

or a salt thereof;
(c) deprotecting the compound of Formula IV or a salt thereof to provide a compound of Formula III:

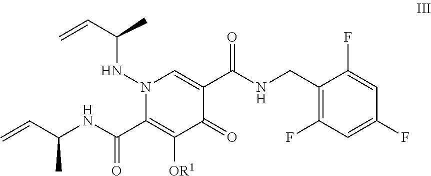

or a salt thereof;
(d) reacting the compound of Formula III or a salt thereof, with
   a methylene reagent, and
   a first acid or a first base,
to provide a compound of Formula II:

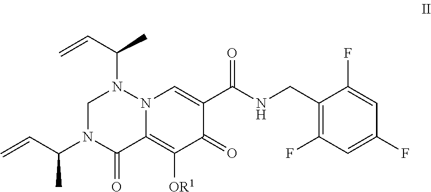

or a salt thereof;
(e) reacting the compound of Formula II or a salt thereof with a first catalyst to provide a compound of Formula I:

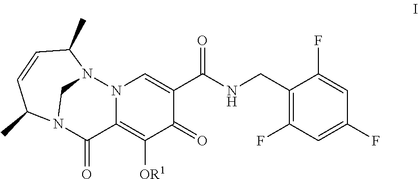

or a salt thereof; and
(f) deprotecting the compound of Formula I, or a salt thereof; to provide the compound of Formula I-A or a salt thereof.

In some embodiments, provided herein is a process for preparing a compound of Formula II:

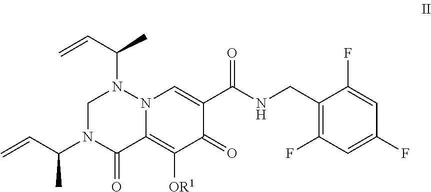

or a salt thereof, comprising:
(a) reacting a compound of Formula VII:

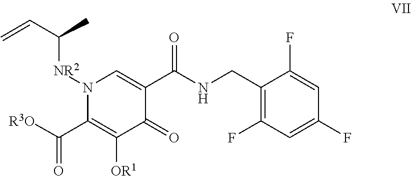

or a salt thereof, wherein R¹ and R³ are each independently hydroxyl protecting groups, and R² is an amine protecting group,
with a first reagent to provide a compound of Formula V:

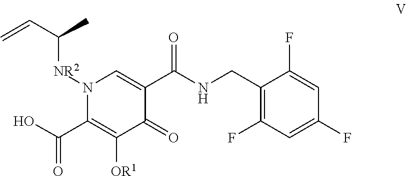

or a salt thereof;
(b) reacting the compound of Formula V or a salt thereof, with:
   a compound of Formula VI:

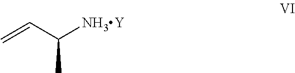

wherein Y is an anion or salt,
a coupling agent, and
a second base,
to provide a compound of Formula IV:

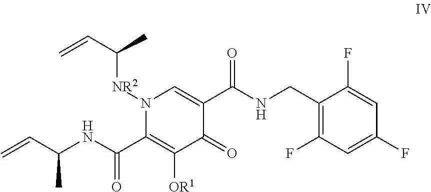

or a salt thereof;
(c) deprotecting the compound of Formula IV or a salt thereof to provide a compound of Formula III:

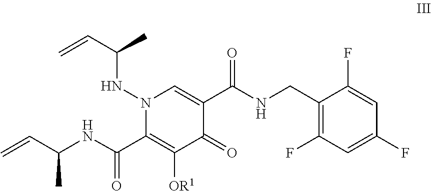

or a salt thereof; and
(d) reacting the compound of Formula III or a salt thereof, with
   a methylene reagent, and
   a first acid or a first base,
to provide the compound of Formula II or a salt thereof.

In some embodiments, provided herein is a process for preparing a compound of Formula III:

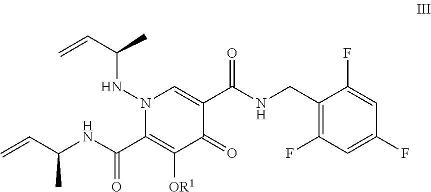

or a salt thereof, comprising:
(a) reacting a compound of Formula VII:

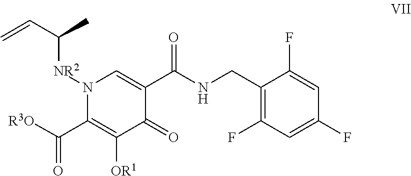

or a salt thereof, wherein R¹ and R³ are each independently hydroxyl protecting groups, and R² is an amine protecting group,
with a first reagent to provide a compound of Formula V:

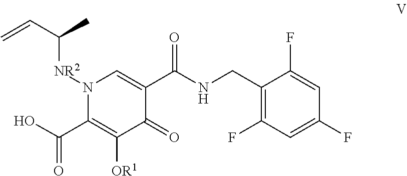

or a salt thereof;
(b) reacting the compound of Formula V or a salt thereof, with:
   a compound of Formula VI:

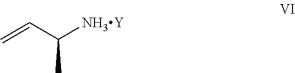

wherein Y is an anion or salt,
a coupling agent, and
a second base,
to provide a compound of Formula IV:

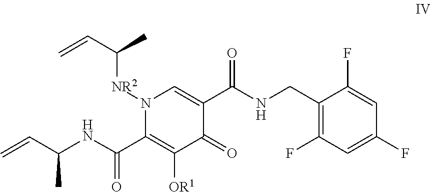

or a salt thereof; and
(c) deprotecting the compound of Formula IV or a salt thereof to provide the compound of Formula III or a salt thereof.

In some embodiments, provided herein is a process for preparing a compound of Formula IV:

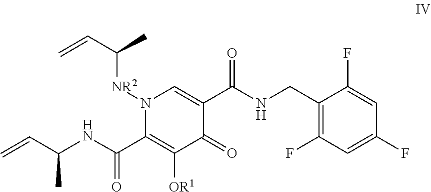

or a salt thereof, comprising:
(a) reacting a compound of Formula VII:

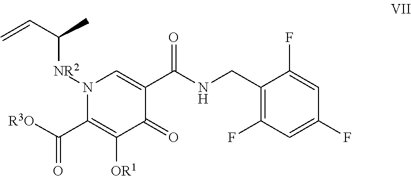

or a salt thereof, wherein R¹ and R³ are each independently hydroxyl protecting groups, and R² is an amine protecting group,
with a first reagent to provide a compound of Formula V:

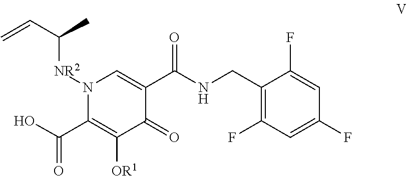

or a salt thereof; and
(b) reacting the compound of Formula V or a salt thereof, with:
   a compound of Formula VI:

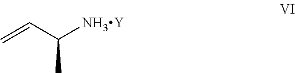

wherein Y is an anion or salt,
a coupling agent, and
a second base,
to provide the compound of Formula IV or a salt thereof.

In some embodiments, provided herein is a process for preparing a compound of Formula V:

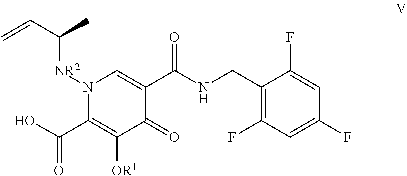

or a salt thereof, comprising:
reacting a compound of Formula VII:

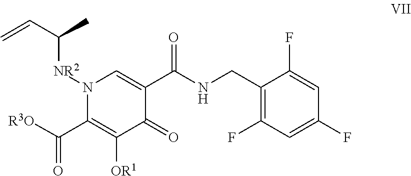

or a salt thereof, wherein R¹ and R³ are each independently hydroxyl protecting groups, and R² is an amine protecting group,
with a first reagent to provide the compound of Formula V or a salt thereof.

In some embodiments of the processes disclosed herein, the compound of Formula VII or a salt thereof is prepared by a process comprising:
(a) reacting a compound of Formula XIII:

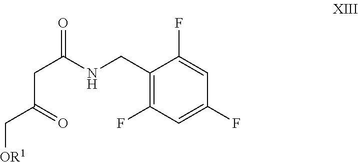

or a salt thereof, wherein R¹ is a hydroxyl protecting group,
with N,N-dimethylformamide dimethyl acetal,
to provide a compound of Formula XII:

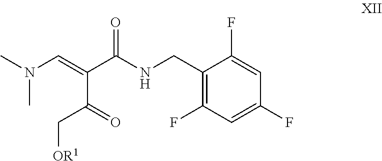

or a salt thereof;
(b) reacting the compound of Formula XII or a salt thereof, with:
a promoter,
a dialkyl oxalate, and
a fifth base,
to provide a compound of Formula X:

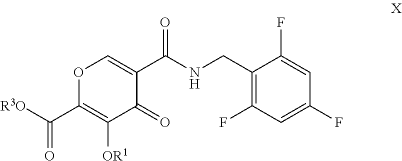

or a salt thereof;
(c) reacting the compound of Formula X or a salt thereof, with a compound of Formula NH₂NHR₂ or a salt thereof,
to provide the compound of Formula VIII:

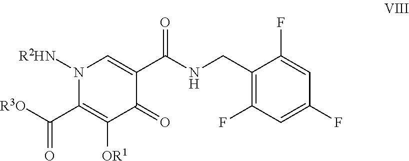

or a salt thereof; and
(d) reacting the compound of Formula VIII or a salt thereof, with:
   a compound of Formula IX:

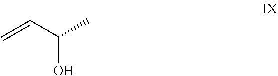

or a salt thereof,
a phosphorus-based reagent, and
an azodicarboxylate,
to provide the compound of Formula VII or a salt thereof.

In some embodiments of the processes disclosed herein, the compound of Formula VII or a salt thereof is prepared by a process comprising:
(a) reacting a compound of Formula XIII:

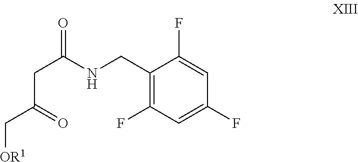

or a salt thereof, wherein R¹ is a hydroxyl protecting group,
with N,N-dimethylformamide dimethyl acetal,
to provide a compound of Formula XII:

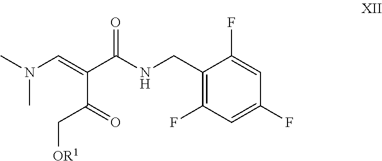

or a salt thereof;
(b) reacting the compound of Formula XII or a salt thereof, with:
   a promoter,
   a dialkyl oxalate, and
   a fifth base,
to provide a compound of Formula X:

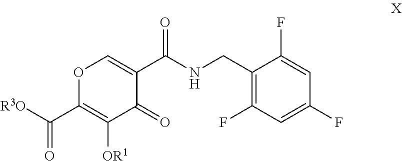

or a salt thereof;
(c) reacting the compound of Formula X or a salt thereof, with a compound of Formula NH₂NHR₂ or a salt thereof,
to provide the compound of Formula VIII:

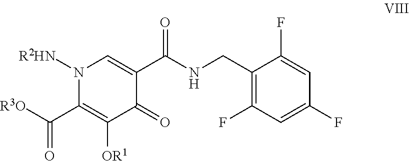

or a salt thereof; and
(e) reacting the compound of Formula VIII or a salt thereof, with:
   a second reagent comprising a crotyl group or an allyl group,
   a second catalyst, and
   a sixth base
to provide the compound of Formula VII or a salt thereof.

### Alternative Process for Preparing the Compound of Formula III

In some embodiments, the process comprises preparing the compound of Formula III or a salt thereof by a process comprising:
reacting a compound of Formula VII-A:

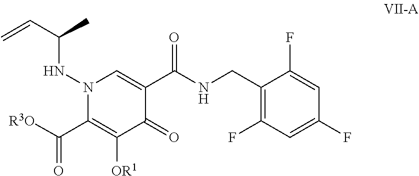

or a salt thereof, with a first reagent to hydrolyze the compound of Formula VII-A; and
reacting the hydrolyzed compound of Formula VII-A with:
   a compound of Formula VI:

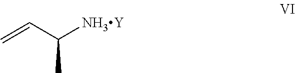

wherein Y is an anion or salt,
a coupling agent, and
a second base,
to provide the compound of Formula III or a salt thereof.

In some embodiments, the first reagent is selected from a third base and a hydrolase enzyme.

In some embodiments, the first reagent is the third base. In some embodiments, the third base comprises an amine, an amidine, a guanidine, a carbonate, a phosphate, a hydroxide, an alkoxide, an amide, or a combination thereof. In some embodiments, the third base is lithium hydroxide.

In some embodiments, Y is selected from tosylate, mesylate, and HCl salt. In some embodiments, Y is tosylate.

In some embodiments, the coupling agent comprises 1-[Bis(dimethylamino)methylene]-1H-1,2,3-triazolo[4,5-b]pyridinium 3-oxid hexafluorophosphate (HATU), an acid chloride, an acid anhydride, a carbonyl diimidazole, an activated ester, or a combination thereof. In some embodiments, the coupling agent is HATU.

In some embodiments, the second base comprises an amine, an amidine, a guanidine, a carboxylate, a carbonate, a metal hydride, a hydroxide, an alkoxide, a basic aromatic compound, or a combination thereof. In some embodiments, the second base comprises an amine. In some embodiments, the second base is N-methyl morpholine.

In some embodiments, the process further comprises preparing the compound of Formula VII-A or a salt thereof by a process comprising:
reacting a compound of Formula VIII-A:

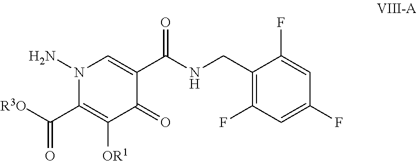

or a salt thereof, with:
   a second reagent comprising a crotyl group or an allyl group,
   a second catalyst, and
   a sixth base,
to provide the compound of Formula VII-A or a salt thereof.

In some embodiments, the second reagent comprises a crotyl group (i.e., but-2-en-1-yl). In some embodiments, the second reagent comprises an allyl group (i.e., but-3-en-2-yl).

In some embodiments, suitable second reagents include crotyl carbonates (e.g., crotyl methyl carbonate, crotyl ethyl carbonate), crotyl esters (e.g., crotyl acetate, crotyl trifluoromethylacetate), crotyl halides (e.g., crotyl chloride, crotyl bromide), crotyl alcohol, 1-methylallyl esters (e.g., 1-methylallyl acetate, 1-methylallyl trifluoroacetate), 1-methylallyl carbonates (e.g., 1-methylallyl methyl carbonate, 1-methylallyl ethyl carbonate), 1-methylallyl halides (e.g., 3-chloro-1-butene, 3-bromo-1-butene), 3-buten-2-ol, or a combination thereof.

In some embodiments, the second reagent is selected from a crotyl carbonate, a crotyl ester, a crotyl halide, crotyl alcohol, a 1-methylallyl ester, a 1-methyl allyl carbonate, a 1-methylallylhalide, and 3-buten-2-ol. In some embodiments, the second reagent is selected from crotyl methyl carbonate, crotyl ethyl carbonate, or a combination thereof. In some embodiments, the second reagent is a crotyl carbonate. In some embodiments, the second reagent is crotyl methyl carbonate. In some embodiments, the second reagent is crotyl methyl carbonate, wherein the crotyl methyl carbonate is a mixture of cis and trans.

In some embodiments, the second catalyst is a transition metal catalyst. In some embodiments, the transition metal catalyst is selected from an iridium compound, a palladium compound, a cobalt compound, and a rhodium compound. In some embodiments, the transition metal catalyst is an iridium compound.

In some embodiments, the second catalyst is formed in situ from a precatalyst and a ligand. In some embodiments, suitable precatalysts include iridium compounds (e.g., bis(1,5-cyclooctadiene)diiridium(I) dichloride, (1,5-cyclooctadiene)(methoxy)iridium(I) dimer), palladium compounds (e.g., tris(dibenzylideneacetone)dipalladium(0) [Pd₂(dba)₃], palladium(II) acetate, allylpalladium(II) chloride dimer), cobalt compounds (e.g., cobalt(II) tetrafluoroborate, cobalt(II) bromide), and rhodium compounds (e.g., chloro(1,5-cyclooctadiene)rhodium(I) dimer, hydroxy(cyclooctadiene)rhodium(I) dimer).

In some embodiments, the precatalyst is selected from an iridium compound, a palladium compound, a cobalt compound, and a rhodium compound. In some embodiments, the precatalyst is an iridium compound. In some embodiments, the precatalyst is bis(1,5-cyclooctadiene)diiridium(I) dichloride or (1,5-cyclooctadiene)(methoxy)iridium(I) dimer. In some embodiments, the precatalyst is bis(1,5-cyclooctadiene)diiridium(I) dichloride.

In some embodiments, a molar ratio of the compound of Formula VIII-A or a salt thereof to the precatalyst is from about 1:0.01 to about 1:0.10. In some embodiments, a molar ratio of the compound of Formula VIII-A or a salt thereof to the precatalyst is from about 1:0.005 to about 1:0.05. In some embodiments, a molar ratio of the compound of Formula VIII-A or a salt thereof to the precatalyst is from about 1:0.01 to about 1:0.03. In some embodiments, a molar ratio of the compound of Formula VIII-A or a salt thereof to the precatalyst is about 1:0.02.

In some embodiments, suitable ligands include phosphoramidites (e.g., (S,S,S)-(+)-(3,5-dioxa-4-phosphacyclohepta[2,1-a:3,4-a′]dinaphthalene-4-yl)bis(1-phenylethyl)amine, (S)-(+)-N-(3,5-Dioxa-4-phosphacyclohepta[2,1-a;3,4-a′]dinaphthalen-4-yl)-dibenzo[b,f]azepine, (S)-(+)-(3,5-Dioxa-4-phosphacyclohepta[2,1-a;3,4-a′]dinaphthalen-4-yl)dimethylamine), and Trost ligands (e.g., (1S,2S)-(−)-1,2-diaminocyclohexane-N,N′-bis(2-diphenylphosphinobenzoyl) [(S,S)-DACH-phenyl Trost ligand], (1S,2S)-(−)-1,2-diaminocyclohexane-N,N′-bis(2-diphenylphosphino-1-naphthoyl) [(S,S)-DACH-naphthyl Trost ligand). In some embodiments, the ligand is selected from a phosphoramidite and a Trost ligand. In some embodiments, the ligand is a phosphoramidite. In some embodiments, the ligand is selected from (S,S,S)-(+)-(3,5-dioxa-4-phosphacyclohepta[2,1-a:3,4-a′]dinaphthalene-4-yl)bis(1-phenylethyl)amine, (S)-(+)-N-(3,5-Dioxa-4-phosphacyclohepta[2,1-a;3,4-a′]dinaphthalen-4-yl)-dibenzo[b,f]azepine, and (S)-(+)-(3,5-Dioxa-4-phosphacyclohepta[2,1-a;3,4-a′]dinaphthalen-4-yl)dimethylamine. In some embodiments, the ligand is (S,S,S)-(+)-(3,5-dioxa-4-phosphacyclohepta[2,1-a:3,4-a′]dinaphthalene-4-yl)bis(1-phenylethyl)amine.

In some embodiments, suitable sixth bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), amides (e.g., lithium bis(trimethylsilyl)amide, potassium bis(trimethylsilyl)amide, lithium diisopropylamide), and basic aromatic compounds (e.g., pyridine, 2,6-lutidine, imidazole). In some embodiments, the sixth base comprises an amine, an amidine, a guanidine, a carboxylate, a carbonate, a metal hydride, a phosphate, a hydroxide, an alkoxide, an amide, a basic aromatic compound. In some embodiments, the sixth base is an amine. In some embodiments, the sixth base comprises n-propylamine, triethylamine, N,N-diisopropylethylamine, 1,4-diazabicyclo[2.2.2]octane (DABCO), quinuclidine, or a combination thereof. In some embodiments, the sixth base is 1,4-diazabicyclo[2.2.2]octane (DABCO).

In some embodiments, the reacting of the compound of Formula VIII-A or a salt thereof is performed in a twelfth solvent. In some embodiments, the twelfth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the twelfth solvent comprises a polar aprotic solvent. In some embodiments, the twelfth solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula VIII-A or a salt thereof is performed in a twelfth solvent comprising N,N-dimethylacetamide.

In some embodiments, the reacting of the compound of Formula VIII-A or a salt thereof is carried out in a temperature range of from about 0° C. to about 80° C. In some embodiments, the reacting of the compound of Formula VIII-A or a salt thereof is carried out in a temperature range of from about 45° C. to about 55° C. In some embodiments, the reacting of the compound of Formula VIII-A or a salt thereof is carried out at a temperature of about 50° C.

### Alternative Process for Preparing the Compound of Formula I-A

In some embodiments, provided herein is a process for preparing a compound of Formula I-A:

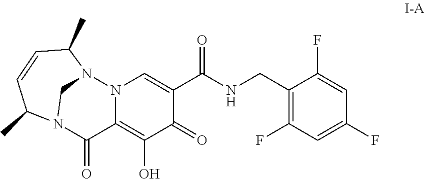

or a salt thereof, comprising deprotecting a compound of Formula I:

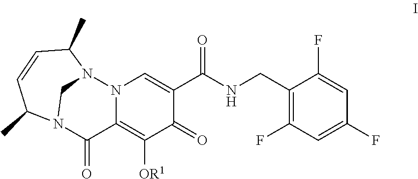

or a salt thereof, wherein R¹ is a hydroxyl protecting group, to provide the compound of Formula I-A or a salt thereof.

In some embodiments, R¹ is selected from C₁₋₄alkyl, benzyl, and —COOR⁴; wherein R⁴ is selected from C₁₋₄alkyl and phenyl. In some embodiments, R¹ is selected from methyl, ethyl, benzyl, acetate, propionate, and benzoate. In some embodiments, R¹ is selected from C₁₋₄alkyl and benzyl. In some embodiments, R¹ is C₁₋₄ alkyl. In some embodiments, R¹ is selected from methyl, ethyl, and benzyl. In some embodiments, R¹ is methyl. In some embodiments, R¹ is ethyl. In some embodiments, R¹ is benzyl. In some embodiments, R¹ is —COOR⁴. In some embodiments, R⁴ is C₁₋₄alkyl. In some embodiments, R⁴ is methyl or ethyl. In some embodiments, R⁴ is phenyl.

In some embodiments, the compound of Formula I is a compound of Formula I-B.

In some embodiments, deprotecting the compound of Formula I or a salt thereof comprises treating the compound of Formula I or a salt thereof with a first deprotecting agent. In some embodiments, suitable first deprotecting agents include metal halide salts (e.g., magnesium bromide, magnesium chloride, lithium chloride, potassium iodide, sodium iodide, calcium chloride, calcium bromide, calcium iodide, copper (I) chloride, copper (II) chloride, zinc chloride), hydrogen halides (e.g., hydrogen chloride, hydrogen bromide, hydrogen iodide), boron reagents (e.g., boron trichloride, boron tribromide), aluminum reagents (e.g., aluminum trichloride, aluminum tribromide), titanium reagents (e.g., titanium tetrachloride, titanium tribromide, titanium tetrabromide), pyridinium halide salts (e.g., pyridinium chloride, pyridinium perbromide, pyridinium iodide), ammonium halides (e.g., ammonium chloride, tetrabutylammonium chloride, tetrabutylammonium iodide), silyl halides (e.g., trimethylsilyl chloride, trimethylsilyl iodide).

In some embodiments, suitable first deprotecting agents include metal halide salts (e.g., magnesium bromide, lithium chloride, potassium iodide, sodium iodide, calcium chloride, copper (I) chloride, copper (II) chloride, zinc chloride), hydrogen halides (e.g., hydrogen chloride, hydrogen bromide, hydrogen iodide), boron reagents (e.g., boron trichloride, boron tribromide), aluminum reagents (e.g., aluminum trichloride, aluminum tribromide), titanium reagents (e.g., titanium tetrachloride, titanium tribromide, titanium tetrabromide), pyridinium halide salts (e.g., pyridinium chloride, pyridinium perbromide, pyridinium iodide), ammonium halides (e.g., ammonium chloride, tetrabutylammonium chloride, tetrabutylammonium iodide), silyl halides (e.g., trimethylsilyl chloride, trimethylsilyl iodide).

In some embodiments, the first deprotecting agent is selected from a metal halide, a hydrogen halide, a boron reagent, an aluminum reagent, a titanium reagent, a pyridinium halide salt, an ammonium halide, and a silyl halide. In some embodiments, the first deprotecting agent is a metal halide. In some embodiments, the first deprotecting agent comprises magnesium bromide, magnesium chloride, lithium chloride, potassium iodide, sodium iodide, calcium chloride, copper (I) chloride, copper (II) chloride, zinc chloride, calcium bromide, calcium iodide, or a combination thereof. In some embodiments, the first deprotecting agent comprises magnesium bromide, lithium chloride, potassium iodide, sodium iodide, calcium chloride, copper (I) chloride, copper (II) chloride, zinc chloride, or a combination thereof. In some embodiments, the first deprotecting agent is lithium chloride. In some embodiments, the first deprotecting agent is calcium bromide. In some embodiments, the first deprotecting agent is magnesium chloride.

In some embodiments, the deprotecting the compound of Formula I or a salt thereof is performed in a second solvent. In some embodiments, the second solvent comprises water, an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the second solvent comprises a polar aprotic solvent. In some embodiments, the second solvent comprises N,N-dimethylformamide, N,N-dimethylacetamide, N-methyl-2-pyrrolidone, N-butyl pyrrolidone (NBP), or a combination thereof. In some embodiments, the deprotecting the compound of Formula I or a salt thereof is performed in a second solvent comprising N-methyl-2-pyrrolidone.

In some embodiments, the second solvent comprises an alcohol. In some embodiments, the second solvent comprises methanol, ethanol, 2-propanol, butanol, glycerol, iso-amyl alcohol, or a combination thereof. In some embodiments, the deprotecting the compound of Formula I or a salt thereof is performed in a second solvent comprising 2-propanol.

In some embodiments, the deprotecting the compound of Formula I or a salt thereof is carried out in a temperature range of from about 0° C. to about 130° C. In some embodiments, the deprotecting the compound of Formula I or a salt thereof is carried out in a temperature range of from about 0° C. to about 100° C. In some embodiments, the deprotecting the compound of Formula I or a salt thereof is carried out in a temperature range of from about 70° C. to about 90° C. In some embodiments, the deprotecting the compound of Formula I or a salt thereof is carried out in a temperature of about 80° C.

In some embodiments, the process further comprises preparing the compound of Formula I or a salt thereof by a process comprising:
reacting a compound of Formula A:

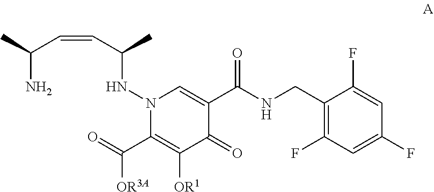

or a salt thereof, wherein R^{3A} is a hydroxyl protecting group, with:
   a methylene reagent, and
   a first acid or a first base,
to provide the compound of Formula I or a salt thereof.

In some embodiments, R^{3A} is selected from C₁₋₄alkyl, C₁₋₄haloalkyl, and benzyl. In some embodiments, R^{3A} is selected from methyl, ethyl, isopropyl, trifluoroethyl, and benzyl. In some embodiments, R^{3A} is C₁₋₄alkyl. In some embodiments, R^{3A} is selected from methyl, ethyl, and isopropyl. In some embodiments, R^{3A} is methyl. In some embodiments, R^{3A} is C₁₋₄haloalkyl. In some embodiments, R^{3A} is trifluoroethyl. In some embodiments, R^{3A} is benzyl.

In some embodiments, R¹ is C₁₋₄alkyl and R^{3A} is selected from C₁₋₄alkyl, C₁₋₄haloalkyl, and benzyl. In some embodiments, R¹ and R^{3A} are each independently C₁₋₄alkyl. In some embodiments, R¹ and R^{3A} are each independently selected from methyl, ethyl, and isopropyl.

In some embodiments, the compound of Formula A is a compound of Formula A-1:

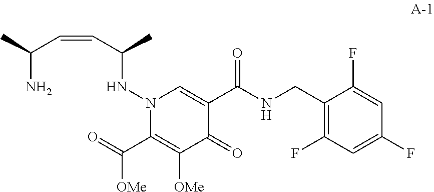 [SMILES: COC(=O)c1c(OC)c(=O)c(C(=O)NCc2c(F)cc(F)cc2F)cn1N[C@H](C)/C=C\[C@H](C)N]

or a salt thereof.

In some embodiments, the methylene reagent is a formaldehyde reagent or a dihalomethane. In some embodiments, suitable formaldehyde reagents include formaldehyde acetals (e.g., dimethoxymethane, diethoxymethane), formaldehyde solution, paraformaldehyde, and 1,3,5-trioxane. In some embodiments, suitable methylene reagents include dihalomethanes (e.g., dibromomethane, diiodomethane).

In some embodiments, the methylene reagent is selected from 1,3,5-trioxane, a formaldehyde acetal, formaldehyde, paraformaldehyde, or a dihalomethane. In some embodiments, the methylene reagent is a formaldehyde reagent. In some embodiments, the methylene reagent is selected from 1,3,5-trioxane, a formaldehyde acetal, formaldehyde, and paraformaldehyde. In some embodiments, the methylene reagent is 1,3,5-trioxane.

In some embodiments, the compound of Formula A or a salt thereof is reacted with the first acid. In some embodiments, suitable first acids include carboxylic acids (e.g., oxalic acid, trichloroacetic acid, trifluoroacetic acid), sulfonic acids (e.g., methanesulfonic acid, benzenesulfonic acid, para-toluenesulfonic acid, camphorsulfonic acid), Lewis acids (e.g., boron trifluoride methyl etherate, boron trifluoride dibutyl etherate, boron trichloride, boron tribromide, aluminum chloride, aluminum bromide), inorganic acids (e.g., hydrochloric acid, sulfuric acid, phosphoric acid, hydrobromic acid), or a combination thereof.

In some embodiments, the first acid comprises a carboxylic acid, a sulfonic acid, a Lewis acid, an inorganic acid, or a combination thereof. In some embodiments, the first acid comprises a carboxylic acid. In some embodiments, the first acid is selected from oxalic acid, trichloroacetic acid, trifluoroacetic acid, or a combination thereof. In some embodiments, the first acid is trifluoroacetic acid.

In some embodiments, the compound of Formula A or a salt thereof is reacted with the first base. In some embodiments, suitable first bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN), etc.), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide, etc.), amides (e.g., lithium bis(trimethylsilyl)amide, potassium bis(trimethylsilyl)amide, lithium diisopropylamide), basic aromatic compounds (e.g., pyridine, 2,6-lutidine, imidazole), or a combination thereof.

In some embodiments, the first base comprises an amine, an amidine, a guanidine, a carboxylate, a carbonate, a metal hydride, a phosphate, a hydroxide, an alkoxide, an amide, a basic aromatic compound, or a combination thereof.

In some embodiments, the reacting of the compound of Formula A or a salt thereof is performed in a third solvent. In some embodiments, the third solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the third solvent comprises a hydrocarbon. In some embodiments, the third solvent comprises toluene, methylcyclohexane, n-heptane, hexanes, limonene, or a combination thereof. In some embodiments, the reacting of the compound of Formula A or a salt thereof is performed in a third solvent comprising toluene.

In some embodiments, the reacting of the compound of Formula A or a salt thereof is carried out in a temperature range of from about 20° C. to about 140° C. In some embodiments, the reacting of the compound of Formula A or a salt thereof is carried out in a temperature range of from about 90° C. to about 110° C. In some embodiments, the reacting of the compound of Formula III or a salt thereof is carried out at a temperature of about 100° C.

### Alternative Process for Preparing the Compound of Formula I

In some embodiments of the process disclosed herein, the process comprises preparing the compound of Formula I or a salt thereof, by a process comprising:
reacting a compound of Formula R-1:

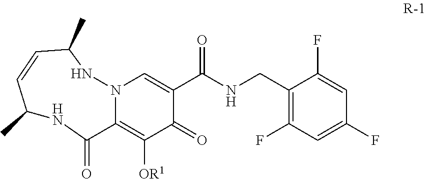

or a salt thereof with:
   a methylene reagent, and
   an eighth acid or a fourteenth base,
to provide the compound of Formula I or a salt thereof, wherein R¹ is a hydroxyl protecting group. In some embodiments, R¹ is selected from C₁₋₄alkyl, benzyl, and —COOR⁴; wherein R⁴ is selected from C₁₋₄alkyl and phenyl, as described herein. In some embodiments, the compound of Formula I is a compound of Formula I-B.

In some embodiments, the compound of Formula R-1 or a salt thereof is a compound of Formula R-1-A:

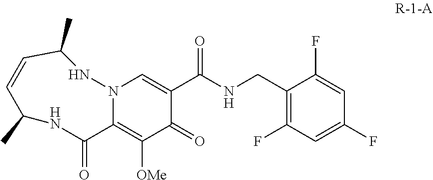 [SMILES: COc1c2n(cc(C(=O)NCc3c(F)cc(F)cc3F)c1=O)N[C@H](C)/C=C\[C@H](C)NC2=O]

or a salt thereof.

In some embodiments, the methylene reagent is a formaldehyde reagent or a dihalomethane. In some embodiments, suitable formaldehyde reagents include formaldehyde acetals (e.g., dimethoxymethane, diethoxymethane), formaldehyde solution, paraformaldehyde, and 1,3,5-trioxane. In some embodiments, suitable methylene reagents include dihalomethanes (e.g., dibromomethane, diiodomethane).

In some embodiments, the methylene reagent is selected from 1,3,5-trioxane, a formaldehyde acetal, formaldehyde, paraformaldehyde, or a dihalomethane. In some embodiments, the methylene reagent is a formaldehyde reagent. In some embodiments, the methylene reagent is selected from 1,3,5-trioxane, a formaldehyde acetal, formaldehyde, and paraformaldehyde. In some embodiments, the methylene reagent is formaldehyde (i.e., formalin). In some embodiments, the methylene reagent is 1,3,5-trioxane.

In some embodiments, the compound of Formula R-1 or a salt thereof is reacted with the eighth acid. In some embodiments, suitable eighth acids include carboxylic acids (e.g., oxalic acid, trichloroacetic acid, trifluoroacetic acid), sulfonic acids (e.g., methanesulfonic acid, benzenesulfonic acid, para-toluenesulfonic acid, camphorsulfonic acid), Lewis acids (e.g., boron trifluoride methyl etherate, boron trifluoride dibutyl etherate, boron trichloride, boron tribromide, aluminum chloride, aluminum bromide), inorganic acids (e.g., hydrochloric acid, sulfuric acid, phosphoric acid, hydrobromic acid), or a combination thereof.

In some embodiments, the eighth acid comprises a carboxylic acid, a sulfonic acid, a Lewis acid, an inorganic acid, or a combination thereof. In some embodiments, the eighth acid is a sulfonic acid. In some embodiments, the eight acid comprises methanesulfonic acid, benzenesulfonic acid, para-toluenesulfonic acid, camphorsulfonic acid, or a combination thereof. In some embodiments, the eighth acid is methanesulfonic acid.

In some embodiments, the eighth acid is an inorganic acid. In some embodiments, the eighth acid comprises hydrochloric acid, sulfuric acid, phosphoric acid, hydrobromic acid, or a combination thereof. In some embodiments, the eighth acid is phosphoric acid.

In some embodiments, the compound of Formula R-1 or a salt thereof is reacted with the fourteenth base. In some embodiments, suitable fourteenth bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN), etc.), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide, etc.), amides (e.g., lithium bis(trimethylsilyl)amide, potassium bis(trimethylsilyl)amide, lithium diisopropylamide), basic aromatic compounds (e.g., pyridine, 2,6-lutidine, imidazole), or a combination thereof.

In some embodiments, the fourteenth base comprises an amine, an amidine, a guanidine, a carboxylate, a carbonate, a metal hydride, a phosphate, a hydroxide, an alkoxide, an amide, a basic aromatic compound, or a combination thereof.

In some embodiments, the reacting of the compound of Formula R-1 or a salt thereof is performed in an eleventh solvent. In some embodiments, the eleventh solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the eleventh solvent comprises an ether. In some embodiments, the eleventh solvent comprises tetrahydrofuran, 2-methyltetrahydrofuran, tert-butyl methyl ether, 1,3-dioxolane, cyclopentyl methyl ether, 1,4-dioxane or a combination thereof. In some embodiments, the reacting of the compound of Formula R-1 or a salt thereof is performed in an eleventh solvent comprising tetrahydrofuran.

In some embodiments, the eleventh solvent comprises a hydrocarbon. In some embodiments, the eleventh solvent comprises toluene, methylcyclohexane, n-heptane, hexanes, limonene, or a combination thereof. In some embodiments, the reacting of the compound of Formula R-1 or a salt thereof is performed in an eleventh solvent comprising toluene.

In some embodiments, the reacting of the compound of Formula R-1 or a salt thereof is carried out in a temperature range of from about 20° C. to about 100° C. In some embodiments, the reacting of the compound of Formula R-1 or a salt thereof is carried out in a temperature range of from about 50° C. to about 90° C. In some embodiments, the reacting of the compound of Formula R-1 or a salt thereof is carried out in a temperature range of from about 50° C. to about 70° C. In some embodiments, the reacting of the compound of Formula R-1 or a salt thereof is carried out at a temperature of about 65° C. In some embodiments, the reacting of the compound of Formula R-1 or a salt thereof is carried out at a temperature of about 80° C.

In some embodiments, the process further comprises preparing the compound of Formula R-1 or a salt thereof by a process comprising:
reacting a compound of Formula R-2:

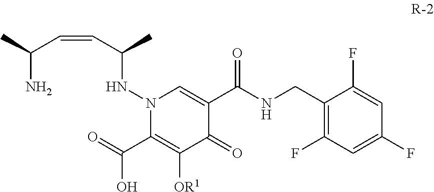

or a salt thereof, with:
   a coupling agent, and
   a fifteenth base,
to provide the compound of Formula R-1 or a salt thereof.

In some embodiments, the compound of Formula R-2 or a salt thereof is a compound of Formula R-2-A:

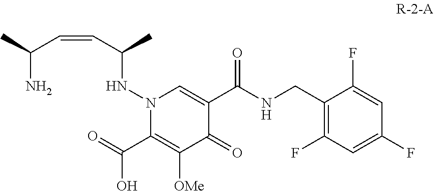 [SMILES: COc1c(C(=O)O)n(N[C@H](C)/C=C\[C@H](C)N)cc(C(=O)NCc2c(F)cc(F)cc2F)c1=O]

or a salt thereof

In some embodiments, suitable coupling agents include an acid chloride (e.g., thionyl chloride, oxalyl chloride, phosphorus(V) oxychloride), a mixed anhydride (e.g., with reagents such as acetic anhydride, pivaloyl chloride, diphenylphosphinic anhydride, propanephosphinic anhydride, boric acid, isobutyl chloroformate, methanesulfonyl chloride, p-toluenesulfonyl chloride), a carbonyl diimidazole, an activated ester, 4-(4,6-dimethoxy-1,3,5-triazin-2-yl)-4-methylmorpholinium chloride, 2-chloro-4,6-dimethoxy-1,3,5-triazine, cyanuric chloride, 1-ethyl-3-(3-dimethylaminopropyl)carbodiimide, benzotriazol-1-yloxytripyrrolidinophosphonium hexafluorophosphate, 1-[bis(dimethylamino)methylene]-1H-1,2,3-triazolo[4,5-b]pyridinium 3-oxide hexafluorophosphate, and other reagents known to promote the coupling of amines with carboxylic acids.

In some embodiments, suitable coupling agents include an acid chloride (e.g., thionyl chloride, oxalyl chloride, phosphorus(V) oxychloride), a mixed anhydride (e.g., with reagents such as acetic anhydride, pivaloyl chloride, diphenylphosphinic anhydride, propanephosphinic anhydride, boric acid, isobutyl chloroformate, methanesulfonyl chloride, p-toluenesulfonyl chloride), organophosphorus compounds (e.g., diphenylphosphinic chloride, diphenyl phosphoryl chloride, bis(2-oxo-3-oxazolidinyl)phosphinic chloride) a carbonyl diimidazole, an activated ester, 4-(4,6-dimethoxy-1,3,5-triazin-2-yl)-4-methylmorpholinium chloride, 2-chloro-4,6-dimethoxy-1,3,5-triazine, cyanuric chloride, 1-ethyl-3-(3-dimethylaminopropyl)carbodiimide, benzotriazol-1-yloxytripyrrolidinophosphonium hexafluorophosphate, 1-[bis(dimethylamino)methylene]-1H-1,2,3-triazolo[4,5-b]pyridinium 3-oxide hexafluorophosphate, bromo-tris-pyrrolidino-phosphonium hexafluorophosphate), and other reagents known to promote the coupling of amines with carboxylic acids.

In some embodiments, the coupling agent comprises 1-[Bis(dimethylamino)methylene]-1H-1,2,3-triazolo[4,5-b]pyridinium 3-oxid hexafluorophosphate (HATU), an acid chloride, an acid anhydride, a carbonyl diimidazole, an activated ester, or a combination thereof. In some embodiments, the coupling agent comprises 1-[Bis(dimethylamino)methylene]-1H-1,2,3-triazolo[4,5-b]pyridinium 3-oxid hexafluorophosphate (HATU), an acid chloride, an acid anhydride, a carbonyl diimidazole, an activated ester, or a combination thereof. In some embodiments, the coupling agent comprises thionyl chloride, oxalyl chloride, phosphorus(V) oxychloride, acetic anhydride, pivaloyl chloride, diphenylphosphinic anhydride, propanephosphinic anhydride, boric acid, isobutyl chloroformate, methanesulfonyl chloride, p-toluenesulfonyl chloride, a carbonyl diimidazole, an activated ester, 4-(4,6-dimethoxy-1,3,5-triazin-2-yl)-4-methylmorpholinium chloride, 2-chloro-4,6-dimethoxy-1,3,5-triazine, cyanuric chloride, 1-ethyl-3-(3-dimethylaminopropyl)carbodiimide, benzotriazol-1-yloxytripyrrolidinophosphonium hexafluorophosphate, HATU, or a combination thereof. In some embodiments, the coupling reagent is a peptide coupling reagent. In some embodiments, the coupling agent is HATU.

In some embodiments, the coupling agent comprises an organophosphorus compound. In some embodiments, the coupling agent comprises diphenylphosphinic chloride, diphenyl phosphoryl chloride, bis(2-oxo-3-oxazolidinyl)phosphinic chloride, or a combination thereof. In some embodiments, the coupling agent comprises diphenyl phosphoryl chloride.

In some embodiments, suitable fifteenth bases include amines (e.g., N-methyl morpholine, triethylamine, N,N-diisopropylethylamine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate, etc.), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), basic aromatic compounds (e.g., pyridine, 2,6-lutidine, imidazole, etc.), or a combination thereof.

In some embodiments, the fifteenth base comprises an amine, an amidine, a guanidine, a carboxylate, a carbonate, a metal hydride, a hydroxide, an alkoxide, a basic aromatic compound, or a combination thereof. In some embodiments, the fifteenth base comprises an amine. In some embodiments, the fifteenth base comprises N-methyl morpholine, triethylamine, N,N-diisopropylethylamine, or a combination thereof. In some embodiments, the fifteenth base is N,N-diisopropylethylamine.

In some embodiments, the reacting of the compound of Formula R-2 or a salt thereof is performed in a twenty-eighth solvent. In some embodiments, the twenty-eighth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the twenty-eighth solvent comprises a polar aprotic solvent. In some embodiments, the twenty-eighth solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula R-2 or a salt thereof is performed in a twenty-eighth solvent comprising N,N-dimethylacetamide.

In some embodiments, the reacting of the compound of Formula R-2 or a salt thereof is carried out in a temperature range of from about 0° C. to about 80° C. In some embodiments, the reacting of the compound of Formula R-2 or a salt thereof is carried out in a temperature range of from about 20° C. to about 30° C. In some embodiments, the reacting of the compound of Formula R-2 or a salt thereof is carried out in at a temperature of about 20° C.

In some embodiments, the process further comprises preparing the compound of Formula R-2 or a salt thereof by a process comprising:
reacting a compound of Formula A:

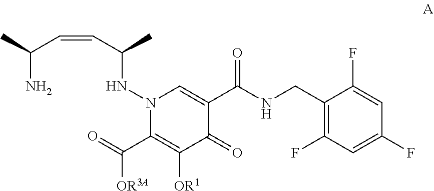

or a salt thereof, wherein R^{3A} is hydrogen or a hydroxyl protecting group, with:
   a reagent selected from a metal hydroxide, a metal halide, and a trialkylsilyl halide; to provide the compound of Formula R-2 or a salt thereof.

In some embodiments, R^{3A} is selected from hydrogen, C₁₋₄alkyl, and benzyl. In some embodiments, R^{3A} is selected from hydrogen, methyl, ethyl, isopropyl, and benzyl. In some embodiments, R^{3A} is methyl. In some embodiments, the compound of Formula A or a salt thereof is the compound of Formula A-1 or a salt thereof.

In some embodiments, the compound of Formula A is reacted with a metal hydroxide. In some embodiments, the metal hydroxide is an alkali metal hydroxide. In some embodiments, the metal hydroxide comprises sodium hydroxide, lithium hydroxide, potassium hydroxide, cesium hydroxide, or a combination thereof. In some embodiments, the metal hydroxide is sodium hydroxide.

In some embodiments, the compound of Formula A is reacted with a metal halide. In some embodiments, the metal halide comprises lithium chloride, magnesium bromide, zinc chloride, sodium iodide, or a combination thereof.

In some embodiments, the compound of Formula A is reacted with a trialkylsilyl halide. In some embodiments, the trialkylsilyl halide comprises trimethylsilyl chloride, trimethylsilyl iodide or a combination thereof.

In some embodiments, the reacting of the compound of Formula A or a salt thereof is performed in a twenty-ninth solvent. In some embodiments, the twenty-ninth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a polar aprotic solvent (e.g., N,N-dimethylformamide, N,N-dimethylacetamide, N-methyl-2-pyrrolidone, N-butyl pyrrolidone (NBP)), an alcohol (e.g., methanol, ethanol, 2-propanol, butanol, iso-amyl alcohol), water, or a combination thereof. In some embodiments, the twenty-ninth solvent comprises an alcohol. In some embodiments, the twenty-ninth solvent comprises methanol, ethanol, 2-propanol, butanol, iso-amyl alcohol, or a combination thereof. In some embodiments, the reacting of the compound of Formula A or a salt thereof is performed in a twenty-ninth solvent comprising methanol.

In some embodiments, the reacting of the compound of Formula A or a salt thereof is carried out in a temperature range of from about 0° C. to about 100° C. In some embodiments, the reacting of the compound of Formula A or a salt thereof is carried out in a temperature range of from about 20° C. to about 70° C. In some embodiments, the reacting of the compound of Formula A or a salt thereof is carried out in at a temperature of about 65° C.

### Alternative Process for Preparing the Compound of Formula R-1

In some embodiments of the process disclosed herein, the process comprises preparing the compound of Formula R-1 or a salt thereof by a process comprising:
reacting a compound of Formula A:

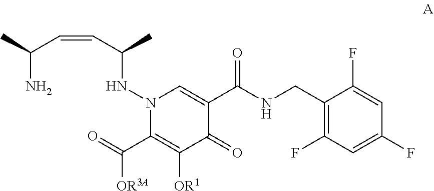

or a salt thereof, wherein R^{3A} is a hydroxyl protecting group, with:
   a sixteenth base;
to provide the compound of Formula R-1 or a salt thereof.

In some embodiments, R^{3A} is selected from C₁₋₄alkyl, C₁₋₄haloalkyl, and benzyl. In some embodiments, R^{3A} is selected from methyl, ethyl, isopropyl, trifluoroethyl, and benzyl. In some embodiments, R^{3A} is methyl. In some embodiments, the compound of Formula A or a salt thereof is the compound of Formula A-1 or a salt thereof.

In some embodiments, suitable sixteenth bases include amines (e.g., N-methyl morpholine, triethylamine, N,N-diisopropylethylamine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carboxylates (e.g., sodium acetate, potassium acetate, sodium propionate, etc.), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide), basic aromatic compounds (e.g., pyridine, 2,6-lutidine, imidazole, etc.), or a combination thereof.

In some embodiments, the sixteenth base comprises an amine, an amidine, a guanidine, a carboxylate, a carbonate, a metal hydride, a hydroxide, an alkoxide, a basic aromatic compound, or a combination thereof. In some embodiments, the sixteenth base is a guanidine. In some embodiments, the sixteenth base comprises 1,5,7-triazabicyclodec-5-ene, 1,1,3,3-tetramethylguanidine, or a combination thereof. In some embodiments, the sixteenth base is 1,5,7-triazabicyclodec-5-ene.

In some embodiments, the reacting of the compound of Formula A or a salt thereof is performed in a thirtieth solvent. In some embodiments, the thirtieth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the thirtieth solvent comprises an ether. In some embodiments, the thirtieth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, reacting of the compound of Formula A or a salt thereof is performed in a thirtieth solvent comprising 2-methyltetrahydrofuran.

In some embodiments, the reacting of the compound of Formula A or a salt thereof is carried out in a temperature range of from about 20° C. to about 110° C. In some embodiments, the reacting of the compound of Formula A or a salt thereof is carried out in a temperature range of from about 50° C. to about 70° C. In some embodiments, the reacting of the compound of Formula A or a salt thereof is carried out in at a temperature of about 60° C.

### Process for Preparing the Compound of Formula A

In some embodiments of the processes disclosed herein, the process comprises preparing the compound of Formula A or a salt thereof by a process comprising:
reacting a compound of Formula X-A:

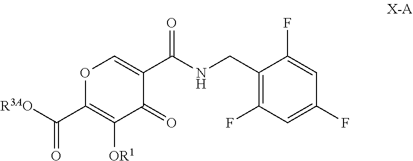

or a salt thereof, with:
   a compound of Formula C:

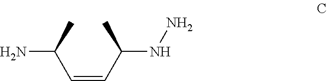

or a salt thereof, and
   a seventh base,
to provide the compound of Formula A or a salt thereof.

In some embodiments, the compound of Formula X-A or a salt thereof is the compound of Formula X-B or a salt thereof.

In some embodiments, the process comprises reacting the compound of Formula X-A with a salt of the compound of Formula C. In some embodiments, the salt of the compound of Formula C is a sulfonic acid salt. In some embodiments, the salt of the compound of Formula C is a methanesulfonic acid salt. In some embodiments, the salt of the compound of Formula C is a toluenesulfonic acid salt.

In some embodiments, suitable seventh bases include tertiary amines (e.g., N-methyl morpholine, N-methyl piperidine, 1,4-dimethylpiperazine, triethylamine, N,N-diisopropylethylamine, 1,4-diazabicyclo[2.2.2]octane (DABCO), quinuclidine), secondary amines (e.g., morpholine, piperidine, pyrrolidine, N,N-diisopropylamine), bis(triethylsilyl)amine (HMDS), anilines (e.g., N,N-dimethylaniline, 1,8-bis(dimethylamino)naphthalene (proton sponge)), pyridines (e.g., pyridine, 2,4,6-collidine, 2,6-lutidine), imidazole, amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carbonates (e.g., M(n)H(2-n)CO₃ such as sodium carbonate, potassium carbonate, cesium carbonate), phosphates (e.g., M(n)H(3-n)PO4 such as trisodium phosphate, disodium hydrogen phosphate, tripotassium phosphate, dipotassium hydrogen phosphate), hydroxides (e.g., sodium hydroxide, potassium hydroxide, cesium hydroxide), and alkoxides (e.g., sodium methoxide, sodium ethoxide, potassium tert-butoxide).

In some embodiments, the seventh base is selected from a tertiary amine, a secondary amine, an aniline, a pyridine, an imidazole, an amidine, a guanidine, a carbonate, a phosphate, a hydroxide, an alkoxide, or a combination thereof. In some embodiments, the seventh base is an amine. In some embodiments, the seventh base is a tertiary amine. In some embodiments, the seventh base comprises N-methyl morpholine, N-methyl piperidine, 1,4-dimethylpiperazine, triethylamine, N,N-diisopropylethylamine, 1,4-diazabicyclo[2.2.2]octane (DABCO), quinuclidine, or a combination thereof. In some embodiments, the seventh base is N-methyl morpholine.

In some embodiments, the reacting of the compound of Formula X-A or a salt thereof is performed in a fourteenth solvent. In some embodiments, the fourteenth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol), a halogenated solvent (e.g., dichloromethane, 1,2-dichloroethane, chlorobenzene, α,α,α-trifluorotoluene), or a combination thereof. In some embodiments, the fourteenth solvent comprises an alcohol. In some embodiments, the fourteenth solvent comprises methanol, ethanol, 2-propanol, butanol, iso-amyl alcohol, or a combination thereof. In some embodiments, the reacting of the compound of Formula X-A or a salt thereof is performed in a fourteenth solvent comprising methanol.

In some embodiments, the reacting of the compound of Formula X-A or a salt thereof is carried out in a temperature range of from about 20° C. to about 100° C. In some embodiments, the reacting of the compound of Formula X-A or a salt thereof is carried out in a temperature range of from about 40° C. to about 60° C. In some embodiments, the reacting of the compound of Formula X-A or a salt thereof is carried out in at a temperature of about 50° C.

In some embodiments, the process further comprises reacting the compound of Formula A with a fourth acid to provide a salt of the compound of Formula A.

In some embodiments, suitable fourth acids include trifluoroacetic acid, hydrochloric acid, hydrobromic acid, acetic acid, oxalic acid, orotic acid, succinic acid, malonic acid, adipic acid, fumaric acid, benzenesulfonic acid, benzoic acid, citric acid, 1,8-naphthalenedisulfonic acid, tartaric acid (L/D and common esters thereof, including dibenzoyl), p-toluenesulfonic acid, methanesulfonic acid, camphorsulfonic acid (S/R), maleic acid, xinafoic acid, and L-pyroglutamic acid.

In some embodiments, the fourth acid is selected from trifluoroacetic acid, hydrochloric acid, hydrobromic acid, acetic acid, oxalic acid, orotic acid, succinic acid, malonic acid, adipic acid, fumaric acid, benzenesulfonic acid, benzoic acid, citric acid, 1,8-naphthalenedisulfonic acid, tartaric acid or an ester thereof (L/D and esters thereof such as dibenzoyl), p-toluenesulfonic acid, methanesulfonic acid, camphorsulfonic acid (S/R), maleic acid, xinafoic acid, and L-pyroglutamic acid.

In some embodiments, the fourth acid is oxalic acid. In some embodiments, the reaction provides an oxalate salt of the compound of Formula A.

In some embodiments, the process further comprises preparing the compound of Formula C or a salt thereof by a process comprising:
deprotecting a compound of Formula D:

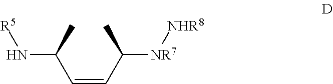

or a salt thereof, wherein:
   R⁵ is selected from hydrogen and R⁹, wherein R⁹ is an amine protecting group; and
   R⁷ and R⁸ are each independently an amine protecting group;
      to provide the compound of Formula C or a salt thereof.

In some embodiments, R⁵ is hydrogen. In some embodiments, R⁵ is R⁹. In some embodiments, R⁵ is an amine protecting group. In some embodiments, R⁵ is tert-butyloxycarbonyl.

In some embodiments, R⁷ is tert-butyloxycarbonyl. In some embodiments, R⁸ is tert-butyloxycarbonyl. In some embodiments, R⁷ and R⁸ are tert-butyloxycarbonyl.

In some embodiments, the compound of Formula D or a salt thereof is a compound of Formula D-2:

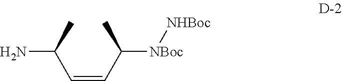 [SMILES: C[C@H](N)/C=C\[C@@H](C)N(NC(=O)OC(C)(C)C)C(=O)OC(C)(C)C]

or a salt thereof.

In some embodiments, deprotecting the compound of Formula D or a salt thereof comprises treating the compound of Formula D or a salt thereof with a fifth acid. In some embodiments, suitable fifth acids include sulfonic acids (e.g., p-toluenesulfonic acid, benzenesulfonic acid, camphorsulfonic acid, trifluoromethanesulfonic acid), sulfuric acid, sulfurous acid, carboxylic acids (e.g., trifluoroacetic acid, trichloroacetic acid, oxalic acid), hydrogen halides (e.g., hydrochloric acid, hydrobromic acid, hydroiodic acid), in situ generated hydrogen halides (e.g., by combination of acetyl chloride and methanol, trimethylsilyl chloride and methanol, thionyl chloride and methanol), phosphoric acid, and phosphorous acid.

In some embodiments, the fifth acid is selected from a sulfonic acid, sulfuric acid, sulfurous acid, a carboxylic acid, a hydrogen halide, phosphoric acid, phosphorous acid, or a combination thereof. In some embodiments, the fifth acid is a sulfonic acid. In some embodiments, the fifth acid is selected from p-toluenesulfonic acid, benzenesulfonic acid, camphorsulfonic acid, trifluoromethanesulfonic acid, or a combination thereof. In some embodiments, the fifth acid comprises methanesulfonic acid. In some embodiments, the fifth acid comprises p-toluenesulfonic acid.

In some embodiments, deprotecting the compound of Formula D or a salt thereof is performed in a fifteenth solvent. In some embodiments, the fifteenth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the fifteenth solvent comprises an alcohol. In some embodiments, the fifteenth solvent comprises methanol, ethanol, 2-propanol, butanol, glycerol, iso-amyl alcohol, or a combination thereof. In some embodiments, deprotecting the compound of Formula D or a salt thereof is performed in a fifteenth solvent comprising methanol. In some embodiments, deprotecting the compound of Formula D or a salt thereof is performed in a fifteenth solvent comprising ethanol.

In some embodiments, deprotecting the compound of Formula D or a salt thereof is carried out in a temperature range of from about 20° C. to about 100° C. In some embodiments, deprotecting the compound of Formula D or a salt thereof is carried out in a temperature range of from about 40° C. to about 60° C. In some embodiments, deprotecting the compound of Formula D or a salt thereof is carried out in a temperature range of from about 45° C. to about 55° C. In some embodiments, deprotecting the compound of Formula D or a salt thereof is carried out at a temperature of about 50° C.

In some embodiments, the process further comprises preparing the compound of Formula D or a salt thereof, wherein the compound of Formula D is a compound of Formula D-1:

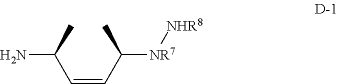

or a salt thereof, by a process comprising:
deprotecting a compound of Formula E:

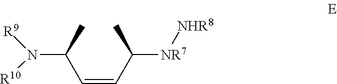

or a salt thereof, wherein:
R⁹ is an amine protecting group and R¹⁰ is hydrogen; or optionally:
R⁹ and R¹⁰ together with the nitrogen atom to which they are attached form a heterocyclic ring protecting group;
   to provide the compound of Formula D or a salt thereof.

In some embodiments, R⁷ and R⁸ are tert-butyloxycarbonyl.

In some embodiments, R⁹ and R¹⁰ together with the nitrogen atom to which they are attached form a heterocyclic ring protecting group.

In some embodiments, R⁹ and R¹⁰ together with the nitrogen atom to which they are attached form phthalimide. In some embodiments, R⁷ and R⁸ are tert-butyloxycarbonyl and R⁹ and R¹⁰ together with the nitrogen atom to which they are attached form phthalimide

In some embodiments, R⁹ is tert-butyloxycarbonyl.

In some embodiments, deprotecting the compound of Formula E or a salt thereof comprises treating the compound of Formula E or a salt thereof with a second deprotecting agent. In some embodiments, suitable second deprotecting agents include hydrazines (e.g., hydrazine, methylhydrazine, phenylhydrazine), amines (e.g., ethanolamine, 1,3-diaminopropane, methylamine, ethylamine, propylamine, hydroxylamine), alkoxides (e.g., potassium methoxide, lithium isopropoxide), thiols (e.g., decanethiol, sodium sulfide), and hydrolase enzymes (e.g., phthalyl amidase, dihydropyrimidinase, hydantoinase, imidase, lipase).

In some embodiments, deprotecting the compound of Formula E or a salt thereof comprises treating the compound of Formula E or a salt thereof with a second deprotecting agent. In some embodiments, suitable second deprotecting agents include hydrazines (e.g., hydrazine, methylhydrazine, phenylhydrazine), amines (e.g., ethanolamine, 1,3-diaminopropane, methylamine, ethylamine, propylamine, hydroxylamine), alkoxides (e.g., potassium methoxide, lithium isopropoxide), thiols (e.g., decanethiol, sodium sulfide), hydrolase enzymes (e.g., phthalyl amidase, dihydropyrimidinase, hydantoinase, imidase, lipase), and hydrides (e.g., sodium borohydride, sodium acetoxyborohydride, sodium cyanoborohydride, lithium borohydride, lithium aluminum hydride).

In some embodiments, the second deprotecting agent is selected from a hydrazine, an amine, an alkoxide, a thiol, a hydrolase enzyme, a hydride, or a combination thereof. In some embodiments, the second deprotecting agent is selected from a hydrazine, an amine, an alkoxide, a thiol, a hydrolase enzyme, or a combination thereof. In some embodiments, the second deprotecting agent is an amine. In some embodiments, the second deprotecting agent comprises ethanolamine, 1,3-diaminopropane, methylamine, ethylamine, propylamine, hydroxylamine, or a combination thereof. In some embodiments, the second deprotecting agent is ethylenediamine. In some embodiments, the second deprotecting agent is a hydride. In some embodiments, the second deprotecting agent comprises sodium borohydride, sodium acetoxyborohydride, sodium cyanoborohydride, lithium borohydride, lithium aluminum hydride, or a combination thereof. In some embodiments, the second deprotecting agent is sodium borohydride.

In some embodiments, the deprotecting the compound of Formula E or a salt thereof is performed further in the presence of a ninth acid. In some embodiments, suitable ninth acids include carboxylic acids (e.g., acetic acid, pivalic acid, formic acid) and strong acids (e.g., methanesulfonic acid, para-toluenesulfonic acid, hydrochloric acid). In some embodiments, the ninth acid comprises a carboxylic acid, a strong acid, or a combination thereof. In some embodiments, the deprotecting the compound of Formula E or a salt thereof is performed in absence of an acid.

In some embodiments, the deprotecting the compound of Formula E or a salt thereof is performed in a sixteenth solvent. In some embodiments, the second solvent comprises water, an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the sixteenth solvent comprises an alcohol. In some embodiments, the sixteenth solvent comprises methanol, ethanol, 2-propanol, butanol, glycerol, iso-amyl alcohol, or a combination thereof. In some embodiments, the deprotecting the compound of Formula E or a salt thereof is performed in a sixteenth solvent comprising methanol.

In some embodiments, the deprotecting the compound of Formula E or a salt thereof is carried out in a temperature range of from about 0° C. to about 100° C. In some embodiments, the deprotecting the compound of Formula E or a salt thereof is carried out in a temperature range of from about 40° C. to about 60° C. In some embodiments, the deprotecting the compound of Formula E or a salt thereof is carried out in a temperature of about 50° C.

### Alternative Process for Preparing the Compound of Formula A

In some embodiments of the processes disclosed herein, the process comprises preparing the compound of Formula A or a salt thereof by a process comprising:
reacting a compound of Formula Q-1:

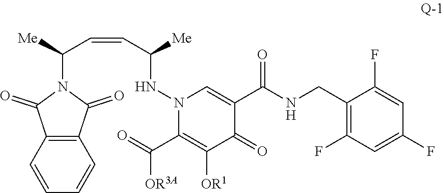

or a salt thereof,
   with a reagent selected from a hydrazine, an amine, an alkoxide, and a hydrolase enzyme; to provide the compound of Formula A or a salt thereof.

In some embodiments, the compound of Formula Q-1 or a salt thereof is a compound of Formula Q-1-A:

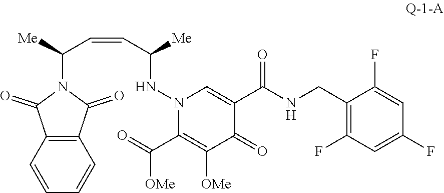 [SMILES: COC(=O)c1c(OC)c(=O)c(C(=O)NCc2c(F)cc(F)cc2F)cn1N[C@H](C)/C=C\[C@H](C)N1C(=O)c2ccccc2C1=O]

or a salt thereof.

In some embodiments, the process comprises reacting the compound of Formula Q-1 with a hydrazine. In some embodiments, the hydrazine is selected from hydrazine, methylhydrazine, phenylhydrazine, or a combination thereof. In some embodiments, the process comprises reacting the compound of Formula Q-1 with hydrazine. In some embodiments, the process comprises reacting the compound of Formula Q-1 with hydrazine hydrate.

In some embodiments, the process comprises reacting the compound of Formula Q-1 with an amine. In some embodiments, the amine is selected from ethanolamine, 1,3-diaminopropane, methylamine, ethylamine, propylamine, hydroxylamine, or a combination thereof.

In some embodiments, the process comprises reacting the compound of Formula Q-1 with an alkoxide. In some embodiments, the alkoxide is selected from potassium methoxide, lithium isopropoxide, or a combination thereof.

In some embodiments, the process comprises reacting the compound of Formula Q-1 with a hydrolase enzyme. In some embodiments, the hydrolase enzyme is selected from phthalyl amidase, dihydropyrimidinase, hydantoinase, imidase, lipase, or a combination thereof.

In some embodiments, the reacting of the compound of Formula Q-1 or a salt thereof is performed in a thirty-first solvent. In some embodiments, the thirty-first solvent comprises water, an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a hydrocarbon (e.g., toluene, methylcyclohexane, n-heptane, hexanes, limonene), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the thirty-first solvent comprises a hydrocarbon. In some embodiments, the thirty-first solvent comprises toluene, methylcyclohexane, n-heptane, hexanes, limonene, or a combination thereof. In some embodiments, the reacting of the compound of Formula Q-1 or a salt thereof is performed in a thirty-first solvent comprising toluene.

In some embodiments, the reacting of the compound of Formula Q-1 or a salt thereof is carried out in a temperature range of from about 0° C. to about 100° C. In some embodiments, the reacting of the compound of Formula Q-1 or a salt thereof is carried out in a temperature range of from about 75° C. to about 95° C. In some embodiments, the reacting of the compound of Formula Q-1 or a salt thereof is carried out in a temperature of about 100° C.

In some embodiments, the process further comprises preparing the compound of Formula Q-1 or a salt thereof by a process comprising:
reacting a compound of Formula Q-2:

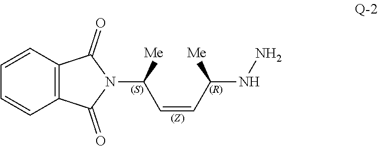

or a salt thereof, with:
a compound of Formula X-A:

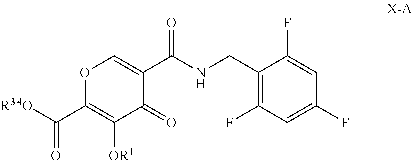

or a salt thereof, and
a thirteenth base;
   to provide the compound of Formula Q-1 or a salt thereof. In some embodiments, the compound of Formula X-A or a salt thereof is a compound of Formula X-B or a salt thereof.

In some embodiments, suitable thirteenth bases include tertiary amines (e.g., N-methyl morpholine, N-methyl piperidine, 1,4-dimethylpiperazine, triethylamine, N,N-diisopropylethylamine, 1,4-diazabicyclo[2.2.2]octane (DABCO), quinuclidine), secondary amines (e.g., morpholine, piperidine, pyrrolidine, N,N-diisopropylamine), bis(triethylsilyl)amine (HMDS)), anilines (e.g., N,N-dimethylaniline, 1,8-bis(dimethylamino)naphthalene (proton sponge)), pyridines (e.g., pyridine, 2,4,6-collidine, 2,6-lutidine), imidazole, amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carbonates (e.g., M(n)H(2-n)CO₃ such as sodium carbonate, potassium carbonate, cesium carbonate), phosphates (e.g., M(n)H(3-n)PO4 such as trisodium phosphate, disodium hydrogen phosphate, tripotassium phosphate, dipotassium hydrogen phosphate), hydroxides (e.g., sodium hydroxide, potassium hydroxide, cesium hydroxide), and alkoxides (e.g., sodium methoxide, sodium ethoxide, potassium tert-butoxide).

In some embodiments, the thirteenth base is selected from a tertiary amine, a secondary amine, an aniline, a pyridine, an imidazole, an amidine, a guanidine, a carbonate, a phosphate, a hydroxide, an alkoxide, or a combination thereof. In some embodiments, the seventh base is an amine. In some embodiments, the seventh base is a tertiary amine. In some embodiments, the thirteenth base comprises N-methyl morpholine, N-methyl piperidine, 1,4-dimethylpiperazine, triethylamine, N,N-diisopropylethylamine, 1,4-diazabicyclo[2.2.2]octane (DABCO), quinuclidine, or a combination thereof. In some embodiments, the thirteenth base is N-methyl morpholine.

In some embodiments, the reacting of the compound of Formula Q-2 or a salt thereof is performed in a thirty-second solvent. In some embodiments, the thirty-second solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol), a halogenated solvent (e.g., dichloromethane, 1,2-dichloroethane, chlorobenzene, α,α,α-trifluorotoluene), or a combination thereof. In some embodiments, the thirty-second comprises an alcohol. In some embodiments, the thirty-second solvent comprises methanol, ethanol, 2-propanol, butanol, iso-amyl alcohol, or a combination thereof. In some embodiments, the reacting of the compound of Formula Q-2 or a salt thereof is performed in a thirty-second solvent comprising methanol.

In some embodiments, the reacting of the compound of Formula Q-2 or a salt thereof is carried out in a temperature range of from about 20° C. to about 100° C. In some embodiments, the reacting of the compound of Formula Q-2 or a salt thereof is carried out in a temperature range of from about 40° C. to about 60° C. In some embodiments, the reacting of the compound of Formula Q-2 or a salt thereof is carried out in at a temperature of about 50° C.

### Process for Preparing the Compound of Formula E

In some embodiments of the processes disclosed herein, the process comprises preparing the compound of Formula E or a salt thereof by a process comprising:
reacting a compound of Formula F:

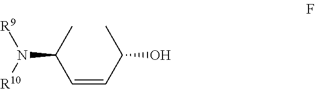

or a salt thereof, with:
a phosphine reagent,
an azodicarboxylate, and
a compound of formula R⁷—NH—NH—R⁸;
   to provide the compound of Formula E or a salt thereof.

In some embodiments of the compound of Formula F, R⁹ and R¹⁰ together with the nitrogen atom to which they are attached form phthalimide.

In some embodiments, the compound of Formula R⁷—NH—NH—R⁸ is

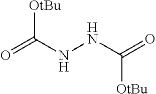 [SMILES: CC(C)(C)OC(=O)NNC(=O)OC(C)(C)C]

In some embodiments, suitable phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the phosphine reagent is selected from a trialkyl phosphine, a triaryl phosphine, (triphenylphosphoranylidene) acetonitrile, and a phosphorane. In some embodiments, the phosphine reagent is a triarylphosphine. In some embodiments, the phosphine reagent comprises triphenylphosphine, tris (2-pyridyl) phosphine, tris-(4-methoxyphenyl) phosphine, or a combination thereof. In some embodiments, the phosphine reagent is triphenylphosphine.

In some embodiments, the azodicarboxylate is selected from di-tert-butyl azodicarboxylate, diisopropyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the azodicarboxylate is di-tert-butyl azodicarboxylate.

In some embodiments, the reacting of the compound of Formula F or a salt thereof is performed in a seventeenth solvent. In some embodiments, the seventeenth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the seventeenth solvent comprises an ether. In some embodiments, the seventeenth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula F or a salt thereof is performed in a seventeenth solvent comprising 2-methyltetrahydrofuran. In some embodiments, the seventeenth solvent comprises an ester. In some embodiments, the seventeenth solvent comprises isopropyl acetate.

In some embodiments, the reacting of the compound of Formula F or a salt thereof is carried out in a temperature range of from about −20° C. to about 50° C. In some embodiments, the reacting of the compound of Formula F or a salt thereof is carried out in a temperature range of from about 0° C. to about 22° C.

In some embodiments, the process further comprises preparing the compound of Formula F or a salt thereof by a process comprising:
reacting a compound of Formula G:

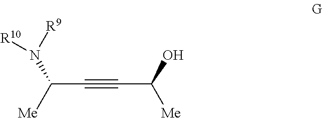

or a salt thereof, with:
a third catalyst, and
a hydrogen source;
   to provide the compound of Formula F or a salt thereof.

In some embodiments, the process comprises preparing the compound of Formula F or a salt thereof by a process comprising reacting a compound of Formula G or a salt thereof, with a third catalyst, and hydrogen; to provide the compound of Formula F or a salt thereof.

In some embodiments of the compound of Formula G, R⁹ and R¹⁰ together with the nitrogen atom to which they are attached form phthalimide.

In some embodiments, suitable third catalysts include palladium catalysts (e.g., palladium, 5 wt % on carbon, palladium, 10 wt % on carbon, palladium, 1 wt % on carbon, palladium, 5 wt % on calcium carbonate, palladium, 5 wt % on aluminum oxide, palladium hydroxide, palladium acetate, palladium acetate/manganese, palladium chloride, palladium trifluoroacetate, palladium propionate, palladium pivalate, tris(dibenzylideneacetone) dipalladium, palladium bromide, palladium (II) bis(acetylacetonate), bis(benzonitrile)palladium dichloride, 20 wt % on carbon, lead poisoned palladium, with, e.g., barium sulfate or calcium carbonate), bimetallic catalysts (e.g., palladium, 4 wt % and platinum, 1 wt % on carbon, palladium, 9 wt % and copper, 1 wt % on carbon, palladium, 4 wt % and copper, 1 wt % on carbon), and platinum catalysts (e.g., platinum on carbon, platinum oxide).

In some embodiments, suitable third catalysts include palladium catalysts (e.g., palladium, 5 wt % on carbon, palladium, 10 wt % on carbon, palladium, 1 wt % on carbon, palladium, 5 wt % on calcium carbonate, palladium, 5 wt % on aluminum oxide, palladium hydroxide, 20 wt % on carbon, lead poisoned palladium), bimetallic catalysts (e.g., palladium, 4 wt % and platinum, 1 wt % on carbon, palladium, 9 wt % and copper, 1 wt % on carbon, palladium, 4 wt % and copper, 1 wt % on carbon), and platinum catalysts (e.g., platinum on carbon, platinum oxide).

In some embodiments, the third catalyst is selected from a palladium catalyst, a platinum catalyst, and a bimetallic catalyst, wherein the bimetallic catalyst comprises two metals selected from palladium, platinum, and copper.

In some embodiments, the third catalyst is a palladium catalyst. In some embodiments, the third catalyst comprises palladium on carbon, palladium on calcium carbonate, or palladium on aluminum oxide. In some embodiments, the third catalyst is selected from 5 wt % palladium on carbon, 10 wt % palladium on carbon, 1 wt % palladium on carbon, 5 wt % palladium on calcium carbonate, 5 wt % palladium on aluminum oxide, 20 wt % palladium on carbon, and lead poisoned palladium.

In some embodiments, the third catalyst is a Lindlar catalyst. In some embodiments, the third catalyst comprises 5 wt. % palladium on lead-poisoned calcium carbonate.

In some embodiments, a molar ratio of the compound of Formula G or a salt thereof to the third catalyst is from about 1:0.001 to about 1:0.05. In some embodiments, a molar ratio of the compound of Formula G or a salt thereof to the third catalyst is from about 1:0.001 to about 1:0.005. In some embodiments, a molar ratio of the compound of Formula G or a salt thereof to the third catalyst is about 1:0.002.

In some embodiments, the hydrogen source comprises hydrogen, formic acid, water, or a combination thereof. In some embodiments, the hydrogen source comprises hydrogen, a mixture of formic acid and potassium carbonate, water, or a combination thereof. In some embodiments, the hydrogen source is hydrogen.

In some embodiments, the reacting of the compound of Formula G or a salt thereof is carried out in the presence of a second additive. In some embodiments, suitable second additives include quinoline, pyridine, triethylamine, carbonates (e.g., lithium carbonate, sodium carbonate, cesium carbonate, potassium carbonate), bicarbonates (e.g., lithium bicarbonate, sodium bicarbonate, cesium bicarbonate, potassium bicarbonate), phosphates (e.g., trisodium phosphate, disodium hydrogen phosphate, tripotassium phosphate, dipotassium hydrogen phosphate), 1,8-diazabicyclo[5.4.0]undec-7-ene, formates (e.g., potassium formate, sodium formate, ammonium formate), acetates (e.g., potassium acetate, sodium acetate, cesium acetate), alkoxides (e.g., potassium tert-butoxide, sodium tert-butoxide), halides (e.g., potassium iodide, lithium chloride, sodium chloride, calcium chloride), and hydroxides (e.g., potassium hydroxide). In some embodiments, the second additive comprises quinoline, pyridine, triethylamine, a carbonate, a bicarbonate, a phosphate, 1,8-diazabicyclo[5.4.0]undec-7-ene, potassium carbonate, potassium formate, sodium formate, ammonium formate, potassium hydroxide, potassium acetate, sodium acetate, cesium acetate, potassium tert-butoxide, sodium tert-butoxide, potassium iodide, lithium chloride, sodium chloride, calcium chloride, or a combination thereof.

In some embodiments, the reacting of the compound of Formula G or a salt thereof is carried out at a pressure of about 1 psi to about 100 psi. In some embodiments, the reacting of the compound of Formula G or a salt thereof is carried out at a pressure of about 25 psi to about 75 psi. In some embodiments, the reacting of the compound of Formula G or a salt thereof is carried out at a pressure of about 50 psi.

In some embodiments, the reacting of the compound of Formula G or a salt thereof is performed in an eighteenth solvent. In some embodiments, the eighteenth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a glycol (e.g., polyethylene glycol 400), a halogenated solvent (e.g., dichloromethane, 1,2-dichloroethane, chlorobenzene, α,α,α-trifluorotoluene), or a combination thereof. In some embodiments, the eighteenth solvent comprises a polar aprotic solvent. In some embodiments, the eighteenth solvent comprises N,N-dimethylformamide, N,N-dimethylacetamide, N-methyl-2-pyrrolidone, N-butyl pyrrolidone (NBP), or a combination thereof. In some embodiments, the reacting of the reacting of the compound of Formula G or a salt thereof is performed in an eighteenth solvent comprising N,N-dimethylformamide.

In some embodiments, the reacting of the compound of Formula G or a salt thereof is performed in an eighteenth solvent. In some embodiments, the eighteenth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene, anisole), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol, 1-butanol), a glycol (e.g., polyethylene glycol 400), a halogenated solvent (e.g., dichloromethane, 1,2-dichloroethane, chlorobenzene, α,α,α-trifluorotoluene), or a combination thereof.

In some embodiments, the reacting of the compound of Formula G or a salt thereof is carried out in a temperature range of from about 0° C. to about 100° C. In some embodiments, the reacting of the compound of Formula G or a salt thereof is carried out in a temperature range of from about 10° C. to about 30° C. In some embodiments, the reacting of the compound of Formula G or a salt thereof is carried out at a temperature of about 20° C.

In some embodiments, the process further comprises preparing the compound of Formula G or a salt thereof by a process comprising:
reacting a compound of Formula H:

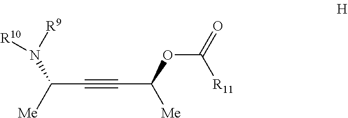

or a salt thereof, wherein R¹¹ is selected from C₁₋₂₀alkyl, phenyl, and —CH₂NH₂;
with a sixth acid or an eighth base,
   to provide the compound of Formula G or a salt thereof.

In some embodiments, R¹¹ is selected from methyl, ethyl, isopropyl, pentyl, heptyl, nonyl, pentadecyl, phenyl, and —CH₂NH₂. In some embodiments, R¹¹ is C₁₋₂₀alkyl. In some embodiments, R¹¹ is selected from methyl, ethyl, isopropyl, pentyl, heptyl, nonyl, and pentadecyl. In some embodiments, R¹¹ is methyl. In some embodiments, R¹¹ is phenyl. In some embodiments, R¹¹ is —CH₂NH₂.

In some embodiments, the compound of Formula H or a salt thereof is a compound of Formula H-2:

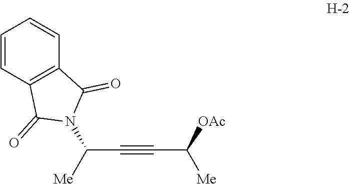 [SMILES: CC(=O)O[C@@H](C)C#C[C@H](C)N1C(=O)c2ccccc2C1=O]

or a salt thereof.

In some embodiments, the compound of Formula H or a salt thereof is reacted with the sixth acid. In some embodiments, suitable sixth acids include mineral acids (e.g., concentrated hydrochloric acid, hydrobromic acid, hydroiodic acid, sulfuric acid), trifluoroacetic acid, and sulfonic acids (e.g., methanesulfonic acid, p-toluenesulfonic acid, benzene sulfonic acid, camphor sulfonic acid).

In some embodiments, the sixth acid is selected from a mineral acid, trifluoroacetic acid, a sulfonic acid, or a combination thereof. In some embodiments, the sixth acid is a sulfonic acid. In some embodiments, the sixth acid comprises methanesulfonic acid, p-toluenesulfonic acid, benzene sulfonic acid, camphor sulfonic acid, or a combination thereof. In some embodiments, the sixth acid is p-toluenesulfonic acid.

In some embodiments, the compound of Formula H or a salt thereof is reacted with the eighth base. In some embodiments, suitable eighth bases include carbonates (e.g., lithium carbonate, sodium carbonate, cesium carbonate, potassium carbonate), bicarbonates (e.g., lithium bicarbonate, sodium bicarbonate, cesium bicarbonate, potassium bicarbonate), metal alkoxides (e.g., sodium methoxide, sodium ethoxide, sodium tert-butoxide, potassium methoxide, potassium ethoxide, potassium tert-butoxide), and amines (e.g., methylamine, diethylamine, isopropylamine, diisopropylethylamine, triethylamine, diisopropylethylamine, 1,8-diazabicyclo[5.4.0]undec-7-ene, 1,4-diazabicyclo[2.2.2]octane, Bis(trimethylsilyl)amine.

In some embodiments, the compound of Formula H or a salt thereof is reacted with the eighth base. In some embodiments, suitable eighth bases include carbonates (e.g., lithium carbonate, sodium carbonate, cesium carbonate, potassium carbonate), bicarbonates (e.g., lithium bicarbonate, sodium bicarbonate, cesium bicarbonate, potassium bicarbonate), metal alkoxides (e.g., sodium methoxide, sodium ethoxide, sodium tert-butoxide, potassium methoxide, potassium ethoxide, potassium tert-butoxide), and amines (e.g., methylamine, diethylamine, isopropylamine, diisopropylethylamine, triethylamine, diisopropylethylamine, 1,8-diazabicyclo[5.4.0]undec-7-ene, 1,4-diazabicyclo[2.2.2]octane, Bis(trimethylsilyl)amine, 1,1,3,3-tetramethylguanidine, triazabicyclodecene (TBD)).

In some embodiments, the eighth base is selected from a guanidine, a carbonate, a bicarbonate, a metal alkoxide, an amine, or a combination thereof.

In some embodiments, the reacting of the compound of Formula H or a salt thereof is performed in a thirty-fourth solvent. In some embodiments, the thirty-fourth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the thirty-fourth solvent comprises an alcohol. In some embodiments, the thirty-fourth solvent comprises methanol, ethanol, 2-propanol, butanol, iso-amyl alcohol, or a combination thereof. In some embodiments, the reacting of the compound of Formula H or a salt thereof is performed in a thirty-fourth solvent comprising methanol.

In some embodiments, the reacting of the compound of Formula H or a salt thereof is carried out in a temperature range of from about 0° C. to about 70° C. In some embodiments, the reacting of the compound of Formula H or a salt thereof is carried out in a temperature range of from about 10° C. to about 30° C. In some embodiments, the reacting of the compound of Formula H or a salt thereof is carried out in at a temperature of about 20° C.

### Alternative Process for Preparing the Compound of Formula F

In some embodiments of the processes disclosed herein, the process further comprises preparing the compound of Formula F or a salt thereof by a process comprising:
reacting a compound of Formula W:

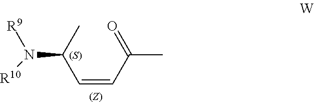

or a salt thereof, with:
a sixth catalyst, and
a reducing agent;
   to provide the compound of Formula F or a salt thereof.

In some embodiments of the compound of Formula W or a salt thereof, R⁹ and R¹⁰ together with the nitrogen atom to which they are attached form a heterocyclic ring protecting group.

In some embodiments, the compound of Formula W or a salt thereof is a compound of Formula W-1:

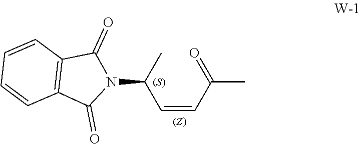

or a salt thereof.

In some embodiments of the compound of Formula F or a salt thereof, R⁹ and R¹⁰ together with the nitrogen atom to which they are attached form a heterocyclic ring protecting group.

In some embodiments, the compound of Formula F or a salt thereof is a compound of Formula F-2:

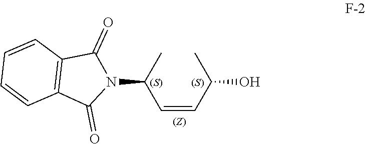

or a salt thereof.

In some embodiments, suitable sixth catalysts include a Corey-Bakshi-Shibata (CBS) catalyst and a transition metal catalyst (e.g., a ruthenium catalyst). In some embodiments, the sixth catalyst is selected from (R)-1-Methyl-3,3-diphenylhexahydropyrrolo[1,2-c][1,3,2]oxazaborole), (R)-(+)-2-methyl-CBS-oxazaborolidine (R)-nBu-CBS, any other derivative of CBS, and RuCl [(R,R)-TSDPEN](mesitylene). In some embodiments, the sixth catalyst is selected from (R)-(+)-2-methyl-CBS-oxazaborolidine and RuCl [(R,R)-TSDPEN](mesitylene). In some embodiments, the sixth catalyst is a CBS catalyst. In some embodiments, the sixth catalyst is selected from (R)-(+)-2-methyl-CBS-oxazaborolidine, (R)-nBu-CBS, and any other derivative of CBS. In some embodiments, the sixth catalyst is (R)-(+)-2-methyl-CBS-oxazaborolidine. In some embodiments, the sixth catalyst is (R)-1-Methyl-3,3-diphenylhexahydropyrrolo[1,2-c][1,3,2]oxazaborole.

In some embodiments, suitable reducing agents include formic acid/triethylamine, NaBH₄, LiBH₄, CeCl₃·7H₂O with NaBH₄, LiAlH₄, MgBu₂ with HBpin, Red-Al, LiAlH(OtBu)₃, BH₃·SMe₂, catecholborane, or combinations thereof. In some embodiments, the reducing agent comprises formic acid and triethylamine, NaBH₄, LiBH₄, CeCl₃·7H₂O with NaBH₄, LiAlH₄, MgBu₂ with HBpin, Red-Al, LiAlH(OtBu)₃, BH₃·SMe₂, catecholborane, or a combination thereof. In some embodiments, the reducing agent comprises catecholborane.

In some embodiments, the reacting of the compound of Formula W or a salt thereof is performed in a forty-third solvent. In some embodiments, the forty-third solvent comprises an ether (e.g., tetrahydrofuran, 2-methyltetrahydrofuran, tert-butyl methyl ether, 1,3-dioxolane, cyclopentyl methyl ether, 1,4-dioxane), a hydrocarbon (e.g., toluene, methylcyclohexane, n-heptane, hexanes, limonene), a chlorinated solvent (e.g., dichloromethane, CHCl₃, CCl₄, chlorobenzene), or a combination thereof. In some embodiments, the forty-third solvent comprises a chlorinated solvent. In some embodiments, the forty-third solvent comprises dichloromethane, CHCl₃, CCl₄, chlorobenzene, or a combination thereof. In some embodiments, the reacting of the compound of Formula W or a salt thereof is performed in a forty-third solvent comprising dichloromethane.

In some embodiments, the reacting of the compound of Formula W or a salt thereof is carried out in a temperature range of from about −78° C. to about 30° C. In some embodiments, the reacting of the compound of Formula W or a salt thereof is carried out in a temperature range of from about −30° C. to about 10° C. In some embodiments, the reacting of the compound of Formula W or a salt thereof is carried out at a temperature of about −10° C.

In some embodiments of the processes disclosed herein, the process further comprises preparing the compound of Formula W or a salt thereof by a process comprising:
reacting a compound of Formula S:

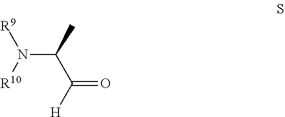

or a salt thereof, with:
a compound of Formula T:

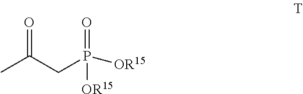

or a salt thereof,
   wherein R¹⁵ is selected from phenyl and C₁₋₄haloalkyl, wherein the phenyl is optionally substituted with 1-4 substituents selected from C₁₋₄alkyl, halo, C₁₋₄ haloalkyl, and cyano;
a seventeenth base, and
a fourth additive;
   to provide the compound of Formula W or a salt thereof.

In some embodiments of the compound of Formula W or a salt thereof, R⁹ and R¹⁰ together with the nitrogen atom to which they are attached form a heterocyclic ring protecting group. In some embodiments, the compound of Formula W or a salt thereof is a compound of Formula W-1 or a salt thereof.

In some embodiments of the compound of Formula S or a salt thereof, R⁹ and R¹⁰ together with the nitrogen atom to which they are attached form a heterocyclic ring protecting group.

In some embodiments, the compound of Formula S or a salt thereof is a compound of Formula S-1:

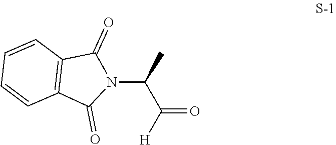

or a salt thereof.

In some embodiments, R¹⁵ is selected from trifluoroethyl, hexafluoroisopropyl, trichloroethyl, phenyl, chlorophenyl (e.g., 4-chlorophenyl), fluorophenyl (e.g., 2-fluorophenyl), trifluoromethylphenyl (e.g., 2-trifluoromethylphenyl), cyanophenyl (e.g., 3-cyanophenyl), and methylphenyl (e.g., 2-methylphenyl). In some embodiments, R¹⁵ is selected from phenyl and C₁₋₄haloalkyl, wherein the phenyl is optionally substituted with halo. In some embodiments, R¹⁵ is selected from trifluoroethyl, trichloroethyl, phenyl, and chlorophenyl (e.g., 4-chlorophenyl). In some embodiments, R¹⁵ is phenyl.

In some embodiments, suitable seventeenth bases include amides (e.g., lithium, sodium, or potassium hexamethyl disilazide), amine bases (e.g., N-methyl morpholine, triethylamine, diisopropylethylamine), amidines (e.g., 1,8-diazabicyclo[5.4.0]undec-7-ene (DBU), 1,5-diazabicyclo[4.3.0]non-5-ene (DBN)), guanidines (e.g., triazabicyclodecene (TBD), 1,1,3,3-tetramethylguanidine), carbonates (e.g., sodium carbonate, potassium carbonate, cesium carbonate), metal hydrides (e.g., lithium hydride, sodium hydride, potassium hydride), phosphates (e.g., trisodium phosphate, tripotassium phosphate), alkoxides (e.g., sodium methoxide, sodium ethoxide, potassium tert-butoxide), basic aromatic compounds (e.g., pyridine, 2,6-lutidine, imidazole), or combinations thereof.

In some embodiments, the seventeenth base is selected from an amide, an amine base, an amidine, a guanidine, a carbonate, a metal hydride, phosphate, an alkoxide, a basic aromatic compound, and combinations thereof. In some embodiments, the seventeenth base is an amide. In some embodiments, the seventeenth base is potassium hexamethyl disilazide.

In some embodiments, suitable fourth additives include ethers (e.g., 18-crown-6, bis(2-methoxyethyl) ether), metal salts (e.g., lithium chloride, magnesium chloride, zinc bromide), or combinations thereof. In some embodiments, the fourth additive is selected from an ether, a metal salt, or a combination thereof. In some embodiments, the fourth additive is selected from an ether, a metal halide, or a combination thereof. In some embodiments, the fourth additive is an ether. In some embodiments, the fourth additive is 18-crown-6.

In some embodiments, the reacting of the compound of Formula S or a salt thereof is performed in a forty-fourth solvent. In some embodiments, the forty-fourth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), a halogenated solvent (e.g., dichloromethane, 1,2-dichloroethane, chlorobenzene, α,α,α trifluorotoluene), water, or a combination thereof. In some embodiments, the forty-fourth solvent comprises an ether. In some embodiments, the forty-fourth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the reacting of the compound of Formula S or a salt thereof is performed in a forty-fourth solvent comprising tetrahydrofuran.

In some embodiments, the reacting of the compound of Formula S or a salt thereof is carried out in a temperature range of from about −100° C. to about 25° C. In some embodiments, the reacting of the compound of Formula S or a salt thereof is carried out in a temperature range of from about −80° C. to about −50° C. In some embodiments, the reacting of the compound of Formula S or a salt thereof is carried out at a temperature of about −60° C.

### Second Alternative Process for Preparing the Compound of Formula F

In some embodiments of the processes disclosed herein, the process comprises preparing the compound of Formula F or a salt thereof by a process comprising:
reacting a compound of Formula W:

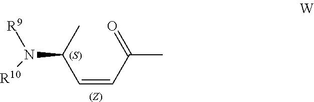

or a salt thereof, with:
a second enzyme in the presence of a second buffer;
   to provide the compound of Formula F or a salt thereof.

In some embodiments of the compound of Formula F or a salt thereof, R⁹ and R¹⁰ together with the nitrogen atom to which they are attached form a heterocyclic ring protecting group. In some embodiments, the compound of Formula F or a salt thereof is a compound of Formula F-2 or a salt thereof.

In some embodiments of the compound of Formula W or a salt thereof, R⁹ and R¹⁰ together with the nitrogen atom to which they are attached form a heterocyclic ring protecting group. In some embodiments, the compound of Formula W or a salt thereof is a compound of Formula W-1 or a salt thereof.

In some embodiments, suitable second enzymes include ketoreductases, alcohol oxidase, and P450. In some embodiments, the second enzyme is selected from a ketoreductase, alcohol oxidase, or P450. In some embodiments, the second enzyme is a ketoreductase. In some embodiments, the second enzyme is KRED NADH 101 (Codexis®).

In some embodiments, suitable second buffers include citrate buffers, phosphate buffers, tris(hydroxymethyl)aminomethane (Tris), and (4-(2-hydroxyethyl)-1-piperazineethanesulfonic acid) (HEPES). In some embodiments, the second buffer is selected from a citrate buffer, a phosphate buffer, tris(hydroxymethyl)aminomethane, and (4-(2-hydroxyethyl)-1-piperazineethanesulfonic acid) (HEPES). In some embodiments, the second buffer is a phosphate buffer. In some embodiments, the second buffer is a sodium phosphate buffer.

In some embodiments, the reacting of the compound of Formula W or a salt thereof is performed in a cosolvent. In some embodiments, the cosolvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the cosolvent comprises a polar protic solvent. In some embodiments, the cosolvent comprises N,N-dimethylformamide, N,N-dimethylacetamide, N-methyl-2-pyrrolidone, N-butyl pyrrolidone (NBP), dimethyl sulfoxide, or a combination thereof. In some embodiments, the reacting of the compound of Formula W or a salt thereof is performed in a cosolvent comprising dimethylsulfoxide.

In some embodiments, the reacting of the compound of Formula W or a salt thereof is carried out in a temperature range of from about 5° C. to about 100° C. In some embodiments, the reacting of the compound of Formula W or a salt thereof is carried out in a temperature range of from about 10° C. to about 30° C. In some embodiments, the reacting of the compound of Formula W or a salt thereof is carried out at a temperature of about 20° C.

In some embodiments, the process further comprises preparing the compound of Formula W or a salt thereof according to a process described herein.

### Third Alternative Process for Preparing the Compound of Formula F

In some embodiments of the processes disclosed herein, the compound of Formula F or a salt thereof is prepared by a process comprising:
reacting a compound of Formula G:

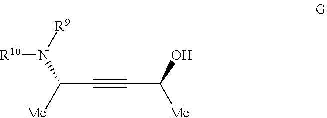

or a salt thereof, with:
a third reducing agent, and
an activator;
   to provide the compound of Formula F or a salt thereof.

In some embodiments, the compound of Formula F or a salt thereof is a compound of Formula F-1:

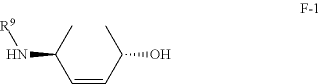

or a salt thereof. In some embodiments of the compound of Formula F-1, R⁹ is tert-butyloxycarbonyl.

In some embodiments, the compound of Formula G or a salt thereof is a compound of Formula G-1:

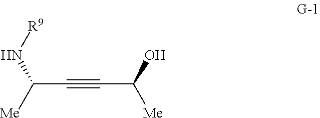

or a salt thereof. In some embodiments of the compound of Formula G-1, R⁹ is tert-butyloxycarbonyl.

In some embodiments of the compound of Formula F or a salt thereof, R⁹ and R¹⁰ together with the nitrogen atom to which they are attached form a heterocyclic ring protecting group. In some embodiments of the compound of Formula F or a salt thereof, R⁹ and R¹⁰ together with the nitrogen atom to which they are attached form phthalimide or saccharin. In some embodiments, the compound of Formula F or a salt thereof is a compound of Formula F-2 or a salt thereof.

In some embodiments of the compound of Formula G or a salt thereof, R⁹ and R¹⁰ together with the nitrogen atom to which they are attached form a heterocyclic ring protecting group. In some embodiments of the compound of Formula G or a salt thereof, R⁹ and R¹⁰ together with the nitrogen atom to which they are attached form phthalimide or saccharin.

In some embodiments, suitable third reducing agents include zinc (e.g., zinc dust, zinc on graphite, Rieke zinc), metals (e.g., iron, manganese), diimide (e.g., o-nitrobenzenesulfonylhydrazide), copper/bis(pinacolato)diboron, imidazolinium salts (e.g., 1,3-Bis-(2,6-diisopropylphenyl)imidazolinium chloride, 1,3-Bis(2,4,6-trimethylphenyl)imidazoliniumchloride,1,3-Bis(2,6-diisopropylphenyl)imidazolium chloride, 1,3-Bis(2,4,6-trimethylphenyl)imidazolium chloride), or combinations thereof.

In some embodiments, the third reducing agent comprises zinc, a metal, a diimide, copper and bis(pinacolato)diboron, an imidazolinium salt, or a combination thereof. In some embodiments, the third reducing agent comprises zinc. In some embodiments, the third reducing agent comprises zinc dust, zinc on graphite, or Rieke zinc. In some embodiments, the third reducing agent comprises zinc dust.

Any suitable activator (e.g., entrainment agent) can be used to activate the third reducing agent. In some embodiments, the activator activates the third reducing agent, wherein the third reducing agent comprises zinc (e.g., by removing zinc oxide on the surface of the zinc). In some embodiments, suitable activators include an organosilicon compound, an organobromine compound, a strong acid, a hydride, or a combination thereof. In some embodiments, the activator is selected from chlorotrimethylsilane, dibromoethane, hydrochloric acid, and diisobutylaluminum hydride. In some embodiments, the activator is chlorotrimethylsilane.

In some embodiments, the reacting of the compound of Formula G or a salt thereof is performed in a forty-fifth solvent. In some embodiments, the forty-fifth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a glycol (e.g., polyethylene glycol 400), a halogenated solvent (e.g., dichloromethane, 1,2-dichloroethane, chlorobenzene, α,α,α-trifluorotoluene), or a combination thereof. In some embodiments, the forty-fifth solvent comprises an alcohol. In some embodiments, the forty-fifth solvent comprises methanol, ethanol, 2-propanol, butanol, iso-amyl alcohol, tert-amyl alcohol, or a combination thereof. In some embodiments, the reacting of the compound of Formula G or a salt thereof is performed in a forty-fifth solvent comprising ethanol.

In some embodiments, the reacting of the compound of Formula G or a salt thereof is carried out in a temperature range of from about 0° C. to about 100° C. In some embodiments, the reacting of the compound of Formula G or a salt thereof is carried out in a temperature range of from about 10° C. to about 30° C. In some embodiments, the reacting of the compound of Formula G or a salt thereof is carried out at a temperature of about 20° C.

### Process for Preparing the Compound of Formula D

In some embodiments of the processes disclosed herein, the process comprises preparing the compound of Formula D or a salt thereof, wherein R⁵ is R⁹, by a process comprising:
reacting a compound of Formula F-1:

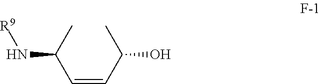

or a salt thereof, wherein R⁹ is an amine protecting group, with:
a phosphine reagent,
an azodicarboxylate, and
a compound of formula R⁷—NH—NH—R⁸;
   to provide the compound of Formula D or a salt thereof.

In some embodiments of the compound of Formula F-1 or a salt thereof, wherein R⁹ is tert-butyloxycarbonyl.

In some embodiments, the compound of Formula R⁷—NH—NH—R⁸ is

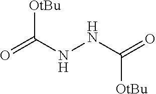 [SMILES: CC(C)(C)OC(=O)NNC(=O)OC(C)(C)C]

In some embodiments, suitable phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the phosphine reagent is selected from a trialkyl phosphine, a triaryl phosphine, (triphenylphosphoranylidene) acetonitrile, and a phosphorane. In some embodiments, the phosphine reagent is a triarylphosphine. In some embodiments, the phosphine reagent comprises triphenylphosphine, tris (2-pyridyl) phosphine, tris-(4-methoxyphenyl) phosphine, or a combination thereof. In some embodiments, the phosphine reagent is triphenylphosphine.

In some embodiments, the azodicarboxylate is selected from di-tert-butyl azodicarboxylate, diisopropyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the azodicarboxylate is di-tert-butyl azodicarboxylate.

In some embodiments, the reacting of the compound of Formula F-1 or a salt thereof is performed in a thirty-fifth solvent. In some embodiments, the thirty-fifth comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a hydrocarbon (e.g., toluene, n-heptane) a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a halogenated solvent (e.g., dichloromethane, 1,2-dichloroethane, chlorobenzene, α,α,α-trifluorotoluene), or a combination thereof. In some embodiments, the thirty-fifth comprises an ether. In some embodiments, the thirty-fifth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula F-1 or a salt thereof is performed in a thirty-fifth solvent comprising 2-methyltetrahydrofuran.

In some embodiments, the reacting of the compound of Formula F-1 or a salt thereof is carried out in a temperature range of from about −20° C. to about 50° C. In some embodiments, the reacting of the compound of Formula F-1 or a salt thereof is carried out in a temperature range of from about 15° C. to about 30° C. In some embodiments, the reacting of the compound of Formula F-1 or a salt thereof is carried out at a temperature range of about 22° C.

In some embodiments, the process further comprises preparing the compound of Formula F-1 or a salt thereof by a process comprising:
reacting a compound of Formula G-1:

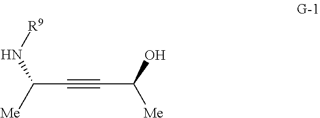

or a salt thereof, with:
a third catalyst,
an aromatic amine or a sulfur-containing reagent; and
hydrogen;
   to provide the compound of Formula F-1 or a salt thereof.

In some embodiments of the compound of Formula G-1, R⁹ is tert-butyloxycarbonyl.

In some embodiments, suitable third catalysts include palladium catalysts (e.g., palladium, 5 wt % on carbon, palladium, 10 wt % on carbon, palladium, 1 wt % on carbon, palladium, 5 wt % on calcium carbonate, palladium, 5 wt % on aluminum oxide, palladium hydroxide, 20 wt % on carbon, lead poisoned palladium), bimetallic catalysts (e.g., palladium, 4 wt % and platinum, 1 wt % on carbon, palladium, 9 wt % and copper, 1 wt % on carbon, palladium, 4 wt % and copper, 1 wt % on carbon), and platinum catalysts (e.g., platinum on carbon, platinum oxide).

In some embodiments, the third catalyst is selected from a palladium catalyst, a platinum catalyst, and a bimetallic catalyst, wherein the bimetallic catalyst comprises two metals selected from palladium, platinum, and copper.

In some embodiments, the third catalyst is a palladium catalyst. In some embodiments, the third catalyst comprises palladium on carbon, palladium on calcium carbonate, or palladium on aluminum oxide. In some embodiments, the third catalyst is selected from 5 wt % palladium on carbon, 10 wt % palladium on carbon, 1 wt % palladium on carbon, 5 wt % palladium on calcium carbonate, 5 wt % palladium on aluminum oxide, 20 wt % palladium on carbon, and lead poisoned palladium.

In some embodiments, the third catalyst is a Lindlar catalyst. In some embodiments, the third catalyst comprises 5 wt. % palladium on lead-poisoned calcium carbonate.

In some embodiments, a molar ratio of the compound of Formula G-1 or a salt thereof to the third catalyst is from about 1:0.001 to about 1:0.05. In some embodiments, a molar ratio of the compound of Formula G-1 or a salt thereof to the third catalyst is from about 1:0.001 to about 1:0.005. In some embodiments, a molar ratio of the compound of Formula G-1 or a salt thereof to the third catalyst is about 1:0.002.

In some embodiments, the reacting of the compound of Formula G-1 or a salt thereof is carried out at a pressure of about 1 psi to about 100 psi. In some embodiments, the reacting of the compound of Formula G-1 or a salt thereof is carried out at a pressure of about 1 psi to about 40 psi. In some embodiments, the reacting of the compound of Formula G-1 or a salt thereof is carried out at a pressure of about 22 psi.

In some embodiments, the compound of Formula G-1 or a salt thereof is reacted with an aromatic amine. In some embodiments, the compound of Formula G-1 or a salt thereof is reacted with an aromatic amine selected from quinoline and pyridine. In some embodiments, the aromatic amine is quinoline.

In some embodiments, the compound of Formula G-1 or a salt thereof is reacted with a sulfur-containing compound. In some embodiments, the compound of Formula G-1 or a salt thereof is reacted with a sulfur-containing compound selected from a thiol and a sulfide.

In some embodiments, the reacting of the compound of Formula G-1 or a salt thereof is performed in a thirty-sixth solvent. In some embodiments, the thirty-sixth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a halogenated solvent (e.g., dichloromethane, 1,2-dichloroethane, chlorobenzene, α,α,α-trifluorotoluene), or a combination thereof. In some embodiments, the thirty-sixth solvent comprises a polar aprotic solvent. In some embodiments, the thirty-sixth solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula G-1 or a salt thereof is performed in a thirty-sixth solvent comprising N,N-dimethylformamide.

In some embodiments, the reacting of the compound of Formula G-1 or a salt thereof is carried out in a temperature range of from about 20° C. to about 100° C. In some embodiments, the reacting of the compound of Formula G-1 or a salt thereof is carried out in a temperature range of from about 10° C. to about 30° C. In some embodiments, the reacting of the compound of Formula G-1 or a salt thereof is carried out at a temperature of about 22° C.

In some embodiments, the process further comprises preparing the compound of Formula G-1 or a salt thereof by a process comprising:
reacting a compound of Formula H having Formula H-1:

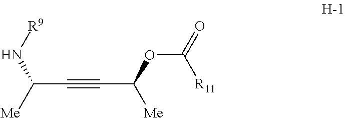

or a salt thereof, wherein R¹¹ is selected from C₁₋₂₀alkyl, phenyl, and —CH₂NH₂;
with a twelfth base,
   to provide the compound of Formula G-1 or a salt thereof.

In some embodiments, R¹¹ is selected from methyl, ethyl, isopropyl, pentyl, heptyl, nonyl, pentadecyl, phenyl, and —CH₂NH₂. In some embodiments, R¹¹ is C₁₋₂₀alkyl. In some embodiments, R¹¹ is selected from methyl, ethyl, isopropyl, pentyl, heptyl, nonyl, and pentadecyl. In some embodiments, R¹¹ is methyl. In some embodiments, R¹¹ is phenyl. In some embodiments, R¹¹ is —CH₂NH₂.

In some embodiments, the compound of Formula H-1 or a salt thereof is a compound of Formula H-1-A:

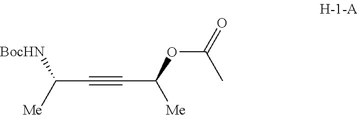 [SMILES: CC(=O)O[C@@H](C)C#C[C@H](C)NC(=O)OC(C)(C)C]

or a salt thereof.

In some embodiments, the compound of Formula H-1 or a salt thereof is reacted with a twelfth base. In some embodiments, suitable twelfth bases include guanidines (e.g., triazabicyclodecene (TBD), 1,1,3,3-tetramethylguanidine), carbonates (e.g., lithium carbonate, sodium carbonate, cesium carbonate, potassium carbonate), bicarbonates (e.g., lithium bicarbonate, sodium bicarbonate, cesium bicarbonate, potassium bicarbonate), metal alkoxides (e.g., sodium methoxide, sodium ethoxide, sodium tert-butoxide, potassium methoxide, potassium ethoxide, potassium tert-butoxide), and amines (e.g., methylamine, diethylamine, isopropylamine, diisopropylethylamine, triethylamine, diisopropylethylamine, 1,8-diazabicyclo[5.4.0]undec-7-ene, 1,4-diazabicyclo[2.2.2]octane, Bis(trimethylsilyl)amine).

In some embodiments, the twelfth base is selected from a guanidine, a carbonate, a bicarbonate, a metal alkoxide, an amine, or a combination thereof. In some embodiments, the twelfth base comprises triazabicyclodecene, tetra-methyl guanidine, or a combination thereof. In some embodiments, the twelfth base is tetra-methyl guanidine.

In some embodiments, the reacting of the compound of Formula H-1 or a salt thereof is performed in a thirty-seventh solvent. In some embodiments, the thirty-seventh solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the thirty-seventh solvent comprises an alcohol. In some embodiments, the thirty-seventh solvent comprises methanol, ethanol, 2-propanol, butanol, iso-amyl alcohol, or a combination thereof. In some embodiments, the reacting of the compound of Formula H-1 or a salt thereof is performed in a thirty-seventh solvent comprising methanol.

In some embodiments, the reacting of the compound of Formula H-1 or a salt thereof is carried out in a temperature range of from about 0° C. to about 30° C. In some embodiments, the reacting of the compound of Formula H-1 or a salt thereof is carried out in a temperature range of from about 10° C. to about 20° C. In some embodiments, the reacting of the compound of Formula H-1 or a salt thereof is carried out in at a temperature of about 20° C.

### Alternative Process for Preparing the Compound of Formula G-1

In some embodiments of the processes disclosed herein, the compound of Formula G-1 or a salt thereof is prepared by a process comprising:
reacting a compound of Formula U-1:

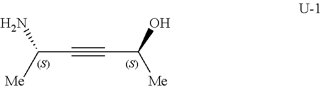

or a salt thereof, with a second amine protecting group reagent to provide the compound of Formula G-1 or a salt thereof.

In some embodiments of the compound of Formula G-1 or a salt thereof, R⁹ is tert-butyloxycarbonyl.

In some embodiments, the second amine protecting group reagent is di-tert-butyl dicarbonate.

In some embodiments, the reacting of the compound of Formula U-1 or a salt thereof is performed in a forty-sixth solvent. In some embodiments, the forty-sixth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a halogenated solvent (e.g., dichloromethane, 1,2-dichloroethane, chlorobenzene, α,α,α-trifluorotoluene), or a combination thereof. In some embodiments, the forty-sixth solvent comprises an ether. In some embodiments, the forty-sixth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the reacting of the compound of Formula U-1 or a salt thereof is performed in a forty-sixth solvent comprising 2-methyltetrahydrofuran.

In some embodiments, the reacting of the compound of Formula U-1 or a salt thereof is carried out in a temperature range of from about 5° C. to about 100° C. In some embodiments, the reacting of the compound of Formula U-1 or a salt thereof is carried out in a temperature range of from about 10° C. to about 30° C. In some embodiments, the reacting of the compound of Formula U-1 or a salt thereof is carried out at a temperature of about 20° C.

In some embodiments, the process further comprising preparing the compound of Formula U-1 or a salt thereof is prepared by a process comprising:
reacting a compound of Formula U-2:

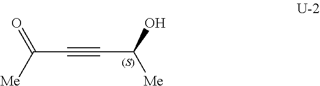

or a salt thereof, with:
a third enzyme in the presence of a third buffer;
   to provide the compound of Formula U-1 or a salt thereof.

In some embodiments, suitable third enzymes include transaminase (e.g., ATA-238 from Codexis®), amine hydrogenase, and imine reductase. In some embodiments, the third enzyme is selected from a transaminase, an amine hydrogenase, an imine reductase, and a combination thereof. In some embodiments, the third enzyme is a transaminase. In some embodiments, the third enzyme is ATA-238, ATA-P1-G05, or ATA-P1-B02.

In some embodiments, suitable third buffers include citrate buffers, phosphate buffers, tris(hydroxymethyl)aminomethane (Tris), and (4-(2-hydroxyethyl)-1-piperazineethanesulfonic acid) (HEPES). In some embodiments, the third buffer is selected from a citrate buffer, a phosphate buffer, a tris(hydroxymethyl)aminomethane (Tris) buffer, and (4-(2-hydroxyethyl)-1-piperazineethanesulfonic acid) (HEPES). In some embodiments, the third buffer is a Tris buffer.

In some embodiments, the reacting of the compound of Formula U-2 or a salt thereof is performed further in the presence of a coenzyme. In some embodiments, the coenzyme is pyridoxal phosphate. In some embodiments, the third enzyme is a transaminase and the coenzyme is pyridoxal phosphate.

In some embodiments, the reacting of the compound of Formula U-2 or a salt thereof is performed in a second cosolvent. In some embodiments, the second cosolvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a halogenated solvent (e.g., dichloromethane, 1,2-dichloroethane, chlorobenzene, α,α,α-trifluorotoluene), or a combination thereof. In some embodiments, the second cosolvent comprises a polar aprotic solvent. In some embodiments, the second cosolvent comprises DMSO, N,N-dimethylacetamide, N-methyl-2-pyrrolidone, N-butyl pyrrolidone (NBP), Limonene, or a combination thereof. In some embodiments, the reacting of the compound of Formula U-2 or a salt thereof is performed in a second cosolvent comprising dimethylsulfoxide.

In some embodiments, the reacting of the compound of Formula U-2 or a salt thereof is carried out in a temperature range of from about 5° C. to about 100° C. In some embodiments, the reacting of the compound of Formula U-2 or a salt thereof is carried out in a temperature range of from about 10° C. to about 30° C. In some embodiments, the reacting of the compound of Formula U-2 or a salt thereof is carried out at a temperature of about 20° C.

In some embodiments, the process further comprising preparing the compound of Formula U-2 or a salt thereof is prepared by a process comprising:
reacting a compound of Formula L:

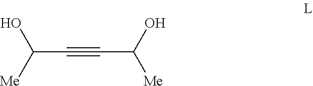

or a salt thereof, with:
a fourth enzyme in the presence of a fourth buffer;
   to provide the compound of Formula U-2 or a salt thereof.

In some embodiments, suitable fourth enzymes include ketoreductase (e.g., KRED-476 from Codexis®), alcohol oxidase, and P450. In some embodiments, the fourth enzyme is selected from a ketoreductase, an alcohol oxidase, P450, and a combination thereof. In some embodiments, the fourth enzyme is a ketoreductase. In some embodiments, the fourth enzyme is KRED-476.

In some embodiments, suitable fourth buffers include citrate buffers, phosphate buffers, tris(hydroxymethyl)aminomethane (Tris), and (4-(2-hydroxyethyl)-1-piperazineethanesulfonic acid) (HEPES). In some embodiments, the fourth buffer is selected from a citrate buffer, a phosphate buffer, tris(hydroxymethyl)aminomethane, and (4-(2-hydroxyethyl)-1-piperazineethanesulfonic acid) (HEPES). In some embodiments, the fourth buffer is a Tris buffer.

In some embodiments, the reacting of the compound of Formula L or a salt thereof comprises reacting the compound of Formula L with a second reducing agent. In some embodiments, the reducing agent is beta-nicotinamide adenine dinucleotide sodium salt.

In some embodiments, the reacting of the compound of Formula L or a salt thereof is performed in a third cosolvent. In some embodiments, the third cosolvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a halogenated solvent (e.g., dichloromethane, 1,2-dichloroethane, chlorobenzene, α,α,α-trifluorotoluene), or a combination thereof. In some embodiments, the third cosolvent comprises a polar aprotic solvent. In some embodiments, the second cosolvent comprises DMSO, N,N-dimethylacetamide, N-methyl-2-pyrrolidone, N-butyl pyrrolidone (NBP), Limonene, or a combination thereof. In some embodiments, the reacting of the compound of Formula L or a salt thereof is performed in a third cosolvent comprising dimethylsulfoxide.

In some embodiments, the reacting of the compound of Formula L or a salt thereof is carried out in a temperature range of from about 5° C. to about 100° C. In some embodiments, the reacting of the compound of Formula L or a salt thereof is carried out in a temperature range of from about 10° C. to about 30° C. In some embodiments, the reacting of the compound of Formula L or a salt thereof is carried out at a temperature of about 20° C.

### Process for Preparing the Compound of Formula H

In some embodiments of the processes disclosed herein, the process comprises preparing the compound of Formula H or a salt thereof by a process comprising:
reacting a compound of Formula J:

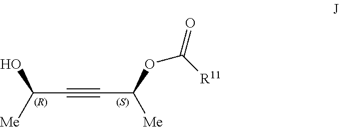

or a salt thereof, with:
a second phosphine reagent,
a second azodicarboxylate, and
an amine protecting group reagent;
   to provide the compound of Formula H or a salt thereof.

In some embodiments of the compound of Formula J, R¹¹ is methyl.

In some embodiments, suitable second phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the second phosphine reagent is selected from a trialkyl phosphine, a triaryl phosphine, (triphenylphosphoranylidene) acetonitrile, and a phosphorane. In some embodiments, the second phosphine reagent is a triarylphosphine. In some embodiments, the second phosphine reagent comprises triphenylphosphine, tris (2-pyridyl) phosphine, tris-(4-methoxyphenyl) phosphine, or a combination thereof. In some embodiments, the second phosphine reagent is triphenylphosphine.

In some embodiments, the second azodicarboxylate is selected from di-tert-butyl azodicarboxylate, diisopropyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the second azodicarboxylate is diisopropyl azodicarboxylate.

In some embodiments, the amine protecting group reagent is an imide. In some embodiments, the amine protecting group reagent is an imide selected from phthalimide, tert-butyl methyl iminodicarbonate, and di-tert-butyliminodicarboxylate. In some embodiments, the amine protecting group reagent is phthalimide.

In some embodiments, the reacting of the compound of Formula J or a salt thereof is performed in a thirty-eighth solvent. In some embodiments, the thirty-eighth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the thirty-eighth solvent comprises an ether. In some embodiments, the thirty-eighth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula J or a salt thereof is performed in a thirty-eighth solvent comprising 2-methyltetrahydrofuran.

In some embodiments, the reacting of the compound of Formula J or a salt thereof is carried out in a temperature range of from about −20° C. to about 50° C. In some embodiments, the reacting of the compound of Formula J or a salt thereof is carried out in a temperature range of from about −5° C. to about 15° C. In some embodiments, the reacting of the compound of Formula J or a salt thereof is carried out at a temperature range of about 5° C.

### Alternative Process for Preparing the Compound of Formula H

In some embodiments of the processes disclosed herein, the process comprises preparing the compound of Formula H or a salt thereof by a process comprising:
reacting a compound of Formula N:

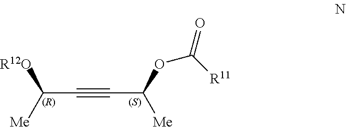

or a salt thereof, wherein:
R¹² is —S(O)₃R¹³ or —OC(═N)R¹⁴;
R¹³ is selected from C₁₋₄alkyl, C₁₋₄haloalkyl, and phenyl optionally substituted with C₁₋₄alkyl; and
R¹⁴ is C₁₋₄haloalkyl;
   with an amine protecting group reagent,
   to provide the compound of Formula H or a salt thereof.

In some embodiments, R¹² is —S(O)₃R¹³. In some embodiments, R¹³ is selected from tolyl, methyl, and trifluoromethyl. In some embodiments, R¹³ is C₁₋₄alkyl. In some embodiments, R¹³ is methyl. In some embodiments, R¹³ is tolyl.

In some embodiments, R¹² is —OC(═N)R¹⁴. In some embodiments, R¹⁴ is selected from trichloromethyl and tribromomethyl.

In some embodiments, the amine protecting group reagent is selected from phthalimide, para-methoxyaniline, ortho, para-dimethoxyaniline, acetamide, 2,5-dimethylpyrrole, 4-methoxybenzylamine, 2,4-dimethoxybenzylamine, 2,2-diphenylamine, allylamine, triphenylmethylamine, di-tert-butyl iminodicarbonate, tert-butyl carbamate, or a salt thereof. In some embodiments, the amine protecting group reagent is selected from phthalimide, para-methoxyaniline, ortho, para-dimethoxyaniline, acetamide, 2,5-dimethylpyrrole, 4-methoxybenzylamine, 2,4-dimethoxybenzylamine, 2,2-diphenylamine, allylamine, triphenylmethylamine, or a salt thereof. In some embodiments, the amine protecting group reagent is phthalimide or a salt thereof. In some embodiments, the amine protecting group reagent is a salt of phthalimide. In some embodiments, the amine protecting group reagent is potassium phthalimide.

In some embodiments, the reacting of the compound of Formula N or a salt thereof is carried out further in the presence of a third additive. In some embodiments, suitable third additives include 18-crown-6, tetrabutylammonium salts (e.g., chloride, bromide, sulfate, iodide), hexamethylphosphoramide, and water. In some embodiments, the third additive comprises 18-crown-6, a tetrabutylammonium salt, hexamethylphosphoramide, water, or a combination thereof. In some embodiments, the third additive comprises 18-crown-6. In some embodiments, the reacting of the compound of Formula N is carried out in the absence of a third additive.

In some embodiments, the reacting of the compound of Formula N or a salt thereof is carried out in the presence of a tenth base. In some embodiments, suitable tenth bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN), etc.), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide, etc.), basic aromatic compounds (e.g., pyridine, 2,6-lutidine, imidazole), or a combination thereof. In some embodiments, the tenth base is selected from an amine, an amidine, a guanidine, a carboxylate, a carbonate, a metal hydride, a phosphate, a hydroxide, an alkoxide, and a basic aromatic compound.

In some embodiments, the reacting of the compound of Formula N or a salt thereof is performed in a twentieth solvent. In some embodiments, the twentieth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the twentieth solvent comprises a polar aprotic solvent. In some embodiments, the twentieth solvent N,N-dimethylformamide, N,N-dimethylacetamide, N-methyl-2-pyrrolidone, N-butyl pyrrolidone (NBP), or a combination thereof. In some embodiments, the reacting of the compound of Formula N or a salt thereof is performed in a twentieth solvent comprising N,N-dimethylacetamide.

In some embodiments, the reacting of the compound of Formula N or a salt thereof is performed in a twentieth solvent. In some embodiments, the twentieth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), water, or a combination thereof. In some embodiments, the twentieth solvent comprises a polar aprotic solvent. In some embodiments, the twentieth solvent N,N-dimethylformamide, N,N-dimethylacetamide, N-methyl-2-pyrrolidone, N-butyl pyrrolidone (NBP), limonene, dimethyl sulfoxide, or a combination thereof.

In some embodiments, the reacting of the compound of Formula N or a salt thereof is carried out in a temperature range of from about 20° C. to about 100° C. In some embodiments, the reacting of the compound of Formula N or a salt thereof is carried out in a temperature range of from about 10° C. to about 30° C.

In some embodiments, the process further comprises preparing the compound of Formula N or a salt thereof by a process comprising:
reacting a compound of Formula J:

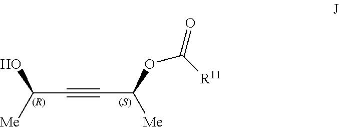

or a salt thereof, with:
an eleventh base, and
a third reagent selected from a sulfonyl chloride, a sulfonyl anhydride, a halonitrile, an imidoyl chloride, or an acid halide;
   to provide the compound of Formula N or a salt thereof.

In some embodiments, the process further comprises preparing the compound of Formula N or a salt thereof by a process comprising: reacting a compound of Formula J or a salt thereof, with an eleventh base, and a third reagent selected from a sulfonyl chloride, a sulfonyl anhydride, a halonitrile, or an imidoyl chloride; to provide the compound of Formula N or a salt thereof.

In some embodiments, the third reagent is a sulfonyl chloride. In some embodiments, the third reagent is selected from methane sulfonyl chloride and 4-toluene sulfonyl chloride. In some embodiments, the third reagent is methane sulfonyl chloride. In some embodiments, the third reagent is benzene sulfonyl chloride, toluene sulfonyl chloride, 4-nitrobenzene sulfonyl chloride, 2-nitrobenzene sulfonyl chloride, 4-bromobenzene sulfonyl chloride, triisopropylbenzene sulfonyl chloride, trimethylbenzene sulfonyl chloride, naphthalene sulfonyl chloride, 2-chlorobenzene sulfonyl chloride, 2,4-dichlorobenzene sulfonyl chloride, or methane sulfonyl chloride.

In some embodiments, the third reagent is a sulfonyl anhydride. In some embodiments, the sulfonyl anhydride is selected from methane sulfonyl anhydride and trifluoromethanesulfonic anhydride. In some embodiments, the third reagent is a sulfonyl anhydride (e.g., hexanoic anhydride, isobutyric anhydride, benzoic anhydride, propionic anhydride, octanoic anhydride, decanoic anhydride).

In some embodiments, the third reagent is a halonitrile. In some embodiments, the halonitrile is selected from trichloroacetonitrile and trifluoroacetonitrile.

In some embodiments, the third reagent is an imidoyl chloride. In some embodiments, the imidoyl chloride is (N-phenyl)trifluoroacetimidoyl chloride.

In some embodiments, the third reagent is an acid halide. In some embodiments, the acid halide is selected from acetyl chloride, acetyl bromide, and benzoyl chloride.

In some embodiments, suitable eleventh bases include amines (e.g., triethylamine, 1,8-diazabicyclo[5.4.0]undec-7-ene, 1,5-diazabicyclo[4.3.0]non-5-ene, 1,4-diazabicyclo[2.2.2]octane, 1,1,3,3-tetramethylguanidine, 2,2,6,6-tetramethylpiperidine, pyridine, 2,6-lutidine, N,N-diisopropylethylamine, quinuclidine, diisopropylamine, N,N-diisopropylethylamine, tributylamine), carbonates (e.g., lithium carbonate, sodium carbonate, cesium carbonate, potassium carbonate), bicarbonates (e.g., lithium bicarbonate, sodium bicarbonate, cesium bicarbonate, potassium bicarbonate), and metal alkoxides (e.g., sodium methoxide, sodium ethoxide, sodium tert-butoxide, potassium methoxide, potassium ethoxide, potassium tert-butoxide).

In some embodiments, the eleventh base is an amine. In some embodiments, the eleventh base comprises triethylamine, 1,8-diazabicyclo[5.4.0]undec-7-ene, 1,5-diazabicyclo[4.3.0]non-5-ene, 1,4-diazabicyclo[2.2.2]octane, 1,1,3,3-tetramethylguanidine, 2,2,6,6-tetramethylpiperidine, pyridine, 2,6-lutidine, N,N-diisopropylethylamine, quinuclidine, diisopropylamine, N,N-diisopropylethylamine, tributylamine, or a combination thereof. In some embodiments, the eleventh base is triethylamine.

In some embodiments, the reacting of the compound of Formula J or a salt thereof is performed in a twenty-first solvent. In some embodiments, the twenty-first solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the twenty-first solvent comprises a polar aprotic solvent. In some embodiments, the twenty-first solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula J or a salt thereof is performed in a twenty-first solvent comprising N,N-dimethylacetamide.

In some embodiments, the reacting of the compound of Formula J or a salt thereof is carried out in a temperature range of from about −15° C. to about 60° C. In some embodiments, the reacting of the compound of Formula J or a salt thereof is carried out in a temperature range of from about −10° C. to about 10° C. In some embodiments, the reacting of the compound of Formula J or a salt thereof is carried out at a temperature of about 0° C.

### Alternative Process for Preparing the Compound of Formula H-1

In some embodiments of the processes disclosed herein, the process comprises preparing the compound of Formula H-1 or a salt thereof by a process comprising:
reacting a compound of Formula P-1:

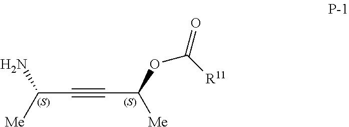

or a salt thereof, with an amine protecting group reagent to provide the compound of Formula H-1 or a salt thereof. In some embodiments of the compound of Formula P-1 or a salt thereof, R¹¹ is methyl.

In some embodiments, the amine protecting group reagent is di-tert-butyl dicarbonate.

In some embodiments, the process further comprises preparing the compound of Formula P-1 or a salt thereof by a process comprising:
reacting a compound of Formula N:

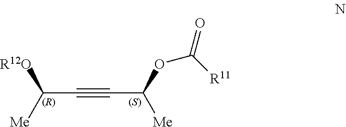

or a salt thereof, wherein:
R¹² is —S(O)₃R¹³; and
R¹³ is selected from C₁₋₄alkyl and phenyl optionally substituted with C₁₋₄alkyl; with:
an azide, and
a third phosphine reagent;
   to provide the compound of Formula P-1 or a salt thereof.

In some embodiments, the azide is selected from sodium azide, diphenylphosphoryl azide, and trimethylsilyl azide. In some embodiments, the azide is sodium azide.

In some embodiments, suitable third phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the third phosphine reagent is selected from a trialkyl phosphine, a triaryl phosphine, (triphenylphosphoranylidene) acetonitrile, and a phosphorane. In some embodiments, the third phosphine reagent is a triarylphosphine. In some embodiments, the third phosphine reagent comprises triphenylphosphine, tris (2-pyridyl) phosphine, tris-(4-methoxyphenyl) phosphine, or a combination thereof. In some embodiments, the third phosphine reagent is triphenylphosphine.

In some embodiments, the reacting of the compound of Formula N or a salt thereof is performed in a twenty-seventh solvent. In some embodiments, the twenty-seventh solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the twenty-seventh solvent comprises a polar aprotic solvent. In some embodiments, the twenty-seventh solvent comprises N,N-dimethylformamide, N,N-dimethylacetamide, N-methyl-2-pyrrolidone, N-butyl pyrrolidone (NBP)), or a combination thereof. In some embodiments the reacting of the compound of Formula N or a salt thereof is performed in a twenty-seventh solvent comprising N,N-dimethylformamide.

In some embodiments, the reacting of the compound of Formula N or a salt thereof is carried out in a temperature range of from about −20° C. to about 50° C. In some embodiments, the reacting of the compound of Formula N or a salt thereof is carried out in a temperature range of from about 10° C. to about 30° C.

### Alternative Process for Preparing the Compound of Formula H-1

In some embodiments of the processes disclosed herein, the process comprises preparing the compound of Formula H-1 or a salt thereof by a process comprising:
reacting a compound of Formula P-2:

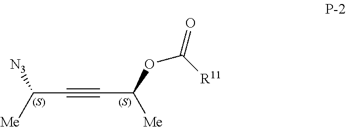

or a salt thereof, with:
a fourth phosphine reagent; and
an amine protecting group reagent;
   to provide the compound of Formula H-1 or a salt thereof. In some embodiments of the compound of Formula P-2 or a salt thereof, R¹¹ is methyl.

In some embodiments, suitable fourth phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the fourth phosphine reagent is selected from a trialkyl phosphine, a triaryl phosphine, (triphenylphosphoranylidene) acetonitrile, and a phosphorane. In some embodiments, the fourth phosphine reagent is a triarylphosphine. In some embodiments, the fourth phosphine reagent comprises triphenylphosphine, tris (2-pyridyl) phosphine, tris-(4-methoxyphenyl) phosphine, or a combination thereof. In some embodiments, the fourth phosphine reagent is triphenylphosphine.

In some embodiments, the amine protecting group reagent is di-tert-butyl dicarbonate.

In some embodiments, the process further comprises preparing the compound of Formula P-2 or a salt thereof by a process comprising:
reacting a compound of reacting a compound of Formula J:

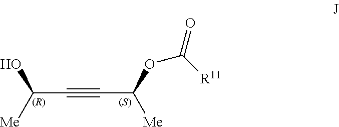

or a salt thereof, with:
an azide,
a third phosphine reagent, and
a third azodicarboxylate;
   to provide the compound of Formula P-2 or a salt thereof.

In some embodiments, the azide is selected from sodium azide, diphenylphosphoryl azide, and trimethylsilyl azide. In some embodiments, the azide is sodium azide.

In some embodiments, suitable third phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the third phosphine reagent is selected from a trialkyl phosphine, a triaryl phosphine, (triphenylphosphoranylidene) acetonitrile, and a phosphorane. In some embodiments, the third phosphine reagent is a triarylphosphine. In some embodiments, the third phosphine reagent comprises triphenylphosphine, tris (2-pyridyl) phosphine, tris-(4-methoxyphenyl) phosphine, or a combination thereof. In some embodiments, the third phosphine reagent is triphenylphosphine.

In some embodiments, the third azodicarboxylate is selected from di-tert-butyl azodicarboxylate, diisopropyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the third azodicarboxylate is diisopropyl azodicarboxylate.

In some embodiments, the reacting of the compound of Formula J or a salt thereof is performed in a thirty-ninth solvent. In some embodiments, the thirty-ninth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the thirty-eighth solvent comprises an ether. In some embodiments, the thirty-ninth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula J or a salt thereof is performed in a thirty-ninth solvent comprising tetrahydrofuran.

In some embodiments, the reacting of the compound of Formula J or a salt thereof is carried out in a temperature range of from about −20° C. to about 50° C. In some embodiments, the reacting of the compound of Formula J or a salt thereof is carried out in a temperature range of from about 10° C. to about 30° C.

### Process for Preparing the Compound of Formula J

In some embodiments of the processes disclosed herein, the process comprises preparing the compound of Formula J or a salt thereof by a process comprising:
reacting a compound of Formula K:

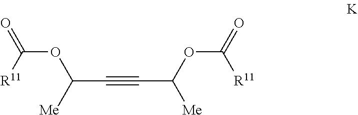

or a salt thereof, with:
   an enzyme, and
   a buffer;
to provide the compound of Formula J or a salt thereof.

In some embodiments of the compound of Formula K or a salt thereof, R¹¹ is selected from C₁₋₂₀ alkyl and phenyl. In some embodiments of the compound of Formula K or a salt thereof, R¹¹ is selected from methyl, hexyl, isopropyl, and benzyl. In some embodiments of the compound of Formula K or a salt thereof, R¹¹ is methyl.

In some embodiments, suitable enzymes include hydrolases (such as Candida antarctica lipase A and B, lipase AK, lipase PS, Candida rugosa lipase, L-092, etc.) Prozomix 4, Prozomix 5, Prozomix 25, Prozomix 26, and Novozyme 435.

In some embodiments, the enzyme is selected from a hydrolase enzyme, Prozomix 4, Prozomix 5, Prozomix 25, Prozomix 26, and Novozyme 435. In some embodiments, the enzyme is a hydrolase enzyme. In some embodiments, the enzyme is selected from L-092, Candida antarctica lipase A and B, lipase AK, lipase PS, Candida rugosa lipase. In some embodiments, the enzyme is L-092 hydrolase enzyme.

In some embodiments, a molar ratio of the enzyme to the compound of Formula K or a salt thereof is about 0.00001:1 to about 2:1. In some embodiments, a molar ratio of the enzyme to the compound of Formula K or a salt thereof is about 0.00001:1 to about 0.01:1. In some embodiments, a molar ratio of the enzyme to the compound of Formula K or a salt thereof is about 0.001:1 to about 0.01:1. In some embodiments, a molar ratio of the enzyme to the compound of Formula K or a salt thereof is about 0.001:1 to about 0.005:1.

In some embodiments, suitable buffers include citrate buffers, phosphate buffers (e.g., potassium phosphate, citrate phosphate), tris(hydroxymethyl)aminomethane (Tris), and (4-(2-hydroxyethyl)-1-piperazineethanesulfonic acid) (HEPES). In some embodiments, suitable buffers include phosphate buffers (e.g., potassium phosphate, citrate phosphate), tris(hydroxymethyl)aminomethane (Tris), and (4-(2-hydroxyethyl)-1-piperazineethanesulfonic acid) (HEPES). In some embodiments, the buffer is selected from a citrate buffer, a phosphate buffer, tris(hydroxymethyl)aminomethane, and (4-(2-hydroxyethyl)-1-piperazineethanesulfonic acid) (HEPES). In some embodiments, the buffer is selected from a phosphate buffer, tris(hydroxymethyl)aminomethane, and (4-(2-hydroxyethyl)-1-piperazineethanesulfonic acid) (HEPES). In some embodiments, the buffer is a phosphate buffer. In some embodiments, the buffer is a potassium phosphate buffer. In some embodiments, the buffer has a concentration of about 0.1M to about 1.5 M.

In some embodiments, the process comprises adjusting a pH of the reaction mixture comprising the compound of Formula K or a salt thereof to a range of about pH 8 to pH 12. In some embodiments, the process comprises adjusting a pH of the reaction mixture comprising the compound of Formula K or a salt thereof to about pH 10.

In some embodiments, the reacting of the compound of Formula K or a salt thereof is carried out at a pH of about 5 to about 10. In some embodiments, the reacting of the compound of Formula K or a salt thereof is carried out at a pH of about 7 to about 10. In some embodiments, the reacting of the compound of Formula K or a salt thereof is carried out at a pH of about 6 to about 8. In some embodiments, the reacting of the compound of Formula K or a salt thereof is carried out at a pH of about 7. In some embodiments, the reacting of the compound of Formula K or a salt thereof is carried out at a pH of about 10.

In some embodiments, the reacting of the compound of Formula K or a salt thereof is carried out in the presence of a metal ion. In some embodiments, the metal ion is an ion of zinc, nickel, calcium, iron, magnesium, or manganese. In some embodiments, the reacting of the compound of Formula K or a salt thereof is carried out in the presence of a metal halide. In some embodiments, the metal ion or metal halide comprises zinc, nickel, calcium, iron, magnesium, or manganese. In some embodiments, the metal halide is a metal chloride. In some embodiments, the metal halide comprises zinc chloride, nickel (II) chloride, calcium chloride, iron (II) chloride, iron (III) chloride, magnesium chloride, manganese(II) chloride, or a combination thereof.

In some embodiments, the reacting of the compound of Formula K or a salt thereof is carried out in the presence of a surfactant. In some embodiments, the surfactant is selected from sodium dodecyl sulfate, polypropylene glycol, and a polyoxyethylene fatty ether. In some embodiments, the surfactant is selected from sodium dodecyl sulfate, polypropylene glycol 2000, and Brij surfactants (e.g., L23, 58, S20, O20, L4, O10, S10, C10).

In some embodiments, the reacting of the compound of Formula K or a salt thereof is performed in a fortieth solvent. In some embodiments, the fortieth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the fortieth solvent comprises dimethylsulfoxide, tert-butyl methyl ether, heptane, cyclopentyl methyl ether, toluene, 2-methylhydrofuran, or a combination thereof.

In some embodiments, the fortieth solvent comprises an ether. In some embodiments, the fortieth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula K or a salt thereof is performed in a fortieth solvent comprising tert-butyl methyl ether.

In some embodiments, the reacting of the compound of Formula K or a salt thereof is carried out in a temperature range of from about 0° C. to about 70° C. In some embodiments, the reacting of the compound of Formula K or a salt thereof is carried out in a temperature range of from about 30° C. to about 50° C. In some embodiments, the reacting of the compound of Formula K or a salt thereof is carried out in a temperature range of from about 20° C. to about 40° C. In some embodiments, the reacting of the compound of Formula K or a salt thereof is carried out at a temperature of about 30° C.

In some embodiments, provided herein is a process for preparing a compound of Formula J or a salt thereof by a process comprising:
reacting a compound of Formula K or a salt thereof, with an enzyme, and a buffer;
to provide the compound of Formula J or a salt thereof.

In some embodiments, provided herein is a process for preparing a compound of Formula I or a salt thereof by a process comprising:
preparing a compound of Formula J or a salt thereof by a process comprising:
reacting a compound of Formula K or a salt thereof, with an enzyme, and a buffer;
to provide the compound of Formula J or a salt thereof.

In some embodiments, the process further comprises preparing the compound of Formula K or a salt thereof by a process comprising:
reacting a compound of Formula L:

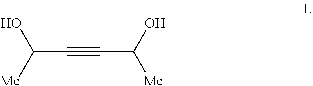

or a salt thereof; with:
a ninth base,
a fourth catalyst; and
a compound of Formula M-1 or Formula M-2:

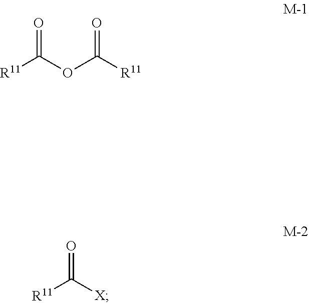

wherein X is a halide;
   to provide the compound of Formula K or a salt thereof.

In some embodiments, the compound of Formula L or a salt thereof is reacted with the compound of Formula M-1. In some embodiments, the compound of Formula M-1 is selected from acetic anhydride, hexanoic anhydride, isobutyric anhydride, benzoic anhydride, propionic anhydride, octanoic anhydride, and decanoic anhydride. In some embodiments, the compound of Formula M-1 is acetic anhydride (i.e., R¹¹ is methyl).

In some embodiments, the compound of Formula L or a salt thereof is reacted with the compound of Formula M-2. In some embodiments, the halide is chloride or bromide. In some embodiments, the compound of Formula M-2 is selected from acetyl chloride, acetyl bromide, and benzoyl chloride.

In some embodiments, suitable ninth bases include carbonates (e.g., lithium carbonate, sodium carbonate, cesium carbonate, sodium bicarbonate), metal alkoxides (e.g., sodium methoxide, sodium tert-butoxide, lithium tert-butoxide), amines (e.g., diethylamine, triethylamine, isopropylamine, diisopropylethylamine, N-methyl morpholine, 1,4-diazabicyclo[2.2.2] octane, Diazabicycloundecene, Bis(trimethylsilyl)amine), metal hydroxides (e.g., sodium hydroxide).

In some embodiments, the ninth base is selected from a carbonate, a metal alkoxide, an amine, a metal hydroxide, or a combination thereof. In some embodiments, the ninth base is an amine. In some embodiments, the ninth base comprises diethylamine, triethylamine, isopropylamine, diisopropylethylamine, N-methyl morpholine, 1,4-diazabicyclo[2.2.2] octane, diazabicycloundecene, bis(trimethylsilyl)amine, or a combination thereof. In some embodiments, the ninth base is triethylamine.

In some embodiments, the fourth catalyst is an aromatic amine. In some embodiments, the fourth catalyst is selected from 4-dimethylaminopyridine, N-methyl imidazole, and pyridine. In some embodiments, the fourth catalyst is 4-dimethylaminopyridine.

In some embodiments, the reacting of the compound of Formula L or a salt thereof is performed in a forty-first solvent. In some embodiments, the forty-first solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the forty-first solvent comprises an ether. In some embodiments, the forty-first solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the reacting of the compound of Formula L or a salt thereof is performed in a forty-first solvent comprising tert-butyl methyl ether.

In some embodiments, the reacting of the compound of Formula L or a salt thereof is carried out in a temperature range of from about 0° C. to about 100° C. In some embodiments, the reacting of the compound of Formula L or a salt thereof is carried out in a temperature range of from about 15° C. to about 25° C. In some embodiments, the reacting of the compound of Formula L or a salt thereof is carried out at a temperature of about 20° C.

### Alternative Process for Preparing the Compound of Formula E

In some embodiments of the processes disclosed herein, the process comprises preparing the compound of Formula E or a salt thereof by a process comprising:
reacting a compound of Formula O-1:

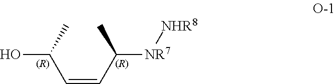

or a salt thereof, with:
an amine protecting group reagent,
a phosphine reagent, and
an azodicarboxylate,
   to provide the compound of Formula E or a salt thereof.

In some embodiments of the compound of Formula O-1, R⁷ and R⁸ are tert-butyloxycarbonyl.

In some embodiments, the amine protecting group reagent is selected from phthalimide, di-tert-butyl-iminodicarboxylate, ethyl 2-((tert-butoxycarbonyl)amino)-2-oxoacetate, methyl 2-((tert-butoxycarbonyl)amino)-2-oxoacetate, tert-butyl methyl iminodicarbonate, and dimethyl iminodicarbonate. In some embodiments, the amine protecting group reagent is phthalimide.

In some embodiments, suitable phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the phosphine reagent is selected from a trialkyl phosphine, a triaryl phosphine, (triphenylphosphoranylidene) acetonitrile, and a phosphorane. In some embodiments, the phosphine reagent is a triarylphosphine. In some embodiments, the phosphine reagent comprises triphenylphosphine, tris (2-pyridyl) phosphine, tris-(4-methoxyphenyl) phosphine, or a combination thereof. In some embodiments, the phosphine reagent is triphenylphosphine.

In some embodiments, the azodicarboxylate is selected from di-tert-butyl azodicarboxylate, diisopropyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the azodicarboxylate is di-tert-butyl azodicarboxylate.

In some embodiments, the reacting of the compound of Formula O-1 or a salt thereof is performed in a twenty-second solvent. In some embodiments, the twenty-second solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the twenty-second solvent comprises an ether. In some embodiments, the twenty-second solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula O-1 or a salt thereof is performed in a twenty-second solvent comprising 2-methyltetrahydrofuran.

In some embodiments, the reacting of the compound of Formula O-1 or a salt thereof is carried out in a temperature range of from about −20° C. to about 50° C. In some embodiments, the reacting of the compound of Formula O-1 or a salt thereof is carried out in a temperature range of from about 0° C. to about 22° C.

In some embodiments, the process further comprises preparing the compound of Formula O-1 or a salt thereof by a process comprising:
reacting a compound of Formula O-2:

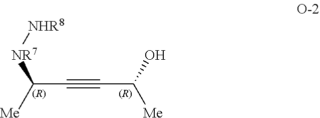

or a salt thereof, with:
a fifth catalyst, and
hydrogen;
   to provide the compound of Formula O-1 or a salt thereof.

In some embodiments, suitable fifth catalysts include palladium catalysts (e.g., palladium, 5 wt % on carbon, palladium, 10 wt % on carbon, palladium, 1 wt % on carbon, palladium, 5 wt % on calcium carbonate, palladium, 5 wt % on aluminum oxide, palladium hydroxide, 20 wt % on carbon, lead poisoned palladium), bimetallic catalysts (e.g., palladium, 4 wt % and platinum, 1 wt % on carbon, palladium, 9 wt % and copper, 1 wt % on carbon, palladium, 4 wt % and copper, 1 wt % on carbon), and platinum catalysts (e.g., platinum on carbon, platinum oxide).

In some embodiments, suitable fifth catalysts include palladium catalysts (e.g., palladium, 5 wt % on carbon, palladium, 10 wt % on carbon, palladium, 1 wt % on carbon, palladium, 5 wt % on calcium carbonate, palladium, 5 wt % on aluminum oxide, palladium hydroxide, palladium acetate, palladium acetate/manganese, palladium chloride, palladium trifluoroacetate, palladium propionate, palladium pivalate, tris(dibenzylideneacetone) dipalladium, palladium bromide, palladium (II) bis(acetylacetonate), bis(benzonitrile)palladium dichloride, 20 wt % on carbon, lead poisoned palladium with, barium sulfate or calcium carbonate), bimetallic catalysts (e.g., palladium, 4 wt % and platinum, 1 wt % on carbon, palladium, 9 wt % and copper, 1 wt % on carbon, palladium, 4 wt % and copper, 1 wt % on carbon), and platinum catalysts (e.g., platinum on carbon, platinum oxide).

In some embodiments, the fifth catalyst is selected from a palladium catalyst, a platinum catalyst, and a bimetallic catalyst, wherein the bimetallic catalyst comprises two metals selected from palladium, platinum, and copper.

In some embodiments, the fifth catalyst is a palladium catalyst. In some embodiments, the fifth catalyst comprises palladium on carbon, palladium on calcium carbonate, or palladium on aluminum oxide. In some embodiments, the fifth catalyst is selected from 5 wt % palladium on carbon, 10 wt % palladium on carbon, 1 wt % palladium on carbon, 5 wt % palladium on calcium carbonate, 5 wt % palladium on aluminum oxide, 20 wt % palladium on carbon, and lead poisoned palladium.

In some embodiments, the fifth catalyst is a Lindlar catalyst. In some embodiments, the fifth catalyst comprises 5 wt. % palladium on lead-poisoned calcium carbonate.

In some embodiments, the hydrogen is from a hydrogen source. In some embodiments, the hydrogen source is the hydrogen gas or a combination of formic acid, potassium carbonate, and water. In some embodiments, the hydrogen source is the hydrogen gas. In some embodiments, the hydrogen source is a combination of formic acid, potassium carbonate, and water.

In some embodiments, the preparation of the compound of Formula O-1 by a process comprising reacting a compound of Formula O-2 or a salt thereof, with the fifth catalyst and hydrogen is done further in presence of an additive. In some embodiments, the additive is selected from the group consisting of quinoline, pyridine, triethylamine, carbonates (such as lithium carbonate, sodium carbonate, cesium carbonate, potassium carbonate), bicarbonates (such as lithium bicarbonate, sodium bicarbonate, cesium bicarbonate, potassium bicarbonate), phosphates M(n)H(3-n)PO4 (such as trisodium phosphate, disodium hydrogen phosphate, tripotassium phosphate, dipotassium hydrogen phosphate, etc.), 1,8-diazabicyclo[5.4.0] undec-7-ene, potassium carbonate, potassium formate, sodium formate, ammonium formate, potassium hydroxide, potassium acetate, sodium acetate, cesium acetate, potassium tert-butoxide, sodium tert-butoxide, potassium iodide, lithium chloride, sodium chloride, calcium chloride, triethylamine, and potassium hydroxide.

In some embodiments, a molar ratio of the compound of Formula O-2 or a salt thereof to the fifth catalyst is from about 1:0.001 to about 1:0.05. In some embodiments, a molar ratio of the compound of Formula O-2 or a salt thereof to the third catalyst is from about 1:0.001 to about 1:0.005. In some embodiments, a molar ratio of the compound of Formula O-2 or a salt thereof to the third catalyst is about 1:0.003.

In some embodiments, the reacting of the compound of Formula O-2 or a salt thereof is carried out at a pressure of about 1 psi to about 100 psi. In some embodiments, the reacting of the compound of Formula O-2 or a salt thereof is carried out at a pressure of about 25 psi to about 75 psi. In some embodiments, the reacting of the compound of Formula O-2 or a salt thereof is carried out at a pressure of about 50 psi.

In some embodiments, the reacting of the compound of Formula O-2 or a salt thereof is performed in a twenty-third solvent. In some embodiments, the twenty-third solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, iso-amyl alcohol, tert-amyl alcohol), a glycol (e.g., polyethylene glycol 400), a halogenated solvent (e.g., dichloromethane, 1,2-dichloroethane, chlorobenzene, α,α,α-trifluorotoluene), or a combination thereof. In some embodiments, the twenty-third solvent comprises a polar aprotic solvent. In some embodiments, the twenty-third solvent comprises N,N-dimethylformamide, N,N-dimethylacetamide, N-methyl-2-pyrrolidone, N-butyl pyrrolidone (NBP), or a combination thereof. In some embodiments, the reacting of the compound of Formula O-2 or a salt thereof is performed in a twenty-third solvent comprising N,N-dimethylformamide.

In some embodiments, the twenty-third solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene, anisole), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol (e.g. 1-butanol), iso-amyl alcohol, tert-amyl alcohol), a glycol (e.g., polyethylene glycol 400), a halogenated solvent (e.g., dichloromethane, 1,2-dichloroethane, chlorobenzene, α,α,α-trifluorotoluene), or a combination thereof.

In some embodiments, the reacting of the compound of Formula O-2 or a salt thereof is carried out in a temperature range of from about 0° C. to about 100° C. In some embodiments, the reacting of the compound of Formula O-2 or a salt thereof is carried out in a temperature range of from about 15° C. to about 30° C. In some embodiments, the reacting of the compound of Formula O-2 or a salt thereof is carried out at a temperature of about 22° C.

In some embodiments, the process further comprises preparing the compound of Formula O-2 or a salt thereof by a process comprising:
reacting a compound of Formula O-3:

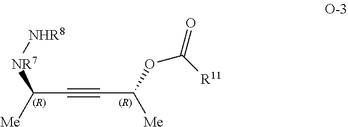

or a salt thereof, wherein R¹¹ is selected from C₁₋₂₀alkyl, phenyl, and —CH₂NH₂; with:
a twelfth base;
   to provide the compound of Formula O-2 or a salt thereof.

In some embodiments of the compound of Formula O-3 or a salt thereof, R¹¹ is selected from methyl, ethyl, isopropyl, pentyl, heptyl, nonyl, pentadecyl, phenyl, and —CH₂NH₂. In some embodiments of the compound of Formula O-3 or a salt thereof, R¹¹ is methyl.

In some embodiments, suitable twelfth bases include guanidines (e.g., triazabicyclodecene (TBD), 1,1,3,3-tetramethylguanidine), carbonates (e.g., lithium carbonate, sodium carbonate, cesium carbonate, potassium carbonate), bicarbonates (e.g., lithium bicarbonate, sodium bicarbonate, cesium bicarbonate, potassium bicarbonate), metal alkoxides (e.g., sodium methoxide, sodium ethoxide, sodium tert-butoxide, potassium methoxide, potassium ethoxide, potassium tert-butoxide), amines (e.g., methylamine, diethylamine, isopropylamine, diisopropylethylamine, triethylamine, diisopropylethylamine, 1,8-diazabicyclo[5.4.0]undec-7-ene, 1,4-diazabicyclo[2.2.2] octane, Bis(trimethylsilyl)amine), metal hydroxides (e.g., sodium hydroxide, potassium hydroxide, cesium hydroxide).

In some embodiments, the twelfth base is a guanidine. In some embodiments, the twelfth base is selected from triazabicyclodecene and tetra-methyl guanidine. In some embodiments, the twelfth base is tetra-methyl guanidine.

In some embodiments, the reacting of the compound of Formula O-3 or a salt thereof is performed in a twenty-fourth solvent. In some embodiments, the twenty-fourth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol, tert-amyl alcohol), a halogenated solvent (e.g., dichloromethane, 1,2-dichloroethane, chlorobenzene, α,α,α-trifluorotoluene), or a combination thereof. In some embodiments, the twenty-fourth solvent comprises an alcohol. In some embodiments, the twenty-fourth solvent comprises methanol, ethanol, 2-propanol, butanol, glycerol, iso-amyl alcohol, tert-amyl alcohol, or a combination thereof. In some embodiments, the reacting of the compound of Formula O-3 or a salt thereof is performed in a twenty-fourth solvent comprising methanol.

In some embodiments, the reacting of the compound of Formula O-3 or a salt thereof is carried out in a temperature range of from about −10° C. to about 80° C. In some embodiments, the reacting of the compound of Formula O-3 or a salt thereof is carried out in a temperature range of from about 10° C. to about 30° C. In some embodiments, the reacting of the compound of Formula O-3 or a salt thereof is carried out at a temperature of about 20° C.

In some embodiments, the process further comprises preparing the compound of Formula O-3 or a salt thereof by a process comprising:
reacting a compound of Formula O-4:

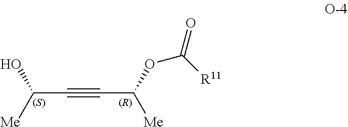

or a salt thereof, with:
a second phosphine reagent,
a second azodicarboxylate, and
a compound of Formula R⁷—NH—NH—R⁸ or methyl (1,3-dioxoisoindolin-2-yl) carbamate;
   to provide the compound of Formula O-3 or a salt thereof.

In some embodiments, suitable second phosphine reagents include triaryl phosphines (e.g., triphenylphosphine, tris (2-pyridyl) phosphine, tris-(4-methoxyphenyl) phosphine), trialkyl phosphines (e.g., tricyclohexyl phosphine, tributyl phosphine, trimethyl phosphine), tris-(dimethylaminophenyl) phosphine, (triphenylphosphoranylidene) acetonitrile, and phosphoranes (e.g., (cyanomethylene) tributylphosphorane).

In some embodiments, the second phosphine reagent is selected from a trialkyl phosphine, a triaryl phosphine, (triphenylphosphoranylidene) acetonitrile, and a phosphorane. In some embodiments, the second phosphine reagent is a triarylphosphine. In some embodiments, the second phosphine reagent comprises triphenylphosphine, tris (2-pyridyl) phosphine, tris-(4-methoxyphenyl) phosphine, or a combination thereof. In some embodiments, the second phosphine reagent is triphenylphosphine.

In some embodiments, the second azodicarboxylate is selected from di-tert-butyl azodicarboxylate, diisopropyl azodicarboxylate, diethyl azodicarboxylate, dimethoxy ethylazodicarboxylate, azodicarboxylic dimorpholide, 1,1-azodicarbonyl dipiperidine, and di-4-chlorobenzyl azodicarboxylate. In some embodiments, the second azodicarboxylate is di-tert-butylazodicarboxylate.

In some embodiments, the compound of Formula R⁷—NH—NH—R⁸ is

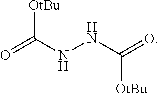 [SMILES: CC(C)(C)OC(=O)NNC(=O)OC(C)(C)C]

In some embodiments, the reacting of the compound of Formula O-4 or a salt thereof is performed in a twenty-fifth solvent. In some embodiments, the twenty-fifth solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP), dimethyl sulfoxide), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), a halogenated solvent (e.g., dichloromethane, 1,2-dichloroethane, chlorobenzene, α,α,α-trifluorotoluene), or a combination thereof. In some embodiments, the twenty-fifth solvent comprises an ether. In some embodiments, the twenty-fifth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments the reacting of the compound of Formula O-4 or a salt thereof is performed in a twenty-fifth solvent comprising 2-methyltetrahydrofuran.

In some embodiments, the reacting of the compound of Formula O-4 or a salt thereof is carried out in a temperature range of from about −20° C. to about 50° C. In some embodiments, the reacting of the compound of Formula O-4 or a salt thereof is carried out in a temperature range of from about 0° C. to about 22° C.

In some embodiments, the process further comprises preparing the compound of Formula O-4 or a salt thereof by a process comprising:
reacting a compound of Formula L:

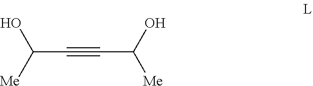

or a salt thereof, with:
an alkyl ester, an alkenyl ester or an oxime ester; and
an enzyme,
   to provide the compound of Formula O-4 or a salt thereof.

In some embodiments, the compound of Formula L or a salt thereof is reacted with an alkenyl ester. In some embodiments, the compound of Formula L or a salt thereof is reacted with an alkenyl ester is selected from vinyl acetate, isopropenyl acetate, and vinyl butyrate. In some embodiments, the alkenyl ester is isopropenyl acetate.

In some embodiments, the compound of Formula L or a salt thereof is reacted with an alkyl ester. In some embodiments, the compound of Formula L or a salt thereof is reacted with an alkyl ester selected from ethyl acetate, methyl acetate, isopropyl acetate, and methyl butyrate.

In some embodiments, the compound of Formula L or a salt thereof is reacted with an oxime ester. In some embodiments, the compound of Formula L or a salt thereof is reacted with an oxime ester is selected from propan-2-one O-acetyl oxime and propan-2-one O-butyryl oxime.

In some embodiments, suitable enzymes include hydrolases (e.g., Candida antarctica lipase A and B, lipase AK, lipase PS, Candida rugosa lipase), Prozomix 4, Prozomix 5, Prozomix 25, Prozomix 26, and Novozyme 435. In some embodiments, the enzyme is selected from a hydrolase enzyme, Prozomix 4, Prozomix 5, Prozomix 25, Prozomix 26, and Novozyme 435. In some embodiments, the enzyme is a hydrolase enzyme. In some embodiments, the enzyme is Lipase AK.

In some embodiments, the reacting of the compound of Formula L or a salt thereof is performed in a twenty-sixth solvent. In some embodiments, the twenty-sixth solvent comprises an ether (e.g., tetrahydrofuran, 2-methyltetrahydrofuran, tert-butyl methyl ether, 1,3-dioxolane, cyclopentyl methyl ether, 1,4-dioxane), a hydrocarbon (e.g., toluene, n-heptane) an alcohol (e.g., methanol, ethanol, 2-propanol, butanol, glycerol, iso-amyl alcohol, tert-amyl alcohol), or a combination thereof. In some embodiments, the twenty-sixth solvent comprises an ether. In some embodiments, the twenty-sixth solvent comprises tetrahydrofuran, 2-methyltetrahydrofuran, tert-butyl methyl ether, 1,3-dioxolane, cyclopentyl methyl ether, 1,4-dioxane, or a combination thereof. In some embodiments, the reacting of the compound of Formula L or a salt thereof is performed in a twenty-sixth solvent comprising tert-butyl methyl ether.

In some embodiments, the reacting of the compound of Formula L or a salt thereof is carried out in a temperature range of from about 10° C. to about 70° C. In some embodiments, the reacting of the compound of Formula L or a salt thereof is carried out in a temperature range of from about 30° C. to about 60° C. In some embodiments, the reacting of the compound of Formula L or a salt thereof is carried out at a temperature of about 50° C.

In some embodiments, provided herein is a process for preparing a compound of Formula I-A:

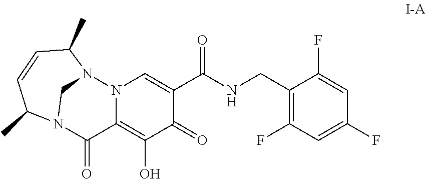

or a salt thereof, comprising:
(a) reacting a compound of Formula A:

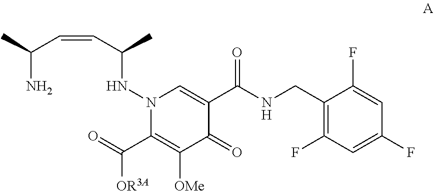

or a salt thereof, wherein R^{3A} is a hydroxyl protecting group, with:
   a methylene reagent, and
   a first acid or a first base,
to provide a compound of Formula I:

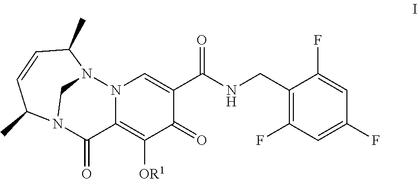

or a salt thereof, wherein R¹ is a hydroxyl protecting group; and
(b) deprotecting the compound of Formula I or a salt thereof, to provide the compound of Formula I-A or a salt thereof.

In some embodiments, provided herein is a process for preparing a compound of Formula I-A:

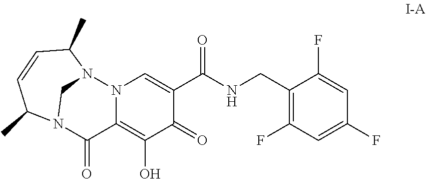

or a salt thereof, comprising:
(a) reacting a compound of Formula R-1:

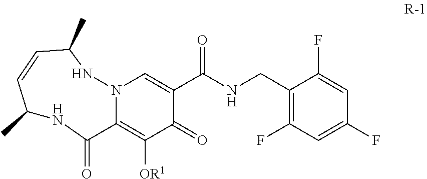

or a salt thereof, with:
   a methylene reagent, and
   an eighth acid or a fourteenth base,
to provide a compound of Formula I:

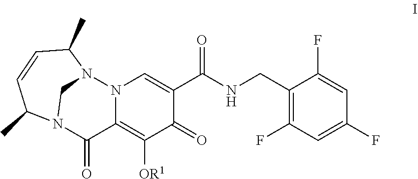

or a salt thereof, wherein R¹ is a hydroxyl protecting group; and
(b) deprotecting the compound of Formula I or a salt thereof, to provide the compound of Formula I-A or a salt thereof.

In some embodiments of the processes disclosed herein, the compound of Formula R-1 or a salt thereof by a process comprising:
(c) reacting a compound of Formula A:

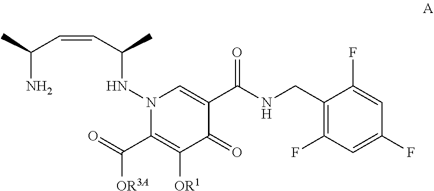

or a salt thereof, wherein R^{3A} is hydrogen or a hydroxyl protecting group, with:
   a reagent selected from a metal hydroxide, a metal halide, and a trialkylsilyl halide;
to provide a compound of Formula R-2:

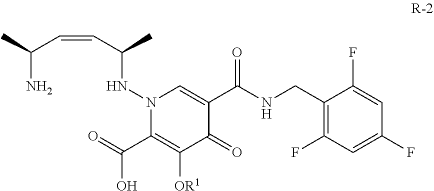

or a salt thereof; and
(d) reacting the compound of Formula R-2 or a salt thereof, with:
   a coupling agent, and
   a fifteenth base,
to provide the compound of Formula R-1 or a salt thereof

In some embodiments of the processes disclosed herein, the process further comprises preparing the compound of Formula R-1 or a salt thereof by a process comprising:
reacting a compound of Formula A:

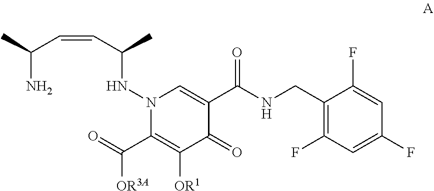

or a salt thereof, wherein R^{3A} is a hydroxyl protecting group, with:
a sixteenth base;
   to provide the compound of Formula R-1 or a salt thereof.

In some embodiments of the processes disclosed herein, the process further comprises preparing the compound of Formula A or a salt thereof by a process comprising:
(e) deprotecting a compound of Formula D:

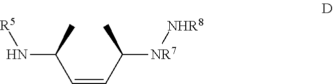

or a salt thereof, wherein R⁵ is hydrogen or R⁹, wherein R⁹ is an amine protecting group, and R⁷ and R⁸ are each independently an amine protecting group;
to provide a compound of Formula C:

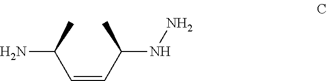

or a salt thereof; and
(f) reacting a compound of Formula X-A:

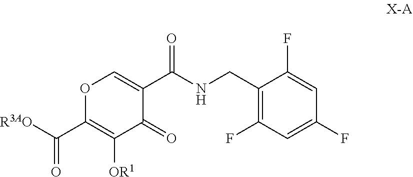

or a salt thereof, with:
   the compound of Formula C or a salt thereof, and
   a seventh base;
to provide the compound of Formula A or a salt thereof.

In some embodiments of the processes disclosed herein, the process further comprises preparing the compound of Formula D or a salt thereof by a process comprising:
(g) deprotecting a compound of Formula E:

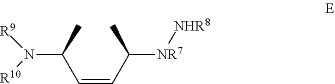

or a salt thereof, wherein:
   R⁷ and R⁸ are each independently an amine protecting group; and
   R⁹ is an amine protecting group and R¹⁰ is hydrogen; or optionally:
   R⁹ and R¹⁰ together with the nitrogen atom to which they are attached form a heterocyclic ring protecting group;
to provide the compound of Formula D or a salt thereof having Formula D-1:

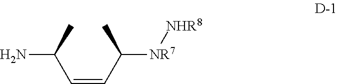

or a salt thereof.

In some embodiments of the processes disclosed herein, the process further comprises preparing the compound of Formula A by a process comprising:
(e) reacting a compound of Formula E:

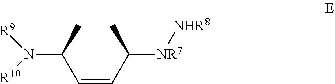

or a salt thereof, wherein R⁹ and R¹⁰ together with the nitrogen atom to which they are attached form phthalimide;
with a seventh acid to provide a compound of Formula Q-2:

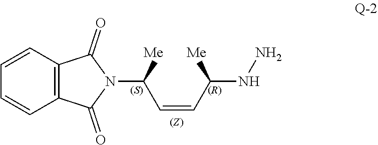

or a salt thereof;
(f) reacting the compound of Formula Q-2 or a salt thereof, with:
a compound of Formula X-A:

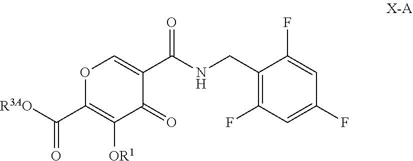

or a salt thereof, and
   a thirteenth base;
to provide a compound of Formula Q-1:

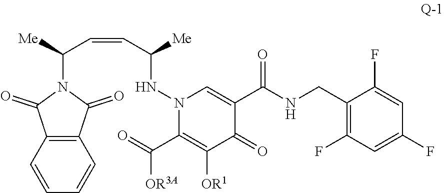

or a salt thereof; and
(g) reacting the compound of Formula Q-1 or a salt thereof, with a reagent selected from a hydrazine, an alkoxide, and a hydrolase enzyme;
to provide the compound of Formula A or a salt thereof.

In some embodiments of the processes disclosed herein, the process further comprises preparing the compound of Formula E by a process comprising:
(h) reacting a compound of Formula H:

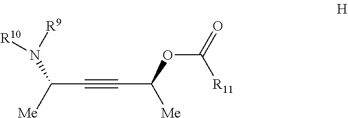

or a salt thereof, wherein R¹¹ is selected from C₁₋₂₀alkyl, phenyl, and —CH₂NH₂;
with a sixth acid or an eighth base,
to provide a compound of Formula G:

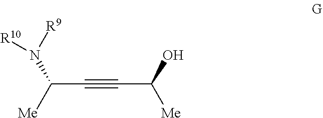

or a salt thereof;
(i) reacting the compound of Formula G or a salt thereof, with:
   a third catalyst, and
   hydrogen;
to provide a compound of Formula F:

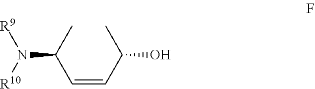

or a salt thereof; and
(j) reacting the compound of Formula F or a salt thereof, with:
   a phosphine reagent,
   an azodicarboxylate, and
   a compound of formula R⁷—NH—NH—R⁸;
to provide the compound of Formula E or a salt thereof.

In some embodiments of the processes disclosed herein, the process further comprises preparing the compound of Formula E by a process comprising:
(h) reacting a compound of Formula F:

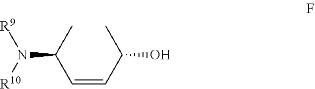

or a salt thereof, with:
   a phosphine reagent,
   an azodicarboxylate, and
   a compound of formula R⁷—NH—NH—R⁸;
to provide the compound of Formula E or a salt thereof.

In some embodiments of the processes disclosed herein, the process further comprises preparing the compound of Formula F by a process comprising:
(i) reacting a compound of Formula S:

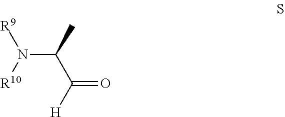

or a salt thereof, with:
   a compound of Formula T:

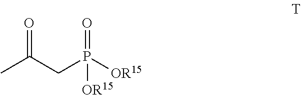

or a salt thereof,
   wherein R¹⁵ is selected from phenyl and C₁₋₄haloalkyl, wherein the phenyl is optionally substituted with 1-4 substituents selected from C₁₋₄alkyl, halo, C₁₋₄ haloalkyl, and cyano;
a seventeenth base, and
a fourth additive;
to provide a compound of Formula W:

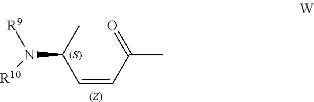

or a salt thereof; and
(j) reacting the compound of Formula W or a salt thereof, with:
   a sixth catalyst, and
   a reducing agent;
to provide the compound of Formula F or a salt thereof.

In some embodiments of the processes disclosed herein, the process further comprises preparing the compound of Formula F by a process comprising:
(i) reacting a compound of Formula W:

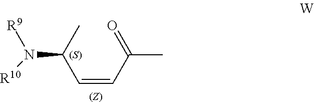

or a salt thereof, with:
   a second enzyme in the presence of a second buffer;
to provide the compound of Formula F or a salt thereof.

In some embodiments of the processes disclosed herein, the process further comprises preparing the compound of Formula F by a process comprising:
(i) reacting a compound of Formula S:

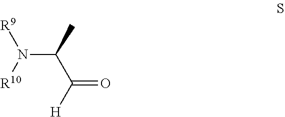

or a salt thereof, with:
   a compound of Formula T:

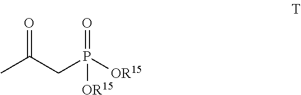

or a salt thereof;
   wherein R¹⁵ is selected from phenyl and C₁₋₄haloalkyl, wherein the phenyl is optionally substituted with 1-4 substituents selected from C₁₋₄alkyl, halo, C₁₋₄ haloalkyl, and cyano;
a seventeenth base, and
a fourth additive;
to provide a compound of Formula W:

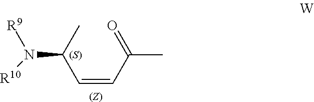

or a salt thereof; and
(j) reacting the compound of Formula W or a salt thereof, with:
   a sixth catalyst, and
   a reducing agent;
to provide the compound of Formula F or a salt thereof.

In some embodiments of the processes disclosed herein, the process further comprises preparing the compound of Formula F by a process comprising:
(i) reacting a compound of Formula G:

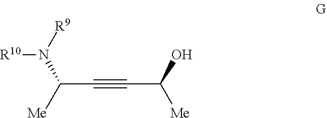

or a salt thereof, with:
   a third reducing agent, and
   an activator;
to provide the compound of Formula F or a salt thereof.

In some embodiments of the processes disclosed herein, the process further comprises preparing the compound of Formula D or a salt thereof by a process comprising:
(g) reacting a compound of Formula H having Formula H-1:

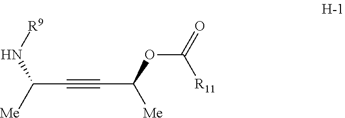

or a salt thereof, wherein:
   R⁹ is an amine protecting group; and
   R¹¹ is selected from C₁₋₂₀alkyl, phenyl, and —CH₂NH₂;
with a twelfth base,
to provide a compound of Formula G-1:

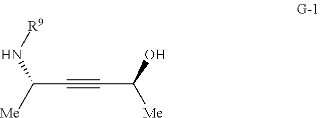

or a salt thereof;
(h) reacting the compound of Formula G-1 or a salt thereof, with:
   a third catalyst,
   an aromatic amine or a sulfur-containing reagent; and
   hydrogen;
to provide the compound of Formula F-1:

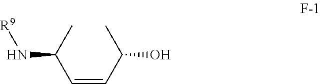

or a salt thereof; and
(i) reacting the compound of Formula F-1 or a salt thereof, with:
   a phosphine reagent,
   an azodicarboxylate, and
   a compound of formula R⁷—NH—NH—R⁸;
to provide the compound of Formula D or a salt thereof.

In some embodiments of the processes disclosed herein, the process further comprises preparing the compound of Formula D or a salt thereof by a process comprising:
(g) reacting a compound of Formula G-1:

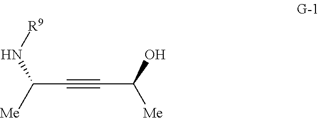

or a salt thereof, with:
   a third catalyst,
   an aromatic amine or a sulfur-containing reagent; and
   hydrogen;
to provide a compound of Formula F-1:

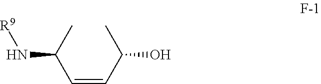

or a salt thereof; and
(h) reacting the compound of Formula F-1 or a salt thereof, with:
   a phosphine reagent,
   an azodicarboxylate, and
   a compound of formula R⁷—NH—NH—R⁸;
to provide the compound of Formula D or a salt thereof.

In some embodiments of the processes disclosed herein, the process further comprises preparing the compound of Formula G or a salt thereof, wherein the compound of Formula G or a salt thereof is a compound of Formula G-1 or a salt thereof, by a process comprising:
(j) reacting a compound of Formula L:

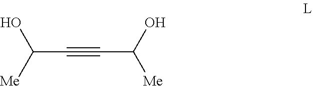

or a salt thereof, with:
   a fourth enzyme in the presence of a fourth buffer;
to provide a compound of Formula U-2:

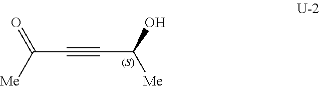

or a salt thereof;
(k) reacting the compound of Formula U-2 or a salt thereof, with:
   a third enzyme in the presence of a third buffer;
to provide a compound of Formula U-1:

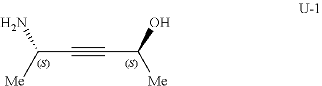

or a salt thereof; and
(l) reacting the compound of Formula U-1 or a salt thereof, with:
   a second amine protecting group reagent to provide the compound of Formula G-1 or a salt thereof.

In some embodiments of the processes disclosed herein, the process further comprises preparing the compound of Formula H or a salt thereof by a process comprising:
(k) reacting a compound of Formula J:

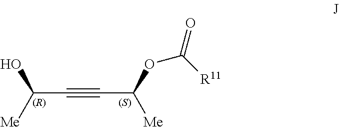

or a salt thereof, with:
   a second phosphine reagent,
   a second azodicarboxylate, and
   an amine protecting group reagent;
to provide the compound of Formula H or a salt thereof.

In some embodiments of the processes disclosed herein, the process further comprises preparing the compound of Formula H or a salt thereof by a process comprising:
(k) reacting a compound of Formula J:

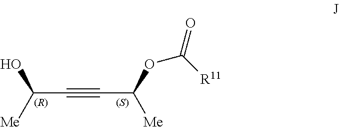

or a salt thereof, wherein:
   R¹² is —S(O)₃R¹³ or —OC(═N)R¹⁴;
   R¹³ is selected from C₁₋₄alkyl, C₁₋₄haloalkyl, and phenyl optionally substituted with C₁₋₄alkyl; and
   R¹⁴ is C₁₋₄haloalkyl;
with:
   an eleventh base, and
   a third reagent selected from methane sulfonyl chloride, an anhydride, or an acid halide;
to provide a compound of Formula N:

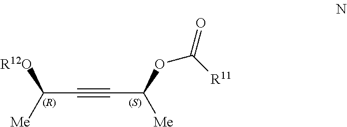

or a salt thereof; and
(l) reacting the compound of Formula N or a salt thereof with an amine protecting group reagent,
to provide the compound of Formula H or a salt thereof.

In some embodiments of the processes disclosed herein, the process further comprises preparing the compound of Formula H-1 or a salt thereof by a process comprising:
(k) reacting a compound of Formula N:

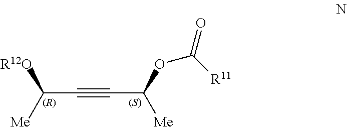

or a salt thereof, wherein:
   R¹² is —S(O)₃R¹³ or —OC(═N)R¹⁴; and
   R¹³ is selected from C₁₋₄alkyl, C₁₋₄haloalkyl, and phenyl optionally substituted with C₁₋₄alkyl;
with:
   an azide, and
   a third phosphine reagent;
to provide a compound of Formula P-1:

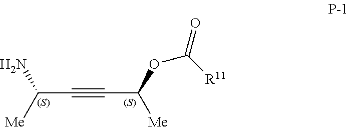

or a salt thereof; and
(l) reacting the compound of Formula P-1 or a salt thereof with an amine protecting group reagent to provide the compound of Formula H-1 or a salt thereof.

In some embodiments of the processes disclosed herein, the process further comprises preparing the compound of Formula H-1 or a salt thereof by a process comprising:
(k) reacting a compound of reacting a compound of Formula J:

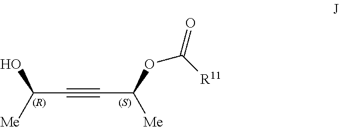

or a salt thereof, with:
   an azide,
   a third phosphine reagent, and
   a third azodicarboxylate;
to provide a compound of Formula P-2:

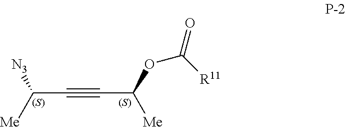

or a salt thereof; and
(l) reacting the compound of Formula P-2 or a salt thereof, with:
   a fourth phosphine reagent; and
   an amine protecting group reagent;
to provide the compound of Formula H-1 or a salt thereof.

In some embodiments of the processes disclosed herein, the process further comprises preparing the compound of Formula J or a salt thereof by a process comprising:
(m) reacting a compound of Formula L:

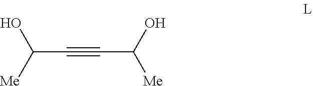

or a salt thereof; with:
   a ninth base,
   a fourth catalyst; and
   a compound of Formula M-1 or Formula M-2:

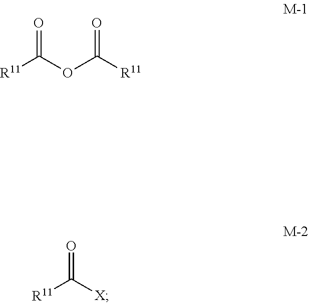

wherein X is a halide;
to provide a compound of Formula K:

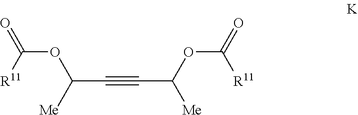

or a salt thereof; and
(n) reacting the compound of Formula K or a salt thereof, with:
   an enzyme, and
   a buffer;
to provide the compound of Formula J or a salt thereof.

In some embodiments of the processes disclosed herein, the process further comprises comprising preparing the compound of Formula E by a process comprising:
(h) reacting a compound of Formula L:

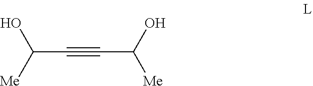

or a salt thereof, with:
   an alkyl ester, an alkenyl ester or an oxime ester; and
   an enzyme,
to provide a compound of Formula O-4:

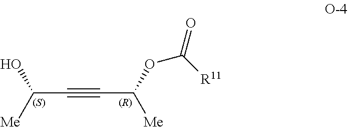

or a salt thereof;
(i) reacting the compound of Formula O-4 or a salt thereof, with:
   a second phosphine reagent,
   a second azodicarboxylate, and
   a compound of Formula R⁷—NH—NH—R⁸ or methyl (1,3-dioxoisoindolin-2-yl) carbamate;
to provide a compound of Formula O-3:

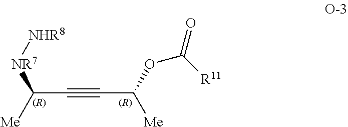

or a salt thereof;
(j) reacting the compound of Formula O-3 or a salt thereof, wherein R¹¹ is selected from C₁₋₂₀alkyl, phenyl, and —CH₂NH₂; with:
   a twelfth base;
to provide a compound of Formula O-2:

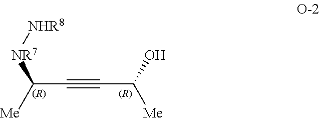

or a salt thereof;
(k) reacting the compound of Formula O-2 or a salt thereof, with:
   a fifth catalyst, and
   hydrogen;
to provide the compound of Formula O-1:

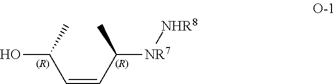

or a salt thereof; and
(l) reacting the compound of Formula O-1 or a salt thereof, with:
   an amine protecting group reagent,
   a phosphine reagent, and
   an azodicarboxylate,
to provide the compound of Formula E or a salt thereof.

### Alternative Process for Preparing the Compound of Formula I

In some embodiments, provided herein is a process comprising preparing a compound of Formula I:

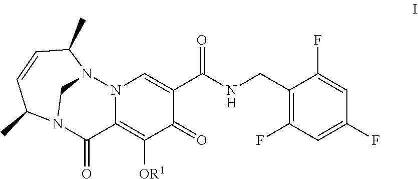

or a salt thereof, wherein R¹ is selected from hydrogen and a hydroxyl protecting group; comprising:
reacting a compound of Formula R-1:

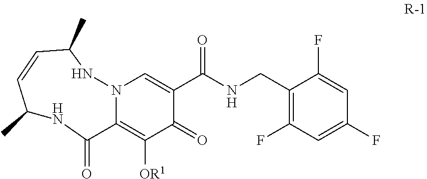

or a salt thereof, with:
   a methylene reagent, and
   an eighth acid or a fourteenth base,
to provide the compound of Formula I or a salt thereof.

In some embodiments, the methylene reagent is a formaldehyde reagent or a dihalomethane. In some embodiments, suitable formaldehyde reagents include formaldehyde acetals (e.g., dimethoxymethane, diethoxymethane), formaldehyde solution, paraformaldehyde, and 1,3,5-trioxane. In some embodiments, suitable methylene reagents include dihalomethanes (e.g., dibromomethane, diiodomethane).

In some embodiments, the methylene reagent is selected from 1,3,5-trioxane, a formaldehyde acetal, formaldehyde, paraformaldehyde, or a dihalomethane. In some embodiments, the methylene reagent is a formaldehyde reagent. In some embodiments, the methylene reagent is selected from 1,3,5-trioxane, a formaldehyde acetal, formaldehyde, and paraformaldehyde. In some embodiments, the methylene reagent is formaldehyde.

In some embodiments, the compound of Formula R-1 or a salt thereof is reacted with the eighth acid. In some embodiments, suitable eighth acids include carboxylic acids (e.g., oxalic acid, trichloroacetic acid, trifluoroacetic acid), sulfonic acids (e.g., methanesulfonic acid, benzenesulfonic acid, para-toluenesulfonic acid, camphorsulfonic acid), Lewis acids (e.g., boron trifluoride methyl etherate, boron trifluoride dibutyl etherate, boron trichloride, boron tribromide, aluminum chloride, aluminum bromide), inorganic acids (e.g., hydrochloric acid, sulfuric acid, phosphoric acid, hydrobromic acid), or a combination thereof.

In some embodiments, the eighth acid comprises a carboxylic acid, a sulfonic acid, a Lewis acid, an inorganic acid, or a combination thereof. In some embodiments, the eighth acid comprises a sulfonic acid. In some embodiments, the eight acid comprises methanesulfonic acid, benzenesulfonic acid, para-toluenesulfonic acid, camphorsulfonic acid, or a combination thereof. In some embodiments, the eighth acid is methanesulfonic acid.

In some embodiments, the compound of Formula R-1 or a salt thereof is reacted with the fourteenth base. In some embodiments, suitable fourteenth bases include amines (e.g., n-propylamine, triethylamine, N,N-diisopropylethylamine, 1,4-diazabicyclo[2.2.2]octane (DABCO), quinuclidine), amidines (e.g., 1,8-diazabicyclo[5.4.0]undec-7-ene (DBU), 1,5-diazabicyclo[4.3.0]non-5-ene (DBN), etc.), guanidines (e.g., triazabicyclodecene (TBD), 1,1,3,3-tetramethylguanidine), carboxylates (e.g., sodium acetate, potassium acetate, sodium propionate), carbonates (e.g., M(n)H(2-n)CO₃ such as sodium carbonate, potassium bicarbonate, cesium carbonate), metal hydrides (e.g., lithium hydride, sodium hydride, potassium hydride), phosphates (e.g., M(n)H(3-n)PO₄ such as trisodium phosphate, disodium hydrogen phosphate, tripotassium phosphate, dipotassium hydrogen phosphate), hydroxides (e.g., sodium hydroxide, potassium hydroxide, cesium hydroxide), alkoxides (e.g., sodium methoxide, sodium ethoxide, potassium tert-butoxide, etc.), amides (e.g., lithium bis(trimethylsilyl)amide, potassium bis(trimethylsilyl)amide, lithium diisopropylamide), basic aromatic compounds (e.g., pyridine, 2,6-lutidine, imidazole), or a combination thereof.

In some embodiments, the fourteenth base comprises an amine, an amidine, a guanidine, a carboxylate, a carbonate, a metal hydride, a phosphate, a hydroxide, an alkoxide, an amide, a basic aromatic compound, or a combination thereof.

In some embodiments, the reacting of the compound of Formula R-1 or a salt thereof is performed in a forty-second solvent. In some embodiments, the forty-second solvent comprises an ether (e.g., tetrahydrofuran, 2-methyltetrahydrofuran, tert-butyl methyl ether, 1,3-dioxolane, cyclopentyl methyl ether, 1,4-dioxane), a nitrile (e.g., acetonitrile, propionitrile, butyronitrile), a hydrocarbon (e.g., toluene, methylcyclohexane, n-heptane, hexanes, limonene), a polar aprotic solvent (e.g., N,N-dimethylformamide, N,N-dimethylacetamide, N-methyl-2-pyrrolidone, N-butyl pyrrolidone (NBP)), an ester (e.g., ethyl acetate, methyl acetate, isopropyl acetate, dimethyl-2-methyl glutarate, propylene carbonate), a ketone (e.g., acetone, 2-butanone, 4-methyl-2-pentanone), an alcohol (e.g., methanol, ethanol, 2-propanol, butanol, glycerol, iso-amyl alcohol), a halogenated solvent (e.g., dichloromethane, 1,2-dichloroethane, chlorobenzene, α,α,α-trifluorotoluene), or a combination thereof. In some embodiments, the forty-second solvent comprises an ether. In some embodiments, the forty-second solvent comprises tetrahydrofuran, 2-methyltetrahydrofuran, tert-butyl methyl ether, 1,3-dioxolane, cyclopentyl methyl ether, 1,4-dioxane, or a combination thereof.

In some embodiments, the reacting of the compound of Formula R-1 or a salt thereof is carried out in a temperature range of from about 20° C. to about 100° C. In some embodiments, the reacting of the compound of Formula R-1 or a salt thereof is carried out in a temperature range of from about 50° C. to about 70° C. In some embodiments, the reacting of the compound of Formula R-1 or a salt thereof is carried out at a temperature of about 65° C.

In some embodiments, the process further comprises preparing the compound of Formula R-1 or a salt thereof by a process comprising reacting a compound of Formula R-2 or a salt thereof with a coupling agent and a fifteenth base to provide the compound of Formula R-1 or a salt thereof, as described herein.

In some embodiments, the process further comprises preparing the compound of Formula R-2 or a salt thereof by a process comprising reacting a compound of Formula A or a salt thereof with a reagent selected from a metal hydroxide, a metal halide, and a trialkylsilyl halide; to provide the compound of Formula R-2 or a salt thereof, as described herein.

In some embodiments, provided herein is a process for preparing a compound of Formula I:

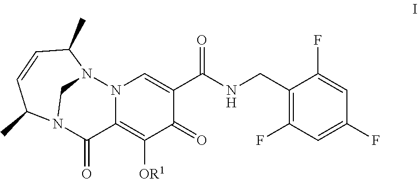

or a salt thereof, wherein R¹ is selected from hydrogen and a hydroxyl protecting group; comprising:
(a) reacting a compound of Formula A:

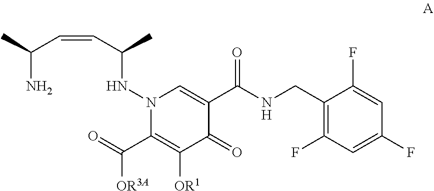

or a salt thereof, wherein R^{3A} is hydrogen or a hydroxyl protecting group, with:
   a reagent selected from a metal hydroxide, a metal halide, and a trialkylsilyl halide;
to provide a compound of Formula R-2:

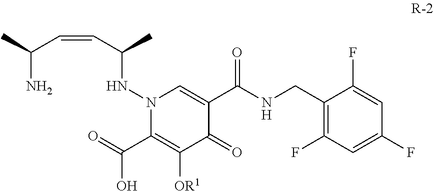

or a salt thereof;
(b) reacting the compound of Formula R-2 or a salt thereof, with:
   a coupling agent, and
   a fifteenth base,
to provide a compound of Formula R-1

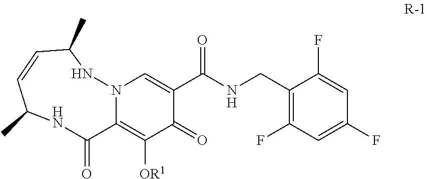

or a salt thereof; and
(c) reacting the compound of Formula R-1 or a salt thereof, with:
   a methylene reagent, and
   an eighth acid or a fourteenth base,
to provide the compound of Formula I or a salt thereof.

### Intermediates

In some embodiments, provided herein are intermediates for the formation of a compound of Formula I (e.g., a compound of Formula I-A) or salts thereof.

Accordingly, in some embodiments, a compound of Formula I:

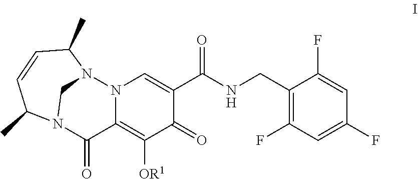

or a salt thereof, wherein R¹ is a hydroxyl protecting group, is provided.

In some embodiments, the compound of Formula I or a salt thereof is a compound of formula I-B:

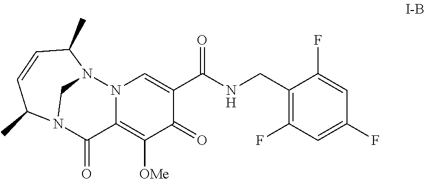

or a salt thereof.

In some embodiments, a compound of Formula II:

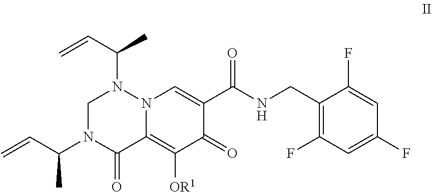

or a salt thereof, wherein R¹ is a hydroxyl protecting group, is provided.

In some embodiments, the compound of Formula II or a salt thereof is a compound of formula II-A:

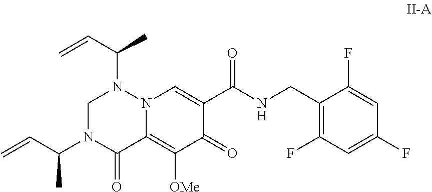 [SMILES: C=C[C@@H](C)N1CN([C@@H](C)C=C)C(=O)c2c(OC)c(=O)c(C(=O)NCc3c(F)cc(F)cc3F)cn21]

or a salt thereof.

In some embodiments, a compound of Formula III:

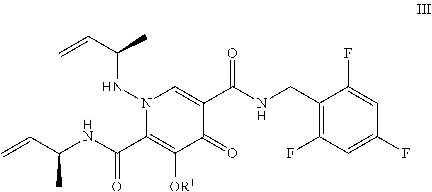

or a salt thereof, wherein R¹ is a hydroxyl protecting group, is provided.

In some embodiments, the compound of Formula III or a salt thereof is a compound of Formula III-A:

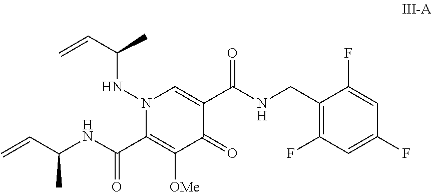 [SMILES: C=C[C@@H](C)Nn1cc(C(=O)NCc2c(F)cc(F)cc2F)c(=O)c(OC)c1C(=O)N[C@@H](C)C=C]

or a salt thereof.

In some embodiments, a compound of Formula IV:

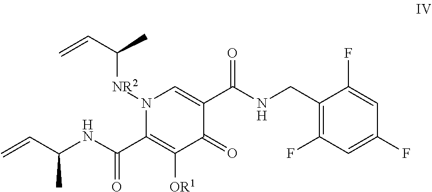

or a salt thereof, wherein R¹ is a hydroxyl protecting group and R² is an amine protecting group, is provided.

In some embodiments, the compound of Formula IV or a salt thereof is a compound of Formula IV-A:

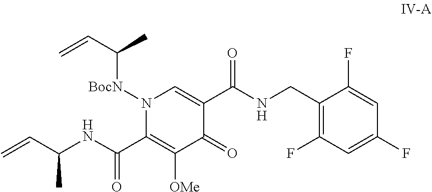 [SMILES: C=C[C@@H](C)N(C(=O)OC(C)(C)C)n1cc(C(=O)NCc2c(F)cc(F)cc2F)c(=O)c(OC)c1C(=O)N[C@@H](C)C=C]

or a salt thereof.

In some embodiments, a compound of Formula V:

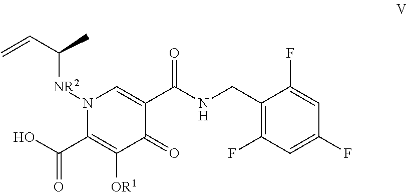

or a salt thereof, wherein R¹ is a hydroxyl protecting group and R² is an amine protecting group, is provided.

In some embodiments, the compound of Formula V or a salt thereof is a compound of Formula V-A:

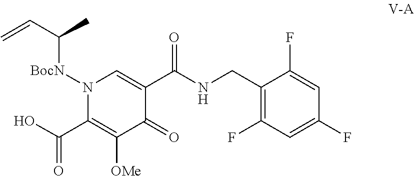 [SMILES: C=C[C@@H](C)N(C(=O)OC(C)(C)C)n1cc(C(=O)NCc2c(F)cc(F)cc2F)c(=O)c(OC)c1C(=O)O]

or a salt thereof.

In some embodiments, a compound of Formula VII:

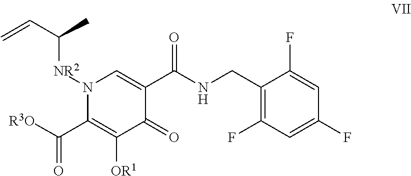

or a salt thereof, wherein R¹ and R³ are each independently hydroxyl protecting groups and R² is hydrogen or an amine protecting group, is provided.

In some embodiments, the compound of Formula VII or a salt thereof is a compound of Formula VII-A:

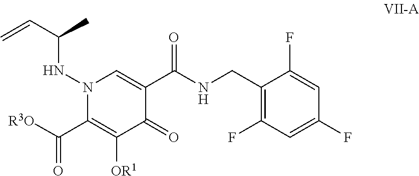

or a salt thereof.

In some embodiments, the compound of Formula VII or a salt thereof is a compound of Formula VII-B:

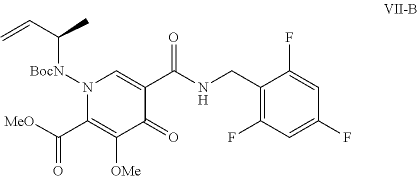 [SMILES: C=C[C@@H](C)N(C(=O)OC(C)(C)C)n1cc(C(=O)NCc2c(F)cc(F)cc2F)c(=O)c(OC)c1C(=O)OC]

or a salt thereof.

In some embodiments, a compound of Formula VIII:

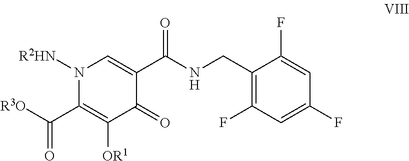

or a salt thereof, wherein R¹ and R³ are each independently hydroxyl protecting groups and R² is hydrogen or an amine protecting group, is provided.

In some embodiments, the compound of Formula VIII or a salt thereof is a compound of Formula VIII-A:

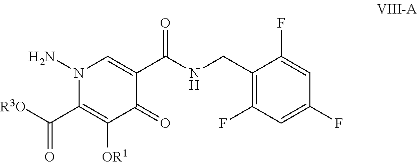

or a salt thereof.

In some embodiments, the compound of Formula VIII or a salt thereof is a compound of Formula VIII-B:

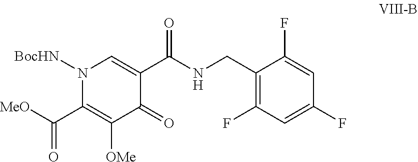 [SMILES: COC(=O)c1c(OC)c(=O)c(C(=O)NCc2c(F)cc(F)cc2F)cn1NC(=O)OC(C)(C)C]

or a salt thereof.

In some embodiments, a compound of Formula X:

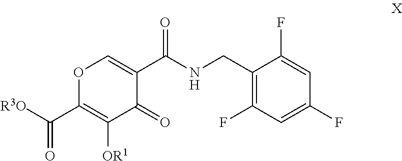

or a salt thereof, wherein R¹ and R³ are each independently hydroxyl protecting groups and R² is hydrogen or an amine protecting group, is provided.

In some embodiments, the compound of Formula X or a salt thereof is a compound of Formula X-A:

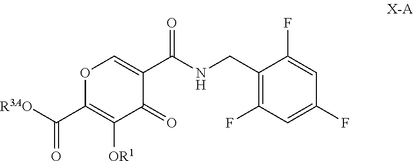

or a salt thereof.

In some embodiments, the compound of Formula X or a salt thereof is a compound of Formula X-B:

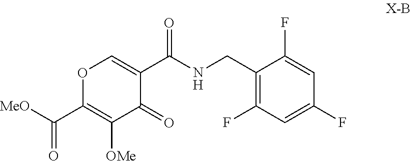 [SMILES: COC(=O)c1occ(C(=O)NCc2c(F)cc(F)cc2F)c(=O)c1OC]

or a salt thereof.

In some embodiments, a compound of Formula XI:

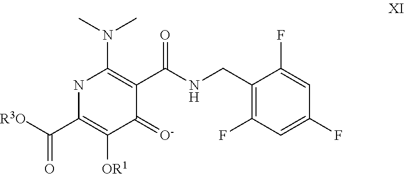

or a salt thereof, wherein R¹ and R³ are each independently hydroxyl protecting groups, is provided.

In some embodiments, the compound of Formula XI or a salt thereof is a compound of Formula XI-A:

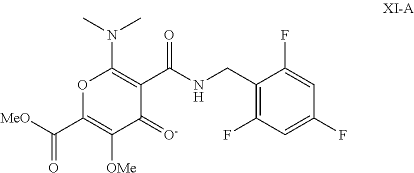

or a salt thereof.

In some embodiments, a compound of Formula XII:

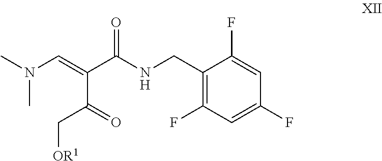

or a salt thereof, wherein R¹ is a hydroxyl protecting group, is provided.

In some embodiments, the compound of Formula XII or a salt thereof is a compound of Formula XII-A:

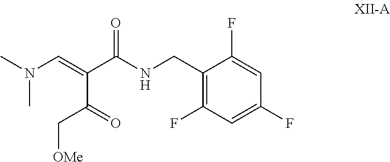 [SMILES: COCC(=O)/C(=C\N(C)C)C(=O)NCc1c(F)cc(F)cc1F]

or a salt thereof.

In some embodiments, a compound of Formula A:

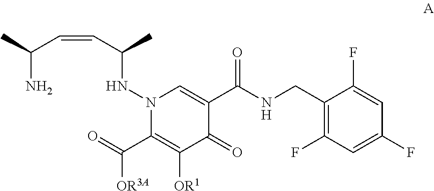

or a salt thereof, wherein R¹ and R^{3A} are independently selected from hydrogen and a hydroxyl protecting group, is provided.

In some embodiments, the compound of Formula A or a salt thereof is a compound of Formula A-1:

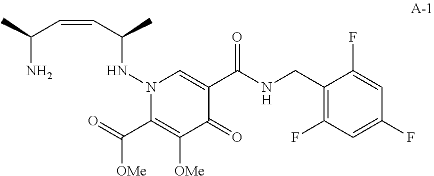 [SMILES: COC(=O)c1c(OC)c(=O)c(C(=O)NCc2c(F)cc(F)cc2F)cn1N[C@H](C)/C=C\[C@H](C)N]

or a salt thereof.

In some embodiments, a compound of Formula R-1:

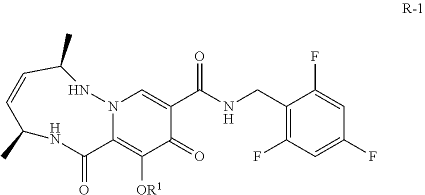

or a salt thereof, wherein R¹ is selected from hydrogen and a hydroxyl protecting group, is provided.

In some embodiments, the compound of Formula R-1 or a salt thereof is a compound of Formula R-1-A:

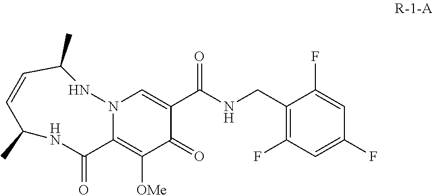 [SMILES: COc1c2n(cc(C(=O)NCc3c(F)cc(F)cc3F)c1=O)N[C@H](C)/C=C\[C@H](C)NC2=O]

or a salt thereof.

In some embodiments, provided herein is a compound of Formula R-2:

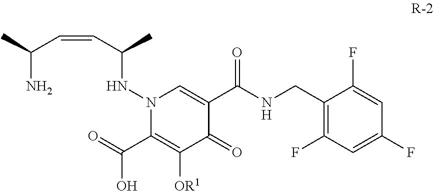

or a salt thereof, wherein R¹ is selected from hydrogen and a hydroxyl protecting group, is provided.

In some embodiments, the compound of Formula R-2 or a salt thereof is a compound of Formula R-2-A:

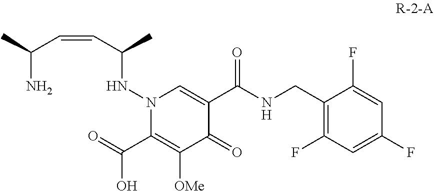 [SMILES: COc1c(C(=O)O)n(N[C@H](C)/C=C\[C@H](C)N)cc(C(=O)NCc2c(F)cc(F)cc2F)c1=O]

or a salt thereof.

In some embodiments, a compound of Formula Q-1:

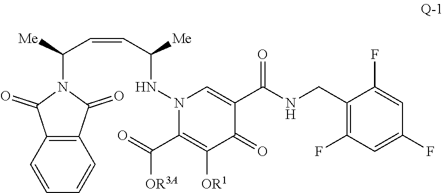

or a salt thereof, wherein R¹ and R^{3A} are each hydroxyl protecting groups, is provided.

In some embodiments, the compound of Formula Q-1 or a salt thereof is a compound of Formula Q-1-A:

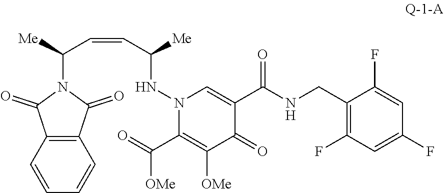 [SMILES: COC(=O)c1c(OC)c(=O)c(C(=O)NCc2c(F)cc(F)cc2F)cn1N[C@H](C)/C=C\[C@H](C)N1C(=O)c2ccccc2C1=O]

or a salt thereof.

In some embodiments, a compound of Formula Q-2:

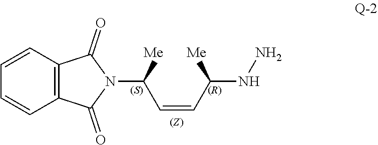

or a salt thereof, is provided.

In some embodiments, a compound of Formula C:

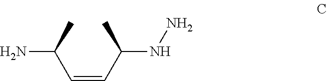

or a salt thereof, is provided.

In some embodiments, a compound of Formula D:

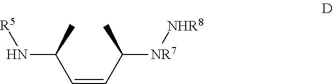

or a salt thereof, wherein:
R⁵ is selected from hydrogen and R⁹, wherein R⁹ is an amine protecting group; and
R⁷ and R⁸ are each independently an amine protecting group;
   is provided.

In some embodiments, the compound of Formula D or a salt thereof is a compound of Formula D-1:

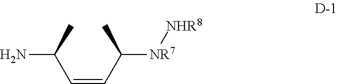

or a salt thereof.

In some embodiments, the compound of Formula D or a salt thereof is a compound of Formula D-2:

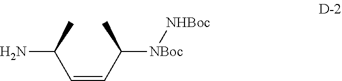 [SMILES: C[C@H](N)/C=C\[C@@H](C)N(NC(=O)OC(C)(C)C)C(=O)OC(C)(C)C]

or a salt thereof.

In some embodiments, a compound of Formula E:

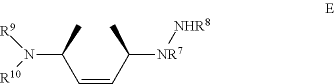

or a salt thereof, wherein:
R⁹ is an amine protecting group and R¹⁰ is hydrogen; or optionally:
R⁹ and R¹⁰ together with the nitrogen atom to which they are attached form a heterocyclic ring protecting group;
   is provided.

In some embodiments, a compound of Formula F:

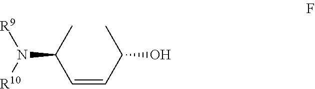

or a salt thereof, wherein:
R⁹ is an amine protecting group and R¹⁰ is hydrogen; or optionally:
R⁹ and R¹⁰ together with the nitrogen atom to which they are attached form a heterocyclic ring protecting group;
   is provided.

In some embodiments, the compound of Formula F or a salt thereof is a compound of Formula F-1:

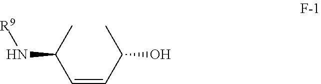

or a salt thereof.

In some embodiments, the compound of Formula F is a compound of Formula F-2:

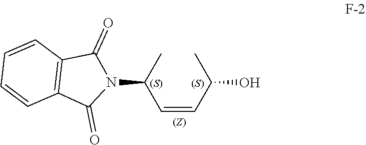

or a salt thereof.

In some embodiments, a compound of Formula G:

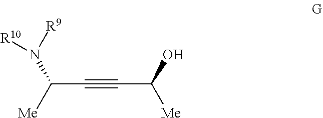

or a salt thereof, wherein:
R⁹ is an amine protecting group and R¹⁰ is hydrogen; or optionally:
R⁹ and R¹⁰ together with the nitrogen atom to which they are attached form a heterocyclic ring protecting group;
   is provided.

In some embodiments, the compound of Formula G or a salt thereof is a compound of Formula G-1:

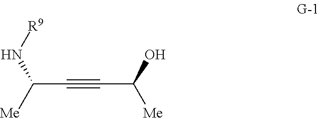

or a salt thereof.

In some embodiments, a compound of Formula H:

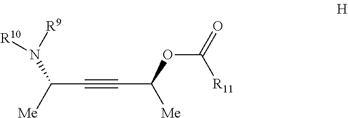

or a salt thereof, wherein:
R⁹ is an amine protecting group and R¹⁰ is hydrogen; or optionally:
R⁹ and R¹⁰ together with the nitrogen atom to which they are attached form a heterocyclic ring protecting group; and
R¹¹ is selected from C₁₋₂₀alkyl, phenyl, and —CH₂NH₂;
   is provided.

In some embodiments, the compound of Formula H or a salt thereof is a compound of Formula H-1:

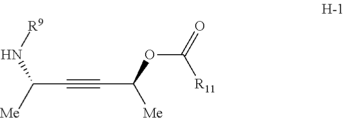

or a salt thereof.

In some embodiments, the compound of Formula H-1 or a salt thereof is a compound of Formula H-1-A:

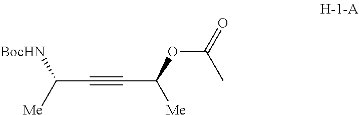 [SMILES: CC(=O)O[C@@H](C)C#C[C@H](C)NC(=O)OC(C)(C)C]

or a salt thereof.

In some embodiments, the compound of Formula H or a salt thereof is a compound of Formula H-2:

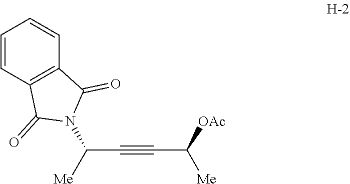 [SMILES: CC(=O)O[C@@H](C)C#C[C@H](C)N1C(=O)c2ccccc2C1=O]

or a salt thereof.

In some embodiments, a compound of Formula J:

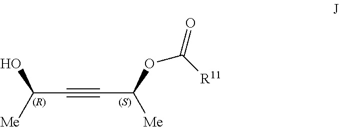

or a salt thereof, wherein R¹¹ is selected from C₁₋₂₀alkyl, phenyl, and —CH₂NH₂; is provided.

In some embodiments, a compound of Formula N:

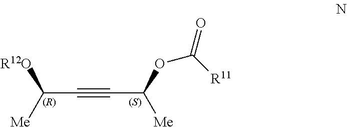

or a salt thereof, wherein:
R¹¹ is selected from C₁₋₂₀alkyl, phenyl, and —CH₂NH₂;
R¹² is —S(O)₃R¹³ or —OC(═N)R¹⁴;
R¹³ is selected from C₁₋₄alkyl, C₁₋₄haloalkyl, and phenyl optionally substituted with C₁₋₄alkyl; and
R¹⁴ is C₁₋₄haloalkyl;
   is provided.

In some embodiments, a compound of Formula P-1:

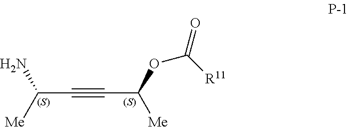

or a salt thereof, wherein:
R¹¹ is selected from C₁₋₂₀alkyl, phenyl, and —CH₂NH₂;
   is provided.

In some embodiments, a compound of Formula P-2:

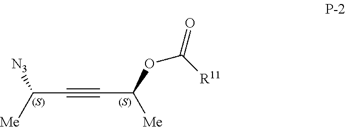

or a salt thereof, wherein:
R¹¹ is selected from C₁₋₂₀alkyl, phenyl, and —CH₂NH₂;
   is provided.

In some embodiments, a compound of Formula K:

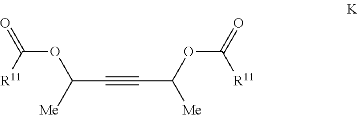

or a salt thereof, is provided.

In some embodiments, a compound of Formula O-1:

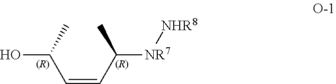

or a salt thereof, wherein R⁷ and R⁸ are each independently an amine protecting group, is provided.

In some embodiments, a compound of Formula O-2:

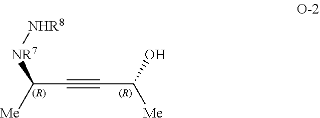

or a salt thereof, wherein R⁷ and R⁸ are each independently an amine protecting group, is provided.

In some embodiments, a compound of Formula O-3:

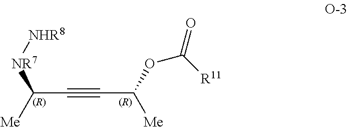

or a salt thereof, wherein:
R⁷ and R⁸ are each independently an amine protecting group; and
R¹¹ is selected from C₁₋₂₀alkyl, phenyl, and —CH₂NH₂;
   is provided.

In some embodiments, a compound of Formula O-4:

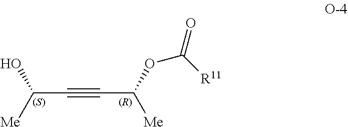

or a salt thereof, wherein R¹¹ is selected from C₁₋₂₀alkyl, phenyl, and —CH₂NH₂; is provided.

In some embodiments, a compound of Formula W:

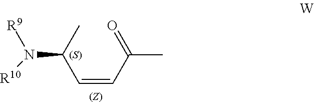

or a salt thereof, wherein:
R⁹ is an amine protecting group and R¹⁰ is hydrogen; or optionally:
R⁹ and R¹⁰ together with the nitrogen atom to which they are attached form a heterocyclic ring protecting group;
   is provided.

In some embodiments, the compound of Formula W or a salt thereof is a compound of Formula W-1:

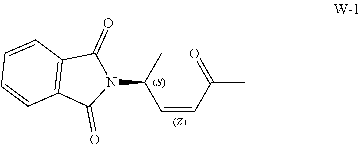

or a salt thereof.

In some embodiments of the processes and compounds disclosed herein, R¹ is selected from C₁₋₄alkyl, benzyl, and —COOR⁴; wherein R⁴ is selected from C₁₋₄alkyl and phenyl. In some embodiments, R¹ is selected from methyl, ethyl, benzyl, acetate, propionate, and benzoate. In some embodiments, R¹ is selected from C₁₋₄alkyl and benzyl. In some embodiments, R¹ is C₁₋₄ alkyl. In some embodiments, R¹ is selected from methyl, ethyl, and benzyl. In some embodiments, R¹ is methyl. In some embodiments, R¹ is ethyl. In some embodiments, R¹ is benzyl. In some embodiments, R¹ is —COOR⁴. In some embodiments, R⁴ is C₁₋₄alkyl. In some embodiments, R⁴ is methyl or ethyl. In some embodiments, R⁴ is phenyl.

In some embodiments of the processes and compounds disclosed herein, R¹ is selected from hydrogen, C₁₋₄alkyl, and benzyl. In some embodiments, R¹ is hydrogen. In some embodiments, R¹ is C₁₋₄ alkyl. In some embodiments, R¹ is selected from hydrogen, methyl, ethyl, and benzyl.

In some embodiments of the processes and compounds disclosed herein, R² is tert-butyloxycarbonyl.

In some embodiments of the compounds disclosed herein, R³ is selected from C₁₋₄alkyl and benzyl. In some embodiments, R³ is selected from methyl, ethyl, and benzyl. In some embodiments, R³ is C₁₋₄alkyl. In some embodiments, R³ is selected from methyl and ethyl. In some embodiments, R³ is methyl. In some embodiments, R³ is ethyl. In some embodiments, R³ is benzyl.

In some embodiments of the processes and compounds disclosed herein, R^{3A} is selected from C₁₋₄alkyl, C₁₋₄haloalkyl, and benzyl. In some embodiments, R^{3A} is selected from methyl, ethyl, isopropyl, trifluoroethyl, and benzyl. In some embodiments, R^{3A} is C₁₋₄alkyl. In some embodiments, R^{3A} is selected from methyl, ethyl, and isopropyl. In some embodiments, R^{3A} is methyl. In some embodiments, R^{3A} is C₁₋₄haloalkyl. In some embodiments, R^{3A} is trifluoroethyl. In some embodiments, R^{3A} is benzyl.

In some embodiments of the processes and compounds disclosed herein, R^{3A} is selected from hydrogen, C₁₋₄alkyl, and benzyl. In some embodiments, R^{3A} is selected from hydrogen, methyl, ethyl, isopropyl, and benzyl.

In some embodiments of the processes and compounds disclosed herein, R⁵ is hydrogen. In some embodiments, R⁵ is R⁹. In some embodiments, R⁵ is an amine protecting group. In some embodiments, R⁵ is tert-butyloxycarbonyl.

In some embodiments of the processes and compounds disclosed herein, R⁷ is tert-butyloxycarbonyl. In some embodiments, R⁸ is tert-butyloxycarbonyl. In some embodiments, R⁷ and R⁸ are tert-butyloxycarbonyl.

In some embodiments of the processes and compounds disclosed herein, R⁹ is tert-butyloxycarbonyl.

In some embodiments of the processes and compounds disclosed herein, R⁹ and R¹⁰ together with the nitrogen atom to which they are attached form a heterocyclic ring protecting group. In some embodiments of the processes and compounds disclosed herein, R⁹ and R¹⁰ together with the nitrogen atom to which they are attached form phthalimide.

In some embodiments of the processes and compounds disclosed herein, R¹¹ is selected from methyl, ethyl, isopropyl, pentyl, heptyl, nonyl, pentadecyl, phenyl, and —CH₂NH₂. In some embodiments, R¹¹ is C₁₋₂₀alkyl. In some embodiments, R¹¹ is selected from methyl, ethyl, isopropyl, pentyl, heptyl, nonyl, and pentadecyl. In some embodiments, R¹¹ is methyl. In some embodiments, R¹¹ is phenyl. In some embodiments, R¹¹ is —CH₂NH₂.

In some embodiments of the processes and compounds disclosed herein, R¹² is —S(O)—₃R¹³. In some embodiments, R¹³ is selected from tolyl, methyl, and trifluoromethyl. In some embodiments, R¹³ is C₁₋₄alkyl. In some embodiments, R¹³ is methyl.

In some embodiments of the processes and compounds disclosed herein, R¹² is —OC(═N)R¹⁴. In some embodiments, R¹⁴ is selected from trichloromethyl and tribromomethyl.

### EXAMPLES

Representative syntheses of compounds of the present disclosure are described in schemes below, and the particular examples that follow. The following examples are merely illustrative, and not intended to limit this disclosure in any way. It is to be understood that individual steps described herein may be combined. It is also to be understood that separate batches of a compound may be combined and carried forth in the next synthetic step.

### Example 1: Preparation of methyl 3-methoxy-4-oxo-5-((2,4,6-trifluorobenzyl)carbamoyl)-4H-pyran-2-carboxylate (X-B)

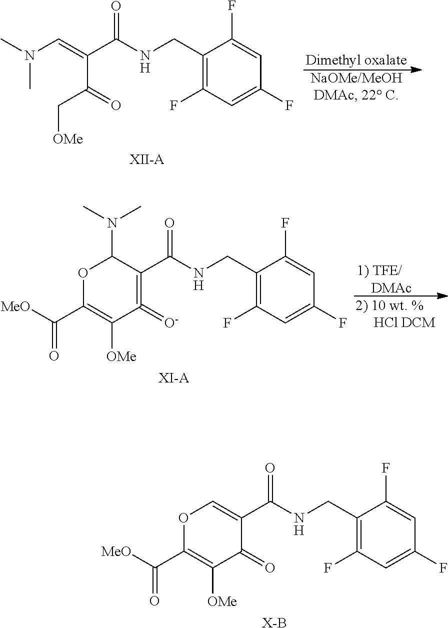

Dimethyl oxalate (5 equiv.) was charged to reactor A followed by N,N-dimethylacetamide (12 V). The mixture was agitated at about 22° C. for about 30 mins. 25% sodium methoxide in methanol (1.5 equiv.) was charged to the reaction mixture followed by a rinse forward with N,N-dimethylacetamide (0.1 V). The reaction mixture was cooled to about 10° C. (E)-2-((dimethylamino)methylene)-4-methoxy-3-oxo-N-(2,4,6-trifluorobenzyl)butanamide (XII-A, 1.00 equiv. scaling factor) was charged to reactor A followed by N,N-dimethylacetamide (0.5 V). The contents were agitated for about 2 hrs at about 10° C. 2,2,2-trifluoroethanol (1.2 V) was added to the reactor A followed by N,N-dimethylacetamide (0.1 V). The contents agitated for about 16 h at about 10° C. 10 wt. % Hydrochloric acid (5 V) was charged to reactor B followed by dichloromethane (15 V) and the contents were adjusted to about 20° C. The contents in reactor A were slowly charged to reactor B maintaining pot temperature about 30° C. The contents of reactor B were agitated for about 30 mins and then allowed to settle for about 30 mins. The bottom organic layer was transferred to reactor A and washed with water 2× (5 V). The organic layer was distilled to about 8 V then solvent swapped to 2-propanol (12 V). The reactor contents were held to about 45° C. for about 1 hr then cooled to about 0° C. over about 3 hrs. The reactor contents were filtered and dried at about 50° C. to afford compound X-B. ¹H NMR (400 MHz, DMSO-d₆) δ 9.38 (t, J=5.8 Hz, 1H), 8.91 (s, 1H), 7.21 (m, 2H), 4.55 (d, J=5.8 Hz, 2H), 3.89 (s, 3H), 3.86 (s, 3H).

### Example 2: Preparation of methyl 1-((tert-butoxycarbonyl)amino)-3-methoxy-4-oxo-5-((2,4,6-trifluorobenzyl)carbamoyl)-1,4-dihydropyridine-2-carboxylate (VIII-B)

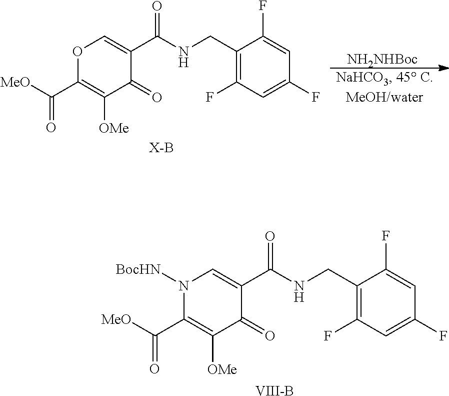

Compound X-B (1.0 equiv, scaling factor), tert-butyl carbazate (1.1 equiv), and sodium bicarbonate (2 equiv) were charged to reactor A followed by methanol (6 V) and water (0.4 V). The mixture was agitated at about 45° C. for about 3 hours. The contents of the reactor were distilled to 6 V and the temperature was adjusted to about 35° C. Water (6 V) was charged over about 1 hour and the temperature was adjusted to about 22° C. over about 1 hour and stirred for about 1 hour. The reactor contents were filtered, washed with water (3 V), and dried at about 50° C. to afford compound VIII-B. ¹H NMR (400 MHz, DMSO-d₆) δ 11.29 (s, 1H), 10.15 (t, J=5.8 Hz, 1H), 8.40 (s, 1H), 7.31-7.12 (m, 2H), 4.55 (d, J=5.8 Hz, 2H), 3.87 (s, 3H), 3.80 (s, 3H), 1.42 (s, 9H).

### Example 3: Preparation of methyl (R)-1-(but-3-en-2-yl(tert-butoxycarbonyl)amino)-3-methoxy-4-oxo-5-((2,4,6-trifluorobenzyl)carbamoyl)-1,4-dihydropyridine-2-carboxylate (VII-B)

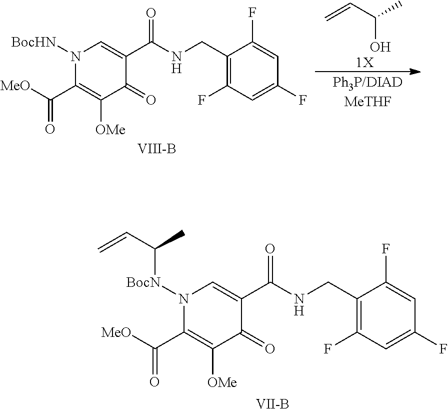

Compound VIII-B (1.0 equiv, scaling factor), 2-methyltetrahydrofuran (8 V), triphenylphosphine (1.5 equiv), and (S)-but-3-en-2-ol (IX, 1.5 equiv) were charged to reactor A. The reactor contents were adjusted to about 0° C. Diisopropyl azodicarboxylate (1.5 equiv) and 2-methyltetrahydrofuran (2 V) were charged to reactor B and the contents were agitated at about 22° C. The contents of reactor B were transferred to reactor A over about 30 minutes. The contents of reactor A were adjusted to about 22° C. and stirred for about 1 hour. The contents were distilled to about 2 V then solvent swapped to toluene (8 V). The contents of reactor A were adjusted to about 0° C. The contents of reactor A were filtered and the filtrate was collected in reactor C. The contents of reactor C were distilled to 2 V and solvent swapped to methanol (9 V). Water (2 V) was charged to reactor C over about 1 hour and stirred at about 22° C. Water (3 V) was charged to reactor C over about 1 hour and then stirred at about 22° C. The contents of reactor C were filtered, the cake was washed with a mixture of MeOH (1.5 V) and water (0.5 V) and dried at about 50° C. to afford compound VII-B. ¹H NMR (400 MHz, DMSO-d₆) δ 10.10 (q, J=5.5 Hz, 1H), 8.40 (d, J=23.9 Hz, 1H), 7.23 (t, J=8.7 Hz, 2H), 5.92 (dddd, J=17.3, 10.5, 9.1, 7.0 Hz, 1H), 5.33-5.16 (m, 2H), 4.81-4.47 (m, 3H), 3.88 (d, J=5.1 Hz, 3H), 3.81 (d, J=1.1 Hz, 3H), 1.40 (d, J=1.3 Hz, 9H), 1.27 (dd, J=29.1, 6.9 Hz, 3H).

### Example 4: Preparation of N²—((S)-but-3-en-2-yl)-1-(((R)-but-3-en-2-yl)amino)-3-methoxy-4-oxo-N⁵-(2,4,6-trifluorobenzyl)-1,4-dihydropyridine-2,5-dicarboxamide (III-A)

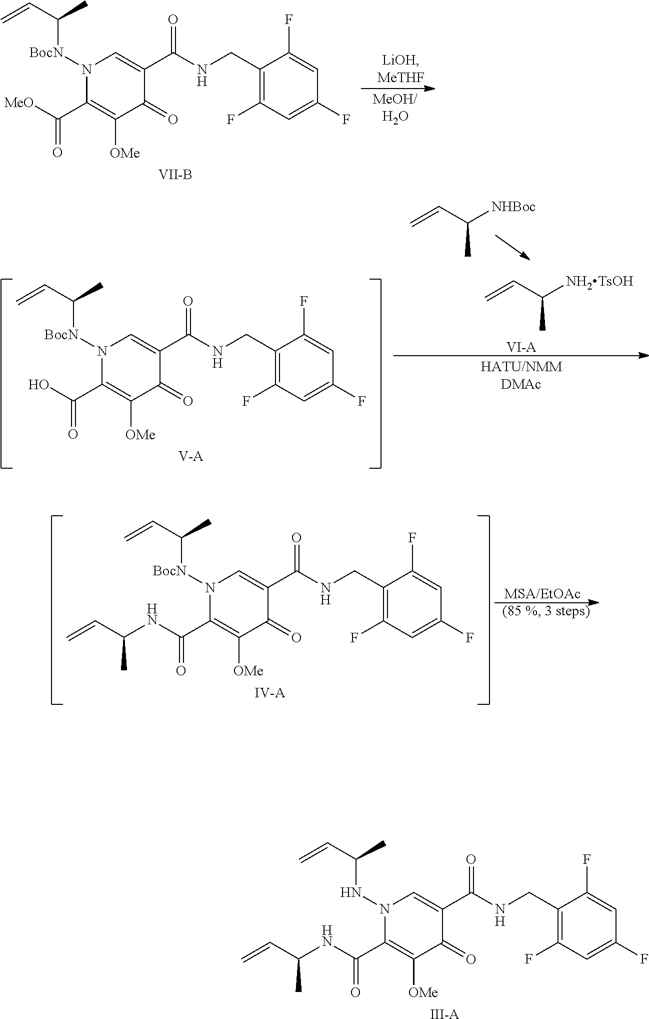

### Step 1: Preparation of (R)-1-(but-3-en-2-yl(tert-butoxycarbonyl)amino)-3-methoxy-4-oxo-5-((2,4,6-trifluorobenzyl)carbamoyl)-1,4-dihydropyridine-2-carboxylic Acid (V-A)

A reactor vessel was charged with compound VII-B (1.0 equiv, scaling factor), cyclopentyl methyl ether (CPME, 2.0 volumes), methanol (4.0 volumes), water (1.0 volumes), lithium hydroxide monohydrate (4.0 equiv), and stirred at about 20° C. for about 4 hours. CPME (6.0 volumes) was added to the reactor. A separate reactor was charged with water (4.1 volumes) and concentrated hydrochloric acid (0.8 volumes), stirred for about 15 minutes, and transferred to the reaction mixture. The layers were separated and the organic layer was washed with water (5.0 volumes). CPME (2.0 volumes) was added and the resulting organic stream was distilled to about 8 volumes at about 35° C. to afford compound V-A.

### Step 2: Preparation of tert-butyl ((R)-but-3-en-2-yl)(2-(((S)-but-3-en-2-yl)carbamoyl)-3-methoxy-4-oxo-5-((2,4,6-trifluorobenzyl)carbamoyl)pyridin-1(4H)-yl)carbamate (IV-A)

To a CPME solution of compound V-A (3.5 volumes), 4-methylmorpholine (5.0 equiv), and (HATU, 1.1 equiv) were added and stirred at about 20° C. for about 30 minutes. (S)-but-3-en-2-aminium tosylate (VI-A, 1.1 equiv) was added and the reaction mixture was stirred at about 20° C. for about 2 hours. In a separate reactor, water (4.6 volumes) and concentrated hydrochloric acid (0.4 volumes) were added, stirred for 15 minutes, and transferred to the reaction mixture. The layers were separated. In a separate reactor, water (4.6 volumes) and concentrated hydrochloric acid (0.4 volumes) were added, stirred for about 15 minutes, and transferred to the reaction mixture. The layers were separated and the organic layer was washed with water (4.0 volumes) to afford compound IV-A.

### Step 3: Preparation of N²—((S)-but-3-en-2-yl)-1-(((R)-but-3-en-2-yl)amino)-3-methoxy-4-oxo-N⁵-(2,4,6-trifluorobenzyl)-1,4-dihydropyridine-2,5-dicarboxamide (III-A)

To a CPME solution of compound IV-A, methanesulfonic acid (3.0 equiv) was added and the reaction was stirred at 35° C. for about 4 hours. In a separate reactor, potassium phosphate tribasic (4.0 equiv) and water (5.0 volumes) were added, stirred for about 15 minutes, and transferred to the reaction mixture. The layers were separated and the organic layer was washed with water (5.0 volumes). CPME (4.0 volumes) was added and the organic stream was distilled to about 8 volumes. Compound III-A seeds (0.0006 equiv) were charged (optional) and the reactor contents were cooled to about 20° C. over about 1 hour. n-Heptane (5.3 volumes) was added over about 1 hour. After aging the slurry for about 1 hour, the slurry was filtered and the cake was washed with pre-mixed n-heptane (1.0 volumes) and CPME (1.0 volumes). The resulting cake was dried at about 50° C. to provide compound III-A. ¹H NMR (400 MHz, DMSO-d₆) δ 10.47 (t, J=5.7 Hz, 1H), 8.70 (d, J=8.2 Hz, 1H), 8.38 (s, 1H), 7.25-7.17 (m, 2H), 6.92 (d, J=7.2 Hz, 1H), 5.84 (ddd, J=17.3, 10.5, 5.0, 1H), 5.71 (ddd, J=17.2, 10.2, 8.5, 1H), 5.25 (dt, J 17.3, 1.4, 1H), 5.12-4.94 (m, 3H), 4.53-4.47 (m, 3H), 3.74 (s, 4H), 1.19 (d, J=6.9 Hz, 3H), 1.04 (d, J=6.5 Hz, 3H).

### Example 5: Preparation of 1-((R)-but-3-en-2-yl)-3-((S)-but-3-en-2-yl)-5-methoxy-4,6-dioxo-N-(2,4,6-trifluorobenzyl)-2,3,4,6-tetrahydro-1H-pyrido[2,1-f][1,2,4]triazine-7-carboxamide (II-A)

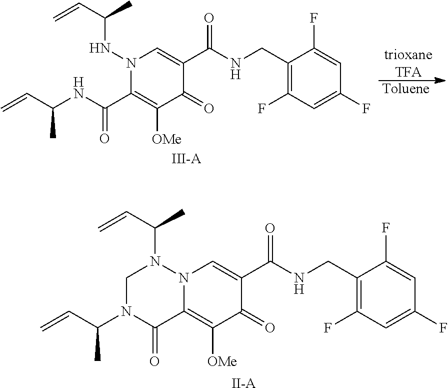

A reaction vessel was charged with compound III-A (scaling factor, 1.0 equiv), 1,3,5-trioxane (1.6 equiv), toluene (8 V), and trifluoroacetic acid (4.2 equiv). The contents were agitated and heated to about 80° C. for about 18 hours. The reaction mixture was then cooled to about 10° C. and isopropyl acetate (3 V) was added. In a separate reactor, a solution of 50% aqueous sodium hydroxide (5.5 equiv) was combined with water (3.5 V). The mixture was then transferred to the reaction mixture and agitated for about 15 minutes. The layers were separated and the organic layer was washed with a 6 wt % solution of sodium chloride (3 V) and water (2 V). The resulting organic solution was distilled to about 5 V. Toluene (10 V) was charged and the resulting solution was distilled to about 5 V. Toluene (5 V) was charged and the mixture was polish filtered. The resulting solution was distilled to about 4 V. The solution was heated to about 45° C. and heptane (2 V) was charged over about 30 minutes. Compound II-A seeds (0.003 eq) were charged and the mixture was agitated for about 3 hours. Heptane (3 V) was charged over about 2 hours. The mixture was then agitated for about 2 hours and then cooled to 20° C. over about 3 hours. The resulting mixture was then agitated for about 12 hours and filtered. The cake was washed with a mixture of heptane (1.2 V) and toluene (0.8 V). The resulting cake was dried at about 50° C. to provide compound II-A. ¹H NMR (400 MHz, Chloroform-d) δ 10.28 (d, J=6.5 Hz, 1H), 8.43 (s, 1H), 6.71-6.58 (m, 2H), 5.86 (ddd, J=16.6, 10.4, 5.6 Hz, 1H), 5.70 (ddd, J=17.0, 10.3, 8.4 Hz, 1H), 5.41-5.24 (m, 4H), 5.17 (br s, 1H), 4.64 (d, J=5.6 Hz, 2H), 4.54 (br s, 2H), 4.06 (s, 3H), 3.71 (br s, 1H), 1.32 (br s, 3H), 1.11 (br s, 3H).

### Example 6: Preparation of (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B)

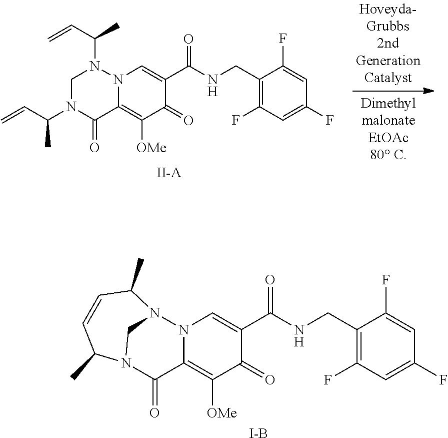

A reaction vessel was charged with compound II-A (scaling factor, 1.0 equiv), ethyl acetate (10 V), and dimethyl maleate (0.15 equiv). The contents were agitated and heated to about 75° C. A separate vessel was charged with Hoveyda-Grubbs Second Generation Catalyst (0.015 equiv) and ethyl acetate (5 V). The contents of this vessel were transferred to the vessel containing compound II-A over about 1 hour. The resulting mixture was agitated and heated to about 75° C. for about 2 hours. The reaction mixture was then cooled to 40° C. and N-acetyl-L-cysteine (2 equiv) was added. The mixture was agitated and heated to about 40° C. for about 12 hours, then washed with 10 wt % aqueous potassium carbonate (2×10 volumes) and 5 wt % aqueous sodium chloride (10 volumes). The resulting solution was filtered through activated carbon and provided compound I-B as a solution in ethyl acetate to be used in the next step. A sample was purified by column chromatography for ¹H NMR analysis. ¹H NMR (400 MHz, CDCl₃) δ 10.30 (t, J=5.8 Hz, 1H), 8.55 (s, 1H), 6.70-6.61 (m, 2H), 5.64-5.58 (m, 1H), 5.57-5.48 (m, 1H), 5.37 (dt, J=11.3, 3.2 Hz, 1H), 4.95 (d, J=14.3 Hz, 1H), 4.71-4.57 (m, 2H), 4.37 (d, J=14.3 Hz, 1H), 4.11 (s, 3H), 3.80-3.71 (m, 1H), 1.39 (d, J=6.7 Hz, 3H), 1.32 (d, J=7.4 Hz, 3H).

### Example 7: Preparation of (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-A)

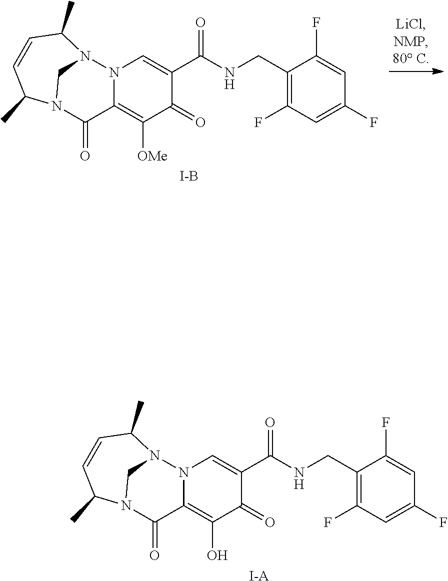

A solution of compound I-B in Ethyl acetate was charged to a reactor. N-methyl-2-pyrrolidinone (3 V) was charged to the solution. The solution was then distilled to about 4V. LiCl (3 equiv.) was then charged before adjusting the temperature to about 80° C. for about 4 hours. The reaction was then cooled to about 20° C. before washing the organic layer with HCl (0.5M, 5 V), NaCl (5%, 10 V), and water (5 V). The organic layer was then distilled to about 6 V before charging 2-propanol (10 V). The solution was then distilled to about 6 V before charging more 2-propanol (2 V). The temperature was then adjusted to about 60° C. for about 1 hour before adjusting to about 20° C. over about 2 hours. The slurry was aged about 1 hour to overnight before putting the slurry through a high shear wet mill. The slurry was then filtered and the cake was rinsed with IPA (2 V) before drying at about 40° C. until adequately dry, affording the compound of Formula I-A. ¹H NMR (400 MHz, Chloroform-d) δ 10.12 (t, J=5.8 Hz, 1H), 8.50 (s, 1H), 6.81-6.52 (m, 2H), 5.62 (dt, J=11.4, 2.3 Hz, 1H), 5.40 (ddt, J=14.4, 11.4, 2.9 Hz, 2H), 4.98 (d, J=14.2 Hz, 1H), 4.73-4.59 (m, 2H), 4.46 (d, J=14.1 Hz, 1H), 1.37 (t, J=7.1 Hz, 6H).

### Example 8: Preparation of (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-A)

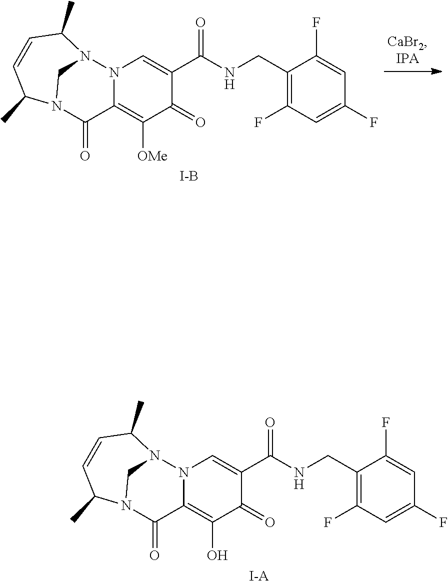

A reaction vessel was charged with (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B, 1.0 mol equiv, scaling factor) and Calcium Bromide Hydrate (0.6 mol equiv, assuming hexahydrate) before diluting with isopropyl alcohol (3 V). The reaction vessel was heated for 1-2 hours at reflux before cooling back to 20° C. The reaction was diluted with Dichloromethane (5 V) before washing with Hydrochloric Acid (1.2 eq, conc) in water (5 V). The organic layer was washed with water (5 V) before charging isopropyl alcohol (10 V) and distilling to 6 V. The mixture was then diluted with isopropyl alcohol (2 V). The slurry is then heated to reflux until homogeneous and cooled to 60° C. (10° C./h) before charging (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-A) seed (0.01 wt %). The seed bed was aged for 1 hour before cooling to 20° C. (10° C./h). The slurry is aged overnight before high shear wet milling and filtration. The cake was washed with isopropyl alcohol (2 V) before drying at 50° C. overnight to yield compound I-A.

### Example 9: Preparation of (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-A)

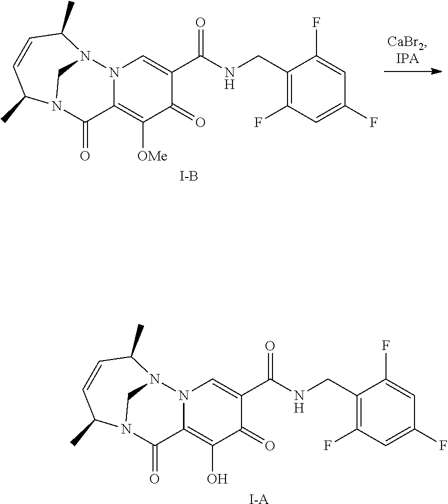

A reaction vessel was charged with (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B, 1.0 mol equiv, scaling factor) and Calcium Bromide Hydrate (0.6 mol equiv, assuming hexahydrate) before diluting with isopropyl alcohol (3 V). The reaction vessel was heated for 1-2 hours at reflux. The reaction was cooled to 65° C. before charging a solution of HCl (1.2 eq, conc) in 10-30% isopropyl alcohol water (1-2V). (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-A) seed was charged (0.01 wt %) and the mixture was aged for 1 hour before charging 10-30% isopropyl alcohol water mixture (1-12 V) over 1-3 hours. The slurry was then cooled to 20° C. and aged overnight before high shear wet mill and filtration. The cake was rinsed with 10-30% isopropyl alcohol water before drying at 50° C. overnight to yield compound I-A.

### Example 10: Preparation of methyl (R)-1-(but-3-en-2-yl(tert-butoxycarbonyl)amino)-3-methoxy-4-oxo-5-((2,4,6-trifluorobenzyl)carbamoyl)-1,4-dihydropyridine-2-carboxylate (VII-B)

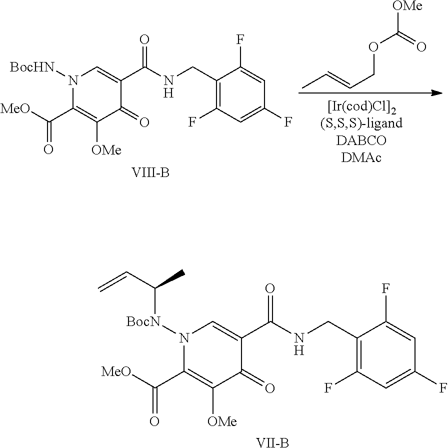

A reaction vessel was charged with bis(1,5-cyclooctadiene)diiridium(I) dichloride (0.04 equiv), (S,S,S)-(+)-(3,5-dioxa-4-phosphacyclohepta[2,1-a:3,4-a′]dinaphthalene-4-yl)bis(1-phenylethyl)amine (0.08 equiv), tetrahydrofuran (13V), and n-propylamine (8 V). The mixture was agitated at about 50° C. for about 30 minutes and then concentrated to dryness. Dimethylacetamide (10 V) was charged, followed by crotyl methyl carbonate (1.5 equiv), and compound VIII-B (scaling factor, 1.0 equiv). The reaction was agitated for about 15 h. Water (20 V) was then added and the reaction was extracted twice with ethyl acetate (10 V). The combined organic layers were dried with sodium sulfate, filtered, and concentrated. ¹H NMR (400 MHz, DMSO-d₆) δ 10.10 (q, J=5.5 Hz, 1H), 8.40 (d, J=23.9 Hz, 1H), 7.23 (t, J=8.7 Hz, 2H), 5.92 (dddd, J=17.3, 10.5, 9.1, 7.0 Hz, 1H), 5.33-5.16 (m, 2H), 4.81-4.47 (m, 3H), 3.88 (d, J=5.1 Hz, 3H), 3.81 (d, J=1.1 Hz, 3H), 1.40 (d, J=1.3 Hz, 9H), 1.27 (dd, J=29.1, 6.9 Hz, 3H).

### Example 11: Preparation of methyl (R)-1-(but-3-en-2-ylamino)-3-methoxy-4-oxo-5-((2,4,6-trifluorobenzyl)carbamoyl)-1,4-dihydropyridine-2-carboxylate (VII-A)

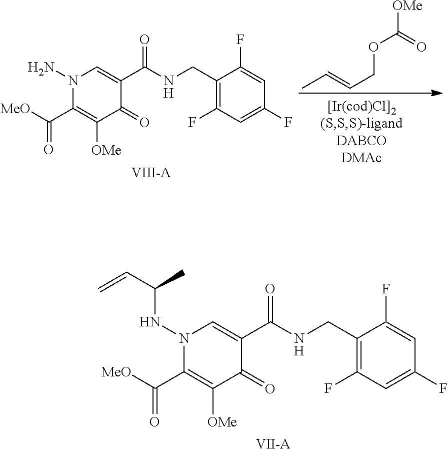

A reaction vessel was charged with bis(1,5-cyclooctadiene)diiridium(I) dichloride (0.02 equiv), (S,S,S)-(+)-(3,5-dioxa-4-phosphacyclohepta[2,1-a:3,4-a′]dinaphthalene-4-yl)bis(1-phenylethyl)amine (0.05 equiv), dimethylacetamide (14V), and 1,4-diazabicyclo[2.2.2]octane (0.15 equiv). The mixture was agitated at about 50° C. for about 30 minutes. The reaction was charged with crotyl methyl carbonate (1.5 equiv) and agitated for about 5 minutes. methyl 1-amino-3-methoxy-4-oxo-5-((2,4,6-trifluorobenzyl)carbamoyl)-1,4-dihydropyridine-2-carboxylate (VIII-A, scaling factor, 1.0 equiv) was then charged. The reaction was agitated at about 50° C. for about 18 h. Water (20 V) was then added and the reaction was extracted twice with ethyl acetate (10 V). The combined organic layers were dried with sodium sulfate, filtered, and concentrated to afford compound VII-A. ¹H NMR (400 MHz, Chloroform-d) δ 10.36 (t, J=5.7 Hz, 1H), 8.57 (d, J=1.0 Hz, 1H), 6.75-6.56 (m, 2H), 5.79-5.56 (m, 2H), 5.20-5.03 (m, 2H), 4.62 (d, J=5.7 Hz, 2H), 3.97 (d, J=1.0 Hz, 3H), 3.93 (d, J=1.0 Hz, 3H), 3.73-3.64 (m, 1H), 1.16 (dd, J=6.4, 1.0 Hz, 3H).

### Example 12: Preparation of hex-3-yne-2,5-diyl Diacetate (K-1)

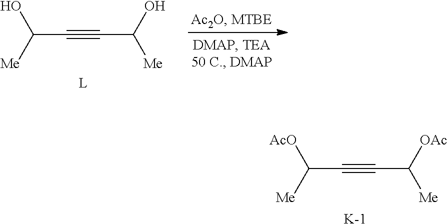

Hex-3-yne-2,5-diol (L, 1.00 equiv., scaling factor) was charged to reactor A, followed by tert-butyl methyl ether (7 V), 4-dimethylaminopyridine (0.01 g/g), and acetic anhydride (3 equiv.). The solution was cooled to about 20° C. and triethylamine (0.5 equiv.) was charged. The reaction was heated to about 50° C. for about 2 hours. The reaction was cooled to about 20° C. and was washed with water (3×5 V). The contents were concentrated at about 35° C. until dry to afford compound K-1.

### Example 13: Preparation of (2S,5R)-5-hydroxyhex-3-yn-2-yl Acetate (J-1)

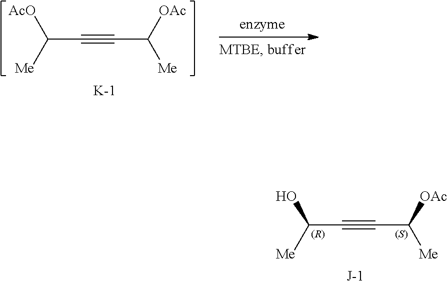

Compound K-1 (1.00 equiv., scaling factor) was charged to reactor A, followed by tert-butyl methyl ether (3 V) and 0.6 M potassium phosphate buffer (7 V). The pH of the mixture was adjusted to about pH 10 using 20 wt % potassium carbonate in water. Enzyme L-092 (0.001 g/g) was charged to reactor A. The mixture was agitated at about 30° C. for about 20 hours. The reaction mixture was cooled to about 20° C. Celite (0.1 g/g) was charged to the reaction mixture, then the mixture was filtered. The filter cake was washed with tert-butyl methyl ether (2.5 V). The layers were separated, and the aqueous layer was washed with tert-butyl methyl ether (2×5 V). The organic layers were charged in reactor A, followed by water (2.5 V). The aqueous layer was removed. The contents were distilled to dryness. This was purified via column chromatography using a 10% ethyl acetate in heptane mixture to 25% ethyl acetate mixture. The purified material was concentrated at about 40° C. to afford compound J-1. ¹H NMR (400 MHz, Chloroform-d) δ 5.45 (qd, J=6.7, 1.4 Hz, 1H), 4.55 (qd, J=6.6, 1.4 Hz, 1H), 2.50 (s, 1H) 2.07 (s, 3H), 1.45 (dd, J=13.7, 6.7 Hz, 6H).

### Example 14: Preparation of (2S,5S)-5-(1,3-dioxoisoindolin-2-yl)hex-3-yn-2-yl Acetate (H-2)

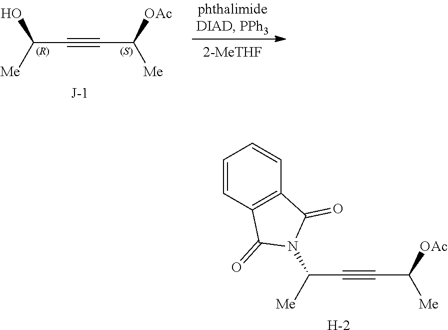

Compound J-1 (1.0 mol equiv, scaling factor) was charged to a reactor followed by 8 V of 2-methyltetrahydrofuran. Phthalimide (1.1 mol equiv) and triphenylphosphine (1.2 mol equiv) were added to the stirring solution. In a separate flask, diisopropyl azodicarboxylate (1.2 mol equiv) was added and diluted with 2 V of 2-methyltetrahydrofuran. Slow addition of the DIAD solution was charged to the reactor at about 5° C. over about 2 hours. About 2 hours after complete addition of the DIAD solution, the solution was seeded with TPPO/DIHD crystals and left to stir for about 16 hours. The reaction was filtered, and the resulting cake was washed with 2 V of 2-methyltetrahydrofuran. The filtrate was distilled to about 4 V at about 50° C. and diluted with 8 V of toluene. The solution was distilled again to about 4 V at about 50° C. and followed by charging 8 V of toluene. The solution was concentrated down to about 9 V and cooled to about 5° C. and seeded with TPPO/DIHD crystals. The solution was left to stir for about 16 hours before filtering off the resulting solids. The cake was washed with about 2 V of toluene. The filtrate was distilled to 4 V at about 45° C. and diluted with 8 V of ethanol. The solution was distilled again to 4 V at about 75° C. and followed by charging 8 V of ethanol. The solution was concentrated down to 9 V and cooled to about 35° C. To the stirring solution, 3 V of water was added and seeded with compound H-2. The solution was aged for about 3 hours. To the resulting slurry, 3 V of water was added over about 3 hours and allowed to age for about 16 hours. The slurry was filtered and the resulting cake was washed with 2 V of ethanol/water (1:2). The solids were dried in vacuum under nitrogen at about 50° C. to afford compound H-2. ¹H NMR (400 MHz, CDCl₃) δ 7.84 (dd, J=5.4, 3.1 Hz, 2H), 7.71 (dd, J=5.5, 3.0 Hz, 2H), 5.46 (qd, J=6.7, 1.8 Hz, 1H), 5.23 (qd, J=7.2, 1.8 Hz, 1H), 2.05 (s, 3H), 1.68 (d, J=7.1 Hz, 3H), 1.47 (d, J=6.6 Hz, 3H).

### Example 15: Preparation of 2-((2S,5S)-5-hydroxyhex-3-yn-2-yl)isoindoline-1,3-dione (G-2)

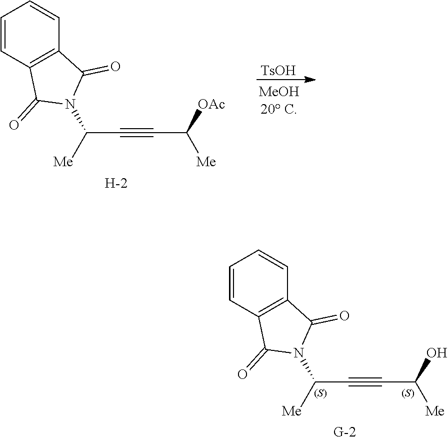

Compound H-2 (1.0 mol equiv, scaling factor) and methanol (3 V) were charged to a reactor. To the stirring solution was charged p-toluenesulfonic acid monohydrate (0.10 mol equiv). The mixture was agitated at about 20° C. for about 24 hours. To the solution was charged toluene (9 V), and the contents of the reactor adjusted to about 5° C. Water (5 V) was charged over about 30 minutes. Agitation was stopped to allow the layers to settle, and the aqueous layer was discharged. To the reactor was charged water (5 V) over about 15 minutes. Agitation was stopped to allow the layers to settle, and the aqueous layer was discharged. The solution was distilled to 4 V at about 40° C. At about 40° C. heptane (3 V) was charged over about 30 minutes. The solution was seeded with compound G-2, then agitated about 12 hours for the seed bed slurry to form. Then heptane (8 V) was charged to the slurry over about 2 hours. The reactor was ramp cooled over about 1 hour from about 40° C. to about 20° C. The slurry was filtered, and the solids washed with a 3:1 heptane:toluene solution (4 V). The solids were dried at about 50° C. in vacuum under nitrogen to afford compound G-2. ¹H NMR (400 MHz, CDCl₃) δ 7.84 (dd, J=5.4, 3.1 Hz, 2H), 7.72 (dd, J=5.5, 3.0 Hz, 2H), 5.23 (qd, J=7.1, 1.8 Hz, 1H), 4.55 (qd, J=6.6, 1.8 Hz, 1H), 2.23 (s, 1H), 1.68 (d, J=7.2 Hz, 3H), 1.44 (d, J=6.6 Hz, 3H).

### Example 16: Preparation of 2-((2S,5S,Z)-5-hydroxyhex-3-en-2-yl)isoindoline-1,3-dione (F-2)

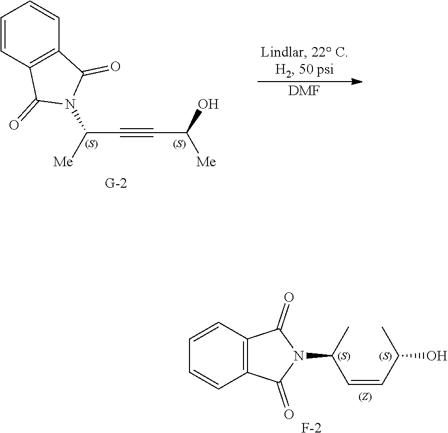

Compound G-2 (1.00 equiv, scaling factor) and palladium on calcium carbonate, lead poisoned (Lindlar catalyst, 0.002 equiv) were charged to a 2 L stainless steel reactor at about 22° C. N,N-dimethylformamide (5 V) was charged at about 22° C. The reaction vessel was sealed, and the atmosphere of the sealed vessel was purged by pressurizing the contents to about 50 psi with nitrogen and venting. This cycle was repeated two times. The atmosphere was exchanged to hydrogen by pressurizing the contents to about 50 psi with hydrogen and venting. The vessel was pressurized with hydrogen about 50 psi and repressurized to about 50 psi as needed over the course of the reaction. After about 48 h, the product mixture was filtered over a pad of celite. The pad of celite was rinsed with N,N-dimethylformamide (1.5 V) and the filtrate was collected. The filtrate was charged to a 5 L reactor and water (2.5 V) was charged. A portion of compound F-2 (0.001 equiv) was added at 22° C. and the resulting slurry was allowed to age for about 2 h. An additional portion of water (10.4 V) was added over about 1 h at about 22° C. The resulting slurry was allowed to age for about 3 h before ramp cooling to about 5° C. over about 1 h. The cooled slurry was allowed to agitate for about 12 h at about 5° C. The aged slurry was collected from the reactor and the solids were collected by filtration. The resulting filter cake was rinsed with water (2 V). The rinsed cake was dried in a vacuum oven with a nitrogen bleed at about 50° C. to afford compound F-2. ¹H NMR (400 MHz, DMSO-d6) δ 8.38-7.53 (m, 4H), 5.75 (ddd, J=10.6, 9.1, 1.2 Hz, 1H), 5.43 (ddd, J=10.9, 8.4, 1.1 Hz, 1H), 5.26-5.05 (m, 1H), 4.80 (d, J=4.2 Hz, 1H), 4.61-4.37 (m, 1H), 1.47 (d, J=7.0 Hz, 3H), 0.96 (d, J=6.3 Hz, 3H).

### Example 17: Preparation of di-tert-butyl 1-((2R,5S,Z)-5-(1,3-dioxoisoindolin-2-yl)hex-3-en-2-yl)hydrazine-1,2-dicarboxylate (E-2)

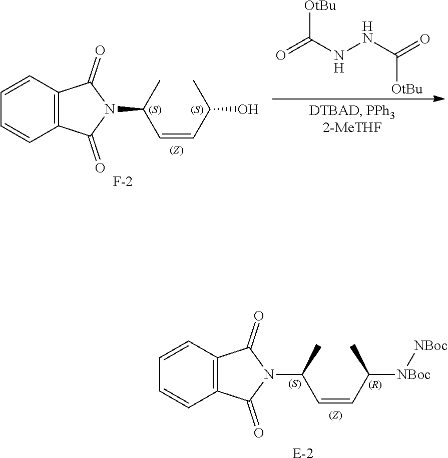

A reaction vessel was charged with compound F-2 (scaling factor, 1.0 equiv), di-tert-butyl hydrazodiformate (0.1 equiv), and triphenylphosphine (1.2 equiv) and 2-methyltetrahydrofuran (10 V) at about 25° C. The contents were agitated and cooled to about 0° C. Di-tert-butyl azodicarboxylate (1.2 equiv) was charged portion wise to the reaction and left to agitate for about 1 hour. The reaction mixture was then warmed up to about room temperature and left to agitate for about 3 hours. Magnesium chloride (2.1 equiv) was then charged to the reaction mixture and the mixture agitated for about 16 hours. Following overnight agitation, the slurry was filtered, and the cake was rinsed with 2-methyltetrahydrofuran (5 V) that had been warmed to about 40° C. The resulting filtrate was washed with water (5 V) twice. The organic layer was then distilled to about 5 V and ethanol (10 V) was charged. The mixture was distilled down to about 5 V again, and ethanol (10 V) was charged. The mixture was distilled down to 8 V and cooled to about 35° C. Water (3.0 V) was over about 2 hours and seeded with compound E-2 (0.005 equiv). The resulting slurry was aged for about 16 hours. After overnight agitation, water (4.0 V) was added to the slurry over about 2 hours. The mixture was cooled to about 20° C. over about 30 minutes and left to agitate for about another 30 minutes. The slurry was filtered, and the resulting cake was rinsed with a 1:1 mixture of ethanol and water (5 V). The solids were left to dry in a vacuum oven with a nitrogen bleed at about 40° C., affording compound E-2. ¹H NMR (400 MHz, CDCl₃): δ 7.81-7.64 (m, 4H), 6.32-6.05 (m, 1H), 5.97 (t, J=8 Hz, 1H), 5.64-5.39 (m, 1H), 5.19 (t, J=8 Hz, 1H), 5.07-4.84 (m, 1H), 1.54-1.15 (m, 24H).

### Example 18: Preparation of di-tert-butyl 1-((2R,5S,Z)-5-aminohex-3-en-2-yl)hydrazine-1,2-dicarboxylate (D-2)

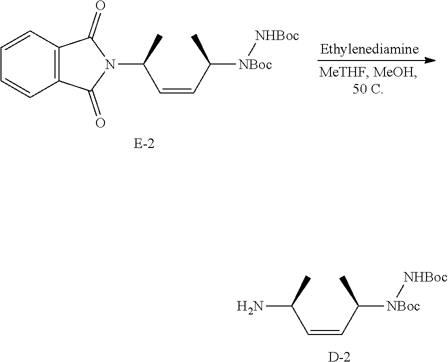

Compound E-2 (1.0 mol equiv, scaling factor), methanol (2.5 V), and 2-methyltetrahydrofuran (2.5 V) were charged to a reactor. To the stirring solution was charged ethylenediamine (3.0 mol equiv). The mixture was agitated at about 50° C. for about 4 hours. The temperature of the reactor was set to about 20° C. To the solution was charged 2-methyltetrahydrofuran (2.5 V), methyl t-butyl ether (5.0 V), and water (7.5 V). The contents of the reactor were filtered, and the filtrate collected into a separate reactor. Agitation of the filtrate was stopped to allow layers to settle. The aqueous layer was discharged. To the reactor was charged water (7.5 V). Agitation was stopped to allow layers to settle. The aqueous layer was discharged. To the reactor was charged isopropyl alcohol (10 V), and the solution was distilled to about 5 V at about 50° C. The temperature of the reactor was set to about 20° C. and water (8 V) was charged over about 30 minutes. The solution was seeded with compound D-2, then agitated about 2 hours for the seed bed slurry to form. Then water (7 V) was charged to the slurry over about 60 minutes and aged for about 2 hours. The slurry was filtered, and the solids washed with a 3:1 water:isopropyl alcohol solution (4 V). The solids were dried at about 50° C. under vacuum to afford compound D-2. ¹H NMR (400 MHz, CDCl₃) δ 6.36 (d, J=39.0 Hz, 1H), 5.29 (dd, J=12.3, 8.5 Hz, 2H), 5.03 (s, 1H), 3.89 (s, 1H), 1.44 (d, J=4.6 Hz, 20H), 1.17 (d, J=6.8 Hz, 3H), 1.09 (d, J=6.3 Hz, 3H).

### Example 19: Preparation of (2S,5R,Z)-5-((3-methoxy-2-(methoxycarbonyl)-4-oxo-5-((2,4,6-trifluorobenzyl)carbamoyl)pyridin-1(4H)-yl)amino)hex-3-en-2-aminium carboxyformate (A-1-A)

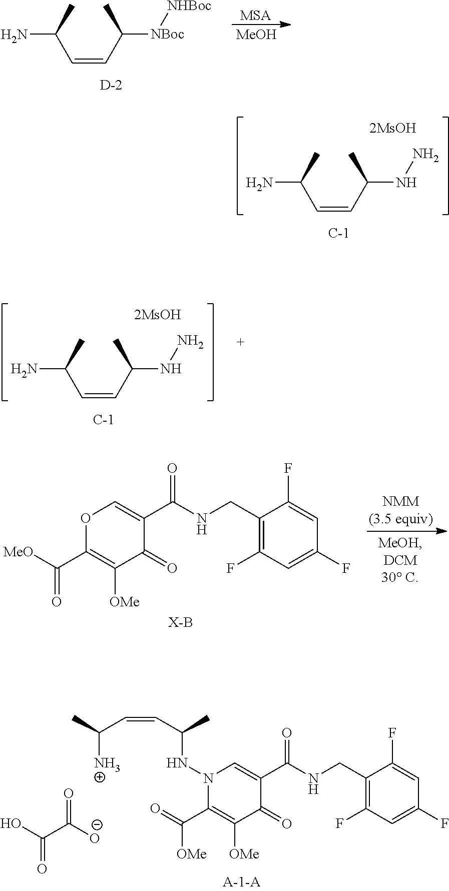

A reaction vessel was charged with compound D-2 (scaling factor, 1.0 equiv), methanesulfonic acid (3.2 equiv), and methanol (8 V). The contents were agitated and heated to about 60° C. for about 6 hours. The reaction mixture was then cooled to about 30° C. Dichloromethane (8 V) and methyl 3-methoxy-4-oxo-5-((2,4,6-trifluorobenzyl)carbamoyl)-4H-pyran-2-carboxylate (X-B, 0.95 equiv) were then charged. 4-methylmorpholine (3.5 equiv) was charged over about 1 hour. The contents were agitated for about 8 hours and then the reaction mixture was cooled to about 20° C. The mixture was then distilled to a minimum volume and 2-methyl tetrahydrofuran (10 V) was charged. A solution of dibasic potassium phosphate (4.0 equiv) in water (10 V) was charged. The layers were separated. The organic layer was washed with a solution of dibasic potassium phosphate (4.0 equiv) in water (10 V). The layers were separated. The organic layer was washed with water (10 V). Oxalic acid (1.1 equiv) was charged. The organic layer was then distilled to a minimum volume and then ethyl acetate (8 V) was charged. The reaction mixture was heated to about 55° C. Heptane (3 V) was then charged and agitated for about 0.5 hours. The mixture was then cooled to about 20° C. over about 2 hours, then agitated for about 12 hours and filtered. The cake was washed with a premixed solution of ethyl acetate (1.4 V) and heptane (0.6 V). The resulting cake was dried at about 50° C. to provide compound A-1-A. ¹H NMR (400 MHz, DMSO-d₆) δ 10.38 (t, J=5.7 Hz, 1H), 8.68 (s, 1H), 8.18 (br s, 3H), 7.33 (d, J=4.1 Hz, 1H), 7.28-7.11 (m, 2H), 5.65-5.33 (m, 2H), 4.69-4.44 (m, 2H), 4.38-4.25 (m, 1H), 4.13 (dt, J=13.3, 6.7 Hz, 1H), 3.90 (s, 3H), 3.79 (s, 3H), 1.22 (d, J=6.5 Hz, 3H), 0.94 (d, J=6.3 Hz, 3H).

### Example 20: Preparation of (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B)

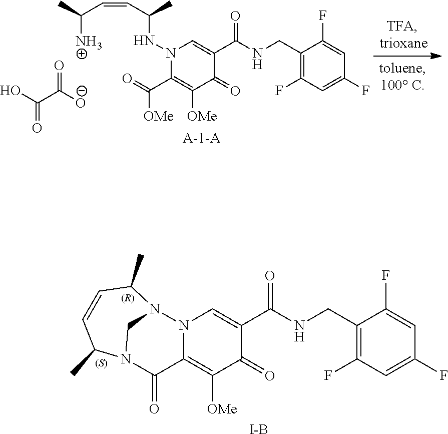

To a reaction vessel equipped with a stir bar is charged with compound A-1-A (1.0 equiv), trioxane (1.2 equiv), toluene (10 V), and trifluoroacetic acid (4.5 equiv). The reaction vessel is sealed and heated to about 100° C. for about 24 hours. Upon reaction completion, about 8V of 10% aqueous sodium bicarbonate and about 5 V of n-butanol were charged to the reaction mixture. The reaction was stirred at about 50° C. until complete dissolution of black solids were observed. The biphasic solution was partitioned, and the aqueous layer was extracted with about 5 V of n-butanol. The biphasic solution was partitioned, the organic layer was washed with about 3 V of process water and concentrated to dryness. Purified via flash column chromatography from 0 to 50% MeOH/DCM. Compound I-B was isolated as a yellow solid. ¹H NMR (400 MHz, CDCl₃) d 10.30 (t, J=5.8 Hz, 1H), 8.55 (s, 1H), 6.70-6.61 (m, 2H), 5.64-5.58 (m, 1H), 5.57-5.48 (m, 1H), 5.37 (dt, J=11.3, 3.2 Hz, 1H), 4.95 (d, J=14.3 Hz, 1H), 4.71-4.57 (m, 2H), 4.37 (d, J=14.3 Hz, 1H), 4.11 (s, 3H), 3.80-3.71 (m, 1H), 1.39 (d, J=6.7 Hz, 3H), 1.32 (d, J=7.4 Hz, 3H).

### Example 21: Preparation of (2S,5R)-5-((methylsulfonyl)oxy)hex-3-yn-2-yl acetate (N-1)

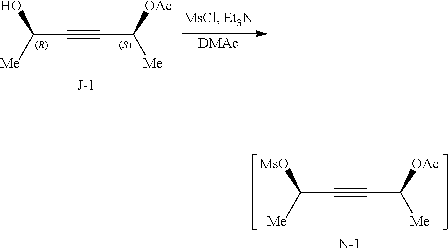

A reaction vessel was charged with compound J-1 (1.0 mol equiv, scaling factor) and diluted with N,N-dimethylacetamide (3 V). Triethylamine (1.2 mol equiv) was added to the stirring reaction and cooled to about 0° C. In a separate vessel, methanesulfonyl chloride (1.2 mol equiv) was diluted with N,N-dimethylacetamide (3 V). The methanesulfonyl chloride solution was charged to the reactor over about 2 hours. After about 1 hour, the reaction solution was filtered. The reactor and cake were washed with tert-butyl methyl ether (10 V) and the resulting filtrate was washed with water (10 V). The aqueous layer was separated and washed with tert-butyl methyl ether (10 V). The organic layers were combined and dried with MgSO₄ before concentrating at about 50° C. to afford intermediate N-1. ¹H NMR (400 MHz, CDCl3) δ 5.43 (qd, J=6.8, 1.4 Hz, 1H), 5.29 (qd, J=6.7, 1.4 Hz, 1H), 3.10 (s, 3H), 2.06 (s, 3H), 1.62 (d, J=6.7 Hz, 3H), 1.48 (d, J=6.8 Hz, 3H).

### Example 22: Preparation of (2S,5S)-5-(1,3-dioxoisoindolin-2-yl)hex-3-yn-2-yl Acetate (H-2)

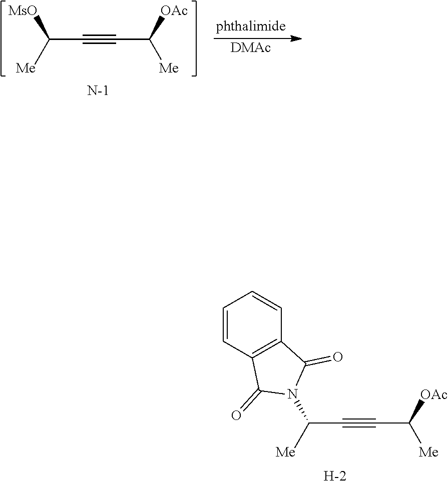

A reaction vessel was charged with Intermediate N-1 (1.0 mol equiv, scaling factor) and diluted with N,N-dimethylacetamide (8 V). Potassium phthalimide (1.2 mol equiv) was charged to the stirring reaction and heated to about 60° C. After about 24 h, the reaction solution was filtered and the filtrate was heated to about 60° C. and diluted with water (13 V). Reaction was cooled to about 22° C. over about 3 h before filtering the solution. The cake was washed with N,N-dimethylacetamide/water (2 V; 1:1). The cake was dried under nitrogen at about 50° C. to afford compound H-2. ¹H NMR (400 MHz, CDCl₃) δ 7.84 (dd, J=5.4, 3.1 Hz, 2H), 7.71 (dd, J=5.5, 3.0 Hz, 2H), 5.46 (qd, J=6.7, 1.8 Hz, 1H), 5.23 (qd, J=7.2, 1.8 Hz, 1H), 2.05 (s, 3H), 1.68 (d, J=7.1 Hz, 3H), 1.47 (d, J=6.6 Hz, 3H).

### Example 23: Preparation of (2S,5S)-5-(1,3-dioxoisoindolin-2-yl)hex-3-yn-2-yl Acetate (H-2)

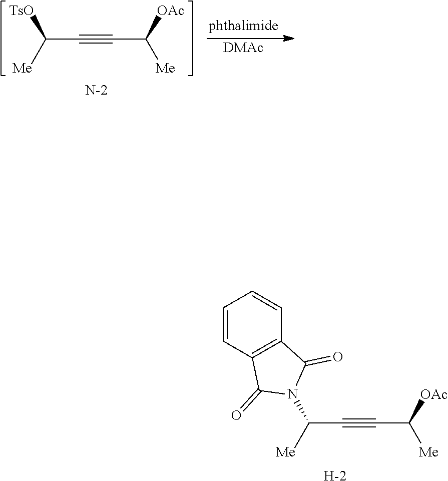

(2S,5R)-5-(tosyloxy)hex-3-yn-2-yl acetate (N-2) was prepared in a similar manner to Example 21 using toluenesulfonyl chloride instead of methanesulfonyl chloride and in the presence of an additive (18-crown-6).

A reaction vessel was charged with compound N-2 (0.0 mol equiv, scaling factor), 18-crown-6 (1.3 equiv) and diluted with N,N-dimethylacetamide (10 V). Potassium phthalimide (1.3 mol equiv) was charged to the stirring reaction and stirred at ambient temperature. After about 24 h, the reaction solution was filtered. Water (3 V) and ethyl acetate (5 V) were added to the reaction and agitated. Layers were partitioned and the aqueous layer was extracted twice more with ethyl acetate (5 V). Washed the organic layer with 5% aqueous sodium chloride (3 V). The organic layer was concentrated to an oil and purified via flash column chromatography using 0 to 100% ethyl acetate/hexane to afford (2S,5S)-5-(1,3-dioxoisoindolin-2-yl)hex-3-yn-2-yl acetate (H-2).

¹H NMR (400 MHz, CDCl₃) δ 7.84 (dd, J=5.4, 3.1 Hz, 2H), 7.71 (dd, J=5.5, 3.0 Hz, 2H), 5.46 (qd, J=6.7, 1.8 Hz, 1H), 5.23 (qd, J=7.2, 1.8 Hz, 1H), 2.05 (s, 3H), 1.68 (d, J=7.1 Hz, 3H), 1.47 (d, J=6.6 Hz, 3H).

### Example 24: Preparation of (2R,5S)-5-hydroxyhex-3-yn-2-yl acetate (O-4-A)

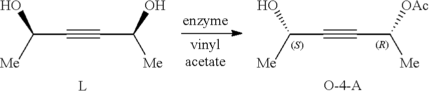

Hex-3-yne-2,5-diol (L, 1.00 equiv., scaling factor) was charged to reactor A, followed by Lipase AK (0.1 g/g), isopropenyl acetate (3 equiv.), and tert-butyl methyl ether (4 V). This was agitated at about 50° C. for about 30 hours. The reaction was cooled to about 20° C. and agitated for about 16 hours. The solution was filtered and the cake was rinsed with tert-butyl methyl ether (4 V). The organics were recharged to reactor A and washed with water (4×4 V). The aqueous layer was removed. The contents were distilled to dryness. This was purified via column chromatography using a 10% ethyl acetate in heptane mixture to 25% ethyl acetate mixture. The purified material was distilled at about 40° C. to afford compound O-4-A. ¹H NMR (400 MHz, DMSO) δ 5.40 (qd, J=6.6, 1.5 Hz, 1H), 5.34 (d, J=5.4 Hz, 1H), 4.39 (tt, J=6.6, 5.1 Hz, 1H), 2.03 (s, 3H), 1.40 (d, J=6.7 Hz, 3H), 1.27 (d, J=6.6 Hz, 3H).

### Example 25: Preparation of di-tert-butyl 1-((2R,5R)-5-acetoxyhex-3-yn-2-yl)hydrazine-1,2-dicarboxylate (O-3-A)

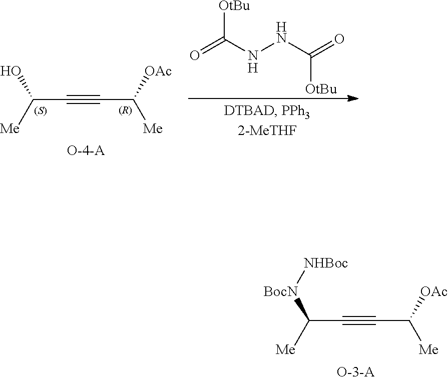

A reaction vessel was charged with compound O-4-A (scaling factor, 1.0 equiv), di-tert-butyl hydrazodiformate (0.1 equiv), and triphenylphosphine (1.2 equiv) and 2-methyltetrahydrofuran (10 V) at about 25° C. The contents were agitated and cooled to about 0° C. Di-tert-butyl azodicarboxylate (1.2 equiv) was charged portion wise to the reaction and left to agitate for about 1 hour. The reaction mixture was then warmed up to about room temperature and left to agitate for about 16 hours. Following overnight agitation, the crude was distilled to dryness and purified via column chromatography using a gradient of eluents from 10% ethyl acetate in heptane mixture going up to 50%. The purified material was distilled down to dryness to afford solid compound O-3-A. ¹H NMR (400 MHz, DMSO-d₆): δ 8.98 (s, 1H), 5.48-5.26 (m, 1H), 5.20-4.59 (m, 1H), 2.01 (s, 3H), 1.51-1.12 (m, 24H)

### Example 26: Preparation of di-tert-butyl 1-((2R,5R)-5-hydroxyhex-3-yn-2-yl)hydrazine-1,2-dicarboxylate (O-2-A)

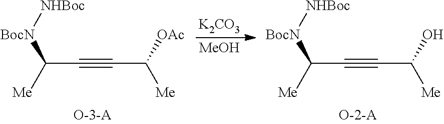

Compound O-3-A (1.0 mol equiv, scaling factor) and methanol (3 V) were charged to a reactor. To the stirring solution was charged potassium carbonate (0.10 mol equiv). The mixture was agitated at about 20° C. for about 4 hours. To the solution was charged toluene (9 V), and the contents of the reactor adjusted to about 5° C. Water (5 V) was charged over about 30 minutes. Agitation was stopped to allow the layers to settle, and the aqueous layer was discharged. To the reactor was charged water (5 V) over about 15 minutes. Agitation was stopped to allow the layers to settle, and the aqueous layer was discharged. The organic solution was purified to afford compound O-2-A. ¹H NMR (400 MHz, CDCl₃) δ 5.12 (s, 1H), 4.51 (q, J=6.7 Hz, 1H), 1.48 (d, J=4.0 Hz, 21H), 1.43 (d, J=6.6 Hz, 3H), 1.37 (d, J=7.0 Hz, 3H).

### Example 27: Preparation of di-tert-butyl 1-((2R,5R,Z)-5-hydroxyhex-3-en-2-yl)hydrazine-1,2-dicarboxylate (O-1-A)

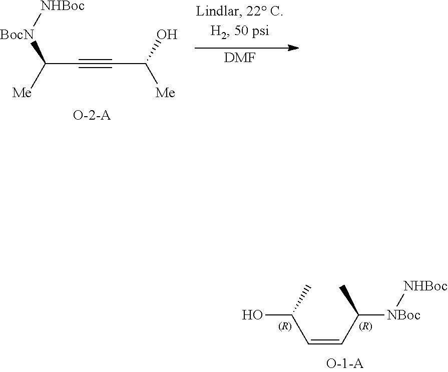

Compound O-2-A (1.00 equiv, scaling factor) and palladium on calcium carbonate, lead poisoned (Lindlar catalyst, 0.003 equiv) were charged to a 200 mL stainless steel HEL AutoMate reactor at about 22° C. N,N-dimethylformamide (5 V) was charged at about 22° C. The reaction vessel was sealed, and the atmosphere of the sealed vessel was purged by pressurizing the contents to about 50 psi with nitrogen and venting. This cycle was repeated two times. The atmosphere was exchanged to hydrogen by pressurizing the contents to about 50 psi with hydrogen and venting. The vessel was pressurized with hydrogen about 50 psi and repressurized to about 50 psi as needed over the course of the reaction. After about 16 h, the product mixture was filtered over a pad of celite. The pad of celite was rinsed with N,N-dimethylformamide (1.5 V) and the filtrate was collected. To the filtrate was charged aqueous sodium chloride solution (about 10% (w/v, 10 V) and methyl-tert-butyl ether (10 V). The resulting biphasic mixture was transferred to a separation funnel and the layers that formed were separated. The organic layer was washed with an additional portion of aqueous sodium chloride solution (about 10% (w/v, 5 V) and the layers that formed were separated. The aqueous layers were discarded, and the organic solution was purified to afford compound O-1-A. ¹H NMR (400 MHz, CDCl₃) δ 6.08 (s, 1H), 5.55-5.43 (m, 1H), 5.38 (t, J=10.1 Hz, 1H), 5.16 (s, 1H), 4.78-4.62 (m, 1H), 1.47 (s, 18H), 1.29-1.21 (m, 6H).

### Example 28: Preparation of di-tert-butyl 1-((2R,5S,Z)-5-(1,3-dioxoisoindolin-2-yl)hex-3-en-2-yl)hydrazine-1,2-dicarboxylate (E-2)

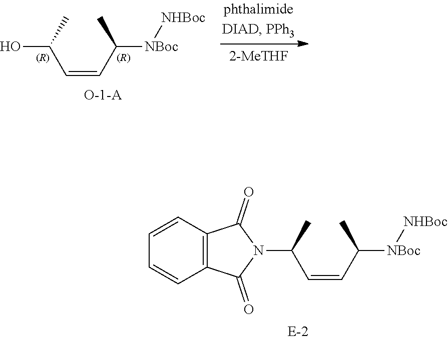

Compound O-1-A (1.0 equiv, scaling factor) was charged to a reactor followed by 6 V of 2-methyltetrahydrofuran. Phthalimide (1.l equiv) and triphenylphosphine (1.2 equiv) were added to the stirring solution. In a separate flask, diisopropyl azodicarboxylate (1.2 equiv) was added and diluted with 2 V of 2-methyltetrahydrofuran. Slow addition of the DIAD solution was charged to the reactor at about 5° C. over about 1 hour. About 2 hours after complete addition of the DIAD solution, the solution was left to stir for about 2 hours. The reaction was filtered, and the resulting cake was washed with 2 V of 2-methyltetrahydrofuran. The filtered organic solution was purified to afford compound E-2. ¹H NMR (400 MHz, CDCl₃) δ 7.77 (dd, J=5.4, 3.1 Hz, 2H), 7.65 (dd, J=5.5, 3.0 Hz, 2H), 6.22 (s, 1H), 5.95 (t, J=10.0 Hz, 1H), 5.51 (s, 1H), 5.17 (t, J=7.8 Hz, 1H), 4.93 (s, 3H), 1.49 (d, J=6.9 Hz, 2H), 1.40 (s, 9H), 1.25 (s, 12H).

### Example 29: Preparation of (2S,5S)-5-azidohex-3-yn-2-yl acetate (P-2-A)

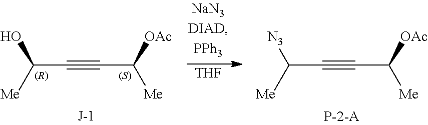

A reaction vessel was charged with compound J-1 (1.0 equiv, scaling factor), triphenylphosphine (1.2 equiv) and tetrahydrofuran (15 V). Cooled reaction mixture to about 0° C. Charged diisopropyl azodicarboxylate (1.2 equiv) followed by sodium azide (1.2 equiv). Allowed mixture to warm to ambient temperature. Reaction was diluted with water and extracted with ethyl acetate. Organic was washed with brine and concentrated in vacuo to an oil (P-2-A). ¹H NMR (400 MHz, CDCl₃) δ 5.50 (qd, J=6.7, 1.8 Hz, 1H), 4.24-4.14 (m, 1H), 2.08 (s, 3H), 1.51 (d, J=6.7 Hz, 3H), 1.42 (d, J=6.9 Hz, 3H).

### Example 30: Preparation of (2S,5S)-5-aminohex-3-yn-2-yl acetate (P-1-A)

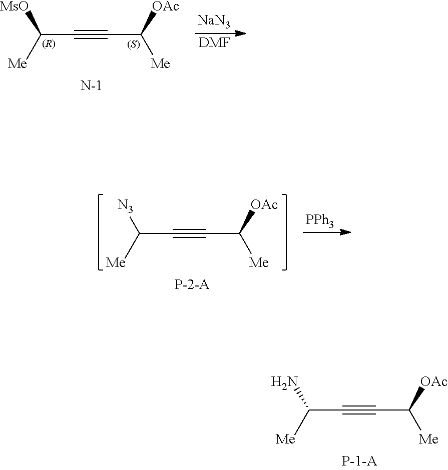

A reaction vessel was charged with compound N-1 (1.0 equiv, scaling factor), dimethylformamide (5 V) and sodium azide (1.2 equiv). After agitating the reaction mixture at about 20° C. for 4 hours triphenylphosphine (1.5 equiv) and water (10 equiv) were charged at about 20° C. Upon reaction completion reaction was diluted with ethyl acetate (26 V). Organic was washed with water (4×17 V), 10% sodium chloride aq. (17 V), and dried over anhydrous magnesium sulfate. Organic was concentrated to dryness and purified by silica gel chromatography eluting with 5% methanol in dichloromethane. Compound P-1-A was isolated as a yellow oil. ¹H NMR (400 MHz, DMSO-d₆) δ 5.38 (qd, J=6.6, 1.7 Hz, 1H), 3.60 (qd, J=6.8, 1.7 Hz, 1H), 2.02 (s, 3H), 1.79 (s, 2H), 1.39 (d, J=6.6 Hz, 3H), 1.19 (d, J=6.8 Hz, 3H).

### Example 31: Preparation of (2S,5S)-5-((tert-butoxycarbonyl)amino)hex-3-yn-2-yl Acetate (H-1-A)

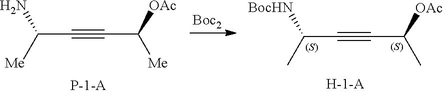

A reaction vessel was charged with compound P-1-A (1.0 equiv, scaling factor), di-tert-butyl dicarbonate (2.0 equiv), tetrahydrofuran (12V) and 10% K₂CO₃ aq (12V). Reaction mixture was agitated at about 20° C. Upon reaction completion charged ethyl acetate (20 V) and water (6 V). Separated bottom aqueous and washed organic with 10% NaCl aq. (12 V). Dried organic over anhydrous MgSO₄, filtered and concentrated in vacuo. Purified crude by silica gel chromatography. Isolated H-1-A as a clear oil. ¹H NMR (400 MHz, DMSO-d₆) δ 7.30 (d, J=8.5 Hz, 1H), 5.37 (qd, J=6.6, 1.7 Hz, 1H), 4.42-4.25 (m, 1H), 2.02 (s, 3H), 1.38 (d, J=4.7 Hz, 12H), 1.24 (d, J=7.0 Hz, 3H).

### Example 32: Preparation of tert-butyl ((2S,5S)-5-hydroxyhex-3-yn-2-yl)carbamate (G-1-A)

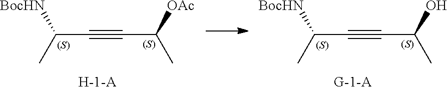

A reaction vessel was charged with compound H-1-A (1.0 equiv, scaling factor), methanol (5 V) and 1, 1, 3, 3-tetramethylguanidine (0.1 equiv). Reaction was agitated at about 20° C. Upon reaction completion, concentrated the contents in vacuo to remove methanol. Charged EtOAc (38 V) and washed organic with 10% Citric Acid aq. (15 V) and 10% NaCl aq. (15 V). Dried organic over anhydrous MgSO₄, filtered and concentrated in vacuo to an oil (G-1-A). ¹H NMR (400 MHz, DMSO-d₆) δ 7.24 (d, J=8.4 Hz, 1H), 5.22 (d, J=5.3 Hz, 1H), 4.33 (q, J=6.1 Hz, 2H), 1.39 (s, 9H), 1.24 (dd, J=6.8, 5.0 Hz, 6H).

### Example 33: Preparation of tert-butyl ((2S,5S,Z)-5-hydroxyhex-3-en-2-yl)carbamate (F-1-A)

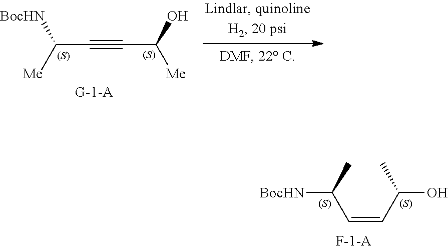

A reaction vessel was charged with compound G-1-A (1.0 equiv, scaling factor) in dimethylformamide (10 V), lindlar catalyst (0.002 equiv) and quinoline (0.02 equiv). Placed reaction vessel into a stainless-steel par reaction. Applied 3 cycles of nitrogen gas by pressurizing to 20 psi and venting the system. Applied 3 cycles of hydrogen gas by pressurizing to 40 psi and venting the system. Pressurized with hydrogen gas to 20 psi and agitated the reaction mixture at about 22° C. overnight. Reaction was diluted with ethyl acetate (30 V), filtered through a pad of celite and rinsed forward with ethyl acetate (75 V). Washed organic with water (45 V). Separated organic and back extracted aqueous with ethyl acetate (30 V). Combined organic layers were washed with water (2×45 V) and 10% sodium chloride aq. (45 V). Dried organic over anhydrous magnesium sulfate, filtered and concentrated in vacuo to afford F-1-A as an off white solid. ¹H NMR (400 MHz, DMSO-d₆) δ 6.84 (d, J=8.3 Hz, 1H), 5.24 (dd, J=11.0, 8.3 Hz, 1H), 5.19-5.09 (m, 1H), 4.59 (d, J=4.3 Hz, 1H), 4.48 (q, J=6.2 Hz, 1H), 4.33 (q, J=7.8 Hz, 1H), 1.37 (s, 9H), 1.12-1.02 (m, 6H).

### Example 34: Preparation of tert-butyl ((2S,5S,Z)-5-hydroxyhex-3-en-2-yl)carbamate (F-1-A)

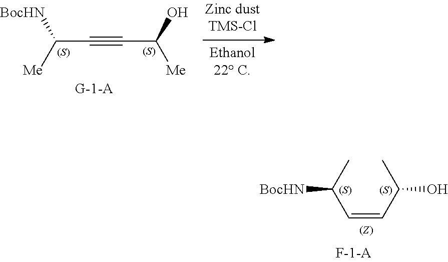

A reaction vessel was charged with Zn dust (5.0 equiv), EtOH (5 V), trichloromethylsilane (0.75 equiv), and heated to 80° C. for no less than 1 hour. Charged tert-butyl ((2S,5S)-5-hydroxyhex-3-yn-2-yl)carbamate (G-1-A, 1.0 equiv, scaling factor) in EtOH (3 V). Stirred for no less than 24 hours. Quenched with 2 V 5% aqueous NaCl solution, filtered slurry over celite, and rinsed forward with EtOAc. Extracted aqueous layer with EtOAc (3 V) twice, dried over magnesium sulfate, and filtered. Purified via silica gel chromatography with mobile phase 0 to 100% EtOAc/Hexane to afford tert-butyl ((2S,5S,Z)-5-hydroxyhex-3-en-2-yl)carbamate (F-1-A) as a clear oil.

¹H NMR (400 MHz, DMSO-d₆) δ 6.84 (d, J=8.3 Hz, 1H), 5.24 (dd, J=11.0, 8.3 Hz, 1H), 5.19-5.09 (m, 1H), 4.59 (d, J=4.3 Hz, 1H), 4.48 (q, J=6.2 Hz, 1H), 4.33 (q, J=7.8 Hz, 1H), 1.37 (s, 9H), 1.12-1.02 (m, 6H).

### Example 35: Preparation of di-tert-butyl 1-((2R,5S,Z)-5-((tert-butoxycarbonyl)amino)hex-3-en-2-yl)hydrazine-1,2-dicarboxylate (D-3)

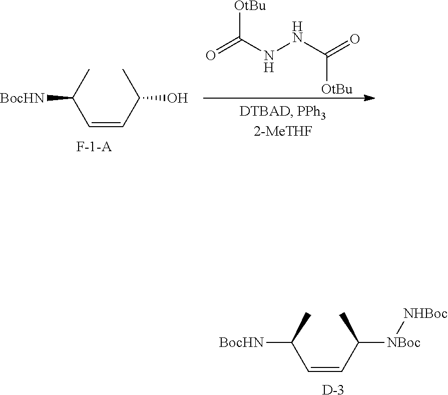

A reaction vessel was charged with compound F-1-A (1.0 equiv, scaling factor), MeTHF (15 V), di-tert-butyl hydrazodicarboxylate (0.1 equiv) and triphenylphosphine (1.2 equiv). Reaction mixture was agitated at about 0° C. Charged di-tert-butyl azodicarboxylate (1.2 equiv). Warmed up reaction mixture to about 23° C. Upon reaction completion charged magnesium chloride (2.0 equiv) and agitated mixture overnight. Filtered slurry through a pad of celite and rinsed forward with QS EtOAc. Washed organic with water (30 V) and 10% NaCl aq (30 V). Dried organic over anhydrous magnesium sulfate, filtered and concentrated in vacuo. Crude was purified by silica gel chromatography to afford compound D-3 as a white solid. ¹H NMR (400 MHz, Methanol-d₄) δ 6.59 (s, 1H), 5.35 (t, J=8.2 Hz, 2H), 5.03 (m, 1H), 4.37 (m, 1H), 1.64-1.34 (m, 27H), 1.20 (dd, J=11.4, 6.8 Hz, 6H).

### Example 36: Preparation of Mesylate Salt of (2S,5R,Z)-5-hydrazineylhex-3-en-2-amine (C-1)

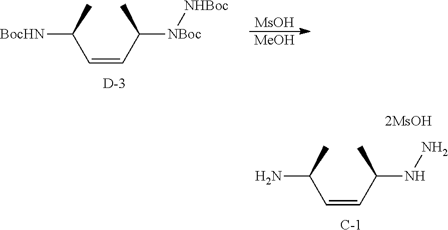

A reaction vessel was charged with compound D-3 (1.0 equiv, scaling factor), MeOH (16 V) and MsOH (4.2 eq). Agitated reaction mixture at about 60° C. Upon reaction completion concentrated reaction in vacuo to give compound C-1. ¹H NMR (400 MHz, DMSO-d₆) δ 7.91 (s, 3H), 7.29 (s, 5H), 5.62 (t, J=10.3 Hz, 1H), 5.44 (t, J=10.4 Hz, 1H), 4.22-4.11 (m, 1H), 3.98 (dd, J=9.8, 6.5 Hz, 1H), 2.40 (s, 9H), 1.22 (d, J=6.6 Hz, 3H), 1.13 (d, J=6.5 Hz, 3H).

### Example 37: Preparation of Toluenesulfonic Acid Salt of (2S,5R,Z)-5-hydrazineylhex-3-en-2-amine (C-2)

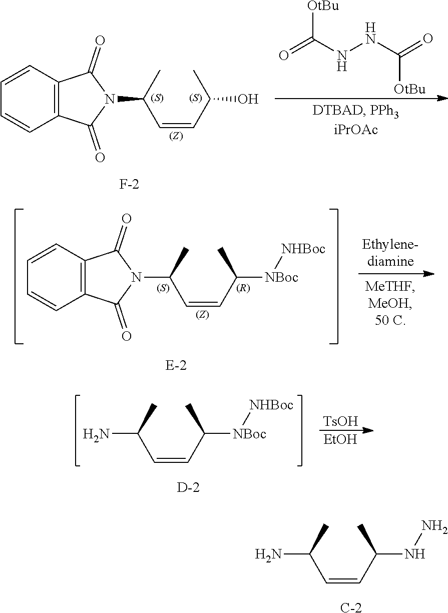

A reaction vessel was charged with 2-((2S,5S,Z)-5-hydroxyhex-3-en-2-yl)isoindoline-1,3-dione (F-2, scaling factor, 1.0 equiv), di-tert-butyl hydrazodiformate (0.08 equiv), triphenylphosphine (1.2 equiv) and isopropyl acetate (10 V) at about 20° C. The contents were agitated and cooled to about 0° C. Di-tert-butyl azodicarboxylate (1.2 equiv) was charged in one portion and the contents were warmed to about 20° C. The contents were allowed to stir for 3 h. Heptane (5 V) was charged, and the contents were cooled to about 0° C. and agitated for 16 h. The slurry was filtered and rinsed forward with a mixture of isopropyl acetate (0.8 V) and heptane (3.2 V) at 0° C. The filtrate and rinse were charged back to the reaction vessel. The contents were heated to about 65° C. and distilled to 4 V. Methanol (15 V) was charged to the reaction vessel and the mixture was distilled to 4 V. The contents were cooled to about 20° C. and 2-methyltetrahydrofuran (4 V) and methanol (1 V) were charged. To the stirring solution was charged ethylenediamine (3.0 mol equiv). The mixture was heated to about 50° C. and agitated for about 3 hours. The temperature of the reactor was set to about 20° C. and to the slurry was charged 2-methyltetrahydrofuran (4 V), methyl t-butyl ether (9 V), and water (14 V). The contents of the reactor were filtered, and the filtrate collected into a separate reactor. Agitation of the filtrate was stopped, the layers were allowed to settle, and the aqueous layer was discharged. To the remaining organic was charged water (14 V). After about 30 m, agitation was stopped, the layers were allowed to settle, and the aqueous layer was discharged. The reactor contents were heated to about 65° C. and then distilled to 6 V. To the reactor was charged ethanol (12 V) and the contents were distilled to 6 V. To the reactor was charged ethanol (12 V) and the contents were distilled to 6 V. To the reactor was charged ethanol (10 V) and the reactor contents were cooled to about 20° C. To the stirring solution was charged para-toluenesulfonic acid monohydrate (3.0 equiv). The reactor contents were heated to about 60° C. After 2 h the (2S,5R,Z)-5-hydrazineylhex-3-en-2-amine toluenesulfonic acid salt (C-2, 0.0002 equiv) was charged and the mixture was agitated for 16 h. The reactor contents were cooled to about 20° C. over 1 h and the mixture was allowed to stir for an additional hour. The reactor contents were filtered, and the filtrate was discarded. To the wet cake was charged ethanol (6 V), the slurry was agitated for 30 min, and then the solvent was filtered off. The reslurry was performed three more times with ethanol (6 V). Finally, the solids were rinsed with ethanol (6 V) and dried at 50° C. with a N₂ sweep to give compound C-2 as a white crystalline solid.

¹H NMR (400 MHz, DMSO-d₆) δ 7.51 (d, J=8.1 Hz, 4H), 7.26-7.09 (d, 4H), 5.66 (t, J=10.3 Hz, 1H), 5.48 (t, J=10.4 Hz, 1H), 4.17 (qd, J=6.6, 2.6 Hz, 1H), 4.00 (s, 1H), 2.30 (s, 6H), 1.24 (d, J=6.6 Hz, 3H), 1.16 (d, J=6.4 Hz, 3H).

### Example 38: Preparation of Toluenesulfonic Acid Salt of (2S,5R,Z)-5-hydrazineylhex-3-en-2-amine (C-2)

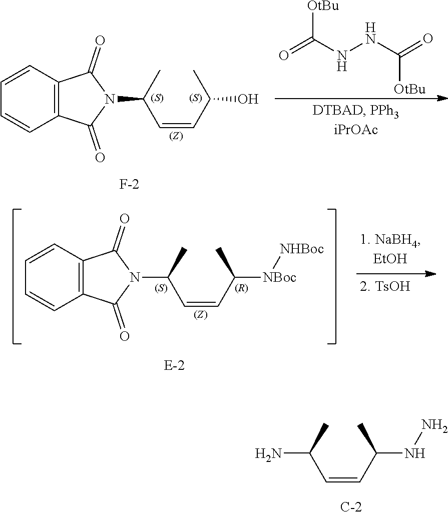

A reaction vessel was charged with 2-((2S,5S,Z)-5-hydroxyhex-3-en-2-yl)isoindoline-1,3-dione (F-2, scaling factor, 1.0 equiv), di-tert-butyl hydrazodiformate (0.08 equiv), triphenylphosphine (1.2 equiv) and isopropyl acetate (10 V) at about 20° C. The contents were agitated and cooled to about 0° C. Di-tert-butyl azodicarboxylate (1.2 equiv) was charged in one portion and the contents were warmed to about 20° C. The contents were allowed to stir for 3 h. Heptane (5 V) was charged, and the contents were cooled to about 0° C. and agitated for 16 h. The slurry was filtered and rinsed forward with a mixture of isopropyl acetate (0.8 V) and heptane (3.2 V) at 0° C. The filtrate and rinse were charged back to the reaction vessel. The contents were heated to about 65° C. and distilled to 4 V. Ethanol (15 V) was charged to the reaction vessel and the mixture was distilled to 4 V. To the reactor was charged ethanol (6 V) and the contents were cooled to about 20° C. To the stirring solution was charged sodium borohydride (2 equiv.) and the contents were allowed to stir for 16 h. To the stirring solution was charged para-toluenesulfonic acid (7 equiv) over about 2 h. The reactor contents were heated to about 60° C. After 2 the (2S,5R,Z)-5-hydrazineylhex-3-en-2-amine toluenesulfonic acid salt (C-2, 0.0002 equiv) was charged and the mixture was agitated for 16 h. The reactor contents were cooled to about 20° C. over 1 h and the mixture was allowed to stir for an additional hour. The reactor contents were filtered, and the filtrate was discarded. To the wet cake was charged ethanol (6 V), the slurry was agitated for 30 min, and then the solvent was filtered off. The reslurry was performed three more times with ethanol (6 V). Finally, the solids were rinsed with ethanol (6 V) and dried at 50° C. with a N₂ sweep to give compound C-2 as a white crystalline solid.

¹H NMR (400 MHz, DMSO) δ 7.51 (d, J=8.1 Hz, 4H), 7.26-7.09 (d, 4H), 5.66 (t, J=10.3 Hz, 1H), 5.48 (t, J=10.4 Hz, 1H), 4.17 (qd, J=6.6, 2.6 Hz, 1H), 4.00 (s, 1H), 2.30 (s, 6H), 1.24 (d, J=6.6 Hz, 3H), 1.16 (d, J=6.4 Hz, 3H).

### Example 39: Preparation of methyl 1-(((2R,5S,Z)-5-(1,3-dioxoisoindolin-2-yl)hex-3-en-2-yl)amino)-3-methoxy-4-oxo-5-((2,4,6-trifluorobenzyl)carbamoyl)-1,4-dihydropyridine-2-carboxylate (Q-1-A)

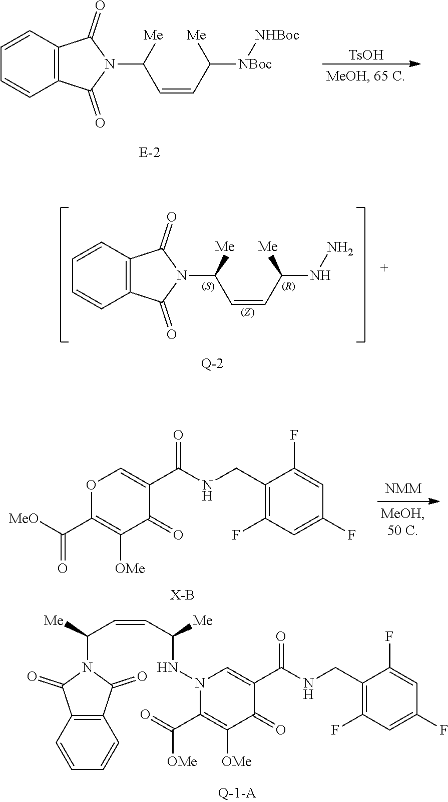

A reaction vessel was charged with compound E-2 (scaling factor, 1.0 equiv), methanol (10 V), and p-toluenesulfonic acid (2.5 equiv). The contents were agitated at about 65° C. for about 4 hours, then adjusted to about 35° C. Compound X-B (1.0 equiv) and N-methylmorpholine (3.5 equiv) were charged and the contents were agitated at about 50° C. for about 20 hours. Toluene (15 V) and water (10 V) were charged and the aqueous layer was separated. Water (10 V) was charged and the aqueous layer was separated. The contents of the reactor were distilled to minimum volume and 2-butanone (8 V) was charged. The contents of the reactor were stirred at about 50° C. and heptane (3 V) was charged and stirred for about 4 hours. Heptane (2 V) was added and stirred for about 1 hour and the slurry was filtered and dried at about 50° C. to afford compound Q-1-A. ¹H NMR (400 MHz, DMSO-d6) δ 9.66 (dd, J=6.6, 4.9 Hz, 1H), 8.16 (s, 1H), 7.70 (dd, J=5.4, 3.1 Hz, 2H), 7.61 (dd, J=5.4, 3.0 Hz, 2H), 7.24-7.09 (m, 3H), 6.08 (t, J=10.5 Hz, 1H), 5.45 (t, J=10.6 Hz, 1H), 4.87 (dq, J=10.2, 6.9 Hz, 1H), 4.28 (dd, J=14.5, 6.6 Hz, 1H), 4.09 (dq, J=16.9, 6.8 Hz, 1H), 3.94-3.82 (m, 4H), 3.76 (s, 3H), 1.35 (d, J=6.8 Hz, 3H), 1.08 (d, J=6.6 Hz, 3H).

### Example 40: Preparation of methyl 1-(((2R,5S,Z)-5-aminohex-3-en-2-yl)amino)-3-methoxy-4-oxo-5-((2,4,6-trifluorobenzyl)carbamoyl)-1,4-dihydropyridine-2-carboxylate (A-1)

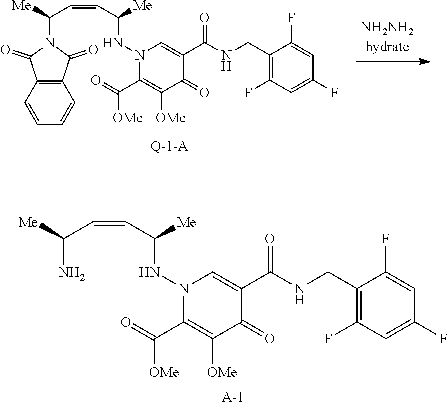

Toluene and hydrazine hydrate was sparged with N₂ prior to reaction setup. A reaction vessel was charged with compound Q-1-A (scaling factor, 1.0 equiv) and toluene (10 V). The contents were agitated at about 85° C. Once the solution reached temperature, hydrazine hydrate (3 equiv) was charged over about 30 minutes. The contents were agitated at about 85° C. for about three hours. Purified via flash chromatography to afford compound A-1. ¹H NMR (400 MHz, DMSO-d₆) δ 10.32 (t, J=5.8 Hz, 1H), 8.55 (s, 1H), 7.27-7.18 (m, 2H), 7.16 (d, J=5.8 Hz, 1H), 5.37 (dd, J=10.9, 9.1 Hz, 1H), 5.11 (ddd, J=10.8, 9.6, 1.0 Hz, 1H), 4.55 (t, J=5.1 Hz, 2H), 4.19 (dt, J=9.7, 6.1 Hz, 1H), 3.90 (s, 3H), 3.77 (s, 3H), 3.65-3.51 (m, 1H), 0.97 (dd, J=10.7, 6.4 Hz, 6H).

### Example 41: Preparation of 1-(((2R,5S,Z)-5-aminohex-3-en-2-yl)amino)-3-methoxy-4-oxo-5-((2,4,6-trifluorobenzyl)carbamoyl)-1,4-dihydropyridine-2-carboxylic acid (R-2-A)

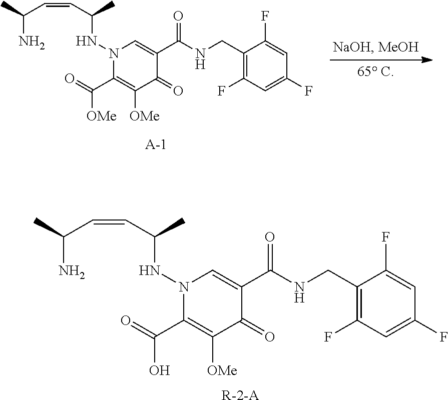

A reaction vessel was charged with compound A-1 (scaling factor, 1.0 equiv), methanol (10 V), and 2N aqueous sodium hydroxide (5.0 equiv). The contents were agitated and heated to about 65° C. for about 30 minutes. The reaction mixture was then cooled to about 20° C. and filtered. The filtrate was concentrated to a minimum volume and concentrated hydrochloric acid was added (3.0 equiv). Ethyl acetate (10 V) was added followed by heptane (4 V). The contents were agitated for about 16 hours and then filtered. The cake was washed with a premixed solution of ethyl acetate (1.4 V) and heptane (0.6 V). The resulting cake was dried at about 50° C. to provide compound R-2-A. ¹H NMR (400 MHz, DMSO-d₆) δ 10.65 (t, J=5.7 Hz, 1H), 8.34 (br s, 3H), 8.20 (s, 1H), 7.35-7.12 (m, 2H), 6.87 (s, 1H), 5.60-5.38 (m, 2H), 4.52 (d, J=5.8 Hz, 2H), 4.24 (t, J=7.9 Hz, 1H), 4.15 (s, 1H), 3.71 (s, 3H), 1.23 (d, J=6.4 Hz, 3H), 1.04 (d, J=6.0 Hz, 3H).

### Example 42: Preparation of (2R,5S,Z)-8-methoxy-2,5-dimethyl-7,9-dioxo-N-(2,4,6-trifluorobenzyl)-1,2,5,6,7,9-hexahydropyrido[1,2-b][1,2,5]triazonine-10-carboxamide (R-1-A)

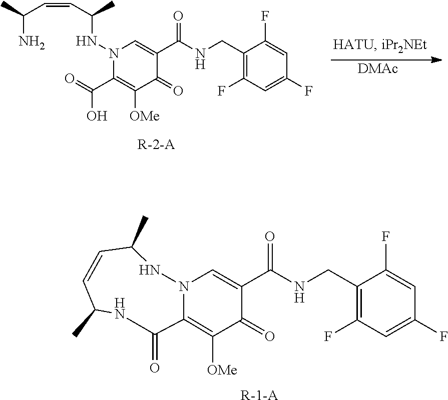

A reaction vessel was charged with compound R-2-A (scaling factor, 1.0 equiv), 1-[Bis(dimethylamino)methylene]-1H-1,2,3-triazolo[4,5-b]pyridinium 3-oxid hexafluorophosphate (HATU) (1.2 equiv), N,N-dimethylacetamide (40 V), and N,N-diisopropylethylamine (5.0 equiv). The contents were agitated for about 2.5 hours. A mixture of water (60 V) and concentrated hydrochloric acid (10.0 equiv) was then added. The reaction mixture was then extracted with isopropyl acetate (10 V×4) and ethyl acetate (10 V×3). The combined organic extracts were washed with water (12 V), dried with sodium sulfate, filtered, and concentrated. The material was purified by column chromatography using ethyl acetate and hexanes (60 to 100%) to afford compound R-1-A. ¹H NMR (400 MHz, DMSO-d₆) δ 10.55 (t, J=5.8 Hz, 1H), 8.41-8.30 (m, 2H), 7.33-7.16 (m, 2H), 7.00 (d, J=2.2 Hz, 1H), 5.47 (ddd, J=10.5, 8.5, 2.0 Hz, 1H), 5.39 (dd, J=10.6, 4.7 Hz, 1H), 4.66 (dd, J=14.5, 6.3 Hz, 1H), 4.45 (dd, J=14.6, 5.3 Hz, 1H), 4.07-3.90 (m, 2H), 3.73 (s, 3H), 1.35 (d, J=7.2 Hz, 3H), 1.10 (d, J=6.4 Hz, 3H).

### Example 43: Preparation of (2R,5S,Z)-8-methoxy-2,5-dimethyl-7,9-dioxo-N-(2,4,6-trifluorobenzyl)-1,2,5,6,7,9-hexahydropyrido[1,2-b][1,2,5]triazonine-10-carboxamide (R-1-A)

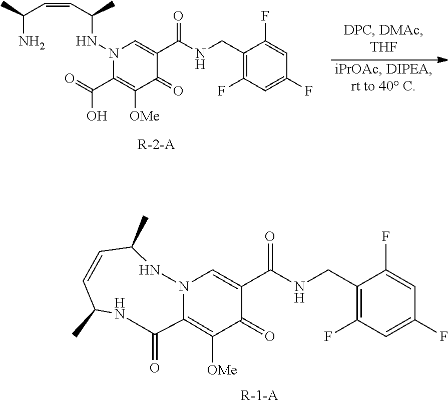

A reaction vessel was charged with 1-(((2R,5S,Z)-5-aminohex-3-en-2-yl)amino)-3-methoxy-4-oxo-5-((2,4,6-trifluorobenzyl)carbamoyl)-1,4-dihydropyridine-2-carboxylic acid (R-2-A, scaling factor, 1.0 equiv), N,N-dimethylacetamide (1.0 V), tetrahydrofuran (8.5 V), and diphenyl chlorophosphate (1.5 equiv). The contents were agitated at about 20° C. for about 16 hours. The contents were then charged slowly into a premixed solution of isopropyl acetate (3.0 V) and N,N-diisopropylethylamine (3.5 equiv) at about 40° C. over about 2 hours. The contents were agitated for about 2.5 hours before adding isopropyl acetate (7.0 V) and concentrating down to 7.0 V at about 30° C. Isopropyl acetate (7.0 V) was added and the solution was concentrated again to 7.0 V before adding isopropyl acetate (7.0 V). At about 20° C., 1M hydrochloric acid in water (3.2 equiv) was added to the solution and the organic and aqueous layers were separated. 10 wt % aqueous potassium carbonate (2.0 equiv) was added over about 30 minutes. The organic layer was washed with water (3.0 V). The organic layer was concentrated down to 3.0 V at about 50° C. before charging methyl tert-butyl ether (1.5 V) over about an hour at about 35° C. (2R,5S,Z)-8-methoxy-2,5-dimethyl-7,9-dioxo-N-(2,4,6-trifluorobenzyl)-1,2,5,6,7,9-hexahydropyrido[1,2-b][1,2,5]triazonine-10-carboxamide (R-1-A) seeds were charged to the reaction vessel and the slurry was left to age for about 16 hours. The slurry was cooled to about 20° C. over about an hour before adding methyl tert-butyl ether (3.0 V) over about an hour. This slurry was left to agitate over about 16 hours before filtering off the solids and washing the cake with methyl tert-butyl ether (2.0 V). The solids were dried in a vacuum oven for about 16 hours at about 50° C. to afford compound R-1-A.

1H NMR (400 MHz, CDCl₃) δ 10.47 (t, J=5.8 Hz, 1H), 8.51 (s, 1H), 6.72-6.60 (m, 2H), 6.41 (d, J=11.1 Hz, 1H), 5.61 (d, J=2.5 Hz, 1H), 5.57-5.40 (m, 2H), 4.73 (dd, J=14.5, 6.2 Hz, 1H), 4.54 (dd, J=14.5, 5.3 Hz, 1H), 3.92-3.74 (m, 5H), 3.21 (s, 2H), 1.47 (d, J=7.2 Hz, 3H), 1.18 (s, 12H).

### Example 44: Preparation of (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B)

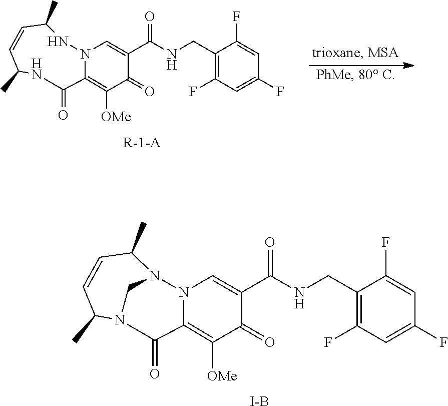

A reaction vessel was charged with compound R-1-A (scaling factor, 1.0 equiv), 1,3,5-trioxane (1.5 equiv), toluene (10 V), and methanesulfonic acid (4.0 equiv). The contents were agitated at about 80° C. for about 2 hours. Water (10 V) was added and the mixture was extracted with ethyl acetate (10 V×4). The combined organic extracts were dried with sodium sulfate, filtered, and concentrated. The material was purified by column chromatography using ethyl acetate and hexanes (45 to 100%) to afford compound I-B. ¹H NMR (400 MHz, Chloroform-d) δ 10.31 (s, 1H), 8.56 (s, 1H), 6.72-6.58 (m, 2H), 5.61 (dt, J=11.4, 2.3 Hz, 1H), 5.53 (dq, J=7.9, 2.8 Hz, 1H), 5.37 (ddd, J=11.3, 3.8, 2.8 Hz, 1H), 4.95 (d, J=14.3 Hz, 1H), 4.64 (qd, J=14.5, 5.7 Hz, 2H), 4.37 (dd, J=14.2, 0.9 Hz, 1H), 4.11 (s, 3H), 3.76 (qq, J=6.6, 3.1 Hz, 1H), 1.39 (d, J=6.7 Hz, 3H), 1.33 (d, J=7.4 Hz, 3H).

### Example 45: Preparation of (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B)

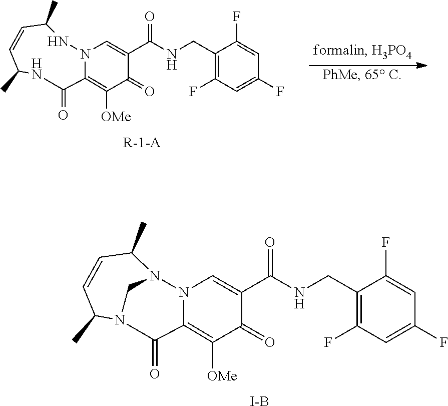

A reaction vessel was charged with (2R,5S,Z)-8-methoxy-2,5-dimethyl-7,9-dioxo-N-(2,4,6-trifluorobenzyl)-1,2,5,6,7,9-hexahydropyrido[1,2-b][1,2,5]triazonine-10-carboxamide (R-1-A, scaling factor, 1.0 equiv.), formalin (5 equiv.), and toluene (10 V). The contents were agitated at about 65° C. for about 18 hours. Phosphoric acid (4 equiv.) was charged to the reaction vessel. The contents were agitated at about 65° C. for about 4 hours. 20 wt % aqueous potassium carbonate (5 equiv.) was added over about 30 minutes. The organic layer was washed with water (3 equiv.). The organic layer was filtered and distilled to about 3 V before charging 2-propanol (4.5 V). The solution was distilled to about 3 V before charging 2-propanol (3 V). The solution was distilled to about 3 V before charging 2-propanol (1.5 V). The solution was distilled to about 3 V before charging 2-propanol (1 V). The temperature was adjusted to about 30° C. GS-1173231 seeds (0.001 S) were charged to the reaction vessel. The slurry was aged for about 18 hours before cooling to −15° C. over about 6 hours. The slurry was filtered and the cake was rinsed with 2-propanol (1 V) before drying at about 50° C. until adequately dry to afford (1S,2R,5S)-8-methoxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (I-B).

¹H NMR (400 MHz, CDCl₃) δ 10.30 (t, J=5.8 Hz, 1H), 8.55 (s, 1H), 6.70-6.61 (m, 2H), 5.64-5.58 (m, 1H), 5.57-5.48 (m, 1H), 5.37 (dt, J=11.3, 3.2 Hz, 1H), 4.95 (d, J=14.3 Hz, 1H), 4.71-4.57 (m, 2H), 4.37 (d, J=14.3 Hz, 1H), 4.11 (s, 3H), 3.80-3.71 (m, 1H), 1.39 (d, J=6.7 Hz, 3H), 1.32 (d, J=7.4 Hz, 3H).

### Example 46: Preparation of (2R,5S,Z)-8-methoxy-2,5-dimethyl-7,9-dioxo-N-(2,4,6-trifluorobenzyl)-1,2,5,6,7,9-hexahydropyrido[1,2-b][1,2,5]triazonine-10-carboxamide (R-1-A)

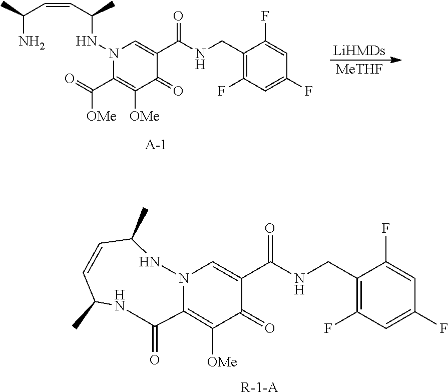

A reaction vessel was charged with compound A-1 (scaling factor, 1.0 equiv), 2-methyltetrahydrofuran (20 V), and lithium bis(trimethylsilyl)amide as a 1 M solution in tetrahydrofuran (3.0 equiv). The contents were agitated for about 2 hours. Water (20 V) was added and the mixture was extracted with ethyl acetate (10 V×3). The combined organic extracts were dried with sodium sulfate, filtered, and concentrated. The material was purified by column chromatography using ethyl acetate and hexanes (60 to 100%) to afford compound R-1-A. ¹H NMR (400 MHz, DMSO-d₆) δ 10.55 (t, J=5.8 Hz, 1H), 8.41-8.30 (m, 2H), 7.33-7.16 (m, 2H), 7.00 (d, J=2.2 Hz, 1H), 5.47 (ddd, J=10.5, 8.5, 2.0 Hz, 1H), 5.39 (dd, J=10.6, 4.7 Hz, 1H), 4.66 (dd, J=14.5, 6.3 Hz, 1H), 4.45 (dd, J=14.6, 5.3 Hz, 1H), 4.07-3.90 (m, 2H), 3.73 (s, 3H), 1.35 (d, J=7.2 Hz, 3H), 1.10 (d, J=6.4 Hz, 3H).

### Example 47: Preparation of (S,Z)-2-(5-oxohex-3-en-2-yl)isoindoline-1,3-dione (W-1)

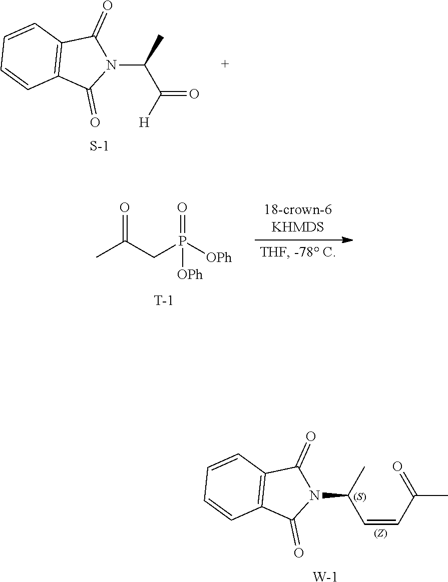

A reaction vessel was charged with 18-crown-6 (1.3 equiv), diphenyl (2-oxopropyl)phosphonate (T-1, 1.07 equiv), and THE (10 V). The contents were agitated at about −78° C. and potassium bis(trimethylsilyl)amide 1M in THE was charged (1.02 equiv). The contents were agitated at about −78° C. for about 1 hour. A solution of (S)-2-(1,3-dioxoisoindolin-2-yl)propanal (S-1, scaling factor, 1.0 equiv) and THE (5 V) was then charged and the contents were agitated at about −78° C. for about 2 hours. MTBE (10 V) was added followed by 1N aqueous HCl (10 V). The aqueous phase was collected and the organic phase was washed with water (10 V). The combined aqueous phases were extracted with MTBE (10 V, ×2). The organic extracts were combined and dried with magnesium sulfate, filtered, and concentrated to afford (S,Z)-2-(5-oxohex-3-en-2-yl)isoindoline-1,3-dione (W-1).

¹H NMR (400 MHz, Chloroform-d) δ 7.82 (dd, J=5.5, 3.1 Hz, 2H), 7.70 (dd, J=5.5, 3.1 Hz, 2H), 6.57 (dd, J=11.4, 8.5 Hz, 1H), 6.20 (dd, J=11.4, 1.4 Hz, 1H), 5.90 (ddd, J=8.5, 7.0, 1.4 Hz, 1H), 2.23 (s, 3H), 1.59 (d, J=7.0 Hz, 3H).

### Example 48: Preparation of 2-((2S,5S,Z)-5-hydroxyhex-3-en-2-yl)isoindoline-1,3-dione (F-2)

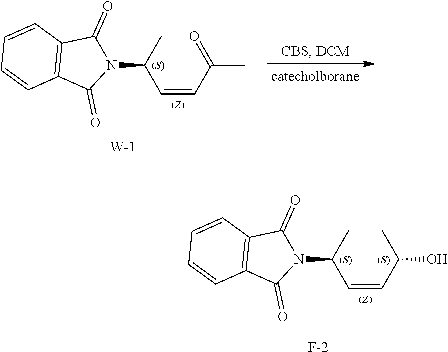

(S,Z)-2-(5-oxohex-3-en-2-yl)isoindoline-1,3-dione (W-1, 1.0 mol equiv, scaling factor) was charged to a vial followed by DCM (16V) and (R)-1-Methyl-3,3-diphenylhexahydropyrrolo[1,2-c][1,3,2]oxazaborole ((R)-Me-CBS, CAS number 112022-83-0, 0.15 eq, 1.0M in Toluene). The contents were agitated and cooled to about −10° C. Catecholborane (1.5 eq, neat) was charged dropwise at −10° C. and reaction mixture was agitated for about 30 minutes.

¹H NMR (400 MHz, DMSO-d6) δ 8.38-7.53 (m, 4H), 5.75 (ddd, J=10.6, 9.1, 1.2 Hz, 1H), 5.43 (ddd, J=10.9, 8.4, 1.1 Hz, 1H), 5.26-5.05 (m, 1H), 4.80 (d, J=4.2 Hz, 1H), 4.61-4.37 (m, 1H), 1.47 (d, J=7.0 Hz, 3H), 0.96 (d, J=6.3 Hz, 3H).

### Example 49: Preparation of 2-((2S,5S,Z)-5-hydroxyhex-3-en-2-yl)isoindoline-1,3-dione (F-2)

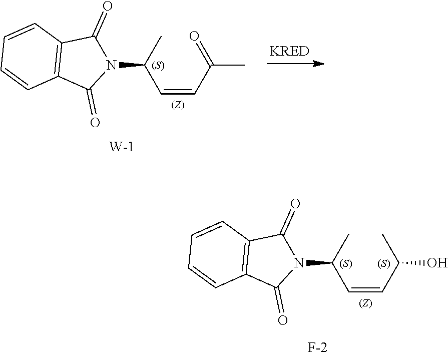

(S,Z)-2-(5-oxohex-3-en-2-yl)isoindoline-1,3-dione (W-1, 1.0 mol equiv, scaling factor) was charged to a vial followed by DMSO (9 V). To a separate reaction vial was charged ketoreductase enzyme (KRED-NADH-101, 1.0 g/g) and reaction buffer (263 mM sodium phosphate, 1.7 mM magnesium sulfate, 1.1 mM NADP+, 1.1 mM NAD+, 80 mM D-glucose, 4.3 U/mL glucose dehydrogenase, pH 7.0). The DMSO solution of compound W-1 was charged to the reaction vial and stirred at about room temperature for about 18 hours to afford -((2S,5S,Z)-5-hydroxyhex-3-en-2-yl)isoindoline-1,3-dione (F-2).

¹H NMR (400 MHz, DMSO-d6) δ 8.38-7.53 (m, 4H), 5.75 (ddd, J=10.6, 9.1, 1.2 Hz, 1H), 5.43 (ddd, J=10.9, 8.4, 1.1 Hz, 1H), 5.26-5.05 (m, 1H), 4.80 (d, J=4.2 Hz, 1H), 4.61-4.37 (m, 1H), 1.47 (d, J=7.0 Hz, 3H), 0.96 (d, J=6.3 Hz, 3H).

### Example 50: Preparation of (2S,5S)-5-aminohex-3-yn-2-ol (U-1)

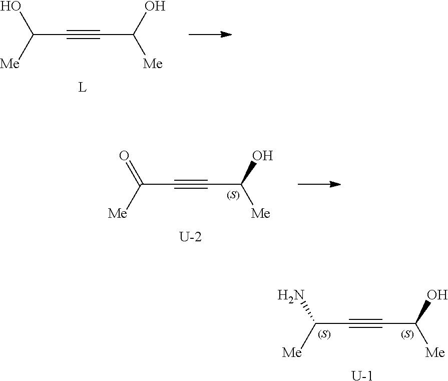

A reaction vessel was charged with hex-3-yne-2,5-diol (L, 1.0 equiv, scaling factor), 0.1 M Tris buffer (pH 7.6, 510 V), acetone (2.5 V), and beta-nicotinamide adenine dinucleotide sodium salt (0.09 equiv). KRED-476 (3.5 g/g) was dissolved in 0.1 M triethanolamine buffer (pH 7.6, 45 V) and added to the reaction. Amine transaminase ATA-P1-G05 (8.0 g/g) and pyridoxal phosphate (PLP) (0.01 eq) were added to a transaminase buffer (pH 7.5, 1.0 M isopropylamine, 0.1 M triethanolamine, 320 V) which was added to the reaction vessel. Reaction mixture was agitated at about 30° C. for about 21 hours. Upon reaction completion charged sodium hydroxide in water (25 wt %, 130 V) and 2-MeTHF (510 V).

¹H NMR (400 MHz, DMSO-d₆): δ 4.41-4.26 (m, 1H), 3.60 (qd, J=6.7, 1.7 Hz, 1H), 1.25 (d, J=6.6 Hz, 3H), 1.19 (d, J=6.7 Hz, 3H).

All references, including publications, patents, and patent documents were incorporated by reference herein, as though individually incorporated by reference. The present disclosure provides reference to various embodiments and techniques. However, it should be understood that many variations and modifications may be made while remaining within the spirit and scope of the present disclosure.

1. A process for preparing a compound of Formula I: 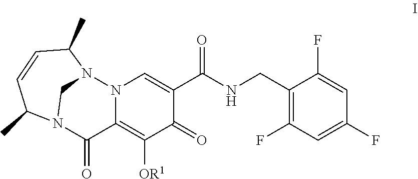
or a salt thereof, wherein R¹ is selected from hydrogen and a hydroxyl protecting group;
comprising reacting a compound of Formula II: 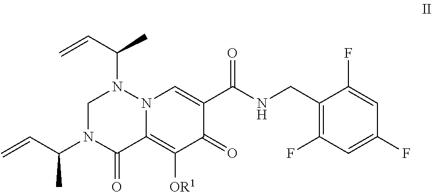
or a salt thereof,
with a first catalyst to provide the compound of Formula I or a salt thereof.

2. The process of claim 1, wherein R¹ is selected from hydrogen, C₁₋₄alkyl, and benzyl.

3. The process of claim 1, wherein R¹ is selected from hydrogen, methyl, ethyl, and benzyl.

4. The process of claim 1, wherein R¹ is methyl.

5. The process of claim 1, wherein the first catalyst is a metathesis catalyst.

6-15. (canceled)

16. The process of claim 1, wherein R¹ is a hydroxyl protecting group; and wherein the process further comprises deprotecting the compound of Formula I, or a salt thereof;
to provide a compound of Formula I-A: 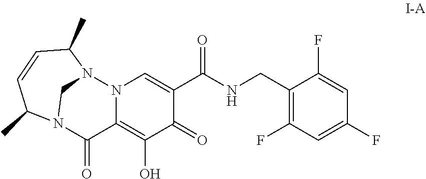
or a salt thereof.

17-22. (canceled)

23. The process of claim 1, further comprising preparing the compound of Formula II or a salt thereof by a process comprising:
reacting a compound of Formula III: 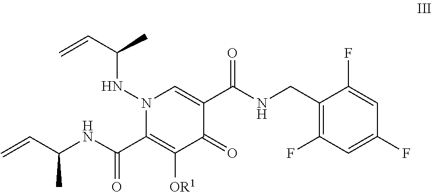
or a salt thereof, with
a methylene reagent, and
a first acid or a first base,
to provide the compound of Formula II or a salt thereof.

24-33. (canceled)

34. The process of claim 23, further comprising preparing the compound of Formula III or a salt thereof by a process comprising:
deprotecting a compound of Formula IV: 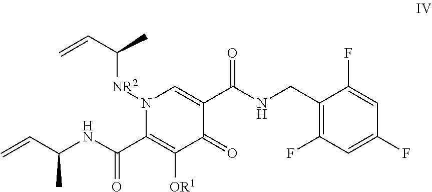
or a salt thereof, wherein R² is an amine protecting group;
to provide the compound of Formula III or a salt thereof.

35-40. (canceled)

41. The process of claim 34, further comprising preparing the compound of Formula IV or a salt thereof by a process comprising:
reacting a compound of Formula V: 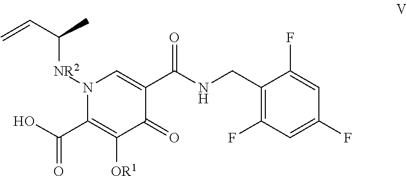
or a salt thereof, with:
a compound of Formula VI: 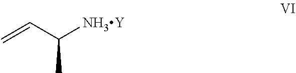
wherein Y is an anion or a salt,
a coupling agent, and
a second base,
to provide the compound of Formula V or a salt thereof.

42-50. (canceled)

51. The process of claim 41, further comprising preparing the compound of Formula V or a salt thereof by a process comprising:
reacting a compound of Formula VII: 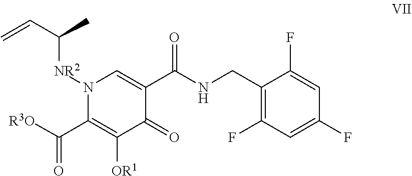
or a salt thereof, wherein R³ is a hydroxyl protecting group;
with a first reagent to provide the compound of Formula V or a salt thereof.

52-60. (canceled)

61. The process of claim 51, further comprising preparing the compound of Formula VII or a salt thereof by a process comprising:
reacting a compound of Formula VIII: 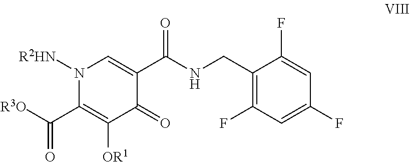
or a salt thereof, with:
a compound of Formula IX: 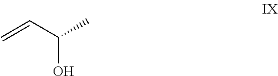
or a salt thereof,
a phosphorus-based reagent, and
an azodicarboxylate,
to provide the compound of Formula VII or a salt thereof.

62-67. (canceled)

68. The process of claim 51, further comprising preparing the compound of Formula VII or a salt thereof by a process comprising:
reacting a compound of Formula VIII: 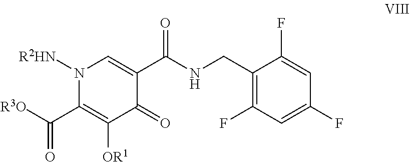
or a salt thereof, with:
a second reagent comprising a crotyl group or an allyl group,
a second catalyst, and
a sixth base
to provide the compound of Formula VII or a salt thereof.

69-83. (canceled)

84. The process of claim 61, further comprising preparing the compound of Formula VIII or a salt thereof by a process comprising:
reacting a compound of Formula X: 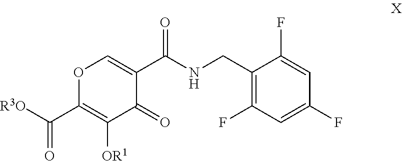
or a salt thereof, with a compound of Formula NH₂NHR₂ or a salt thereof,
to provide the compound of Formula VIII or a salt thereof.

85-89. (canceled)

90. The process of claim 84, further comprising preparing the compound of Formula X or a salt thereof by a process comprising:
reacting a compound of Formula XII: 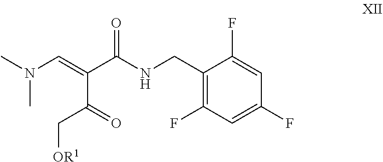
or a salt thereof, with:
a promoter,
a dialkyl oxalate, and
a fifth base,
to provide the compound of Formula X or a salt thereof.

91-103. (canceled)

104. The process of claim 90, further comprising preparing the compound of Formula XII or a salt thereof by a process comprising:
reacting a compound of Formula XIII: 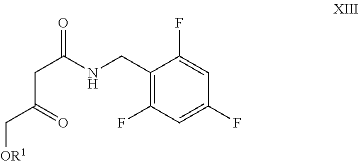
or a salt thereof, with N,N-dimethylformamide dimethyl acetal,
to provide the compound of Formula XII or a salt thereof.

105. A process for preparing a compound of Formula I: 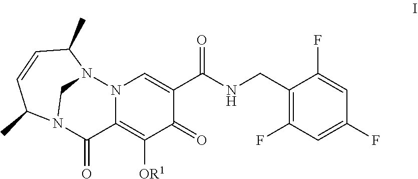
or a salt thereof, wherein R¹ is selected from hydrogen and a hydroxyl protecting group, comprising:
(a) reacting a compound of Formula VII: 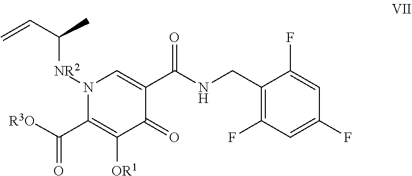
or a salt thereof, wherein R² is an amine protecting group and R³ is a hydroxyl protecting group, with a first reagent to provide a compound of Formula V: 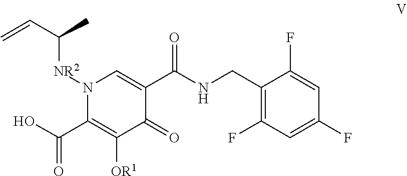
or a salt thereof;
(b) reacting the compound of Formula V or a salt thereof, with:
a compound of Formula VI: 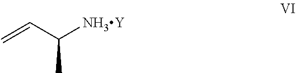
wherein Y is an anion or salt,
a coupling agent, and
a second base,
to provide a compound of Formula IV: 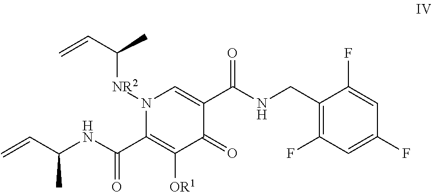
or a salt thereof;
(c) deprotecting the compound of Formula IV or a salt thereof to provide a compound of Formula III: 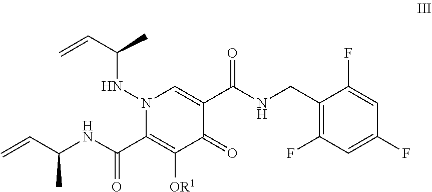
or a salt thereof;
(d) reacting the compound of Formula III or a salt thereof, with
a methylene reagent, and
a first acid or a first base, to provide a compound of Formula II: 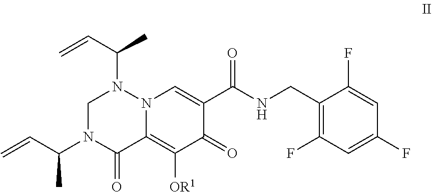
or a salt thereof; and
(e) reacting the compound of Formula II or a salt thereof;
with a first catalyst to provide the compound of Formula I or a salt thereof.

106. A process for preparing a compound of Formula I-A: 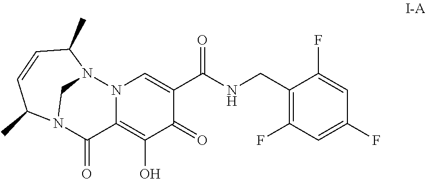
or a salt thereof, comprising:
(a) reacting a compound of Formula VII: 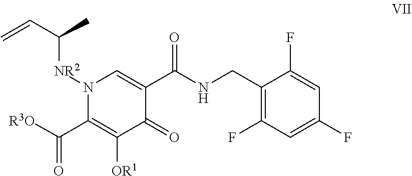
or a salt thereof, wherein R¹ and R³ are each independently hydroxyl protecting groups, and R² is an amine protecting group,
with a first reagent to provide a compound of Formula V: 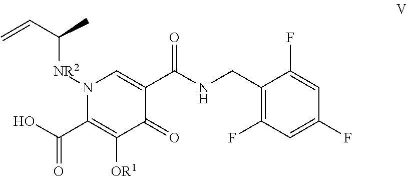
or a salt thereof;
(b) reacting the compound of Formula V or a salt thereof, with:
a compound of Formula VI: 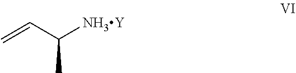
wherein Y is an anion or salt,
a coupling agent, and
a second base,
to provide a compound of Formula IV: 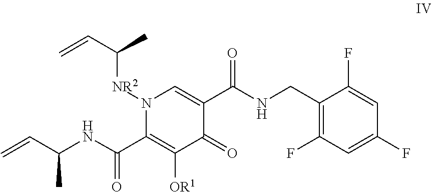
or a salt thereof;
(c) deprotecting the compound of Formula IV or a salt thereof to provide a compound of Formula III: 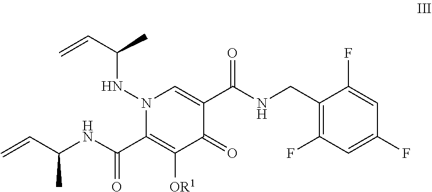
or a salt thereof;
(d) reacting the compound of Formula III or a salt thereof, with
a methylene reagent, and
a first acid or a first base, to provide a compound of Formula II: 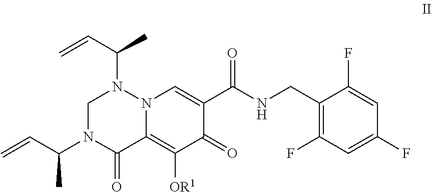
or a salt thereof;
(e) reacting the compound of Formula II or a salt thereof;
with a first catalyst to provide a compound of Formula I: 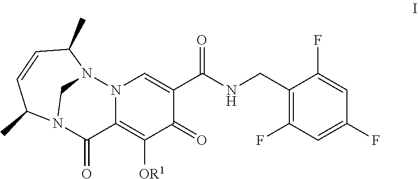
or a salt thereof; and
(f) deprotecting the compound of Formula I, or a salt thereof; to provide the compound of Formula I-A or a salt thereof.

107. A process for preparing a compound of Formula II: 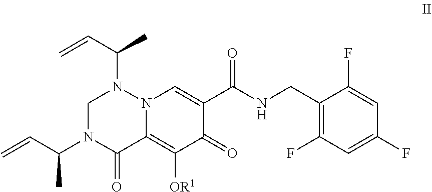
or a salt thereof, comprising:
(a) reacting a compound of Formula VII: 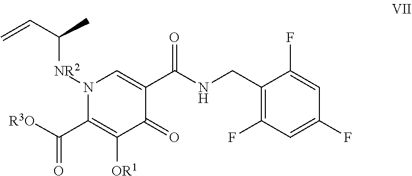
or a salt thereof, wherein R¹ and R³ are each independently hydroxyl protecting groups, and R² is an amine protecting group,
with a first reagent to provide a compound of Formula V: 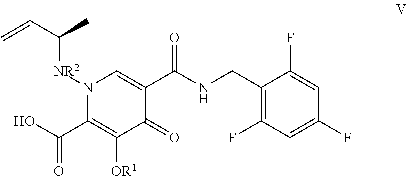
or a salt thereof;
(b) reacting the compound of Formula V or a salt thereof, with:
a compound of Formula VI: 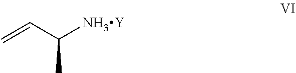
wherein Y is an anion or salt,
a coupling agent, and
a second base,
to provide a compound of Formula IV: 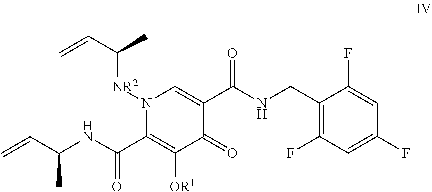
or a salt thereof;
(c) deprotecting the compound of Formula IV or a salt thereof to provide a compound of Formula III: 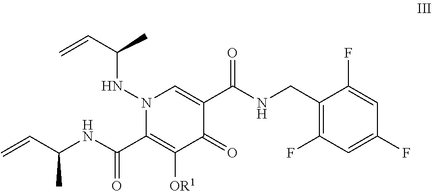
or a salt thereof; and
(d) reacting the compound of Formula III or a salt thereof, with
a methylene reagent, and
a first acid or a first base, to provide the compound of Formula II or a salt thereof.

108. A process for preparing a compound of Formula III: 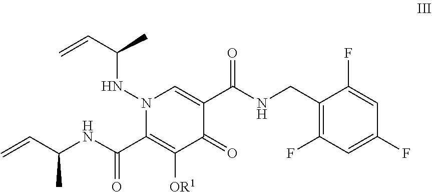
or a salt thereof, comprising:
(a) reacting a compound of Formula VII: 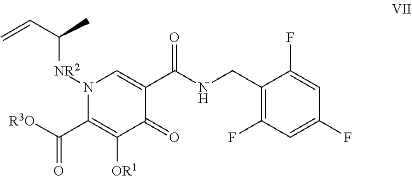
or a salt thereof, wherein R¹ and R³ are each independently hydroxyl protecting groups, and R² is an amine protecting group,
with a first reagent to provide a compound of Formula V: 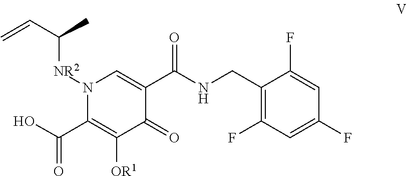
or a salt thereof;
(b) reacting the compound of Formula V or a salt thereof, with:
a compound of Formula VI: 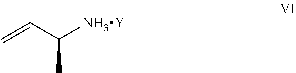
wherein Y is an anion or salt,
a coupling agent, and
a second base,
to provide a compound of Formula IV: 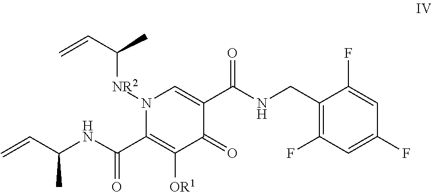
or a salt thereof; and
(c) deprotecting the compound of Formula IV or a salt thereof to provide the compound of Formula III or a salt thereof.

109. A process for preparing a compound of Formula IV: 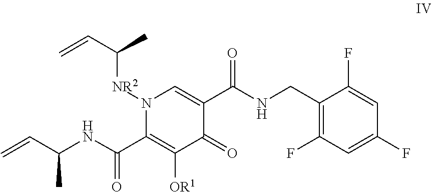
or a salt thereof, comprising:
(a) reacting a compound of Formula VII: 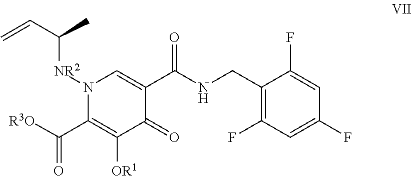
or a salt thereof, wherein R¹ and R³ are each independently hydroxyl protecting groups, and R² is an amine protecting group,
with a first reagent to provide a compound of Formula V: 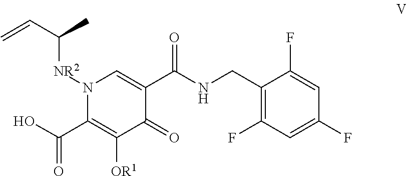
or a salt thereof; and
(b) reacting the compound of Formula V or a salt thereof, with:
a compound of Formula VI: 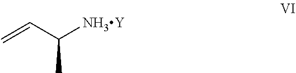
wherein Y is an anion or salt,
a coupling agent, and
a second base,
to provide the compound of Formula IV or a salt thereof.

110. A process for preparing a compound of Formula V: 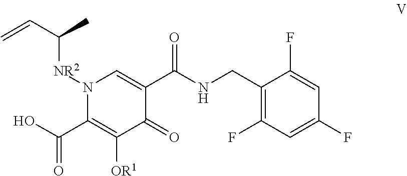
or a salt thereof, comprising:
reacting a compound of Formula VII: 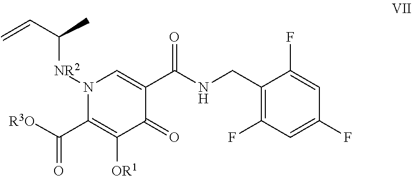
or a salt thereof, wherein R¹ and R³ are each independently hydroxyl protecting groups, and R² is an amine protecting group,
with a first reagent to provide the compound of Formula V or a salt thereof.

111-152. (canceled)

153. A process for preparing a compound of Formula I-A: 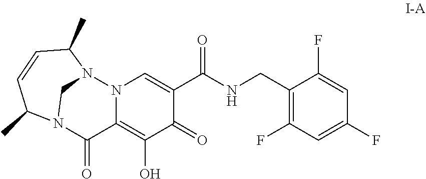
or a salt thereof, comprising deprotecting a compound of Formula I: 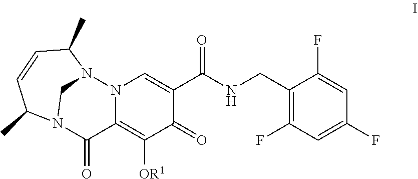
or a salt thereof, wherein R¹ is a hydroxyl protecting group, to provide the compound of Formula I-A or a salt thereof.

154-545. (canceled)

546. A process for preparing a compound of Formula I-A: 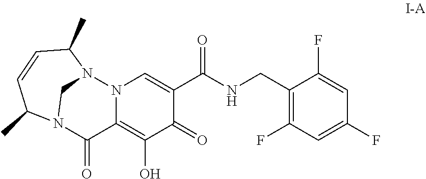
or a salt thereof, comprising:
(a) reacting a compound of Formula A: 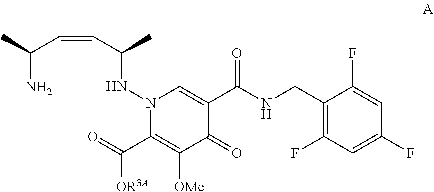
or a salt thereof, wherein R^{3A} is a hydroxyl protecting group, with:
a methylene reagent, and
a first acid or a first base,
to provide a compound of Formula I: 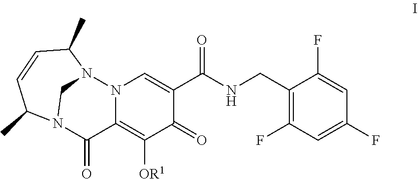
or a salt thereof, wherein R¹ is a hydroxyl protecting group; and
(b) deprotecting the compound of Formula I or a salt thereof, to provide the compound of Formula I-A or a salt thereof.

547. A process for preparing a compound of Formula I-A: 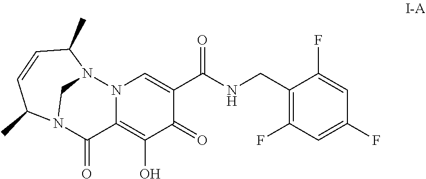
or a salt thereof, comprising:
(a) reacting a compound of Formula R-1: 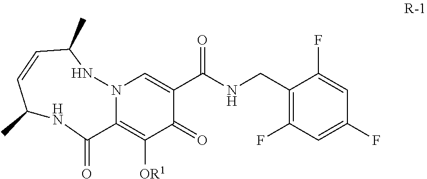
or a salt thereof, with:
a methylene reagent, and
an eighth acid or a fourteenth base,
to provide a compound of Formula I: 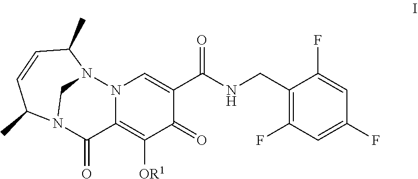
or a salt thereof, wherein R¹ is a hydroxyl protecting group; and
(b) deprotecting the compound of Formula I or a salt thereof, to provide the compound of Formula I-A or a salt thereof.

548-599. (canceled)

600. A compound of
(a) Formula I, II, III, IV, or V: 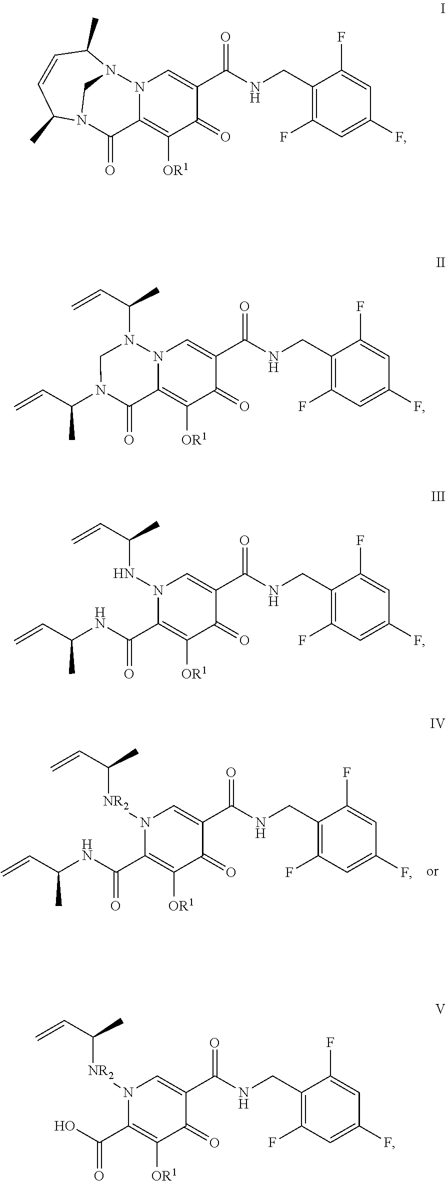
or a salt thereof, wherein R¹ is a hydroxyl protecting group, and R² is an amine protecting group;
(b) Formula VII, VIII, or Q-1: 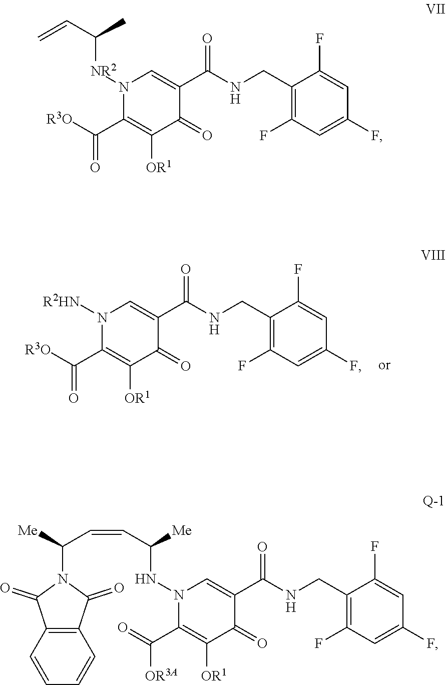
or a salt thereof, wherein R¹, R^{3A} and R³ are each independently hydroxyl protecting groups and R² is hydrogen or an amine protecting group; or
(c) Formula A, R-1, or R-2: 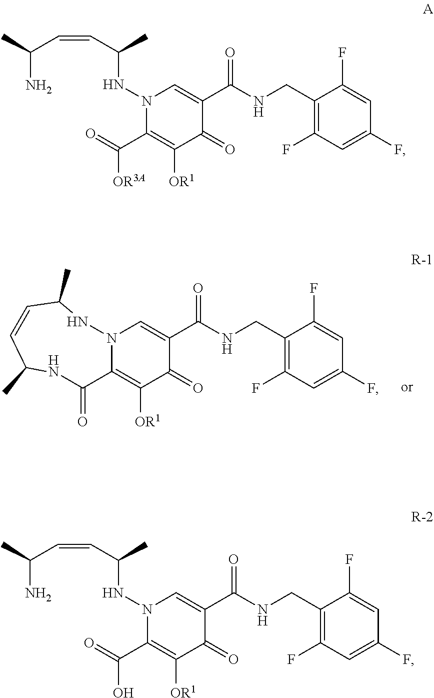
or a salt thereof, wherein R¹ and R^{3A} are independently selected from hydrogen and a hydroxyl protecting group.
